(19) United States
(12) Patent Application Publication
(10) Pub. No.: US 2026/0297596 A1
(43) Pub. Date: Oct. 1, 2026

(54) ARTIFICIAL SECOVIRIDAE SATELLITE RNAs

(71) Applicant: FLAGSHIP PIONEERING INNOVATIONS VII, LLC, CAMBRIDGE, MA (US)

(72) Inventors: David George Blumsack, Peabody, MA (US); Fu Chyun Chu, Arlington, MA (US); Elizabeth Jane Antonelli Dennis, Putnam, CT (US); Jean-Pierre Baptiste Séhi Glouzon, Cambridge, MA (US); Mehmet Ali Halac, Philadelphia, PA (US); Yumeng Hao, Cambridge, MA (US); Arjun Devang Khakhar, Fort Collins, CO (US); Kevin Klicki, Watertown, MA (US); James Michael Kremer, Cambridge, MA (US); Jayashree Kumar, Somerville, MA (US); Katherine Michelle LaTourrette, Cambridge, MA (US); Barry Andrew Martin, Newport, RI (US); Shankar Raj Pant, Arlington, MA (US); Derek Thomas Rothenheber, Somerville, MA (US); Michka Gabrielle Sharpe, Cambridge, MA (US); Aditya Sushil Kumar Singh, Brookline, MA (US); Arjun Subedi, Lexington, MA (US); Phu Tri Tran, Belmont, MA (US); Paveena Vichyavichien, Deerfield Beach, FL (US); Reid Evan William Warsaba, Cambridge, MA (US); Kaixi Zhao, Lansdale, PA (US)

(21) Appl. No.: 19/479,444

(22) PCT Filed: May 3, 2024
(86) PCT No.: PCT/US2024/027672

§ 371 Date: Oct. 28, 2025

Related U.S. Application Data
(60) Provisional application No. 63/499,723, filed on May 3, 2023.

(51) Int. Cl. C12N 15/82; C12N 9/12

(52) U.S. Cl. CPC C12N 15/8203; C12N 9/127; C12N 15/8218; C12N 15/8271; C12N 2770/32043; C12N 2840/203; C12Y 207/07048

(57) ABSTRACT

Synthetic Secoviridae satellite RNA molecules and satellite particles containing the same are disclosed. Synthetic Secoviridae satellite RNA molecules which contain internal heterologous RNA virus (HRV) amplicons are also disclosed. Also disclosed are methods of using the Secoviridae satellite RNA molecules and satellite particles containing the same to change plant phenotypes, improve plant stress resistance, and improve plant pest and pathogen resistance.

### CROSS-REFERENCE TO RELATED APPLICATIONS

This application claims priority to provisional application U.S. Ser. No. 63/499,723, filed May 3, 2023, which is hereby incorporated herein by reference in its entirety.

### INCORPORATION OF SEQUENCE LISTING

The instant application contains a Sequence Listing which has been submitted electronically in XML file format and is hereby incorporated by reference in its entirety. The XML file, created on Apr. 29, 2024, is named P14420WO00.xml and is 1,640,954 bytes in size. The XML file, created on May 2, 2023, is named P14420US00.xml, which is 1,071,295 bytes in size and was filed in provisional application U.S. Ser. No. 63/499,723 on May 3, 2023, is also incorporated herein by reference in its entirety.

### BACKGROUND

There is need in the art for modifying polynucleotides for improving phenotypes and genotypes of organisms; in particular, for agricultural applications to improve plants such as crop plants.

Secoviridae is a family of RNA viruses that includes the subfamily Comovirinae as well as unassigned genera including Cheravirus and Stralarivirus. Members of Comovirinae are RNA viruses having a bipartite, linear, single-stranded genome. Genome segments of about 6 to about 8 kilobases (kb) (RNA segment 1) and about 4 to about 7 kb (RNA segment 2) are encapsidated separately.

### SUMMARY

This disclosure provides recombinant RNA molecules comprising from 5′ terminus to 3′ terminus: (a) a 5′ RNA replication element recognized by a Secoviridae RNA-dependent RNA polymerase (RdRP); (b) a cargo RNA molecule (cargo RNA sequence); and (c) a 3′ RNA replication element recognized by the RdRP; wherein the 5′ RNA replication element, the cargo RNA molecule, and the 3′ RNA replication element are operably linked, and wherein the cargo RNA molecule is heterologous to the 5′ RNA replication element and the 3′ RNA replication element. In certain embodiments, (i) the 5′ RNA replication element and the 3′ RNA replication element are obtained from the same Secoviridae genome or from Secoviridae genomes having at least 85%, 90%, 95%, 98%, or 99% sequence identity to one another and which are optionally related; (ii) the 5′ RNA replication element, the 3′ RNA replication element, and the RdRP are obtained from the same Secoviridae genome or from Secoviridae genomes having at least 85%, 90%, 95%, 98%, or 99% sequence identity to one another and which are optionally related; or (iii) the 5′ RNA replication element, the 3′ RNA replication element, and/or the RdRP coding region are obtained from different Secoviridae genomes, and the members of each respective set of the 5′ RNA replication elements, 3′ RNA replication elements, and/or RdRP coding regions have at least 85%, 90%, 95%, 98%, or 99% sequence identity to one another. In certain embodiments, the RNA molecule comprises: at least one heterologous RNA virus (HRV) amplicon in sense or antisense orientation to the first 5′ RNA replication element comprising: I. (i) a heterologous RNA virus (HRV) 5′ replication region (HRV 5′RR); (ii) the cargo RNA molecule; and (iii) the heterologous RNA virus (HRV) 3′ RNA replication region (HRV 3′RR); wherein the HRV 5′ RR and HRV 3′ RR HRV are recognized by a heterologous RNA virus RNA-dependent RNA polymerase (hrvRdRP); and wherein the HRV 5′RR, cargo RNA molecule, and HRV 3′RR are operably linked; or II. a heterologous RNA virus (HRV) subgenomic promoter operably linked to the cargo RNA molecule; wherein the subgenomic promoter is recognized by a heterologous RNA virus RNA-dependent RNA polymerase (hrvRdRP). Agricultural formulations as well as bacterial, fungal, plant, insect, and invertebrate animal cells comprising the herein disclosed recombinant RNAs are also provided.

Also provided are expression systems comprising: (a) an RNA molecule comprising the herein disclosed recombinant RNA molecules; and (b) a cell containing the recombinant RNA molecule and an RdRP protein that recognizes the 5′ and 3′ RNA replication elements of the recombinant RNA molecule. In certain embodiments, the cell further comprises one or more of: (i) a viral capsid protein (CP); (ii) an RNA-binding protein (RBP) that binds to the RNA molecule, optionally wherein the RBP binds to an RNA effecter; (iii) an RNA cleavage agent that cleaves the RNA molecule; (iv) a second RNA-dependent RNA polymerase (2^{nd} RdRP) protein that recognizes an HRV 5′ or 3′ replication region and/or a subgenomic promoter in the RNA molecule; (v) a viral movement protein (MP); (vi) a heterologous RNA virus (HRV); or (vii) an hrvRdRP, optionally wherein the hrvRdRP recognizes the HRV 5′ or 3′ replication region and/or the subgenomic promoter.

Also provided are methods of providing a synthetic Secoviridae satellite RNA to a plant, comprising contacting the plant with any of the herein disclosed recombinant RNA molecules.

Also provided are methods of establishing a synthetic Secoviridae satellite RNA in a plant cell, comprising: providing to a plant cell any of the herein disclosed recombinant RNA molecules, wherein the plant cell comprises an RdRP protein that recognizes the 5′ RNA replication element and 3′ RNA replication element, wherein the RNA molecule optionally comprises an encapsidation recognition element (ERE) and is or can be encapsidated by a capsid protein, whereby the RdRP protein catalyzes synthesis of the synthetic Secoviridae satellite RNA from the recombinant RNA molecule.

Also provided are methods of obtaining a phenotypic change in a plant or plant cell, comprising: providing to a plant or plant cell any of the herein disclosed recombinant RNA molecules, wherein the cargo RNA molecule comprises RNA that effects a phenotypic change in the plant or plant cell in comparison to a plant or plant cell lacking the recombinant RNA, wherein the plant or plant cell comprises an RdRP protein that recognizes the 5′ RNA replication element and 3* RNA replication element and catalyzes synthesis of a synthetic Secoviridae RNA from the recombinant RNA molecule, and wherein and the cargo RNA molecule effects the phenotypic change. In certain embodiments, the methods further comprise providing an hrvRdRP to the plant which recognizes the HRV 5′ or 3′ replication region and/or the subgenomic promoter in the synthetic Secoviridae satellite RNA, optionally wherein the hrvRdRP is provided by introducing a recombinant DNA or RNA encoding the hrvRdRP into the plant or a part thereof.

Also provided are methods of manufacturing a synthetic Secoviridae satellite particle, comprising combining any of the herein disclosed recombinant RNA molecules with a viral capsid protein, wherein the recombinant RNA molecule comprises an encapsidation recognition element (ERE), and wherein the ERE provides for encapsidation of the RNA by the viral capsid protein.

Also provided are plant propagules comprising any of the herein disclosed recombinant RNA molecules and a Secoviridae RdRP, optionally wherein the plant propagule further comprises a heterologous RNA virus RdRP which recognizes an HRV 5′ or 3′ replication region and/or a subgenomic promoter in the synthetic Secoviridae satellite RNA. In certain embodiments, the plant propagule further comprises a heterologous RNA virus RdRP which recognizes an HRV 5′ or 3′ replication region and/or a subgenomic promoter in the synthetic Secoviridae satellite RNA, and the heterologous RNA virus RdRP is an Alphaflexivirus, Betaflexivirus, Bromovirus, Celavirus. Closterovirus, Comovirus, Potexvirus, Potyvirus, Tobamovirus, Tombusvirus, Tospoviridae. Trivirinae, Tymovirus, Varicosavirus, or Secoviridae RdRP.

Also provided are plants comprising any of the herein disclosed recombinant RNA molecules and a Secoviridae RdRP, optionally wherein the plant propagule further comprises a heterologous RNA virus RdRP which recognizes an HRV 5′ or 3′ replication region and/or a subgenomic promoter in the synthetic Secoviridae satellite RNA.

Secoviridae satellite systems that are self-replicating when introduced into a plant or plant cell, comprising: (1) any of the herein disclosed recombinant Secoviridae satellite RNAs (e.g., recombinant RNA molecules); and (2) an exogenous Secoviridae that is capable of replication in the plant or plant cells and that encodes the Secoviridae RdRP that recognizes the 5′ and 3′ replicase recognition sequences in the recombinant Secoviridae satellite RNA, optionally wherein the Secoviridae satellite system further comprises a heterologous RNA virus RdRP which recognizes an HRV 5′ or 3′ replication region and/or a subgenomic promoter in the synthetic Secoviridae satellite RNA.

### BRIEF DESCRIPTION OF THE DRAWINGS

FIG. 1 shows a non-limiting embodiment of a structure of a Secoviridae satellite construct. In this embodiment, the 5′ RNA replication element is labelled “5′ RRE” and the 3′ RNA replication element is labeled “3′ RRE.”

FIG. 2 shows non-limiting embodiments of a Secoviridae satellite construct containing a heterologous RNA virus (HRV) amplicon comprising: (i) a heterologous RNA virus (HRV) 5′ replication region (HRV 5′RR); (ii) the cargo RNA molecule; and (iii) the heterologous RNA virus (HRV) 3′ RNA replication region (HRV 3′RR). Both sense and antisense orientations of the HRV amplicon relative to the Secoviridae 5′ RNA replication element are shown. In certain embodiments, at least one cleavable sequence is located; (i) 5′ to the 5′ RNA replication element or 3′ to the 3′ RNA replication element; and/or (ii) between the 3′ end of the 5′ RNA replication element and the HRV amplicon and/or between the HRV amplicon and the 5′ end of the 3′ RNA replication element. Wherein the cleavable sequence is optionally a self-cleaving ribozyme, a self-cleaving inducible ribozyme, or an siRNA or miRNA recognition site. In certain embodiments, a subgenomic promoter and/or an IRES is/are operably linked to the cargo RNA.

FIG. 3 shows non-limiting embodiments of a Secoviridae satellite construct containing a heterologous RNA virus (HRV) amplicon comprising a heterologous RNA virus (HRV) subgenomic promoter (HRV sgp) which is recognized by a heterologous RNA virus RNA-dependent RNA polymerase (hrvRdRP) and which is operably linked to the cargo RNA molecule. Embodiments where the HRV sgp and cargo RNA are in sense and antisense orientation relative to the Secoviridae 5′ RRE are shown.

FIG. 4 depicts a Secoviridae satellite construct comprising heterologous RNA virus (HRV) subgenomic promoters (HRV sgp) with: (i) one HRV sgp operably linked to a cargo RNA; and (ii) one HRV sgp operably linked to RNA encoding an hrvRdRP which recognizes both of the HRV sgp (i.e., can drive expression of the operably linked hrvRdRP and cargo RNA). In certain embodiments, an IRES is operably linked to the cargo RNA and/or an IRES is operably linked to the RNA encoding the hrvRdRP.

FIG. 5 depicts non-limiting embodiments of a Secoviridae satellite construct containing a heterologous RNA virus (HRV) amplicon comprising a heterologous RNA virus (HRV) subgenomic promoter (HRV sgp) which is recognized by a heterologous RNA virus RNA-dependent RNA polymerase (hrvRdRP) and which is operably linked to the cargo RNA molecule flanked by HRV 5′RR and HRV 3′ RR. Embodiments where the HRV sgp and cargo RNA are in sense or antisense orientation relative to the Secoviridae 5′ RRE are shown. The HRV 5′ RR and 3′ RR which flank the cargo RNA provide for hrvRdRP-mediated replication of an RNA comprising from 5′ to 3′ the HRV 5′ RR, cargo RNA, and HRV 3′ RR. In certain embodiments, the HRV 5′ RR and 3′ RR are flanked by ribozymes.

FIG. 6 depicts a commensal satellite with a cargo RNA molecule including an HRV (“HRV1”, e.g., tobacco mosaic virus, TMV) amplicon designed to be amplified by the HRV (“HRV1”, e.g.. TMV) RdRP binding to either the replication regions or to the subgenomic promoter, where the commensal satellite is a Secoviridae satellite. The resulting transcripts include RNA encoding the HRV RdRP (“HRV1 RdRP”, solid squares), which can further amplify the HRV amplicon, as well as RNA encoding another cargo (solid circles). In the absence of the commensal virus, no amplification of the commensal satellite occurs.

FIG. 7 depicts a commensal satellite with a cargo RNA molecule including an HRV (HRV1, e.g., tobacco mosaic virus, TMV) amplicon designed to be amplified by the HRV RdRP (“HRV1 RdRP”, solid squares) binding to the HRV1 replication regions, where the commensal satellite is a Secoviridae satellite. The resulting transcripts include RNA encoding the HRV (HRV1) RdRP which can further amplify the HRV amplicon. The HRV1 amplicon includes sequence for a HRV2 amplicon (indicated in italicized text), encoding a coding and/or noncoding cargo (solid circles) and designed to be amplified in the presence of a second acute viral RdRP (“HRV2 RdRP”, hexagonal symbol), which can be provided, e.g., by introduction of a second acute virus (“HRV2”, e.g., cowpea mosaic virus, CPMV) into the plant. In the absence of the commensal virus, no amplification of the commensal satellite occurs.

FIG. 8 depicts a commensal satellite with a cargo RNA molecule including an HRV (“HRV1”, e.g., tobacco mosaic virus, TMV) amplicon designed to be amplified by the HRV (“HRV1”, e.g., TMV) RdRP binding to either of two subgenomic promoters, where the commensal satellite is a Secoviridae satellite. The resulting transcripts include RNA encoding the HRV RdRP (“HRV1 RdRP”, solid squares), which can further amplify the HRV amplicon, as well as RNA encoding a noncoding RNAi cargo, the sense and antisense strands of which are formed during the amplification process to yield a double-stranded RNA molecule (dsRNA) for silencing of a target gene. In the absence of the commensal virus, no amplification of the commensal satellite occurs.

### DETAILED DESCRIPTION

### Definitions

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “F₁,” “F₂,” and the like refer to filial generations of plants or seed obtained from a parent plant which has been selfed or that has been crossed to another plant.

As used herein, the term “heterologous”, when used to describe a first element in reference to a second element means that the first element and second element do not exist in nature disposed as described. For example, a heterologous nucleic acid molecule or sequence is a nucleic acid molecule or sequence that (a) is not native to a cell in which it is expressed. (b) is linked or fused to a nucleic acid molecule or sequence with which it is not linked to or fused to in nature, or with which it is not linked to or fused to in nature in the same way, (c) has been altered or mutated by the hand of man relative to its native state, or (d) has altered expression as compared to its native expression levels under similar conditions. For example, a “heterologous promoter” is used to drive transcription of a sequence that is not one that is natively transcribed by that promoter (e.g., a eukaryote promoter used to drive transcription of a DNA molecule encoding a Secoviridae RNA sequence); thus, a “heterologous promoter” sequence can be included in an expression construct by a recombinant nucleic acid technique. In embodiments, a recombinant polynucleotide such as those provided by this disclosure includes genetic sequences of two or more different viruses of the family Secoviridae, which genetic sequences are “heterologous” in that they would not naturally occur together. In some embodiments “heterologous” refers to a molecule or to a discrete part of a molecule; for example, referring to a cargo RNA molecule (e.g., a nucleic acid such as a protein-encoding RNA, an ssRNA, a regulatory RNA, an interfering RNA, or a guide RNA), which can be part of a larger molecule, or referring to a structure (e.g., structures including a promoter (e.g., for a DNA dependent RNA polymerase) or subgenomic promoter (e.g., for an RNA-dependent RNA polymerase), an RNA effecter, RNA cleavage agent recognition site, or a polynucleotide comprising or encoding an expression-enhancing element, encapsidation recognition element (ERE), selectable or scoreable marker, DNA aptamer, RNA aptamer; a transcription factor binding site, internal ribosome entry site (IRES), DNA spacer, an RNA cleavage agent recognition site, tRNA-like element, or a transcript-stabilizing or transcript-destabilizing RNA sequence) that is not found naturally in a plant virus of the family Secoviridae.

As used herein, the terms “include,” “includes,” and “including” are to be construed as at least having the features to which they refer while not excluding any additional unspecified features. The terms “comprise”, “comprises”, and “comprising” mean “include”, “includes”, and “including”, respectively.

As used herein, the term “internal ribosome entry site” or “IRES” refers to a sequence (e.g., an RNA sequence) capable of recruiting a ribosome and translation machinery to initiate translation from an RNA sequence. An IRES element is generally between 100-800 nucleotides. An appropriate IRES can be obtained from plant and plant viral IRES sequences such as encephalomyocarditis virus IRES (ECMV), maize hsp101 IRES 5′UTR, crucifer infecting tobamovirus crTMV CR-CP 148 IRES, tobacco etch virus (TEV) IRES 5′UTR and hibiscus chlorotic ringspot virus (HCRSV) IRES. In addition, in embodiments, an IRES sequence is derived from non-plant eukaryotic virus sequences that include but are not limited to: acute bee paralysis virus (ABPV), classical swine fever virus (CSFV), coxsackievirus B3 virus (CVB3), encephalomyocarditis virus (ECMV), enterovirus 71 (E71), hepatitis A virus (HAV), human rhinovirus (HRV2), human rhinovirus (HRV2), human lymphotropic virus (HTLV), polyoma virus (PV), and Zea mays (ZmHSPI10). Examples of IRES sequences useful in the compositions and methods described herein are shown in Table 5.

As used herein, the phrase “operably linked” refers to a juxtaposition wherein the components so described are in a relationship permitting them to function in their intended manner. For instance, a promoter is operably linked to a coding sequence if the promoter provides for transcription or expression of the coding sequence.

As used herein the term “percent identity” refers to percent (%) sequence identity with respect to a reference polynucleotide or polypeptide sequence following alignment by standard techniques. Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32(5): 1792-1797, 2004). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul et al. (1990) J. Mol. Biol., 215:403-410). As an illustration, the percent sequence identity of a given nucleic acid or amino acid sequence, A, to, with, or against a given nucleic acid or amino acid sequence. B, (which can alternatively be phrased as a given nucleic acid or amino acid sequence, A that has a certain percent sequence identity to, with, or against a given nucleic acid or amino acid sequence, B) is calculated as follows: 100 multiplied by (the fraction X/Y), where X is the number of nucleotides or amino acids scored as identical matches by a sequence alignment program (e.g., BLAST) in that program's alignment of A and B, and where Y is the total number of nucleotides or amino acids in B.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, mature or immature embryos, flowers, fruits, shoots, leaves, roots, stems, and explants). In embodiments, a plant tissue or plant organ is or includes a seed, protoplast, callus, or any other group of plant cells that is organized into a structural or functional unit. In embodiments, a plant cell or tissue culture is capable of regenerating a plant having the physiological and morphological characteristics of the plant from which the cell or tissue was obtained, and of regenerating a plant having substantially the same genotype as the plant. Regenerable cells in a plant cell or tissue culture can include embryos, protoplasts, meristematic cells, callus, pollen, leaves, anthers, roots, root tips, flowers, and/or stalks. In contrast, some plant cells are not capable of being regenerated to produce plants and are referred to herein as “non-regenerable” plant cells.

As used herein, the term “transcriptome” refers to the sum total of all RNA molecules expressed in a cell. Such RNA molecules include mRNAs, tRNAs, ribosomal RNAs, miRNAs, viral RNAs (both genomic and sub-genomic), and long non-coding RNAs.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Unless otherwise stated, nucleic acid sequences described herein are given, when read from left to right, in the 5′ to 3′ direction. Nucleic acid sequences may be provided as DNA or as RNA, as specified. Furthermore, because of known codon degeneracy, different nucleic acid sequences can encode the same polypeptide sequence, and such modified nucleic acid sequences (e.g., for the purposes of codon optimization for a given species) are within the scope of the present disclosure. Where a term is provided in the singular, it also contemplates aspects of the invention described by the plural of that term.

This disclosure provides, inter alia, recombinant polynucleotides (e.g., recombinant DNA, recombinant RNA, recombinant ssRNAs, recombinant dsRNAs, recombinant vectors, etc.) including one or more sequences of or derived from a virus of the family Secoviridae; in particular, a 5′ or 3′ RNA replication element that is recognized by a Secoviridae RNA-dependent RNA polymerase (RdRP). This disclosure is further related to methods of making and using such recombinant polynucleotides, for example, by employing such recombinant polynucleotides to express a heterologous cargo sequence in a plant and optionally thereby modifying expression of an endogenous target sequence and/or genotype or phenotype of the plant. In embodiments, the virus of the family Secoviridae is a commensal virus of the family Secoviridae, that is, a virus of the family Secoviridae that is endemic or native to a given eukaryote host (such as a host plant) without causing apparent negative effects on the host (i.e., is considered non-pathogenic), is often present at a persistent but low population (i.e., low viral titer), and is often vertically transmitted to succeeding generations of the host.

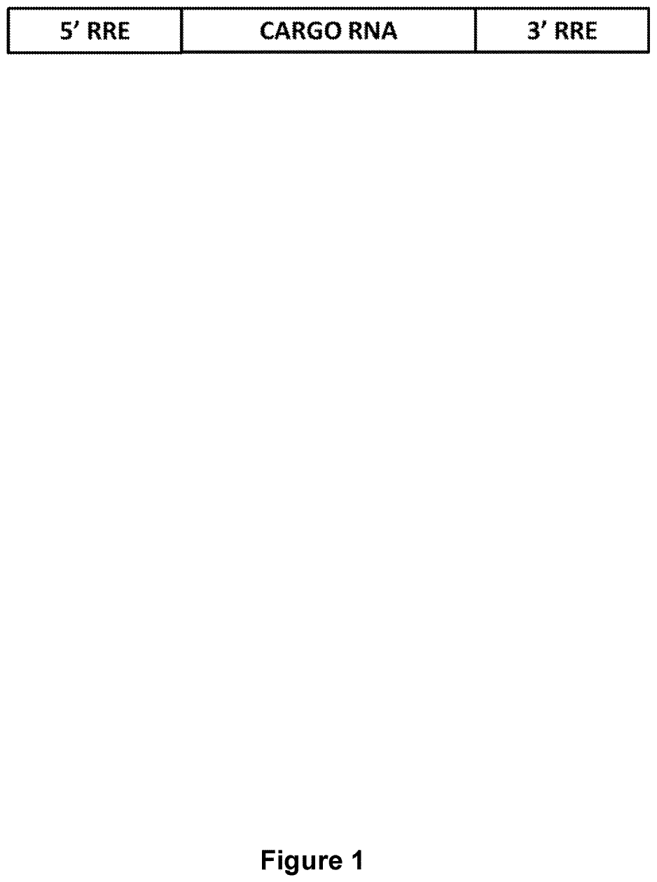

In one aspect, this disclosure is related to a recombinant DNA molecule that includes a promoter that is functional in a cell and is operably linked to a DNA sequence encoding an RNA molecule. The RNA molecule includes, in 5′ to 3′ order: (a) a 5′ RNA replication element that is capable of being recognized by a Secoviridae RNA-dependent RNA polymerase (RdRP); (b) a cargo RNA sequence; and (c) a 3′ RNA replication element that is capable of being recognized by the Secoviridae RdRP. FIG. 1 shows an embodiment of a generalized structure of a DNA polynucleotide encoding a Secoviridae satellite, where in certain embodiments the 5′ RNA replication element corresponds to the 5′ untranslated region (UTR) of a virus of the family Secoviridae and where the 3′ RNA replication element corresponds to the 3′ untranslated region (UTR) of a virus of the family Secoviridae. In some embodiments, the 5′ RNA replication element and/or the 3′ RNA replication element include nucleotides that extend into the predicted coding sequence or open reading frame of the virus of the family Secoviridae.

Recombinant DNA molecules provided herein can include a promoter that is functional in a cell (e.g., a bacterial cell, a plant cell, a fungal cell, or an animal cell) and is operably linked to a DNA sequence encoding an RNA molecule (e.g. a 5′ RNA replication element, a cargo RNA sequence; and a 3′ RNA replication element; a ribozyme, an intron, or a RNA encoding a protein (e.g., a capsid, movement, RdRP, or an RdRP protein that recognizes an HRV 5′ or 3′ replication region and/or a subgenomic promoter).

In embodiments, a promoter functional in a plant cell provides for systemic gene expression, or alternatively for cell-, tissue-, or organ-specific gene expression, or expression that is inducible by external signals or agents (for example, light-, pathogen-, wound-, stress-, or hormone-inducible elements, or chemical inducers) or elements that are capable of cell-cycle regulated gene transcription; such elements may be located in the 5′ or 3′ regions of the native gene or engineered into a polynucleotide.

Promoters include those from viruses, bacteria, fungi, animals, and plants. Suitable promoters can be used to drive expression by any RNA polymerase (e.g., RNA pol I, pol II, or pol III) Embodiments of promoters include those from cauliflower mosaic virus (e.g., p35S), bacteriophage (e.g., pT7), and plants (e.g., pATUBQ10). In certain embodiments, the promoter is operably linked to nucleotide sequences encoding multiple guide RNAs, wherein the sequences encoding guide RNAs are separated by a cleavage site such as a nucleotide sequence encoding a microRNA recognition/cleavage site or a self-cleaving ribozyme (see, e.g., Ferr6-D′Amaré and Scott (2010) Cold Spring Harbor Perspectives Biol.. 2:a003574). In certain embodiments, the promoter is a pol II promoter operably linked to a nucleotide sequence encoding the RNA. In certain embodiments, the promoter operably linked to one or more polynucleotides encoding elements of a genome-editing system is a constitutive promoter that drives DNA expression in plant cells. In certain embodiments, the promoter drives DNA expression in the nucleus or in an organelle such as a chloroplast or mitochondrion. Examples of constitutive promoters active in plant cells include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and a nopaline synthase (NOS) and octopine synthase (OCS) promoter from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PDK) promoter, which is active in the chloroplasts of mesophyll cells. In some embodiments, the promoter is heterologous to the cell it is functional in and/or to the other elements to which the promoter is operably linked. All of the patent publications referenced in this paragraph are incorporated herein by reference in their entirety.

Embodiments of recombinant polynucleotides provided herein comprise or encode RNA molecules containing 5′ and 3′ RNA replication elements recognized by a Secoviridae RNA-dependent RNA polymerase (RdRP). In certain embodiments, recognition by a Secoviridae RdRP is identified in an in vitro RdRP assay (e.g., an assay adapted from Horiuchi et al. Plant Cell Physiol. 42(2):197-203, 2001). In certain embodiments, recognition by a Secoviridae RdRP is identified by an in vivo RdRP assay wherein an RNA comprising 5′ and 3′ RNA replication elements is introduced into a cell comprising the RdRP, and replication of the RNA is assayed (e.g., by an RT-PCR assay or an assay for a reporter gene encoded by a cargo RNA located in the RNA comprising 5′ and 3′ RNA replication elements). In certain embodiments, cells comprising the RdRP are engineered by introducing a gene or RNA molecule encoding the RdRP into the cell. In other embodiments, the cell comprising the RdRP is a cell which contains a virus of the family Secoviridae which expresses the RdRP; in such embodiments the Secoviridae virus can be one that is native to or is known to naturally occur in the cell, or it can be a non-native Secoviridae virus, or alternatively a recombinant virus of any suitable viral family that is engineered to express the Secoviridae RdRP. In certain embodiments, the recombinant polynucleotides (e.g., recombinant DNAs or recombinant RNAs) comprise a 5′ RNA replication element and a 3′ RNA replication element obtained from the same Secoviridae genome or from Secoviridae genomes having at least 85%, 90%, 95%, 98%, or 99% sequence identity to one another and which are optionally related. In embodiments, the Secoviridae genomes having at least 85%, 90%, 95%, 98%, or 99% sequence identity to one another are taxonomically related, e.g., genomes classified as belonging to the same genus, family, and/or order. In certain embodiments, the recombinant polynucleotides (e.g., recombinant DNAs or recombinant RNAs) comprise a 5′ RNA replication element, a 3′ RNA replication element, and an RdRP are obtained from the same Secoviridae genome or from Secoviridae genomes having at least 85%, 90%, 95%, 98%, or 99% sequence identity to one another and which are optionally taxonomically related, e.g.. genomes classified as belonging to the same genus, family, and/or order. Non-limiting examples of a 5′ RNA replication element and a 3′ RNA replication element from the same Secoviridae genome and the corresponding RdRP protein that recognizes that 5′ RNA replication element and 3′ RNA replication element include those set forth in each row of Table 19. In certain embodiments, the recombinant polynucleotides (e.g., recombinant DNAs or recombinant RNAs) comprise a 5′ RNA replication element, a 3′ RNA replication element, and/or an RdRP coding region are obtained from two Secoviridae genomes wherein the members of each pair of the 5′ RNA replication elements, 3′ RNA replication elements, and RdRP coding regions of the two Secoviridae genomes have at least 85%, 90%, 95%, 98%, or 99% sequence identity to one another. In certain embodiments, the recombinant polynucleotides (e.g., recombinant DNAs or recombinant RNAs) comprise a 5′ RNA replication element and a 3′ RNA replication element obtained from distinct Secoviridae genomes. In certain embodiments, the recombinant polynucleotides (e.g., recombinant DNAs or recombinant RNAs) comprise a 5′ RNA replication element, a 3′ RNA replication element, and an RdRP are obtained from distinct Secoviridae genomes. In certain embodiments, the distinct Secoviridae genomes will have less than 85%, 80%, 75%, or 70% sequence identity to one another. In certain embodiments, the distinct Secoviridae genomes will have 50%, 60%, or 65% to any one of 70%, 75%, 80%, or 84% sequence identity to one another.

In certain embodiments, the combination of 5′ RNA replication elements, 3′ RNA replication elements, and RdRP set forth in a single row of Table 19, or variants thereof having at least 85%, 90%, 95%, 98%, or 99% sequence identity to the 5′ RNA replication element, 3′ RNA replication elements, and RdRP, or variants thereof wherein the secondary structures of the RNA replication elements are conserved, are used together in an expression system, plant cell, plant propagule, plant, or method provided herein. In certain embodiments, the 5′ RNA replication elements and 3′ RNA replication elements in a given row of Table 19 or variants thereof are operably linked to a cargo RNA and replicated by the corresponding RdRP or variant thereof in the row. In certain embodiments, the combination of 5′ RNA replication elements, 3′ RNA replication elements, and RdRP set forth in a given row of Table 19 (i.e., SEQ ID NO: 467, 469, 471, respectively) or aforementioned or otherwise disclosed variants thereof are used in a dicot plant cell-based expression system, dicot plant cell, dicot plant propagule, dicot plant, or related dicot plant-based method provided herein. In certain embodiments, the aforementioned dicot is a member of the genus Arabidopsis, Brassica, Capsicum, Cucumis, Cucurbita, Cynara, Fragaria, Gossypium, Malus, Nicotiana, Ribes, Solanum, Vaccinium, Vitis, or Glycine. In certain embodiments, the combination of 5′ RNA replication elements, 3′ RNA replication elements, and RdRP set forth in a given row of Table 19 (i.e., SEQ ID NO: 467, 469, 471, respectively) or aforementioned or otherwise disclosed variants thereof are used in a monocot plant cell-based expression system, monocot plant cell, monocot plant propagule, monocot plant, or related monocot plant-based method provided herein. In certain embodiments, the aforementioned monocot is a member of the genus Avena, Hordeum, Oryza. Secale, Triticum, Sorghum, or Zea.

Examples of DNA molecules which encode RNA molecules comprising or containing 5′ and 3′ RNA replication elements recognized by a Secoviridae RdRP are set forth in Table 1. DNA molecules which encode RNAs comprising or containing 5′ RNA replication elements recognized by a Secoviridae RdRP include SEQ ID NO: 467, 468, 512, 546-551, and 631. DNA molecules which encode RNAs comprising or containing 3′ RNA replication elements recognized by a Secoviridae RdRP include SEQ ID NO: 469, 470, 520, 521, and 552-557.

Structural features (e.g., dsRNA hairpins and ssRNA loops) identified in Secoviridae 5′ and 3′ RNA replication elements are shown in Table 1 by way of dot bracket notation. The dot bracket notation provided in Table 1 was generated using RNA Fold software for predicting RNA secondary structure based on minimum free energy predictions of base pair probabilities. A dot ‘.’ signifies an unpaired base and a bracket ‘(‘ or’)’ represents a paired base. Dot bracket notation is further described in Mattei et al., Nucleic Acids Research, 42(10): 6146-6157, 2014; Ramlan and Zauner In: International Workshop on Computing With Biomolecules, E. Csuhaj-Varju, R. Freund. M. Oswald and K. Salomaa (Eds.), 27 Aug. 2008, Wien, Austria, pp. 75-86, From: Austrian Computer Society. 2008; and Hofacker et al., Monatshefte fir Chemie Chem. Monthly, 125: 167-188, 1994. Such structural features can range in size from 20, 30, or 40 to about 500 nucleotides (nt). These structural features are useful for designing engineered polynucleotide sequences that function as Secoviridae RNA replication elements and/or for constructing variants of the sequences set forth in SEQ ID NOs: 467-470, 512, 520, 521, 546-557, and 631 that function as 5′ and 3′ RNA replication elements. In certain embodiments, one of more residues in the RNA secondary structure set forth in Table 1 or in equivalent RNAs are substituted with distinct nucleotides which maintain the RNA secondary structure (e.g., presence or absence of base pairing). In certain embodiments the RNA secondary structure set forth in Table 1 or in equivalent RNAs, the RNA secondary structure is maintained by making substitutions in the nucleotide sequence that result in no changes in the position of base-paired nucleotides or non-base-paired nucleotides. In certain embodiments the RNA secondary structure set forth in Table 1 or in equivalent RNAs is maintained by substituting nucleotides in the secondary structure which are not base paired with nucleotides which will not base pair, and/or by substituting nucleotides in the secondary structure which are base paired with nucleotides which will base pair. In this context, it is understood that, in some embodiments, maintaining the RNA secondary structure need not be absolute (e.g., the structure is partially maintained). In certain embodiments, a dsRNA structure is partially maintained when one, two, three or more nucleotides, particularly at the 5′ end and/or 3′ end of a hairpin-forming structure are substituted with nucleotides which do not base pair and thus reduce the total length of dsRNA in the structure. In certain embodiments, an unpaired RNA structure is partially maintained when one, two, three or more nucleotides, particularly at the 5′ end and/or 3′ end of a loop structure are substituted with nucleotides which base pair and thus reduce the total length of ssRNA in the loop structure. The ability of such partially maintained secondary structures to be recognized by the corresponding Secoviridae RdRP is monitored by in vitro or in vivo assays. Embodiments of Secoviridae satellite RNAs include those where the 5′ RNA replication element includes one or more of these 5′ structural features and/or wherein the 3′ RNA replication element includes one or more of these 3′ structural features. In certain embodiments, the 5′ RNA replication elements comprise an RNA encoded by a DNA having at least 70%, 75%, 80%, 85%, 90%, 95%, 98%, 99%, or 100% sequence identity to SEQ ID NO: 467, 468, 512, 546-551, or 631, optionally wherein the encoded RNA maintains or partially maintains a corresponding structural feature set forth in Table 1. In certain embodiments, the 3′ RNA replication elements comprise an RNA encoded by a DNA having at least 70%, 75%, 80%, 85%, 90%, 95%, 98%, 99%, or 100% sequence identity to SEQ ID NO: 469, 470, 520, 521, or 552-557, wherein the encoded RNA optionally maintains or partially maintains a corresponding structural feature set forth in Table 1.

Recombinant polynucleotides (e.g., recombinant DNA, recombinant RNA, recombinant ssRNAs, recombinant dsRNAs, recombinant vectors, etc.) provided herein can also comprise or encode additional RNA elements.

Embodiments of additional RNA elements include RNAs encoding a Secoviridae capsid or coat protein (CP). Examples of DNA sequences encoding Secoviridae CP include the corresponding sequences of Secoviridae genomes set forth in Table 1. Examples of DNA sequences encoding Secoviridae CP also include the sequence of the SEQ ID NO: 474, 476, and 478 as well as DNA sequences having at least 85%, 90%, 95%, 98%, or 99% identity thereto. Examples of DNA sequences encoding a Secoviridae CP also include the sequence of the DNA encoding the CP disclosed in SEQ ID NO: 473, 475, and 477 as well as DNA sequences having at least 85%, 90%, 95%, 98%, or 99% identity thereto. Examples of DNA sequences encoding a Secoviridae CP and Secoviridae CP sequences also include the sequences set forth in Table 17 as well as the DNA and protein sequences having at least 85%, 90%, 95%, 98%, or 99% identity thereto.

Embodiments of additional RNA elements include RNAs encoding a Secoviridae RdRP. Examples of DNA sequences encoding Secoviridae RDRP include the corresponding sequences of Secoviridae genomes set forth in Table 1. Examples of DNA sequences encoding Secoviridae RdRP also include the sequence of SEQ ID NO: 472, 539, and 565-571 as well as DNA sequences having at least 85%, 90%, 95%, 98%, or 99% identity thereto. Examples of DNA sequences encoding Secoviridae RdRP also include the sequence of the DNA encoding the Secoviridae RdRP disclosed in SEQ ID NO: 471, 530, and 558-564 as well as DNA sequences having at least 85%, 90%, 95%, 98%, or 99% identity thereto. Examples of DNA sequences encoding Secoviridae RdRP and Secoviridae RdRP protein sequences also include the sequences set forth in Table 18 as well as the DNA and protein sequences having at least 85%, 90%, 95%, 98%, or 99% identity thereto.

Embodiments of additional RNA elements include RNAs encoding a viral movement protein (MP). In certain embodiments, the cargo RNA comprises an RNA encoding a viral MP. Without being bound to hypothesis or theory, the viral movement protein is believed to bind to the RNA and to assist its movement (and thus the movement of the cargo RNA) throughout the plant, e.g., via the plasmodesmata. Viral MPs include movement proteins identified from tobacco mosaic virus (TMV), cowpea mosaic virus, potato leafroll virus, tomato spotted wilt virus, and tomato mosaic virus. MPs from a variety of viruses are described in Table 3.

Embodiments of additional RNA elements include tRNA-like sequences (TLS). TLS can trigger mobility of otherwise nonmobile RNAs, assisting to increase systemic delivery of the RNA molecule. TLS include tRNAs and tRNA-like sequences identified from other genetic elements, e.g., mRNAs. An isoleucine tRNA encoded by SEQ ID NO: 466 is an example of a useful tRNA-like sequence. Other mobile RNAs including TLS identified in Arabidopsis which are useful for building polynucleotides are described in Table 4. In assembling Table 4, mobile mRNA sequences were downloaded from the PLAMOM database for Arabidopsis. The tRNA “seed alignment” from the RFAM database was downloaded in stockholm format (multiple sequence alignment+secondary structure). A covariance model was created with INFERNAL for the tRNA stockholm alignment. PLAMOM mRNA sequences were scanned for significant similarity to tRNAs based on primary and secondary structure features. mRNA sequences with significant hits (E-val<1) were then saved to a fasta file. In one embodiment, such a tRNA-like sequence includes a tRNA-like sequence from an Arabidopsis Flowering Time T (FT) mRNA. In some embodiments, the RNA molecule includes at least one RNA encoding a viral MP, a tRNA-like sequence from an Arabidopsis FT mRNA, and an encapsidation recognition element (ERE) comprising TMV-OAS. In some embodiments, the RNA molecule includes a tRNA-like sequence encoded by a DNA sequence selected from the group consisting of SEQ ID NOs: 76-123, and 466. In some embodiments, the RNA molecule includes a modified tRNA-like sequence that has at least 90%, 95%, 98%, or 99% sequence identity to a scaffold tRNA-like sequence encoded by a DNA sequence selected from the group consisting of SEQ ID NOs: 76-123, and 466 and that maintains the secondary structure of the scaffold tRNA-like sequence.

Embodiments of additional RNA elements include RNAs encoding a viral capsid protein (CP). Such capsid proteins are also sometimes referred to as coat proteins, with both capsid and coat proteins being referred to as “CP.”. In embodiments, the capsid protein is heterologous to the virus of the family Secoviridae. In embodiments, the capsid protein is a Secoviridae capsid protein. In certain embodiments, the cargo RNA comprises an RNA encoding a viral CP. The CP can be provided, e.g., by co-expression of a recombinant construct encoding the CP or by native expression by a virus endogenous to or introduced into a plant cell. Encapsidation of an RNA molecule by the CP is achieved provided it contains an encapsidation recognition element (ERE), e.g., an origin of assembly sequence (OAS). Table 2 describes several OAS and CP sequences from a variety of viruses useful in engineering constructs which provide for RNA encapsidation. In embodiments, the OAS is positioned near the 3′ end of a construct, e.g., within the 3′ region of a cargo RNA or 3′ to a cargo RNA. For example, in some embodiments, the OAS is found 5′ to the 3′ RNA replication elements (e.g., the 3′ RNA replication elements set forth in Table 1). In embodiments, a TMV-OAS positioned at the 3′ end of the RNA molecule is recognized by the TMV capsid protein, leading to assembly of a TMV virion around the RNA.

Embodiments wherein the recombinant RNAs are complexed with RNA binding proteins (RBPs) are also provided herein. Embodiments of RBPs include RNA recognition motifs (RRMs) such as: (i) Lys/Arg-Gly-Phe/Tyr-Gly/Ala-Phe/Tyr-Val/Ile/Leu-X-Phe/Tyr, where X can be any amino acid (SEQ ID NO: 464); (ii) Ile/Val/Leu-Phe/Tyr-Ile/Val/Leu-X-Asn-Leu, where X can be any amino acid (SEQ ID NO: 465). Such RBP and RRM include those disclosed in Maris et al. 2005, doi.org/10.1111/j.1742-4658.2005.04653.x.

Embodiments of additional RNA elements include at least one ribozyme. Ribozymes include self-cleaving ribozyme, a ligand-responsive ribozyme (aptazyme), a trans-cleaving ribozyme designed to cleave a target sequence (e.g., a trans-cleaving hammerhead ribozyme designed to cleave the pepper phytoene desaturase (PDS) sequence (the RNA encoded by SEQ ID NO: 421), a hepatitis delta virus (HDV) ribozyme (the RNA encoded by SEQ ID NO: 423), or a hammerhead ribozyme (the RNA encoded by SEQ ID NO: 420). In various embodiments, multiple ribozymes are included in a polynucleotide. Useful ribozymes include Twister, Hammerhead, Hairpin, and other ribozymes. Non-limiting examples of useful ribozymes include those provided in Table 14. In certain embodiments, such a ribozyme (e.g., a self-cleaving ribozyme) is located 5′ to the 5′ RNA replication element and/or 3′ to the 3′ RNA replication element in the recombinant RNA. In certain embodiments, such a ribozyme (e.g., a self-cleaving ribozyme) is located 5′ to the HRV 5′ RNA replication region and/or 3′ to the HRV 3′ RNA replication region in a recombinant RNA comprising an imbedded heterologous RNA virus (HRV) amplicon.

Embodiments of additional RNA elements include intronic sequences. Examples of intronic sequences that can be included in the recombinant polynucleotides provided herein are described in Table 6. In certain embodiments, intronic sequences are placed in a 5′UTR downstream of a promoter (e.g., a promoter active in plant cells) used to drive expression of a recombinant RNA. In certain embodiments, intronic sequences are placed 5′ to a 5′ RNA replication element, in a cargo RNA, or 3′ to a 3′ RNA replication element.

Embodiments of recombinant polynucleotides and additional RNA elements include subgenomic promoters recognized by an RNA-dependent RNA polymerase (RdRP) and/or RNA molecules encoding an RNA-dependent RNA polymerase (RdRP). Examples of such subgenomic promoters and RdRP include a Brome Mosaic Virus subgenomic promoter and RdRP (Siegal et al. 1998, doi: 10.1073/pnas.95.20.11613), barley yellow dwarf virus (BYDV) sgRNA1, sgRNA2, and sgRNA3 subgenomic promoters and RdRP (Koev and Miller; J Virol. 2000 July; 74(13):5988-96. Doi: 10.1128/jvi.74.13.5988-5996.2000), Alternanthera mosaic virus (AltMV-MU) sgp1, sgp2, and sgp3 subgenomic promoters and RdRP (Putlvaev et al., Biochemistry (Mosc).; 80(8):1039-46DOI: 10.1134/5000629791508009X). Additional examples of subgenomic promoters are provided in Table 16. In certain embodiments, such subgenomic promoters are placed either 5′ and/or 3′ to an RNA molecule comprising a 5′ RNA replication element, a cargo RNA, and a 3′ RNA replication element to permit production of either or both + and − strands of the RNA molecule when the RdRP is provided. In certain embodiments, such subgenomic promoters are operably linked to a cargo RNA molecule and/or to any additional RNA element to permit production of the corresponding cargo and/or additional RNA when the RdRP is provided. In certain embodiments, the subgenomic promoters are operably linked to a cargo RNA comprising an HRV-inhibitory RNA or to a cargo RNA that encodes a protein which inhibits infection, movement, transmission, and/or replication of the HRV. In certain embodiments, the subgenomic promoters are operably linked to a cargo RNA comprising an RNA having at least 20 contiguous nucleotides having an identical or complementary sequence to a segment of equivalent length of the genomic RNA of the HRV. In certain embodiments, the subgenomic promoters are operably linked to a cargo RNA comprising an RNA having at least 20 contiguous nucleotides having an identical or complementary sequence to a segment of equivalent length of the genomic RNA of the HRV which does not encode the hrvRdRP.

Embodiments of other optional elements in the recombinant polynucleotides provided herein include: a) a discrete expression cassette including a second promoter operably linked to a DNA sequence to be transcribed, and optionally a terminator element (see, e.g., a NOS or CaMV35S terminator); (b) an expression-enhancing element (e.g., a DNA encoding an expression-enhancing intronic sequence); (c) a DNA or RNA sequence encoding a marker (e.g., a selectable marker such as DNA or RNA encoding an antibiotic resistance or herbicide resistance sequence; DNA encoding a scorable marker or detectable label (e.g., a beta-glucuronidase, fluorescent protein, luciferase, etc.); (d) a DNA aptamer; (e) a DNA or RNA sequence encoding an RNA aptamer; (f) T-DNA left and right border DNA sequences; (g) a spacer DNA sequence; (h) a DNA sequence encoding a transcription factor binding site; (i) a DNA sequence encoding a localization sequence (e.g., DNA encoding a targeting peptide, such as a nuclear localization signal (NLS), a mitochondrial localization signal, or a plastid localization signal); or (j) a DNA sequence encoding at least one sequence-specific recombinase recognition site (SSRRS: e.g., a pair of sequence-specific recombinase recognition sites that are recognized by a given recombinase, such as LOX sites recognized by a CRE recombinase); and (k) a DNA sequence encoding a transcript-stabilizing or transcript-destabilizing sequence (see, e.g., US Published Patent Application 2007/0011761, incorporated herein by reference in its entirety; Geisberg et al. (2014) Cell, 156: 812-824).

Provided herein are recombinant polynucleotides comprising a cargo RNA molecule or comprising DNA encoding a cargo RNA molecule. In some embodiments, the recombinant polynucleotide includes a single cargo RNA molecule. In other embodiments, the recombinant polynucleotide includes at least two cargo RNA molecules, e.g., 2, 3, 4, 5, 6, 7, 8, 9, 10, or more than 10 cargo RNA molecules; in embodiments, the at least two cargo RNA molecules are the same (e.g., multiple copies of a non-coding RNA sequence or multiple copies of a RNA sequence encoding a polypeptide) or are different (e.g., two or more different non-coding RNA sequences, or two or more different coding RNA sequences, or combinations of non-coding and coding cargo RNA sequences).

In certain embodiments, a cargo RNA molecule is up to about 8 kilobases (kb) in length. Cargo RNA molecules can range in length from any one of about 20 nucleotides (nt), 100 nt, 200 nt, 300 nt, 400 nt, 500 nt, 600 nt, 700 nt, 800 nt, or 900 nt to any one of about 1 kb, 2 kb, 3 kb, 4 kb, 5 kb, 6 kb, 7 kb, 8 kb, 9 kb, 10 kb, 11 kb, 12 kb, 13 kb, or 14 kb in length. Other lengths of the cargo RNA molecule are less than or equal to 100 nucleotides (nt) can range in length from any one of about 20 nt, 30 nt, or 40 nt to any one of about 50 nt, 60 nt, 70 nt, 80 nt, 90 nt, or 100 nt in length. Recombinant RNAs comprising a cargo RNA of up to about 8 kb in length can in certain embodiments be encapsidated by a Secoviridae capsid protein. In such embodiments, cargo RNAs can range in length from any one of about 20 nt, 100 nt, 200 nt, 300 nt, 400 nt, 500 nt, 600 nt, 700 nt, 800 nt, or 900 nt to any one of about 1 kb, 2 kb, 3 kb, 4 kb, 5 kb, 6 kb, 7 kb, or 8 kb in length. Recombinant RNAs comprising a cargo RNA of up to about 3 kb, 4 kb, 5 kb, 6 kb, 7 kb, 8 kb, 9 kb, 10 kb, 11 kb, 12 kb, 13 kb, or 14 kb in length can in certain embodiments be encapsidated by a heterologous viral capsid protein set forth in Table 2. In certain embodiments, recombinant RNAs comprising a cargo RNA of up to about 14 kb and encapsidated by a heterologous viral capsid protein can comprise an OAS element set forth in Table 2 and be encapsidated by a corresponding capsid protein set forth in Table 2. In some embodiments, the cargo RNA molecule is greater than 14 kb, for example. 15 kb, 16 kb, 17 kb, 18 kb, 19 kb, or even 20 kb. In embodiments, the cargo RNA molecule includes: (a) at least one coding sequence, (b) at least one non-coding sequence, or (c) both at least one coding sequence and at least one non-coding sequence. Such cargo RNA molecules include combinations of coding/non-coding sequence: multiple non-coding/coding sequences; as well as aptamers, ribozymes, and other elements as is described herein. In embodiments, the cargo RNA molecule includes (a) a coding sequence to be expressed in a plant, and (b) at least one non-coding sequence that modifies expression or translation of the coding sequence, such as a recognition and cleavage sequence for an siRNA or miRNA that is endogenously expressed in the plant (see, e.g.. U.S. Pat. Nos. 8,334,430, 9,139,838, 9,976,152, 10,793,869, 10,876,126) and can bind to and cleave an RNA transcript containing the recognition and cleavage sequence; in such embodiments, it is possible to achieve spatially or temporally or developmentally specific expression of the coding sequence in the plant. In embodiments, a coding or non-coding cargo RNA can be optimized for expression in plants by methods which include using codons which occur more frequently in plant genes and/or by eliminating polyadenylation sites in the cargo RNA (e.g., as described in at least U.S. Pat. Nos. 5,380,831, 5,689,052, and 7,741,118, which are each incorporated herein by reference in their entireties).

In embodiments, the cargo RNA molecule includes at least one coding sequence (e.g., a translatable sequence). In some embodiments, the coding sequence is accordingly a protein or a polypeptide such as those described in this disclosure's working examples. In some embodiments, a cargo RNA comprises a selectable marker RNA encoding an antibiotic resistance or herbicide resistance polypeptide sequence or a scorable marker RNA encoding a scorable marker protein (e.g., a beta-glucuronidase, fluorescent protein, luciferase, etc.). Examples of selectable marker/selection agent combinations include glyphosate-resistant EPSPS enzymes and/or glyphosate oxidases/glyphosate, a bialaphos resistance (bar) or phosphinothricin acyl transferase (pat) enzyme/glufosinate, or a neomycin phosphotransferase (npt)/neomycin or kanamycin. Examples of scorable markers include P-glucuronidase (GUS), luciferase, and fluorescent proteins such as green fluorescent protein (GFP), yellow fluorescent protein (YFP), and cyan fluorescent protein (CFP). In embodiments, the cargo RNA sequence encodes at least one protein or polypeptide that provides a desirable trait in a plant in which the protein or polypeptide is expressed. Non-limiting examples of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of beneficial insects (such as honeybees and silkworms) or for decreasing the fitness of pest invertebrates (such as aphids, caterpillars, beetle larvae, and mites). Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens. e.g., antimicrobial polypeptides or antifingal polypeptides for controlling diseases in plants, or pesticidal polypeptides (e.g., insecticidal polypeptides and/or nematicidal polypeptides) for controlling invertebrate pests such as insects or nematodes. Embodiments of antimicrobial polypeptides include cathelicidins, cecropins, beta-defensins, amphibian antimicrobial peptides (e.g., aurein-like peptides, esculentin, gaegurin, brevinin, rugosin, ranatuerin, ranacyclin, uperin, ocellatin, grahamin, nigrocin, dermoseptin, temporin, bombinin, maximin), enterocins, ponicerins, megourins, apidaecins, abaecins, attacin, bacteriocins and lantibiotics, dermcidin, formaecin, halocidins, lactocin, tachystatins, and some insecticidal toxins produced by spiders and scorpions. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see., e.g., the “AtTFDB” database listing the transcription factor families identified in the model plant Arabidopsis thaliana), publicly available at agris-knowledgebase[dot]org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast or fungal mating pheromones, invertebrate reproductive and larval signaling pheromones, see, e.g., Altstein (2004) Peptides, 25:1373-1376). Embodiments of agriculturally useful polypeptides confer a beneficial agronomic trait, e.g., herbicide tolerance, insect control, modified yield, increased fungal or oomycte disease resistance, increased virus resistance, increased nematode resistance, increased bacterial disease resistance, plant growth and development, modified starch production, modified oils production, high oil production, modified fatty acid content, high protein production, fruit ripening, enhanced animal and human nutrition, production of biopolymers, environmental stress resistance, pharmaceutical peptides (e.g., hormones, enzymes, transcription factors, antigens, antibodies, or antibody fragments) and secretable peptides, improved processing traits, improved digestibility (e.g., reduced levels of toxins or reduced levels of compounds with “anti-nutritive” qualities such as lignins, lectins, and phytates), enzyme production, flavor, nitrogen fixation, hybrid seed production, fiber production, and biofuel production. Non-limiting examples of agriculturally useful polypeptides include polypeptides that confer herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; 5,763,241; 10,017,549; 10,233,217; 10,487,123; 10,494,408; 10,494,409; 10,611,806; 10,612,037; 10,669,317; 10,827,755; 11,254,950; 11,267,849; 11,130,965; 11,136,593; and 11,180,774), fungal disease resistance (U.S. Pat. Nos. 6,653,280; 6,573,361; 6,506,962; 6.316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; 5,958,745; and 6,946,588), environmental stress resistance (U.S. Pat. No. 6,072,103), pharmaceutical peptides (e.g., hormones, enzymes, transcription factors, antigens, antibodies, or antibody fragments) and secretable peptides (U.S. Pat. Nos. 6,812,379; 6,774,283; 6,140,075; and 6.080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700). In certain embodiments, the cargo RNA encodes one or more small signaling peptides (SSPs), also called peptide honnones, which are an attractive option for use as cargoes in RNA commensal satellites due to their small size (5-75 amino acids) and potency. In certain embodiments, SSPs result from processing longer precursor polypeptides (derived from ORF regions). In other embodiments, SSPs originate from a wider range of sources including intergenic/intronic regions, long non-coding RNAs, pri-miRNAs, and 5′ and 3′ UTRs of mRNAs. Non-limiting examples of SSPs include miPEP172c, miPEP171d, BomiPEP397a, AtmiPEP397a, BvmiPEP164b, and AtmiPEP164b peptides set forth in Table 13.

In some embodiments, the RNA molecule further includes an internal ribosome entry site (IRES) located 5′ and immediately adjacent to the at least one coding sequence. In yet other embodiments, the cargo RNA molecule includes multiple coding sequences, and the RNA molecule further includes an IRES located 5′ and immediately adjacent to each of the coding sequences (e.g., open translational reading frames encoding a protein of interest. Useful IRES sequences include those depicted in Table 5.

In certain embodiments, the cargo RNA molecule includes a non-coding sequence such as those described in this disclosure's working examples. Such non-coding sequences include a hairpin RNA (hpRNA); an RNA that forms multiple stem-loops; an RNA pseudoknot; an RNA sequence that forms at least partially double-stranded RNA; a small interfering RNA (siRNA) or siRNA precursor; a microRNA (miRNA) or miRNA precursor; a ribozyme; a ligand-responsive ribozyme (aptazyme); an RNA aptamer; or a long noncoding RNA (lncRNA). In certain embodiments, the cargo RNA includes a selectable or scorable RNA marker, such as an RNA aptamer or a regulatory RNA, such as an siRNA or siRNA precursor (see, e.g., U.S. Pat. Nos. 8,404,927, 8,455,716, 9,777,288, 10,378,012), a miRNA or a miRNA precursor (see, e.g., U.S. Pat. Nos. 8,410,334, 8,395,023, 9,708,620), a trans-acting siRNA or trans-acting siRNA precursor (see, e.g., U.S. Pat. Nos. 8,030,473, 8,476,422, 8,816,061, 9,018,002), a phased sRNA or phased sRNA precursor (see, e.g., U.S. Pat. No. 8,404,928), an siRNA or miRNA decoy (see, e.g., U.S. Pat. Nos. 8,946,511, 9,873,888), an siRNA or miRNA cleavage blocker (see, e.g., U.S. Pat. No. 9,040,774), an siRNA or miRNA recognition and cleavage sequence (see, e.g., U.S. Pat. Nos. 8,334,430, 9,139,838, 9,976,152, 10,793,869, 10,876,126), a riboswitch (see, e.g. U.S. Patent Application Public. No. 20130102651; U.S. Pat. Nos. 6,630,306; 6,949,379), or a ribozyme. Suitable RNA aptamers include those that exhibit fluorescence upon binding a molecule. For example, the fluorescent RNA aptamer can be the Broccoli RNA aptamer. Other fluorescent RNA aptamers that can be used include, but are not limited to, Spinach, Spinach2, Carrot, Radish, Corn, Red Broccoli, Orange Broccoli, and Broccoli Fluorets. Other useful RNA aptamers that can be used include those provided in Table 15. Suitable regulatory RNAs can be used to down-regulate (i.e., silence) the expression of a marker gene. For example, phytoene desaturase (PDS) is widely used as a marker gene because silencing of the gene yields a photobleached phenotype. Regulatory RNAs such as decoys or cleavage blockers can also be used to interfere with endogenous small RNA-regulated pathways, resulting in a visible phenotype; see, e.g., U.S. Pat. Nos. 8,946,511, 9,873,888, 9,040,774).

Antiviral cargo RNAs, and in particular antiviral cargo RNAs directed against viral pathogens are provided herein. In certain embodiments, the antiviral cargo RNAs comprise a heterologous RNA Virus (HRV)-inhibitory RNA or encode an HRV-inhibitory protein, wherein the HRV-inhibitory RNA or protein inhibits infection, movement, transmission, and/or replication of the HRV. Target viral pathogens include an Alphaflexivirus, Betaflexivirus, Bromovirus, Celavirus, Closterovirus, Comovirus, Potexvirus, Potyvirus, Tobamovirus, Tombusvirus, Tospoviridac, Trivirinac, Tymovirus, Varicosavirus, or Secoviridae. In certain embodiments the target viral pathogen is Cucumber Mosaic Virus, Brome mosaic virus, Citrus tristeza virus, Beet yellows virus, Cowpea mosaic virus, Potato virus X; Pepper mottle virus, Bean yellow mosaic virus, Barley stripe mosaic virus, Wheat stripe mosaic virus, Rice yellow mottle virus, Maize dwarf mosaic virus, zucchini yellow mosaic virus, watermelon mosaic virus, sugarcane mosaic virus, Tobacco mosaic virus, Tomato mosaic virus. Tomato brown rugose fruit virus. Turnip vein-clearing virus, Pepper mild mottle virus, Turnip crinkle virus, Tomato bushy stunt virus, Tomato spotted wilt virus, watermelon bud necrosis virus, Turnip yellow mosaic virus, Spinach latent virus, Olive latent virus 2, Citrus yellow vein clearing virus, Potato latent virus, Apple stem grooving virus, Citrus leaf blotch virus, Apple latent spherical virus. Soybean latent spherical virus, Celery latent virus, Black grass varicosavirus-like virus, Maize suscal virus. Horseradish latent virus, Bean latent virus, Rice latent virus 1, or Rice latent virus 2. In certain embodiments, the targeted viral pathogen is a heterologous RNA virus disclosed in Table 7. In certain embodiments, antiviral inhibitory RNAs (RNAi sequences) used as cargo RNAs are obtained for a chosen target gene of a viral pathogen using siRNA/miRNA prediction tools (see, e.g., on the world wide web internet site “zhaolab[dot]org/pssRNAit/). Other examples of non-coding RNA sequences having antiviral activity (e.g., dsRNA molecules which produce miRNA or siRNA) that can be used as antiviral cargo RNAs include those disclosed in U.S. Pat. No. 8,455,716, which is incorporated herein by reference in its entirety. Non-limiting examples of viral targets for antiviral cargo RNA molecules include the viral genes and genomes provided in Table 7 as well as other variants of those viral sequences. In certain embodiments, cargo RNAs encoding antiviral proteins are provided. Non-limiting examples of antiviral proteins include the N protein (Whitham, S. et al. Cell 78, 1101-1115 (1994)) and endogenous plant viral resistance proteins provided in Table 8.

Antifungal cargo RNAs, and in particular antifungal cargo RNAs directed against plant fungal pathogens, are provided herein. Target fungal pathogens include Botrytis, Fusarium. Magnaporthe, Phytophthora, Rhizoctonia, Sclerotinia, and Verticillium sp. In certain embodiments, the antifungal cargo RNA comprises a non-coding RNA sequence having antifungal activity (e.g., dsRNA molecules which produce miRNA or siRNA) and in particular a dsRNA directed against a fungal pathogen target gene. In certain embodiments, such antifungal cargo RNAs comprising dsRNA-mediated control of fungal pathogens are modeled after those described in Qiao et al., 2021, doi: 10.1111/pbi.13589; Duanis-Assaf, et al., 2022, DOI: 10.1111/pbi.13708; Yang et al., 2022, doi: 10.3389/fmicb.2021.660976; Sundaresha et al., 2021, doi: 10.20944/preprints202102.0280.v1; and Gaffar et al., 2019, doi: 10.3389/fmicb.2019.01662. Non-limiting examples of antifungal cargo RNAi targets are provided in Table 10. In certain embodiments, antifungal inhibitory RNAs (RNAi sequences) used as cargo RNAs are obtained for a chosen target gene of a fungal pathogen (e.g., a fungal pathogen gene set forth in Table 10) using siRNA/miRNA prediction tools (see, e.g., on the world wide web internet site “zhaolab[dot]org/pssRNAit/). In other embodiments, antifungal cargo RNAs encode antifungal proteins. Useful antifumgal proteins include nodule-specific cysteine-rich antimicrobial peptides (Vellivelli et al., 2020, doi: 10.1073/pnas.2003526117), defensins (Asano et al., 2013, doi: 10.1371/journal.ppat.1003581), the conidial germination-inhibiting antifungal peptides disclosed in International Patent Application publication WO2023/004435, which is incorporated by reference herein, including their homodimers, heterodimers, and fusions with signal or cell-penetrating peptides (e.g., the sequences provided in Tables 4 and 5 of WO2023/004435); the various antifungal antimicrobial peptides disclosed in De Cesare et al. (2020) mBio 11:e02123-20; doi.org/10.1128/mBio.02123-20, including Table 1 (supplemental material, journals[dot]asm[dot]org/doi/suppl/10.1128/mbio.02123-20/suppl_file/mbio.02123-20-st001[dot]pdf), and other antifungal proteins provided in Table 11.

Insecticidal cargo RNAs, and in particular insecticidal or insect inhibitory cargo RNAs directed against insects are provided herein. Target insects include sucking insects (e.g., heteropteran and homopteran insects including aphids, whiteflies, and plant bugs), caterpillars (e.g., lepidopteran insects including fall army, black cutworm, corn earworm, soybean looper, and velvetbean caterpillar), beetles (e.g., coleopteran insects including Colorado Potato Beetle and corn rootworms), and flies (e.g., dipteran insects including Ceratitis capitata). Insecticidal or insect inhibitory cargo RNAs provided herein can be directed against insects at various stages of their development (e.g., embryonic, larval, pupal, or adult stages). In certain embodiments, the insecticidal or insect inhibitory cargo RNA comprises a non-coding RNA sequence having insecticidal or insect inhibitory activity (e.g., dsRNA molecules which produce miRNA or siRNA) and in particular a dsRNA directed against an insect target gene. In certain embodiments, such insecticidal cargo RNAs comprising dsRNA-mediated control of insects comprise or are modeled after those described in U.S. Pat. Nos. 11,091,770 and 11,186,837, which are each incorporated herein by reference in their entireties. Non-limiting examples of insecticidal or insect-inhibitory cargo RNAi targets are provided in Table 9. Non-limiting examples of insecticidal cargo RNAi targets include insect Actin, SNF7, Tyrosine hydroxylase, C002, Hunchback, V-ATPase subunit A, COPI coatomer beta prime subunit, ribosomal protein L19, and ubiquitin C genes. In certain embodiments, insecticidal or insect inhibitory RNAs (RNAi sequences) used as cargo are obtained for a chosen target gene of an insect (e.g., an insect gene set forth in Table 9 or U.S. Pat. Nos. 11,091,770 and 11,186,837) using siRNA/miRNA prediction tools (see, e.g., on the world wide web internet site “zhaolab[dot]org/pssRNAit/). In other embodiments, insecticidal cargo RNAs encode insecticidal proteins. Useful insecticidal proteins encoded by insecticidal cargo RNAs include native and modified Bacillus thuringiensis Cry, vegetative insecticidal proteins (VIP), and Cyt proteins (Palma et al. 2014, doi: 10.3390/toxins6123296; U.S. Pat. No. 11,267,849, incorporated herein by reference in its entirety) as well as insecticidal or insect-inhibitory proteins provided in Table 9.

Cargo RNAs can also encode “resistance” or “R” genes which confer resistance to certain arthropods, bacteria such as Pseudomonas sp.. Xanthomonas sp., and Erwinia sp., and fungal pathogens including Cochiobolus, Blumeria, Fusarium, Melampsora, and Magnaporthe sp. Non-limiting examples of R genes encoded by cargo RNAs include those provided in Table 12.

In some embodiments, the cargo RNA molecule comprises a CRISPR guide RNA, e.g., a crRNA, gRNA, or sgRNA. CRISPR-associated endonuclcases such as Cas9, Cas12, and Cas13 endonucleases are used as genome editing tools in different plants; see, e.g., Wolter et al. (2019) BMC Plant Biol., 19:176-183); Aman et al. (2018) Genome Biol., 19:1-10. CRISPR/Cas9 requires a two-component crRNA:tracrRNA “guide RNA” (“gRNA”) that contains a targeting sequence (the “CRISPR RNA” or “crRNA” sequence) and a Cas9 nuclease-recruiting sequence (tracrRNA). Efficient Cas9 gene editing is also achieved with the use of a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example. Cong et al. (2013) Science, 339:819-823; Xing et al. (2014) BMC Plant Biol., 14:327-340. Chemically modified sgRNAs have been demonstrated to be effective in genome editing; see, for example. Hendel et al. (2015) Nature Biotechnol., 985-991. Commercial manufacturers of CRISPR nucleases and guide RNAs provide algorithms for designing guide RNA sequences; see, e.g., guide design tools provided by Integrated DNA Technologies at www[dot]idtdna[dot]com/pages/products/crispr-genome-editing/alt-r-crispr-cas9-system. Some Cas nucleases, including Cas12a and Cas13, do not require a tracrRNA.

Also provided herein are recombinant polynucleotides wherein the 5′ and 3′ RNA replication elements of the virus of the family Secoviridae flank an internal sequence wherein the cargo RNA is operably linked to one or more elements of a heterologous RNA virus (HRV).

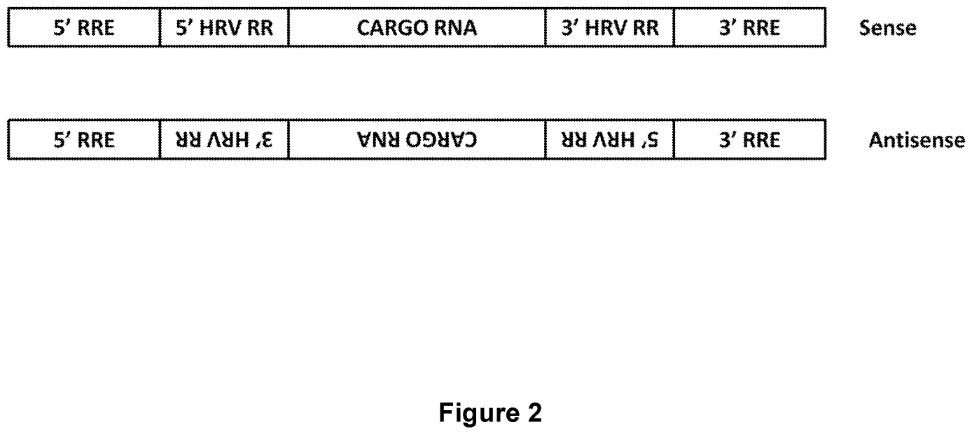

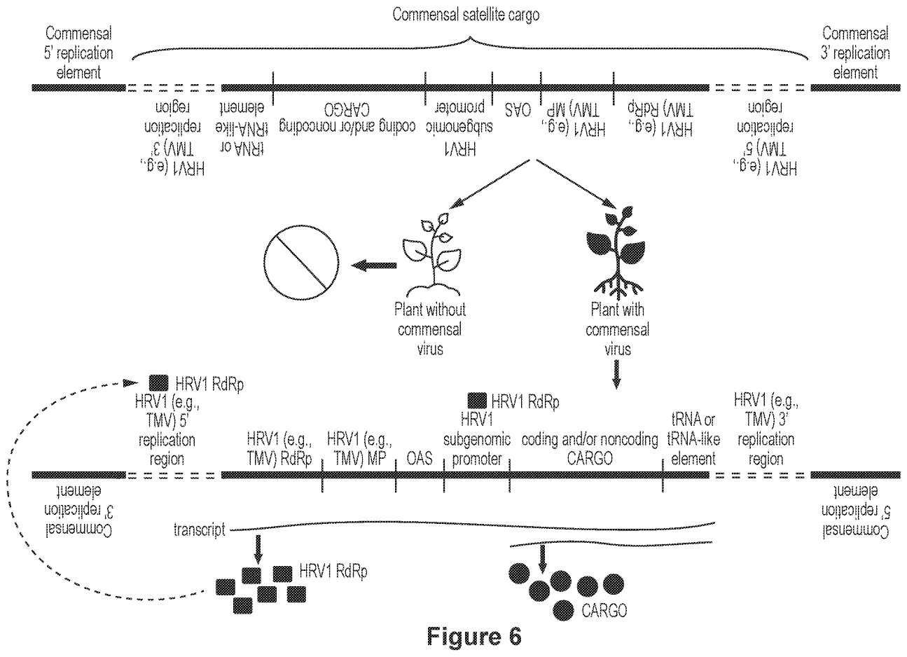

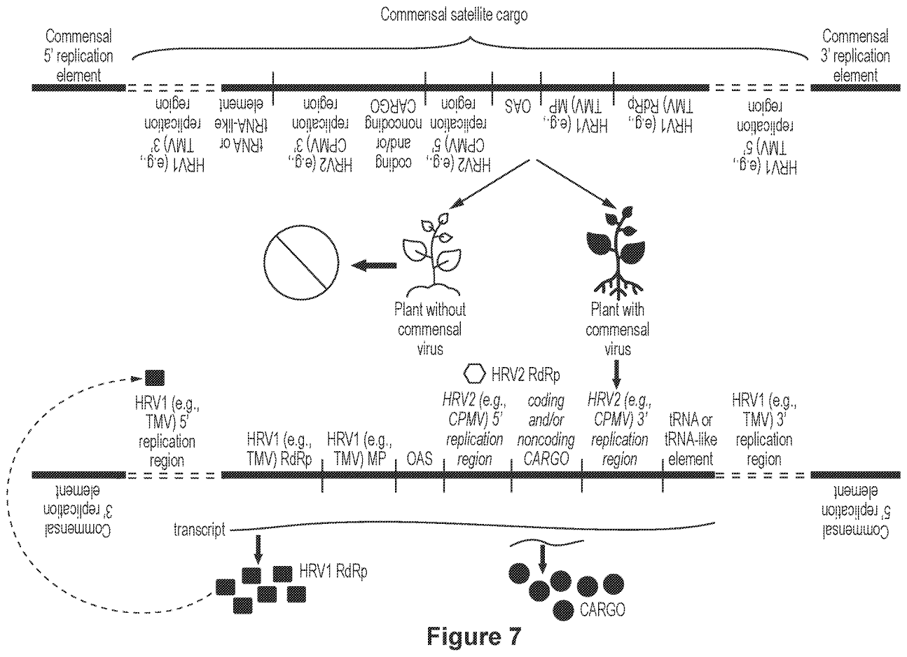

In certain embodiments, cargo RNAs are imbedded within a heterologous RNA virus (HRV) amplicon comprising; (i) an HRV 5′ replication region (HRV 5′ RR); (ii) the cargo RNA molecule; and (iii) the heterologous RNA virus (HRV) 3′ RNA replication region (HRV 3′RR), wherein (i), (ii), and (iii) are operably linked. An illustrative example of a Secoviridae satellite construct with such an HRV amplicon is shown in FIG. 2. Amplification of such an HRV amplicon in plants comprising a Secoviridae satellite construct, the virus of the family Secoviridae, and an HRV RdRP is illustrated in FIG. 6. Examples of HRV 5′ replication regions (5′RR), 3′ replication regions (3′RR), and corresponding HRV RNA-dependent RNA Polymerases (RdRP) that recognize such replication regions are set forth in Table 7. In certain embodiments where the cargo RNA encodes a protein, an internal ribosome entry-site (IRES; e.g. an IRES in Table 5) is typically operably linked to the coding cargo RNA. In certain embodiments one or more self-cleaving or inducible ribozymes are operably linked to the 5′ end of the HRV 5′ RR and to the 3′ end of the HRV 3′ RR. In certain embodiments, the HRV amplicon further comprises a subgenomic promoter which is operably linked to the cargo RNA molecule. Examples of subgenomic promoters include a subgenomic promoter of the HRV and/or a Brome Mosaic Virus subgenomic promoter (Siegal et al. 1998, doi: 10.1073/pnas.95.20.11613), barley yellow dwarf virus (BYDV) sgRNA1, sgRNA2, and sgRNA3 subgenomic promoters (Koev and Miller; J Virol. 2000 July; 74(13):5988-96, doi: 10.1128/jvi.74.13.5988-5996.2000), and an Alternanthera mosaic virus (AltMV-MU) sgp1, sgp2, or sgp3 subgenomic promoter (Putlyaev et al., Biochemistry (Mosc).; 80(8):1039-46, doi: 10.1134/S000629791508009X). Further examples of subgenomic promoters include those in Table 16. Such HRV amplicons can be in the sense or antisense orientation with respect to the Secoviridae 5′ RNA replication element. When the HRV amplicon is oriented in the sense orientation relative to the Secoviridae 5′ RNA replication element, the HRV 5′ RR and 3′ RR are present in the recombinant RNA molecule in the sense orientation, as in the corresponding sequences found in the plus (+) strand of the HRV genomic RNA. When the HRV amplicon is oriented in the antisense orientation relative to the Secoviridae 5′ RNA replication element, the HRV 5′ RR and 3′ RR are present in the recombinant RNA molecule in antisense orientation, as in the corresponding sequences found in the negative (−) strand of the HRV genomic RNA. Under certain conditions where a plant cell or plant containing the recombinant RNA containing the HRV amplicon are provided with an RNA-dependent RNA polymerase that recognizes the HRV 5′ RR and 3′RR (hrvRdRP), the HRV amplicon undergoes amplification (e.g., hrvRdRP-mediated replication). Such hrvRdRP can be provided by sources that include: (i) infection by the HRV; (ii) introduction by vector-mediated delivery of a polynucleotide encoding the hrvRdRP (e.g., Agrobacterium-mediated delivery or viral vector mediated delivery); or (iii) introduction of a nucleic acid encoding the hrvRdRP. A non-limiting example of a recombinant satellite RNA that contains HRV 5′RR and 3′RR sequence pairs that are respectively recognized by two distinct HRV RdRP (“HRV1 RdRP” and “HRV2 RdRP”) is illustrated in FIG. 7. Amplification (e.g., an increase in copy number of the HRV amplicon) provides for additional copies of the cargo RNA and an enhancement of desirable phenotypes conferred by the cargo RNA (e.g., increased antiviral, antifungal, or insecticidal activity in comparison to control plants lacking the amplified cargo RNA or lacking the cargo RNA).

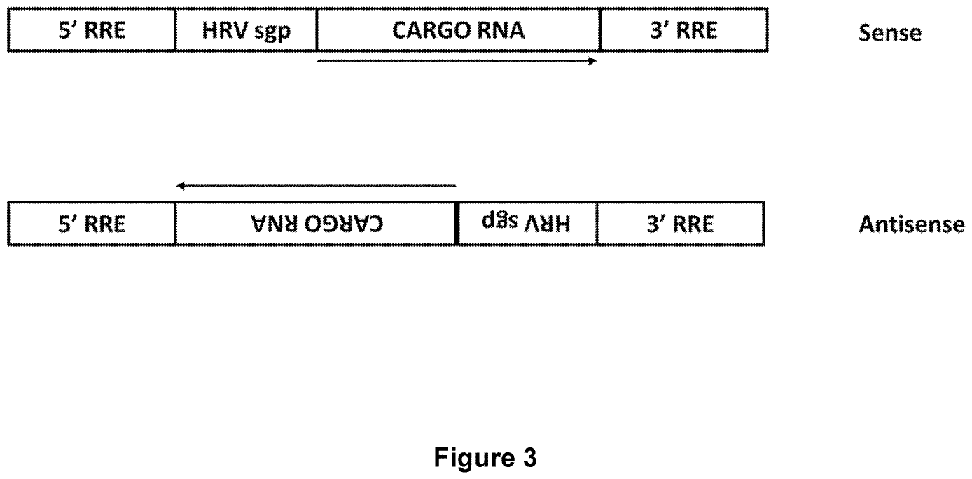

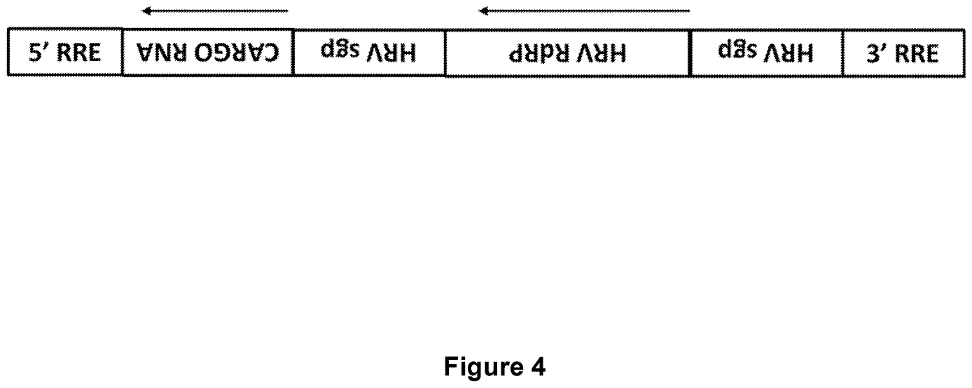

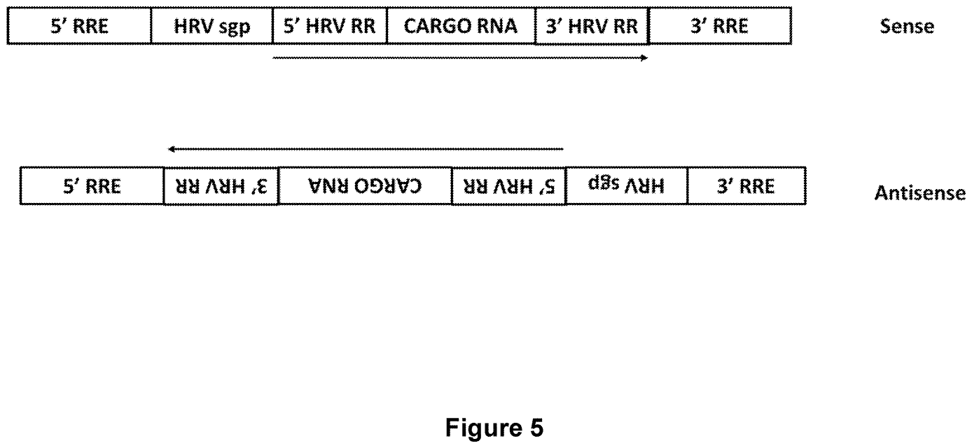

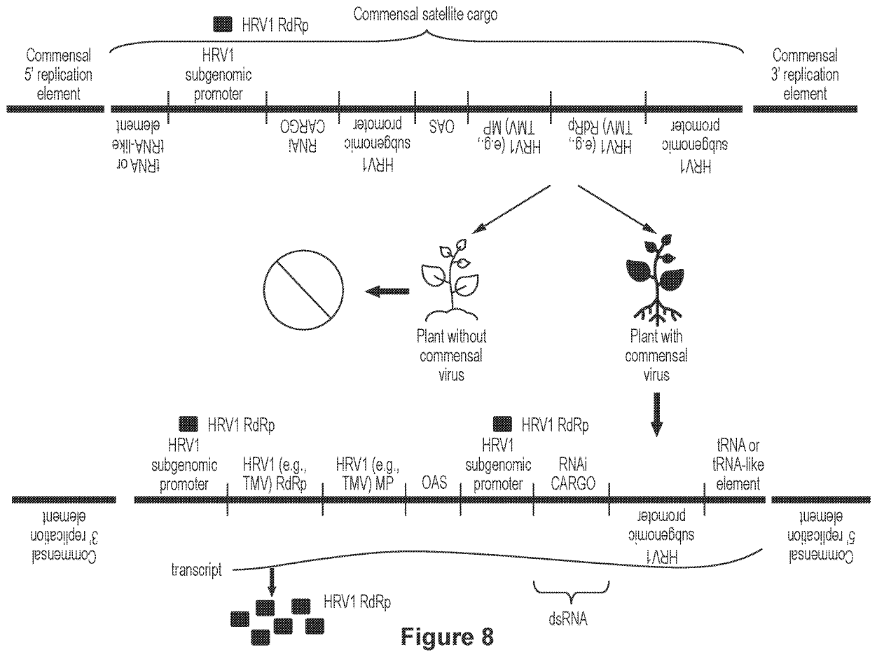

In other embodiments, the recombinant nucleotides provided herein comprise Secoviridae 5′ and 3′ RNA replication elements flanking a heterologous RNA virus (HRV) subgenomic promoter operably linked to the cargo RNA molecule: wherein the subgenomic promoter is recognized by a heterologous RNA virus RNA-dependent RNA polymerase (hrvRdRP). An illustrative example of a Secoviridae satellite construct with subgenomic promoters in sense or antisense orientation relative to the 5′ RRE is shown in FIG. 3. Another illustrative example where subgenomic promoters drive expression of an HRV RdRP and a dsRNA cargo in plants comprising a Secoviridae satellite construct is shown in FIG. 8. In certain embodiments where the cargo RNA linked to the subgenomic promoter encodes a protein, an internal ribosome entry site (IRES; e.g. an IRES in Table 5) is typically operably linked to the cargo RNA. Embodiments of subgenomic promoters include a subgenomic promoter of the HRV and/or a Brome Mosaic Virus subgenomic promoter (Siegal et al. 1998, doi: 10.1073/pnas.95.20.11613), barley yellow dwarf virus (BYDV) sgRNA1, sgRNA2, and sgRNA3 subgenomic promoters (Koev and Miller; J Virol. 2000 July; 74(13):5988-96. Doi: 10.1128/jvi.74.13.5988-5996.2000), and an Alternanthera mosaic virus (AltMV-MU) sgp1, sgp2, or sgp3 subgenomic promoter (Putlyaev et al., Biochemistry (Mosc).:80(8):1039-46DOI: 10.1134/S000629791508009X). Further subgenomic promoters include those in Table 16. Such subgenomic promoters and operably linked cargo RNAs can be in the sense or antisense orientation with respect to the Secoviridae 5′ RNA replication element. Expression of cargo RNAs from subgenomic promoters provides for additional copies of the cargo RNA and an enhancement of desirable phenotypes conferred by the cargo RNA (e.g., increased antiviral, antifingal, or insecticidal activity in comparison to control plants lacking the additional expressed cargo RNA or lacking the cargo RNA). When the subgenomic promoters and operably linked cargo RNAs are oriented in the sense orientation relative to the Secoviridae 5′ RNA replication element, the subgenomic promoters and operably linked cargo RNAs are present in the recombinant RNA molecule as a sense strand where the subgenomic promoter is recognized by the hrvRdRP to produce the desired cargo RNA. When the subgenomic promoters and operably linked cargo RNAs are oriented in the sense orientation relative to the Secoviridae 5′ RNA replication element in the recombinant RNA molecule (positive strand), the subgenomic promoter can be recognized by the hrvRdRP to produce the desired cargo RNA. When the HRV amplicon is oriented in the antisense orientation relative to the Secoviridae 5′ RNA replication element in the recombinant RNA molecule, the subgenomic promoter cannot be recognized by the hrvRdRP to produce the desired cargo RNA. However, the negative strand of the recombinant RNA molecule produced by the Secoviridae RdRP would contain the subgenomic promoters and operably linked cargo RNA in a sense orientation where the subgenomic promoter can be recognized by the hrvRdRP to produce the desired cargo RNA. In certain embodiments, the HRV amplicons further comprise a HRV 5′ RR and 3′ RR which flank the cargo RNA and provide for hrvRdRP-mediated replication of an RNA comprising from 5′ to 3′ the HRV 5′ RR, cargo RNA, and HRV 3′ RR (e.g., as illustrated in the non-limiting example of FIG. 5). Under certain conditions where a plant cell or plant containing the recombinant RNA containing the subgenomic promoters and operably linked cargo RNA are provided with an RNA-dependent RNA polymerase that recognizes the subgenomic promoter (hrvRdRP), an RNA encoding the cargo molecule can be produced (e.g., via hrvRdRP-mediated synthesis of the cargo RNA from the subgenomic promoter). Such hrvRdRP can be provided by sources that include: (i) infection by the HRV; (ii) introduction by vector-mediated delivery (e.g., Agrobacterium-mediated delivery or viral vector mediated delivery); (iii) introduction of a nucleic acid encoding the hrvRdRP; or (iv) inclusion of a cargo RNA in the recombinant nucleotides comprising Secoviridae 5′ and 3′ RNA replication elements. In one particular embodiment, the subgenomic promoter and operably linked cargo RNA are present in the recombinant RNA molecule as the antisense strand, and the cargo RNA encodes both an hrvRdRP and a second coding or non-coding RNA where both the hrvRdRP and a second coding or non-coding RNA are operably linked to a subgenomic promoter recognized by the hrvRdRP. In certain embodiments, an IRES is operably linked to the RNA encoding the hrvRdRP. Production of the negative strand of the recombinant nucleotides comprising Secoviridae 5′ and 3′ RNA replication elements results in an RNA where the subgenomic promoters recognized by the hrvRdRP can drive expression of the HRV RdRP and a second coding or non-coding RNA. An illustrative example of a Secoviridae satellite construct with subgenomic promoters in antisense orientation relative to the 5′ RRE and driving expression of both an hrvRdRP that recognizes the subgenomic promoters and a second cargo RNA is shown in FIG. 4.

In a related aspect, an RNA molecule including at least one HRV amplicon is amplified directly by the hrvRdRP (e.g., without initial or further amplification by the Secoviridae RdRP). In one embodiment, the HRV amplicon includes, in 5′ to 3′ order, (i) a heterologous RNA virus (HRV) 5′ replication region (HRV 5 ′RR); (ii) a cargo RNA molecule; and (iii) a heterologous RNA virus (HRV) 3′ RNA replication region (HRV 3′RR); wherein the HRV 5′ RR and HRV 3′ RR HRV are recognized by a heterologous RNA virus RNA-dependent RNA polymerase (hrvRdRP); and wherein the HRV 5 ′RR, cargo RNA molecule, and HRV 3′RR are operably linked. In another embodiment, the HRV amplicon includes, in 5′ to 3′ order, a subgenomic promoter that is operably linked to (v) a cargo RNA molecule, wherein the subgenomic promoter is recognized by the hrvRdRP. Such HRV amplicons can be provided in either an isolated form or in a composition. In embodiments, the RNA molecule including the HRV amplicon can be provided to a plant, for example, by transcription in the plant from a recombinant DNA molecule encoding the RNA molecule that is transiently expressed in the plant or that is stably integrated into the plant's genome, or by delivery to the plant of an exogenous RNA molecule including the HRV amplicon, for example, by contacting a surface of the plant with the exogenous RNA molecule including the HRV amplicon, or by introducing the exogenous RNA molecule including the HRV amplicon into the plant's vascular system (e.g., by injection, infusion, petiole uptake, root uptake). In embodiments, the cargo RNA molecule includes at least one antiviral RNA (e.g., an antiviral inhibitory RNA or an RNA encoding an antiviral polypeptide) that provides the plant with resistance to at least one viral pathogen (which in some instances can be the heterologous RNA virus itself). Such embodiments are useful as antiviral treatments for plants, to prevent or decrease the severity of infection of a plant by a viral pathogen.

In certain embodiments, plants including those of the family Amaranthaceae that comprise Secoviridae satellite RNAs containing HRV amplicons disclosed herein can exhibit control of HRV that include Amaranthus leaf mottle virus, Beet black scorch virus, Beet chlorosis virus, Beet cryptic virus 1, Beet cryptic virus 2, Beet cryptic virus 3, Beet mild yellowing virus, Beet mosaic virus, Beet necrotic yellow vein virus, Beet pseudoyellows virus, Beet ringspot virus, Beet soil-borne mosaic virus, Beet soil-borne virus, Beet virus Q, Beet western yellows virus, Beet yellow stunt virus, Beet yellows virus, Spinach amalgavirus 1, Spinach latent virus, and Spinach temperate virus. In other embodiments, plants such as those of the family Amaranthaceae that comprise an Apple latent spherical virus satellite RNA containing an HRV amplicon disclosed herein can exhibit control of HRV that include Amaranthus leaf mottle virus, Beet black scorch virus, Beet chlorosis virus, Beet cryptic virus 1, Beet cryptic virus 2, Beet cryptic virus 3, Beet mild yellowing virus, Beet mosaic virus, Beet necrotic yellow vein virus, Beet pseudoyellows virus, Beet ringspot virus, Beet soil-borne mosaic virus. Beet soil-borne virus, Beet virus Q, Beet western yellows virus, Beet yellow stunt virus, Beet yellows virus, Spinach amalgavirus 1. Spinach latent virus, Spinach temperate virus. In certain embodiments, plants including those of the family Fabaceae that comprise Secoviridae satellite RNAs containing HRV amplicons disclosed herein can exhibit control of HRV that include Bean common mosaic necrosis virus, Bean common mosaic virus, Bean leafroll virus, Bean mild mosaic virus, Bean necrotic mosaic orthotospovirus, Bean pod mottle virus, Bean rugose mosaic virus, Bean yellow disorder virus, Bean yellow mosaic virus, Broad bean mottle virus, Broad bean necrosis virus, Broad bean stain virus, Broad bean true mosaic virus, Broad bean wilt virus 1, Broad bean wilt virus 2, Faba bean polerovirus 1, Southern bean mosaic virus, Southern cowpea mosaic virus, Soybean dwarf virus, Soybean latent spherical virus, Soybean mosaic virus, Soybean scleroulivirus 1, Soybean scleroulivirus 2, Soybean vein necrosis orthotospovirus, Soybean yellow common mosaic virus, Soybean yellow mottle mosaic virus, Soybean-associated deltaflexivirus 1, Lupinus mosaic virus. Peanut clump virus, Peanut mottle virus, Peanut stunt virus, Peanut yellow mosaic virus, Pea early-browning virus. Pea enation mosaic virus 1, Pea enation mosaic virus 2, Pea green mottle virus. Pea mild mosaic virus. Pea seed-borne mosaic virus, Pea stem necrosis virus, and Pea streak virus. In other embodiments, plants such as those of the family Fabaceae that comprise an Apple latent spherical virus satellite RNA containing an HRV amplicon disclosed herein can exhibit control of HRV that include Bean common mosaic necrosis virus. Bean common mosaic virus. Bean leafroll virus, Bean mild mosaic virus, Bean necrotic mosaic orthotospovirus, Bean pod mottle virus, Bean rugose mosaic virus, Bean yellow disorder virus, Bean yellow mosaic virus, Broad bean mottle virus, Broad bean necrosis virus, Broad bean stain virus, Broad bean true mosaic virus, Broad bean wilt virus 1, Broad bean wilt virus 2, Faba bean polerovirus 1, Southern bean mosaic virus, Southern cowpea mosaic virus, Soybean dwarf virus, Soybean latent spherical virus, Soybean mosaic virus, Soybean scleroulivirus 1, Soybean scleroulivirus 2, Soybean vein necrosis orthotospovirus, Soybean yellow common mosaic virus, Soybean yellow mottle mosaic virus, Soybean-associated deltaflexivirus 1, Lupinus mosaic virus, Peanut clump virus, Peanut mottle virus, Peanut stunt virus, Peanut yellow mosaic virus, Pea early-browning virus, Pea enation mosaic virus 1, Pea enation mosaic virus 2, Pea green mottle virus, Pea mild mosaic virus, Pea seed-borne mosaic virus, Pea stem necrosis virus, and Pea streak virus. In certain embodiments, plants including those of the family Cucurbitaceae that comprise Secoviridac satellite RNAs containing HRV amplicons disclosed herein can exhibit control of HRV that include Cucumber Bulgarian latent virus, Cucumber fruit mottle mosaic virus, Cucumber green mottle mosaic virus, Cucumber leaf spot virus, Cucumber mosaic virus, Cucumber mottle virus, Cucumber necrosis virus, Cucumber soil-borne virus, Cucumber vein yellowing virus. Cucumber vein-clearing virus, Cucumis melo alphaendornavirus, Cucurbit aphid-borne yellows virus, Cucurbit mild mosaic virus, Cucurbit vein banding virus, Cucurbit yellow stunting disorder virus, Melon aphid-borne yellows virus, Melon mild mottle virus, Melon necrotic spot virus, Melon rugose mosaic virus, Melon severe mosaic orthotospovirus, Melon tenuivirus, Melon yellow spot orthotospovirus, Melon yellowing-associated virus. Wild cucumber mosaic virus, and Wild melon banding virus. In other embodiments, plants such as those of the family Cucurbitaceae that comprise an Apple latent spherical virus satellite RNA containing an HRV amplicon disclosed herein can exhibit control of HRV that include Cucumber Bulgarian latent virus, Cucumber fruit mottle mosaic virus, Cucumber green mottle mosaic virus, Cucumber leaf spot virus, Cucumber mosaic virus, Cucumber mottle virus, Cucumber necrosis virus, Cucumber soil-borne virus, Cucumber vein yellowing virus, Cucumber vein-clearing virus, Cucumis melo alphaendornavirus, Cucurbit aphid-borne yellows virus, Cucurbit mild mosaic virus, Cucurbit vein banding virus, Cucurbit yellow stunting disorder virus, Melon aphid-borne yellows virus, Melon mild mottle virus, Melon necrotic spot virus, Melon rugose mosaic virus, Melon severe mosaic orthotospovirus, Melon tenuivirus, Melon yellow spot orthotospovirus, Melon yellowing-associated virus, Wild cucumber mosaic virus, and Wild melon banding virus. In certain embodiments, plants including those of the family Rosaceae that comprise Secoviridae satellite RNAs containing HRV amplicons disclosed herein can exhibit control of HRV that include Apple associated luteovirus, Apple chlorotic leaf spot virus, Apple dimple fruit viroid, Apple hammerhead viroid, Apple latent spherical virus, Apple luteovirus 1, Apple mosaic virus. Apple rubodvirus 1, Apple rubodvirus 2, Apple scar skin viroid, Apple stem grooving virus, Apple stem pitting virus, Tulare apple mosaic virus, and Pear blister canker viroid. In other embodiments, plants such as those of the family Rosaceae that comprise an Apple latent spherical virus satellite RNA containing an HRV amplicon disclosed herein can exhibit control of HRV that include Apple associated luteovirus, Apple chlorotic leaf spot virus, Apple dimple fruit viroid, Apple hammerhead viroid, Apple latent spherical virus, Apple luteovirus 1, Apple mosaic virus, Apple nubodvirus 1. Apple nubodvirus 2, Apple scar skin viroid. Apple stem grooving virus, Apple stem pitting virus, Tulare apple mosaic virus, and Pear blister canker viroid. In certain embodiments, plants including those of the family Rutaceae that comprise Secoviridae satellite RNAs containing HRV amplicons disclosed herein can exhibit control of HRV that include Citrus tristeza virus, Citrus vein enation virus, Citrus exocortis viroid, Hop stunt viroid, Citrus leprosis viruses, Citrus psorosis virus, Citrus yellow vein clearing virus, Citrus bent leaf viroid, Citrus dwarfing viroid, Citrus bark-cracking viroid, Citrus yellow vein-associated virus, Citrus leaf rugose virus, Citrus variegation virus, Citrus yellow mottle-associated virus, Indian citrus ringspot virus, Citrus leaf blotch virus, Citrus sudden death-associated virus, and Citrus coguvirus. In other embodiments, plants such as those of the family Rutaceae that comprise an Apple latent spherical virus satellite RNA containing an HRV amplicon disclosed herein can exhibit control of HRV that include Citrus tristeza virus, Citrus vein enation virus, Citrus exocortis viroid, Hop stunt viroid, Citrus leprosis viruses, Citrus psorosis virus, Citrus yellow vein clearing virus, Citrus bent leaf viroid, Citrus dwarfing viroid, Citrus bark-cracking viroid, Citrus yellow vein-associated virus, Citrus leaf rugose virus, Citrus variegation virus, Citrus yellow mottle-associated virus, Indian citrus ringspot virus. Citrus leaf blotch virus, Citrus sudden death-associated virus, and Citrus coguvirus. In certain embodiments, plants including those of the family Solanaceae that comprise Secoviridae satellite RNAs containing HRV amplicons disclosed herein can exhibit control of HRV that include Tomato apical stunt viroid. Tomato aspermy virus, Tomato black ring virus, Tomato blistering mosaic tymovirus. Tomato bushy stunt virus, Tomato chlorosis virus. Tomato chlorotic dwarf viroid. Tomato chlorotic spot orthotospovirus, Tomato fruit blotch virus, Tomato infectious chlorosis virus, Tomato mild mottle virus, Tomato mosaic virus, Tomato necrotic streak virus, Tomato necrotic stunt virus, Tomato planta macho viroid, Tomato spotted wilt orthotospovirus, Tomato torrado virus, Tomato yellow ring orthotospovirus, Tomato zonate spot orthotospovirus, Peru tomato mosaic virus, Wild tomato mosaic virus, Wild potato mosaic virus, Andean potato latent virus, Andean potato mild mosaic virus, Andean potato mottle virus, Colombian potato soil-borne virus, Potato aucuba mosaic virus, Potato black ringspot virus, Potato latent virus, Potato leafroll virus, Potato mop-top virus, Potato necrosis virus, Potato spindle tuber viroid, Potato virus A, Potato virus B, Potato virus H, Potato virus M, Potato virus P, Potato virus S, Potato virus T, Potato virus U, Potato virus V. Potato virus X, Potato virus Y, Potato yellow blotch virus, Potato yellow vein virus, Eggplant latent viroid, Eggplant mosaic virus, Eggplant mottled crinkle virus, Bell pepper alphaendornavirus, Bell pepper mottle virus, Belladonna mottle virus, Paprika mild mottle virus, Hot pepper alphaendornavirus, Moroccan pepper virus, Obuda pepper virus, Pepper chat fruit viroid. Pepper chlorotic spot orthotospovirus, Pepper cryptic virus 1, Pepper cryptic virus 2, Pepper mild mottle virus. Pepper mottle virus, Pepper ringspot virus, Pepper severe mosaic virus. Pepper vein yellows virus 1. Pepper vein yellows virus 2. Pepper vein yellows virus 3, Pepper vein yellows virus 4, Pepper vein yellows virus 5, Pepper vein yellows virus 6, Pepper veinal mottle virus, Tobacco albetovirus 1, Tobacco albetovirus 2. Tobacco albetovirus 3. Tobacco bushy top virus, Tobacco etch virus, Tobacco latent virus, Tobacco mild green mosaic virus. Tobacco mosaic virus. Tobacco mosqueado virus. Tobacco mottle leaf curl virus, Tobacco mottle virus, Tobacco necrosis virus A. Tobacco necrosis virus D, Tobacco necrotic dwarf virus, Tobacco rattle virus, Tobacco ringspot virus, Tobacco streak virus, Tobacco vein banding mosaic virus, Tobacco vein distorting virus, Tobacco vein mottling virus, Tobacco virtovirus 1, Tobacco virus 1, Ethiopian tobacco bushy top virus, Cucumber mosaic virus, and Pepino mosaic virus. In other embodiments, plants such as those of the family Solanaceae that comprise an Apple latent spherical virus satellite RNA containing an HRV amplicon disclosed herein can exhibit control of HRV that include Tomato apical stunt viroid, Tomato aspermy virus, Tomato black ring virus, Tomato blistering mosaic tymovirus, Tomato bushy stunt virus, Tomato chlorosis virus, Tomato chlorotic dwarf viroid, Tomato chlorotic spot orthotospovirus, Tomato fruit blotch virus, Tomato infectious chlorosis virus, Tomato mild mottle virus, Tomato mosaic virus, Tomato necrotic streak virus, Tomato necrotic stunt virus, Tomato planta macho viroid, Tomato spotted wilt orthotospovirus, Tomato torrado virus, Tomato yellow ring orthotospovirus, Tomato zonate spot orthotospovirus, Peru tomato mosaic virus, Wild tomato mosaic virus, Wild potato mosaic virus, Andean potato latent virus, Andean potato mild mosaic virus, Andean potato mottle virus, Colombian potato soil-borne virus, Potato aucuba mosaic virus, Potato black ringspot virus, Potato latent virus. Potato leafroll virus. Potato mop-top virus, Potato necrosis virus. Potato spindle tuber viroid. Potato virus A, Potato virus B, Potato virus H, Potato virus M, Potato virus P, Potato virus S, Potato virus T, Potato virus U, Potato virus V, Potato virus X, Potato virus Y, Potato yellow blotch virus, Potato yellow vein virus. Eggplant latent viroid, Eggplant mosaic virus. Eggplant mottled crinkle virus, Bell pepper alphaendornavirus. Bell pepper mottle virus. Belladonna mottle virus. Paprika mild mottle virus, Hot pepper alphaendornavirus, Moroccan pepper virus, Obuda pepper virus, Pepper chat fruit viroid, Pepper chlorotic spot orthotospovirus, Pepper cryptic virus 1, Pepper cryptic virus 2, Pepper mild mottle virus, Pepper mottle virus, Pepper ringspot virus, Pepper severe mosaic virus, Pepper vein yellows virus 1, Pepper vein yellows virus 2, Pepper vein yellows virus 3, Pepper vein yellows virus 4, Pepper vein yellows virus 5, Pepper vein yellows virus 6, Pepper veinal mottle virus, Tobacco albetovirus 1, Tobacco albetovirus 2, Tobacco albetovirus 3, Tobacco bushy top virus, Tobacco etch virus, Tobacco latent virus, Tobacco mild green mosaic virus, Tobacco mosaic virus, Tobacco mosqueado virus, Tobacco mottle leaf curl virus, Tobacco mottle virus, Tobacco necrosis virus A, Tobacco necrosis virus D, Tobacco necrotic dwarf virus. Tobacco rattle virus. Tobacco ringspot virus, Tobacco streak virus, Tobacco vein banding mosaic virus, Tobacco vein distorting virus, Tobacco vein mottling virus, Tobacco virtovirus 1, Tobacco virus 1, Ethiopian tobacco bushy top virus, Cucumber mosaic virus, and Pepino mosaic virus. In certain embodiments, plants including those of the family Caricaceae that comprise Secoviridae satellite RNAs containing HRV amplicons disclosed herein can exhibit control of HRV that include Papaya leaf distortion mosaic virus, Papaya lethal yellowing virus. Papaya mosaic virus, and Papaya ringspot virus. In other embodiments, plants such as those of the family Caricaceae that comprise an Apple latent spherical virus satellite RNA containing an HRV amplicon disclosed herein can exhibit control of HRV that include Papaya leaf distortion mosaic virus, Papaya lethal yellowing virus, Papaya mosaic virus, and Papaya ringspot virus. In certain embodiments, plants including those of the family Brassicaceae that comprise Secoviridae satellite RNAs containing HRV amplicons disclosed herein can exhibit control of HRV that include Arabidopsis thaliana Arti virus, Arabidopsis thaliana Athila virus, Arabidopsis thaliana AtRE1 virus, Arabidopsis thaliana Endovir virus, Arabidopsis thaliana evelknievel virus, Arabidopsis thaliana Tal virus, Arabidopsis thaliana Tat4 virus, Brassica oleracea Melmoth virus, Turnip crinkle virus, Turnip mosaic virus, Turnip rosette virus, Turnip vein-clearing virus, Turnip yellow mosaic virus, Turnip yellows virus, and Radish mosaic virus. In other embodiments, plants such as those of the family Brassicaceae that comprise an Apple latent spherical virus satellite RNA containing an HRV amplicon disclosed herein can exhibit control of HRV that include Arabidopsis thaliana Art1 virus, Arabidopsis thaliana Athila virus, Arabidopsis thaliana AtRE1 virus, Arabidopsis thaliana Endovir virus, Arabidopsis thaliana evelknievel virus, Arabidopsis thaliana Tal virus, Arabidopsis thaliana Tat4 virus, Brassica oleracea Melmoth virus, Turnip crinkle virus, Turnip mosaic virus, Turnip rosette virus, Turnip vein-clearing virus, Turnip yellow mosaic virus, Turnip yellows virus, and Radish mosaic virus. Without seeking to be limited by theory, RNA molecules that contain HRV amplification sequences (such as the HRV amplicons described herein which contain either (1) a pair of HRV 5′ and 3′ RNA replication regions, or (2) a subgenomic promoter that is recognized by the hrvRdRP) potentially also serve as a “sponge” or “decoy” that reduces the corresponding hrvRdRP's efficiency in recognizing and amplifying the HRV viral genome itself, thus potentially decreasing a pathogenic HRV's deleterious effects on an infected plant.

RNA polynucleotides comprising at least one cleavable sequence are provided. In certain embodiments, the at least one cleavable sequence is located: (i) 5′ to the 5′ RNA replication element or 3′ to the 3′ RNA replication element; and/or (ii) between the 3′ end of the 5′ RNA replication element and the HRV amplicon and/or between the HRV amplicon and the 5′ end of the 3′ RNA replication element. In certain embodiments, the cleavable sequence is a self-cleaving ribozyme (e.g., a hammerhead ribozyme; Tang and Breaker. Proc Natl Acad Sci USA. 2000 May 23; 97(11):5784-9. doi: 10.1073/pnas.97.11.5784), a self-cleaving inducible ribozyme, or an siRNA or miRNA recognition site.

In an embodiment, a cargo RNA molecule that is integrated into a polynucleotide includes at least one CRISPR guide RNA; release of the guide RNA is mediated, e.g., by flanking DR sequences, ribozyme sequences, or other self-cleaving or trans-cleaving RNAs, or by cleavage by an endogenous ribonuclease. The corresponding Cas nuclease can be provided by separate or concurrent delivery, e.g., by co-delivery with a vector or polynucleotide, or by transient or stable expression of the corresponding Cas nuclease in the cell to which the polynucleotide is delivered. For many Cas nucleases, guide sequence designs are constrained by the requirement that the DNA target sequence (to which the crRNA is designed to be complementary) must be adjacent to a proto-spacer adjacent motif (“PAM”) sequence that is recognized by the specific Cas nuclease to be employed. Cas nucleases recognize specific PAM sequences and there is a diversity of nucleases and corresponding PAM sequences; see, e.g., Smakov et al. (2017) Nature Reviews Microbiol., doi:10.1038/nrmicro.2016.184. For example, Cas9 nucleases cleave dsDNA, require a GC-rich PAM sequence located 3′ to the DNA target sequence to be targeted by the crRNA component of the guide RNA, and cleave leaving blunt ends. Cas12a nucleases cleave dsDNA, require a T-rich PAM sequence located 5′ to the DNA target sequence to be targeted by the crRNA component of the guide RNA, and cleave leaving staggered ends with a 5′ overhang. Cas13 nucleases cleave single-stranded RNAs and do not require a PAM sequence; instead, Cas13 nuclease are guided to their targets by a single crRNA with a direct repeat (“DR”). In practice, the crRNA component of a guide RNA is generally designed to have a length of between 17-24 nucleotides (frequently 19, 20, or 21 nucleotides) and exact complementarity (i. e., perfect base-pairing) to the targeted gene or nucleic acid sequence that is itself adjacent to a PAM motif (when required by the Cas nuclease). A crRNA component having less than 100% complementarity to the target sequence can be used (e. g., a crRNA with a length of 20 nucleotides and between 1-4 mismatches to the target sequence) but this increases the potential for off-target effects.

Non-limiting examples of effective guide RNA design are found, e.g., in US Patent Application Publications US 2019/0032131, 2015/0082478, and 2019/0352655, which are each incorporated by reference in their entirety herein. For the purposes of gene editing, CRISPR “arrays” can be designed to include one or multiple guide RNA sequences corresponding to one or more desired target DNA sequence(s); see, for example, Cong et al. (2013) Science, 339:819-823; Ran et al. (2013) Nature Protocols, 8:2281-2308.

In certain embodiments, the 5′ RNA replication element includes a 5′ UTR element of a Secoviridae genome (e.g., a Secoviridae genome including a Secoviridae 5′ UTR set forth in Table 1). In embodiments, the 5′ RNA replication element further includes a genomic sequence of the Secoviridae that is natively located 3′ to and optionally adjacent or immediately adjacent to the 5′ UTR sequence. In embodiments, the 3′ RNA replication element includes a 3′ UTR sequence of a Secoviridae genome (e.g., a Secoviridae genome including a Secoviridae 3′ UTR set forth in Table 1). In embodiments, the 3′ RNA replication element further includes a genomic sequence of the Secoviridae that is natively located 5′ to and optionally adjacent or immediately adjacent to the 3′ UTR sequence.

In other embodiments, the RNA molecule further includes at least one RNA molecule encoding a viral MP. In certain embodiments, the at least one RNA molecule encoding an MP is located (a) before the cargo RNA molecule, (b) after the cargo RNA molecule, or (c) both before and after the cargo RNA molecule. In embodiments, the at least one RNA sequence encoding an MP includes at least two RNA sequences encoding different MPs or a single RNA sequence encoding multiple copies of MPs.

In some embodiments, the recombinant DNA molecule further includes a discrete expression cassette including a second promoter that is functional in the cell and is operably linked to a DNA sequence encoding at least one viral movement protein, and optionally a terminator element.

In some embodiments, the RNA molecule further includes an encapsidation recognition element (ERE), where the ERE is located close to or adjacent to the 3′ RNA replication element, and optionally wherein the 3′ RNA replication element includes a 3′ UTR sequence of the virus of the family Secoviridae. In embodiments, the ERE includes a viral OAS such as a tobacco mosaic virus OAS (TMV-OAS) or an OAS set forth in Table 2.

In embodiments, the RNA molecule further includes at least one tRNA-like sequence (TLS), and wherein the at least one tRNA-like sequence includes a tRNA-like sequence from an Arabidopsis FT mRNA (e.g. a TLS in an Arabidopsis FT mRNA of Table 4). In some embodiments, the RNA molecule includes a tRNA-like sequence encoded by a DNA sequence selected from the group consisting of SEQ ID NOs: 76-123, and 466. In some embodiments, the RNA molecule includes a modified tRNA-like sequence that has at least 90% sequence identity to a scaffold tRNA-like sequence encoded by a DNA sequence selected from the group consisting of SEQ ID NOs: 76-123, and 466 and that maintains the secondary structure of the scaffold tRNA-like sequence. In still other embodiments, the RNA molecule further includes at least one RNA encoding a viral MP, a tRNA-like sequence from an Arabidopsis FT mRNA, and an encapsidation recognition sequence including TMV-OAS.

In some embodiments, the cargo RNA molecule is up to about 8 kb in length. In embodiments, the cargo RNA molecule includes: (a) at least one coding sequence, (b) at least one non-coding sequence, or (c) both at least one coding sequence and at least one non-coding sequence. In other embodiments, the cargo RNA molecule includes at least one coding sequence, and wherein the RNA molecule further includes an internal ribosome entry site (IRES) located 5′ and immediately adjacent to the at least one coding sequence. In other embodiments, the cargo RNA molecule includes multiple coding sequences. and wherein the RNA molecule further includes an IRES located 5′ and immediately adjacent to each of the coding sequences.

In other embodiments, the cargo RNA molecule includes at least one non-coding sequence, and wherein the at least one non-coding sequence is selected from the group consisting of a hairpin RNA (hpRNA); an RNA that forms multiple stem-loops; an RNA pseudoknot; an RNA sequence that forms at least partially double-stranded RNA; a small interfering RNA (siRNA) or siRNA precursor; a microRNA (miRNA) or miRNA precursor; a ribozyme; a ligand-responsive ribozyme (aptazyme); an RNA aptamer; and a long noncoding RNA (lncRNA).

In embodiments, a DNA sequence encoding at least one ribozyme is provided. In embodiments, the at least one ribozyme is located 5′ to the 5′ RNA replication element or 3′ to the 3′ RNA replication element. In embodiments, a DNA sequence encoding at least one ligand-responsive ribozyme (aptazyme) is provided. In embodiments, the at least one ligand-responsive ribozyme is located 5′ to the 5′ RNA replication element or 3′ to the 3′ RNA replication element.

Recombinant RNA molecules comprising the aforementioned or otherwise disclosed 5′ RNA replication elements, a cargo RNA molecule(s), and 3′ RNA replication elements, as well as additional aforementioned or otherwise disclosed elements are also provided herein. In certain embodiments, the recombinant RNA molecules are produced by a recombinant DNA molecule provided herein. In certain embodiments, the recombinant RNA molecules are produced by an in vivo or in vitro (e.g., cell free) RNA replication process through the action of a RdRP acting on: (i) 5′ and 3′ RNA replication elements; and/or (ii) a subgenomic promoter.

Expression systems comprising the recombinant polynucleotides are also provided. Such expression systems include both cell-based and cell free expression systems. In certain embodiments, cell-free expression system can include (a) an RNA molecule comprising, in 5′ to 3′ order: (i) a 5′ RNA replication element; (ii) a cargo RNA molecule; and (iii) a 3′ RNA replication element; and, optionally, further comprising at least one additional RNA or other element. Embodiments of additional elements include at least one RNA encoding a viral MP, at least one tRNA-like sequence, an OAS, an RdRP protein that recognizes the 5′ and 3′ RNA replication elements, a subgenomic promoter, and/or an RdRP that recognizes an HRV 5′ or 3′ replication region and/or a subgenomic promoter. In some embodiments, the RdRP protein is provided by a virus of the family Secoviridae, e.g. a virus of the family Secoviridae that is endogenous to a cell in which expression is desired. In certain embodiments, cell-based expression system can include (a) a recombinant DNA molecule including a heterologous promoter that is functional in a cell and is operably linked to a DNA sequence encoding an RNA molecule comprising, in 5′ to 3′ order: (i) a 5′ RNA replication element; (ii) a cargo RNA molecule; and (iii) a 3′ RNA replication element; and, optionally, further comprising at least one additional RNA or other element. Embodiments of additional elements include at least one RNA encoding a viral MP, at least one tRNA-like sequence, an OAS, an RdRP protein that recognizes the 5′ and 3′ RNA replication elements, a subgenomic promoter, and/or an RdRP that recognizes the subgenomic promoter. In certain embodiments, cell-based expression system can include (a) a recombinant RNA molecule comprising, in 5′ to 3′ order: (i) a 5′ RNA replication element; (ii) a cargo RNA molecule; and (iii) a 3′ RNA replication element; and, optionally, further comprising at least one additional RNA or other element. Embodiments of additional RNA elements include at least one RNA encoding a viral MP, at least one tRNA-like sequence, an OAS, an RdRP protein that recognizes the 5 and 3′ RNA replication elements, a subgenomic promoter, and/or an RdRP that recognizes an HRV 5′ or 3′ replication region and/or a subgenomic promoter. In some embodiments, the RdRP protein is provided by a virus of the family Secoviridae. In some embodiments, an RdRP that recognizes an HRV 5′ or 3′ replication region and/or a subgenomic promoter is provided by a heterologous RNA virus (HRV) or by another nucleic acid introduced into the cell (e.g., by a vector or other recombinant nucleic acid). In still other embodiments, the 5′ RNA replication element and the 3′ RNA replication element are obtained from the same virus of the family Secoviridae and/or from the same Secoviridae genome (e.g., both obtained from the same Secoviridae capsid genome or both obtained from the same Secoviridae genome) or from Secoviridae genomes having at least 85%, 90%, 95%, 98%, or 99% sequence identity to one another and which are optionally related. In some embodiments, a cell used in the expression system is a bacterial cell, a plant cell, a fungal cell, or an animal cell (e.g., an insect cell). In embodiments, a cell used in the expression system endogenously contains a virus of the family Secoviridae having a genome that encodes an RdRP that recognizes the 5′ and 3′ RNA replication elements. In some embodiments, the expression system further includes a viral capsid protein that is recognized by the encapsidation recognition element and encapsidates the RNA molecule. In some embodiments, the viral capsid protein is: (a) expressed by the recombinant DNA molecule in the cell (e.g., where the recombinant DNA molecule further includes a discrete expression cassette comprising a second promoter operably linked to a DNA sequence encoding the viral capsid protein, and optionally a terminator element), (b) co-expressed by a second recombinant DNA molecule in the cell; (c) provided exogenously to the cell; or (d) expressed by a virus in the cell. In embodiments, the RdRP protein is heterologous to the cell. In embodiments, the RdRP protein is provided exogenously to the cell. In certain embodiments, the RdRP protein that recognizes the 5′ and 3′ RNA replication elements is endogenously expressed in the plant cell by the virus of the family Secoviridae (e.g., where the virus of the family Secoviridac occurs naturally in the plant cell). In embodiments, the virus of the family Secoviridae is native to or endemic to the plant cell. In embodiments, the virus of the family Secoviridae that is endemic to the plant cell is non-pathogenic. In embodiments, the virus of the family Secoviridae that is endemic to the plant cell is non-pathogenic and commensal. In embodiments, the virus of the family Secoviridae is an exogenously introduced virus of the family Secoviridae (i.e., not endemic or native to the host, but artificially introduced). For example, a virus of the family Secoviridae natively found in one plant species, variety, or germplasm can be introduced, with or without a corresponding recombinant Secoviridae satellite RNA, into a different plant species, variety, or germplasm. In embodiments, a complete self-replicating Secoviridae satellite system is introduced into a plant or plant cells, wherein the self-replicating Secoviridae satellite system includes: (1) a recombinant Secoviridae satellite RNA comprising, from 5′ terminus to 3′ terminus: (a) a 5′ RNA replication element recognized by a Secoviridae RNA-dependent RNA polymerase (RdRP); (b) a cargo RNA molecule; and (c) a 3′ RNA replication element recognized by the RdRP; wherein the 5′ RNA replication element, the cargo RNA molecule, and the 3′ RNA replication element are operably linked, and wherein the promoter and the cargo RNA molecule are heterologous to the 5′ RNA replication element and the 3′ RNA replication element; and (2) an exogenous virus of the family Secoviridae (e.g., a virus of the family Secoviridae that is not endemic or native to the plant or plant cells) that is capable of replication in the plant or plant cells and that encodes the Secoviridae RdRP that recognizes the 5′ and 3 replicase recognition sequences in the recombinant Secoviridae satellite RNA. In embodiments, the Secoviridae RdRP comprises a protein having at least 85%, 90%, 95%, 97%, or 99% sequence identity to SEQ ID NO: 471, 530, or 558-564. In embodiments, the recombinant DNA molecule or recombinant RNA molecule further comprises at least one RNA encoding a viral MP, a tRNA-like sequence from an Arabidopsis FT mRNA, and an encapsidation recognition sequence including a TMV-OAS.

Cells comprising any of the aforementioned or otherwise disclosed recombinant polynucleotides (e.g., recombinant DNAs, recombinant RNAs, or vectors comprising or encoding the same) are provided herein. Cells comprising the recombinant polynucleotides include prokaryotic (e.g., a bacterium, such as a bacterium capable of transforming a eukaryotic cell) or eukaryotic (e.g., a plant cell, fungal cell, or animal cell such as an insect cell) cells. In certain embodiments, the cells are bacterial cells capable of transforming a plant cell (e.g., an Agrobacterium sp., a Sinorhizobium sp., a Mesorhizobium sp., Bradyrhizobium sp., Rhizobium sp., or an Ensifer sp. cell. Bacterial cells capable of transforming a plant cell suitable for use with the recombinant polynucleotides provided herein include Agrobacterium sp., a Sinorhizobium sp., Mesorhizobium sp., Bradvrhizobium sp., Rhizobium sp., or an Ensifer sp. cell are disclosed in US patent application publications US20170369898 and US20180312854, each incorporated herein by reference in their entireties.

Vectors suitable for maintenance, propagation, and/or expression of the recombinant polynucleotides in the aforementioned prokaryotic or eukaryotic cells are also provided herein. Such vectors can comprise any of the aforementioned or otherwise disclosed recombinant polynucleotides, recombinant DNA molecules, and recombinant RNA molecules as well as those polynucleotides molecules described in the Examples. In some embodiments, the bacterium that mediates the plant transfonnation is an Agrobacterium sp., a Sinorhizobium sp., a Mesorhizobium sp., Bradyrhizobium sp., Rhizobium sp., or an Ensifer sp. In embodiments where an Agrobacterium sp. is used, the vector includes T-DNAs flanking the recombinant DNA molecule encoding the recombinant RNA molecule (e.g., as described in US patent application publications US20170369898 and US20180312854, each incorporated herein by reference in their entireties). In some embodiments, the vector is contained within a plant cell or within a bacterial cell (Agrobacterium sp., a Sinorhizobium sp., a Mesorhizobium sp., Bradyrhizobium sp., Rhizobium sp., or an Ensifer sp. cell).

Viral particles comprising any of the aforementioned or otherwise disclosed recombinant RNA molecules are also provided. In one embodiment, the recombinant RNA is introduced into a host or production plant by using Agrobacterium-mediated transformation with a polynucleotide comprising (5′ to 3′): (i) a promoter which is operably linked to a viral MP coding sequence and a TLS element, flanked by Secoviridae 5′ and 3′ RNA replication elements; (ii) a promoter which is operably linked to a cargo RNA molecule and a TLS element, flanked by Secoviridae 5′ and 3′ RNA replication elements; (iii) a promoter operably linked to an RdRP coding sequence; and (iv) a promoter operably linked to a CP encoding sequence. Heterologous promoters independently drive expression of the capsid protein and the cargo as depicted. The RNA expressed from the polynucleotide includes an OAS. A host plant is transformed for production of the synthetic Secoviridae satellite RNA and satellite particles comprising the encapsidated RNA. The expressed and encapsidated Secoviridae satellite RNA is subsequently isolated from leaf material or other tissue of the host plant, purified (and, if desired, formulated) for high pressure spraying onto plants that endogenously contain the corresponding virus of the family Secoviridae or a recombinant (e.g., stably transformed or transiently expressed in the plant) source of the Secoviridae RdRP for subsequent expression and replication of the Secoviridae satellite RNA and satellite particles comprising the same in encapsidated form. In certain embodiments, spraying with the encapsidated satellite particles with certain cargo RNA molecules can be used to modify the plant as desired. The presence of a movement protein and/or tRNA-like sequences facilitates systemic movement throughout the plant receiving the high-pressure spray satellite particles comprising the desired recombinant RNA molecules comprising the encoded MP and cargo RNA. In certain embodiments, plants without a systemic virus of the family Secoviridae which provides the Secoviridae RdRP can further comprise a recombinant DNA or RNA molecule which encodes and provides the RdRP., e.g., stably integrated into the plant's genome or transiently expressed in the plant. A plant that transgenically or transiently expresses a Secoviridae RdRP is also useful for evaluating recombinant Secoviridae satellites in planta.

Target plants and plant cells used as hosts for synthetic Secoviridae satellite RNAs (e.g., recombinant RNAs) provided herein include both monocot and dicot plants and plant cells which can support Secoviridae replication. In embodiments, the virus of the family Secoviridae is endogenous to (endemic to or natively found in) the plant or plant cell. In other embodiments, the virus of the family Secoviridae is introduced to and becomes established in the plant or plant cell. Embodiments include row crop plants, fruit-producing plants and trees, vegetables, trees, and ornamental plants including ornamental flowers, shrubs, trees, groundcovers, and turf grasses. In certain embodiments, the host plants and plant cells for synthetic Secoviridae satellite RNAs include row crop plants, fruit-producing plants and trees, vegetables, trees, and ornamental plants including ornamental flowers, shrubs, trees, groundcovers, and turf grasses. In embodiments, the host plants and plant cells for synthetic Secoviridae satellite RNAs include commercially important cultivated crops, trees, and plants, including: alfalfa (Medicago sativa), almonds (Prunus dulcis), apples (Malus x domestica), apricots (Prunus armeniaca, P. brigantine, P. mandshurica, P. mume, P. sibirica), asparagus (Asparagus officinalis), bananas (Musa spp.), barley (Hordeum vulgare), beans (Phaseolus spp.), blueberries and cranberries (Vaccinium spp.), cacao (Theobroma cacao), canola and rapeseed or oilseed rape, (Brassica napus), Polish canola (Brassica rapa), and related cruciferous vegetables including broccoli, kale, cabbage, and turnips (Brassica carinata, B. juncea, B. oleracea, B. napus, B. nigra, and B. rapa, and hybrids of these), carnation (Dianthus caryophyllus), carrots (Daucus carota sativus), cassava (Manihot esculentum), cherry (Prunus avium), chickpea (Cicer arietinum), chicory (Cichorium intybus), chili peppers and other capsicum peppers (Capsicum annuum, C. frutescens, C. chinense, C. pubescens, C. baccatum), chrysanthemums (Chrysanthemum spp.), coconut (Cocos nucifera), coffee (wild and domesticated Coffea spp. including Coffea arabica, Coffea canephora, and Coffea liberica), cotton (Gossypium hirsutum L.), cowpea (Vigna unguiculata and other Vigna spp.), fava beans (Vicia faba), cucumber (Cucumis sativus), currants and gooseberries (Ribes spp.), date (Phoenix dactylifera), duckweeds (family Lemnoideae), eggplant or aubergine (Solanum melongena), eucalyptus (Eucalyptus spp.), flax (Linum usitatissumum L.). geraniums (Pelargonium spp.), grapefruit (Citrus x paradisi), grapes (_{Vitus} spp.) including wine grapes (Vitus vinifera and hybrids thereof), guava (Psidium guajava), hops (Humulus lupulus), hemp and cannabis (Cannabis sativa and Cannabis spp.), irises (Iris spp.), lemon (Citrus limon), lettuce (Lactuca sativa), limes (Citrus spp.), maize (Zea mays L.), mango (Mangifera indica), mangosteen (Garcinia mangostana), melon (Cucumis melo), millets (Setaria spp.. Echinochloa spp., Eleusine spp., Panicum spp., Pennisetum spp.), oats (Avena sativa), oil palm (Ellis quineensis), olive (Olea europaea), onion (Allium cepa) and other alliums (Allium spp.), orange (Citrus sinensis), papaya (Carica papava), peaches and nectarines (Prunus persica), pear (Pyrus spp.), pea (Pisa sativum), peanut (Arachis hypogaea), peonies (Paeonia spp.), petunias (Petunia spp.), pineapple (Ananas comosus), plantains (Musa spp.), plum (Prunus domestica), poinsettia (Euphorbia pulcherrima), poplar (Populus spp.), potato (Solanum tuberosum), pumpkins and squashes (Cucurbita pepo, C. maxima, C. moschata), rice (Oryza sativa L.), roses (Rosa spp.), rubber (Hevea brasiliensis), rye (Secale cereale), safflower (Carthamus tinctorius L), sesame seed (Sesame indium), sorghum (Sorghum bicolor), soybean (Glycine max L.), strawberries (Fragaria spp., Fragaria x ananassa), sugar beet (Beta vulgaris), sugarcanes (Saccharum spp.), sunflower (Helianthus annuus), sweet potato (Ipomoea batatas), tangerine (Citrus tangerina), tea (Camellia sinensis), tobacco (Nicotiana tabacum L.), tomato (Solanum lycopersicum or Lycopersicon esculentum), tulips (Tulipa spp.), walnuts (Juglans spp. L.), watermelon (Citrullus lanatus), wheat (Triticum aestivum), and yams (Discorea spp.). Wild relatives of domesticated plants are also of interest. In certain embodiments, the host plant or plant cells for synthetic Secoviridae satellite RNAs is a dicot plant or plant cell selected from the genera Brassica, Capsicum, Cucumis, Cucurbita, Gossypium, Nicotiana, Solanum, or Glycine. In certain embodiments, the host plant or plant cells for synthetic Secoviridae satellite RNAs is a monocot plant or plant cell selected from the genera Avena, Hordeum, Oryza, Secale. Triticum, Sorghum, or Zea. In certain embodiments, monocot target plants and plant cells used as hosts for synthetic Secoviridae satellite RNAs (e.g., recombinant RNAs) provided herein include oats (Avena sativa), barley (Hordeum vulgare), rice (Oryza sativa, Oryza glaberrima, Oryza rufipogen), rye (Secale cereale), wheat (Triticum aestivum), sorghum (Sorghum bicolor), and maize (Zea mays) plants and plant cells.

Methods of using or use of any of the aforementioned or otherwise disclosed recombinant polynucleotides, expression systems, cells, and/or vectors to: (i) provide a synthetic Secoviridae satellite RNA to a plant cell, (ii) obtain a phenotypic change in a plant or plant cell; (ii) increase a plant's resistance of a pest or pathogen, (iii) increase a plant's resistance to stress, (iv) express a polypeptide in a plant or plant cell, and/or (v) manufacture a synthetic Secoviridae virus particle are also provided. In certain embodiments of the methods, the recombinant RNA molecule or a formulation thereof is provided by contacting the plant or plant cell with the recombinant RNA molecule or formulation thereof. In other embodiments, the recombinant RNA molecule is provided by expressing in the plant or plant cell a DNA molecule that encodes the recombinant RNA molecule or a formulation. In other embodiments, the recombinant RNA molecule is provided by contacting the plant or plant cell with cells (such as bacterial cells) which comprise a DNA molecule that encodes the recombinant RNA molecule and are capable of transforming the plant or plant cell. In other embodiments, the recombinant RNA molecule is provided by contacting the plant or plant cell with a satellite particle comprising an encapsidated recombinant RNA molecule or a formulation thereof. In embodiments, the 5′ RNA replication element has a nucleotide sequence obtained or derived from a Secoviridae genomic sequence; and/or the 3′ RNA replication element has a nucleotide sequence obtained or derived from a Secoviridae genomic sequence. In certain embodiments, the 5′ and/or 3′ RNA replication element can be obtained from the corresponding Secoviridae genomic sequence by synthesizing or cloning a copy of the corresponding Secoviridae genomic sequence. In certain embodiments, the 5′ and/or 3′ RNA replication element can be derived from the corresponding Secoviridae genomic sequence by synthesizing a copy of a modified Secoviridae genomic sequence or sequences. Such modifications of Secoviridae genomic sequences present in a derived sequence include: (i) substitutions of nucleotides which maintain the RNA secondary structure; (ii) substitution of nucleotides based on a consensus obtained by alignment of 5′ or 3′ RNA replication elements; (iii) insertions, deletions, and/or substitution of nucleotides to facilitate assembly and/or operable linkage to other elements in the satellite RNA which include cargo RNA molecules, tRNA-like elements, encapsidation recognition element (ERE), RNA encoding a viral movement protein (MP), IRES elements, an HRV 5′RR, HRV 3′RR, and/or HRV subgenomic promoter; or (iv) any combination of (i). (ii), or (iii). In certain embodiments, the plant cell includes the virus of the family Secoviridae, and the RdRP protein is provided to the plant cell by the virus of the family Secoviridae. In embodiments, the virus of the family Secoviridae is endemic to the plant cell. In embodiments, the virus of the family Secoviridae that is endemic to the plant cell is non-pathogenic and/or commensal to the plant cell. In other embodiments, the virus of the family Secoviridae is exogenously provided to the plant cell. In some embodiments, the RdRP protein is exogenously provided to the plant cell. In embodiments, the recombinant RNA molecule is produced in a fermentation system. In embodiments, the recombinant RNA molecule is provided to the plant cell by transcribing in the plant cell a recombinant DNA construct including a promoter functional in the plant cell and operably linked to a DNA sequence encoding the recombinant RNA molecule. In embodiments, the recombinant RNA molecule further includes an encapsidation recognition element (ERE), and the plant cell further includes a viral capsid protein (CP) capable of encapsidating the synthetic Secoviridae satellite RNA. In embodiments, wherein the viral capsid protein is exogenously provided to the plant cell. In other embodiments, the recombinant DNA construct further includes a DNA sequence encoding a viral capsid protein. Still in other embodiments, the recombinant DNA construct further includes a second promoter functional in the plant cell and operably linked to the DNA sequence encoding the viral capsid protein. In embodiments, the viral capsid protein is expressed in the plant cell and encapsidates the synthetic Secoviridae satellite RNA. In yet other embodiments, the plant cell includes the virus of the family Secoviridae, and the virus of the family Secoviridae provides to the plant cell: (a) the RdRP protein, (b) the viral capsid protein, or (c) both the RdRP protein and the viral capsid protein. In certain embodiments, the methods can further comprise a first step of providing a population of plants comprising the plant cells comprising: (i) the virus of the family Secoviridae which provides the RdRP; or (ii) recombinant polynucleotide molecule that encodes the RdRP; and then providing the recombinant RNA molecule to the plants comprising the plant cells. In certain embodiments, the methods can further comprise the step of determining if the plant cell comprises a virus of the family Secoviridae which can provide the RdRP. In embodiments wherein it is determined that the plant cell comprises the virus of the family Secoviridae which can provide the RdRP, the virus of the family Secoviridae, the RdRP protein, and/or the recombinant polynucleotide encoding the RdRP is optionally not exogenously provided to the plant cell. In other embodiments wherein it is determined that the plant cell does not comprise the virus of the family Secoviridae which can provide the RdRP and the virus of the family Secoviridae is exogenously provided to the cell, the RdRP protein or the recombinant polynucleotide encoding the RdRP is exogenously provided to the plant cell, or a combination of the virus of the family Secoviridae, RdRP protein, or polynucleotide encoding the RdRP is exogenously provided to the plant cell. In embodiments, a complete self-replicating Secoviridae satellite system is introduced into a plant or plant cells, wherein the self-replicating Secoviridae satellite system includes: (1) a recombinant Secoviridac satellite RNA comprising, from 5′ terminus to 3′ terminus: (a) a 5′ RNA replication element recognized by a Secoviridae RNA-dependent RNA polymerase (RdRP); (b) a cargo RNA molecule; and (c) a 3′ RNA replication element recognized by the RdRP; wherein the 5′ RNA replication element, the cargo RNA molecule, and the 3′ RNA replication element are operably linked, and wherein the promoter and the cargo RNA molecule are heterologous to the 5′ RNA replication element and the 3′ RNA replication element; and (2) an exogenous virus of the family Secoviridae (e.g., a virus of the family Secoviridae that is not endemic or native to the plant or plant cells) that is capable of replication in the plant or plant cells and that encodes the Secoviridae RdRP that recognizes the 5′ and 3′ replicase recognition sequences in the recombinant Secoviridae satellite RNA. In embodiments, the Secoviridae RdRP comprises a protein having at least 85%, 90%, 95%, 97%, or 99% sequence identity to SEQ ID NO: 471, 530, or 558-564. The presence or absence of a virus of the family Secoviridae in a target plant can be determined by an RNA detection assay (e.g., an RT-PCR assay) using nucleic acid probes and/or primers which can detect any part of a Secoviridae genome including a 5′ RNA replication element, a CP and/or RdRP coding region, and/or a 3′ RNA replication element. Such probes and primers include those which detect any of the 5′ or 3′ RNA replication elements set forth in Table 1 or having significant sequence identity thereto (e.g., at least about 80%, 85%, 90%, 95%, 98%, or 99% of a length of at least about 18, 20, 30, 40 or 50 nt). The presence or absence of a virus of the family Secoviridae in a target plant can be determined by a protein detection assay (e.g., an immunoassay) directed to a Secoviridae CP or RdRP (e.g., a CP or RdRP encoded by or homologous to a CP or RdRP encoded by a Secoviridae genome disclosed in Table 1). Target plants and plant cells used in the methods include all aforementioned target plants and plant cell hosts for synthetic Secoviridae satellite RNAs (e.g., recombinant RNAs).

In certain embodiments of any of the aforementioned or otherwise disclosed methods, the recombinant RNA that effects: (i) a phenotypic change in the plant or plant cell; (ii) increases a plant's resistance to a pest or pathogen; or (iii) increases a plant's resistance to stress can include an RNA for modulating a target gene's expression relative to the target gene's expression in a control plant or plant cell not provided with the recombinant RNA molecule, and the phenotypic change, increased resistance to the pest or pathogen, or increased resistance to stress is a result of the modulation. In embodiments, the modulation is (a) an increase of the target gene's expression; or (b) a decrease of the target gene's expression. In some embodiments, expression of the target gene is increased by up to about 1%, 2%, 3%, %, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a control plant or plant cell lacking the recombinant RNA). In certain embodiments, expression of the target gene is increased by up to about 2-, 3-, 4-, 5-, 6-, 8-, 9-, 10-fold, or more relative to a reference level (e.g., a level found in a control plant or plant cell lacking the recombinant RNA molecule). In some embodiments, expression of the target gene is decreased by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100% relative to a reference level (e.g., a level found in a level found in a control plant or plant cell lacking the recombinant RNA). In certain embodiments, expression of the target gene is decreased by up to about 2-, 3-, 4-, 5-, 6-, 8-, 9-, 10-fold, or more relative to a reference level (e.g., a level found in a control plant or plant cell lacking the recombinant RNA molecule). Modulation of target gene expression can be effected by one or more RNAs for modifying the genome, epigenome, and/or transcriptome of the plant or plant cell. RNAs for modifying the genome include gRNAs recognized by CAS nucleases, RNAs encoding TALENs or artificial zinc finger proteins (aZFN). RNAs for modifying the epigenome include RNAs which provide RNA directed DNA methylation such as in promoter regions of target genes (Matzke and Mosher (2014), doi: 10.1038/nrg3683). Embodiments of an RNA for modifying the transcriptome include one or more RNAs that comprise any of: a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased siRNA or phased siRNA precursor (see, e.g., U.S. Pat. No. 8,404,928): a ribozyme; a ligand-responsive ribozyme (aptazyme); an RNA aptamer; or a long noncoding RNA (lncRNA).

In any of the aforementioned or otherwise disclosed methods wherein a phenotypic change is obtained or effected in a plant or plant cell, the cargo RNA molecule can comprise an RNA that effects a phenotypic change in the plant or plant cell in comparison to a plant or plant cell lacking the recombinant RNA. In certain embodiments, phenotypes that are changed include developmental rate, growth rate, size, yield (e.g., intrinsic yield), vigor, photosynthetic capability, flavor, starch production, protein content, carbohydrate content, oil content, fatty acid content, lipid content, digestibility, biomass, shoot length, root length, root architecture, seed set, seed weight, seed quality (e.g., nutritional content), germination, fruit set, rate of fruit ripening, production of biopolymers, production of fibers, production of biofuels, production of pharmaceutical peptides (e.g., hormones, enzymes, transcription factors, antigens, antibodies, or antibody fragments), production of secretable peptides, enzyme production, improved processing traits, or amount of harvestable produce. In some embodiments, phenotypes that are changed include taste, appearance, or shelf-life of a product harvested from the plant. In other embodiments, phenotypes that are changed include flower size, flower color, flower patterning, flower morphology including presence or absence of stamens, flower number, flower longevity, flower fragrance, leaf size, leaf color, leaf patterning, leaf morphology, plant height, or plant architecture.

In any of the aforementioned or otherwise disclosed methods wherein a plant's resistance to a pest or pathogen is increased, the recombinant RNA can comprise an RNA that inhibits expression of a gene of the pest or pathogen and/or inhibits replication of the genome of the pest or pathogen. In certain embodiments, the pest or pathogen is selected from the group comprising: a bacterium, a virus other than a virus of the family Secoviridae, a fungus, an oomycete, and an invertebrate (e.g., an arthropod or a nematode). Target viruses other than a virus of the family Secoviridae include; (i) positive-strand RNA viruses in the Bromoviridae, Closteroviridae, Luteoviridae, or Potyviridae family; (ii) negative strand RNA viruses in the Bunvaviridae and Rhabdoviridae family; (iii) dsDNA viruses in the family Caulimoviridae; and (iv) ssDNA viruses in the family Geminiviridae. Target arthropods pests include coleopteran and lepidopteran insects. Target fungal pathogens include Magnaporthe spp., Botrytis spp., Puccinia spp.; Fusarium spp., Blumeria spp., Mycosphaerella spp., Colletotrichum spp., Ustilago spp., Melampsora spp., Phakopsora spp., Phytophthora spp., and Rhizoctonia spp. In embodiments, the cargo RNA molecule effects an increase in the plant's resistance to a pest or pathogen, relative to that in a plant not provided with the recombinant RNA molecule.

In any of the aforementioned or otherwise disclosed methods wherein a plant's resistance to stress is increased, the recombinant RNA can comprise an RNA that targets a plant gene which provides such resistance. In embodiments, the RNA that effects an increase in the plant's resistance to stress in the plant or plant cell includes an RNA for modulating the target gene's expression relative to the target gene's expression in a control plant or plant cell not provided with the recombinant RNA molecule, and wherein the increase stress resistance is a result of the modulation. In embodiments, the modulation is (a) an increase of the target gene's expression; or (b) a decrease of the target gene's expression. In still other embodiments, the RNA that effects an increase in the plant's resistance to stress in the plant or plant cell comprises a messenger RNA encoding a protein which confers the stress resistance. In still other embodiments, the messenger RNA includes an RNA sequence absent in the transcriptome of the plant or plant cell lacking the recombinant RNA. In embodiments, the stress includes at least one abiotic stress selected from the group including: nutrient stress, light stress, drought stress, heat stress, and cold stress. In other embodiments, the stress includes at least one biotic stress selected from the group including: crowding, shading, and allelopathy (e.g., resulting from allelopathic chemicals including a juglone produced by walnut trees).

In any of the aforementioned or otherwise disclosed methods wherein an exogenous polypeptide (e.g., an exogenous polypeptide) is expressed in a plant or plant cell, the cargo RNA can encode the exogenous polypeptide. In embodiments, the polypeptide is isolated (e.g., separated from at least one other cellular components such as a carbohydrate, a lipid, or another protein) or polypeptide is purified.

In any of the aforementioned or otherwise disclosed methods wherein a synthetic Secoviridae satellite particle is manufactured, such manufacture can occur in either a cell-based system or a cell-free system. Cell-based methods of manufacturing a synthetic Secoviridac satellite particle can comprise: (a) providing to a plant cell the recombinant RNA molecule, wherein the recombinant RNA molecule comprises an encapsidation recognition element (ERE), and wherein the plant cell comprises an RdRP protein that recognizes the 5′ RNA replication element and 3′ RNA replication element catalyzes synthesis of a synthetic Secoviridac satellite RNA from the recombinant RNA molecule and a viral capsid protein, wherein the ERE provides for encapsidation of the RNA by the viral capsid protein; and optionally isolating the synthetic Secoviridae satellite particle from the plant cell, a plant comprising the plant cell, or from media in which the plant cell or the plant had been grown. Cell-free methods of manufacturing a synthetic Secoviridae satellite particle include methods where the recombinant RNA molecule is combined with a viral capsid protein in a vessel, wherein the recombinant RNA molecule comprises an ERE, and wherein the ERE provides for encapsidation of the RNA by the viral capsid protein in the vessel; optionally wherein the method further comprises isolating the synthetic Secoviridae satellite particle from uncombined RNA and/or viral capsid protein in the vessel. In certain embodiments of either cell-based or cell-free methods, the synthetic satellite particle is isolated (e.g., separated from at least one other cellular components such as an organelle, a membrane, a carbohydrate, a lipid, or another protein) or is purified. In certain embodiments, the methods can further comprise formulating the synthetic Secoviridae satellite particle.

Synthetic Secoviridae satellite particles comprising the recombinant RNA, including those made by the aforementioned methods are also provided. Methods of providing any of the aforementioned synthetic Secoviridae satellite particles to a plant, including contacting (e.g., spraying, dusting, injecting, soaking, etc.) the plant with the synthetic Secoviridae satellite particle or a formulation thereof are also provided.

The recombinant polynucleotides, cells comprising the same, and synthetic Secoviridae satellite particles described herein can be formulated either in pure form (e.g., the composition contains only the recombinant polynucleotide) or together with one or more additional formulation components to facilitate application or delivery of the compositions. In embodiments, the additional formulation component includes, e.g., a carrier (i.e., a component that has an active role in delivering the active agent (e.g., recombinant polynucleotide); for example, a carrier can encapsulate, covalently or non-covalently modify, or otherwise associate with the active agent in a manner that improves delivery of the active agent) or an excipient (e.g., a delivery vehicle, adjuvant, diluent, surfactant, stabilizer, or tonicity agent). In some embodiments, the composition is formulated for delivery to a plant.

In some aspects, the disclosure provides a formulation comprising any of the compositions described herein. In some embodiments, the formulation is a liquid, a gel, or a powder. In some embodiments, the formulation is configured to be sprayed on plants, to be injected into plants (see, e.g., U.S. Pat. No. 11,844,318) or otherwise introduced into the vascular system of a plant, to be rubbed on leaves, to be soaked into plants, to be coated onto plants, or be coated on seeds, or to be delivered through root uptake (e.g., in a hydroponic system or via soil).

Depending on the intended objectives and prevailing circumstances, the composition can be formulated into emulsifiable concentrates, suspension concentrates, directly sprayable or dilutable solutions, coatable pastes, diluted emulsions, spray powders, soluble powders, dispersible powders, wettable powders, dusts, granules, encapsulations in polymeric substances, microcapsules, foams, aerosols, carbon dioxide gas preparations, tablets, resin preparations, paper preparations, nonwoven fabric preparations, or knitted or woven fabric preparations. In some instances, the composition is a liquid. In some instances, the composition is a solid. In some instances, the composition is an aerosol, such as in a pressurized aerosol can.

In some instances, the recombinant polynucleotide makes up about 0.1% to about 100% of the composition, such as any one of about 0.01% to about 100%, about 1% to about 99.9%, about 0.1% to about 10%, about 1% to about 25%, about 10% to about 50%, about 50% to about 99%, or about 0.1% to about 90% of active ingredients (e.g., recombinant polynucleotides). In some instances, the composition includes at least any of 0.1%, 0.5%, 1%, 5%, 10%, 20%, 30%, 40%, 50%, 60%, 70%, 80%, 90%, 95%, or more active ingredients (e.g., recombinant polynucleotides). In some instances, the concentrated agents are preferred as commercial products, the final user normally uses diluted agents, which have a substantially lower concentration of active ingredient.

In some embodiments, the composition is formulated for topical delivery to a plant. In some embodiments, the topical delivery is spraying, leaf rubbing (e.g., with or without an abrasive), soaking, coating (e.g., coating using micro-particulates or nano-particulates), or delivery through root uptake (e.g., delivery in a hydroponic system or by a root drench).

In some embodiments, the composition further comprises a carrier and/or an excipient. In other embodiments, the composition does not comprise a carrier or excipient, e.g., comprises a naked polynucleotide (e.g., a naked RNA).

In some embodiments, the recombinant polynucleotide is delivered at a concentration of at least 0.1 grams per acre, e.g., at least 0.1, 1, 2, 5, 10, 15, 20, 25, 30, 35, 40, 45, or 50 grams per acre. In some embodiments, less than 120 liters per acre is delivered, e.g., less than 110, 100, 90, 80, 70, 60, 50, 40, 30, 20, 15, 10, 5, or 2 liters per acre or less than 1 liter per acre.

In some aspects, the formulation comprises a carrier. In some embodiments the formulation is an emulsion or a reverse emulsion, a liquid, or a gel. In embodiments, the formulation includes a carrier that serves as a physical support (e.g., solid or semi-solid surfaces or matrices, powders, or particles or nanoparticles). In embodiments, the active agent is encapsulated or enclosed in or attached to or complexed with a carrier including a liposome, vesicle, micelle, or other fluid compartment. In embodiments, the active agent is encapsulated or enclosed in or attached to or complexed with a carrier including a naturally occurring or synthetic, branched or linear polymer (e.g., pectin, agarose, chitin, chitosan, DEAE-dextran, polyvinylpyrrolidone (“PVP”), or polyethylenimine (“PEI”). In embodiments the carrier includes cations or a cationic charge, such as cationic liposomes or cationic polymers such as polyamines (e.g., spermine, spermidine, putrescine). In embodiments, the carrier includes a polypeptide such as an enzyme, (e.g., cellulase, pectolyase, maccroenzyme, pectinase), a cell penetrating or pore-forming peptide (e.g., poly-lysine, poly-arginine, or polyhomoarginine peptides).

Non-limiting examples of carriers include cationic liposomes and polymer nanoparticles such as those reviewed by Zhang et al. (2007) J. Controlled Release, 123:1-10, and the cross-linked multilamellar liposomes described in US Patent Application Publication 2014/0356414 A1, incorporated by reference in its entirety herein. In embodiments, the carrier includes a nanomaterial, such as carbon or silica nanoparticles, carbon nanotubes, carbon nanofibers, or carbon quantum dots. Non-limiting examples of carriers include particles or nanoparticles (e.g., particles or nanoparticles made of materials such as carbon, silicon, silicon carbide, gold, tungsten, polymers, or ceramics) in various size ranges and shapes, magnetic particles or nanoparticles (e.g., silenceMag Magnetotransfection™ agent, OZ Biosciences, San Diego, CA), abrasive or scarifying agents, needles or microneedles, matrices, and grids.

In certain embodiments, particulates and nanoparticulates are useful in delivery of the polynucleotide composition or the nuclease or both. Useful particulates and nanoparticles include those made of metals (e.g., gold, silver, tungsten, iron, cerium), ceramics (e.g., aluminum oxide, silicon carbide, silicon nitride, tungsten carbide), polymers (e.g., polystyrene, poly diacetylene, and poly(3,4-ethylenedioxythiophene) hydrate), semiconductors (e.g., quantum dots), silicon (e.g., silicon carbide), carbon (e.g., graphite, graphene, graphene oxide, or carbon nanosheets, nanocomplexes, or nanotubes), and composites (e.g., polyvinylcarbazole/graphene, polystyrene/graphene, platinum/graphene, palladium/graphene nanocomposites). In certain embodiments, such particulates and nanoparticulates are further covalently or non-covalently functionalized, or further include modifiers or cross-linked materials such as polymers (e.g., linear or branched polyethylenimine, poly-lysine), polynucleotides (e.g., DNA or RNA), polysaccharides, lipids, polyglycols (e.g., polyethylene glycol, thiolated polyethylene glycol), polypeptides or proteins, and detectable labels (e.g., a fluorophore, an antigen, an antibody, or a quantum dot). In various embodiments, such particulates and nanoparticles are neutral, or carry a positive charge, or carry a negative charge.

Embodiments of compositions including particulates include those formulated, e.g., as liquids, colloids, dispersions, suspensions, aerosols, gels, and solids. Embodiments include nanoparticles affixed to a surface or support, e.g., an array of carbon nanotubes vertically aligned on a silicon or copper wafer substrate. Embodiments include polynucleotide compositions including particulates (e.g., gold or tungsten or magnetic particles) delivered by a Biolistic-type technique or with magnetic force. The size of the particles used in Biolistics is generally in the “microparticle” range, for example, gold microcarriers in the 0.6, 1.0, and 1.6 micrometer size ranges (see, e.g., instruction manual for the Helios® Gene Gun System, Bio-Rad, Hercules, CA; Randolph-Anderson et al. (2015) “Submicron gold particles are superior to larger particles for efficient Biolistic® transformation of organelles and some cell types”, Bio-Rad US/EG Bulletin 2015), but successful Biolistics delivery using larger (40-48 nanometer) nanoparticles has been reported in cultured animal cells; see O'Brian and Lummis (2011) BMC Biotechnol., 11:66-71.

Other embodiments of useful particulates are nanoparticles, which are generally in the nanometer (nm) size range or less than 1 micrometer, e.g., with a diameter of less than about 1 nm, less than about 3 nm, less than about 5 nm, less than about 10 nm, less than about 20 nm, less than about 40 nm, less than about 60 nm, less than about 80 nm, and less than about 100 nm. Specific, non-limiting embodiments of nanoparticles commercially available (all from Sigma-Aldrich Corp., St. Louis, MO) include gold nanoparticles with diameters of 5, 10, or 15 nu: silver nanoparticles with particle sizes of 10, 20, 40, 60, or 100 nm; palladium “nanopowder” of less than 25 nm particle size: single-, double-, and multi-walled carbon nanotubes, e.g., with diameters of 0.7-1.1, 1.3-2.3, 0.7-0.9, or 0.7-1.3 nm, or with nano tube bundle dimensions of 2-10 nm by 1-5 micrometers, 6-9 nm by 5 micrometers. 7-15 nm by 0.5-10 micrometers, 7-12 nm by 0.5-10 micrometers, 110-170 nm by 5-9 micrometers, 6-13 nm by 2.5-20 micrometers. Embodiments include polynucleotide compositions including materials such as gold, silicon, cerium, or carbon, e.g., gold or gold-coated nanoparticles, silicon carbide whiskers, carborundum, porous silica nanoparticles, gelatin/silica nanoparticles, nanoceria or cerium oxide nanoparticles (CNPs), carbon nanotubes (CNTs) such as single-, double-, or multi-walled carbon nanotubes and their chemically functionalized versions (e.g., carbon nanotubes functionalized with amide, amino, carboxylic acid, sulfonic acid, or polyethylene glycol moieties), and graphene or graphene oxide or graphene complexes; see, for example, Wong et al. (2016) Nano Lett., 16: 1161-1172; Giraldo et al. (2014) Nature Materials, 13:400-409; Shen et al. (2012) Theranostics, 2:283-294: Kim et al. (2011) Bioconjugate Chem., 22:2558-2567; Wang et al. (2010) J. Am. Chem. Soc. Comm., 132:9274-9276; Zhao et al. (2016) Nanoscale Res. Lett.. 11: 195-203; and Choi et al. (2016) J. Controlled Release, 235:222-235. See also, for example, the various types of particles and nanoparticles, their preparation, and methods for their use, e.g., in delivering polynucleotides and polypeptides to cells, disclosed in US Patent Application Publications 2010/0311168, 2012/0023619, 2012/0244569, 2013/0145488, 2013/0185823, 2014/0096284, 2015/0040268, 2015/0047074, and 2015/0208663, all of which are incorporated herein by reference in their entirety.

In some aspects, the composition includes an excipient, e.g., a delivery vehicle, adjuvant, diluent, surfactant, stabilizer, or tonicity agent or a combination thereof. In some embodiments, the excipient is a crop oil concentrate, a vegetable oil concentrate, a modified vegetable oil, a nitrogen source, a deposition (drift control) and/or retention agent (with or without ammonium sulfate and/or defoamer), a compatibility agent, a buffering agent and/or acidifier, a water conditioning agent, a basic blend, a spreader-sticker and/or extender, an adjuvant plus foliar fertilizer, an antifoam agent, a foam marker, a scent, or a tank cleaner and/or neutralizer. In some embodiments, the excipient is an adjuvant described in the Compendium of Herbicide Adjuvants (Young et al. (2016). Compendium of Herbicide Adjuvants (13^{th} ed.), Purdue University).

Examples of delivery vehicles and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. Further examples of delivery vehicles include, but are not limited to, solid or liquid excipient materials, solvents, stabilizers, slow-release excipients, colorings, and surface-active substances (surfactants). In some instances, the excipient (e.g., delivery vehicle) is a stabilizing vehicle. In embodiments, the stabilizing vehicle includes, e.g., an epoxidized vegetable oil, an antifoaming agent, e.g. silicone oil, a preservative, a viscosity regulator, a binding agent, or a tackifier. In some instances, the stabilizing vehicle is a buffer suitable for the recombinant polynucleotide. In some instances, the composition is microencapsulated in a polymer bead delivery vehicle. In some instances, the stabilizing vehicle protects the recombinant polynucleotide against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

In some instances, the composition provided herein includes an adjuvant. Adjuvants are agents that do not possess the polynucleotide activity, but impart beneficial properties to a formulation. For example, adjuvants are either pre-mixed in the formulation or added to a spray tank to improve mixing or application or to enhance performance. They are used extensively in products designed for foliar applications. Adjuvants can be used to customize the formulation to specific needs and compensate for local conditions. Adjuvants can be designed to perform specific functions, including wetting, spreading, sticking, reducing evaporation, reducing volatilization, buffering, emulsifying, dispersing, reducing spray drift, and reducing foaming. No single adjuvant can perform all these functions, but compatible adjuvants often can be combined to perform multiple functions simultaneously.

Among nonlimiting examples of adjuvants included in the formulation are binders, dispersants and stabilizers, specifically, for example, casein, gelatin, polysaccharides (e.g., starch, gum arabic, cellulose derivatives, alginic acid, etc.), lignin derivatives, bentonite, sugars, synthetic water-soluble polymers (e.g., polyvinyl alcohol, polyvinylpyrrolidone, polyacrylic acid, etc.), PAP (acidic isopropyl phosphate), BHT (2,6-di-t-butyl-4-methylphenol), BHA (a mixture of 2-t-butyl-4-methoxyphenol and 3-t-butyl-4-methoxyphenol), vegetable oils, mineral oils, fatty acids and fatty acid esters.

In embodiments, the compositions provided herein are in a liquid formulation. Liquid formulations are generally mixed with water, but in some instances are used with crop oil, diesel fuel, kerosene, or other light oil as an excipient. The amount of active ingredient (e.g., recombinant polynucleotides) often ranges from about 0.5 to about 80 percent by weight.

In embodiments, an emulsifiable concentrate formulation contains a liquid active ingredient, one or more petroleum-based solvents, and an agent that allows the formulation to be mixed with water to form an emulsion. Such concentrates can be used in agricultural, ornamental and turf, forestry, structural, food processing, livestock, and public health pest formulations. In embodiments, these are adaptable to application equipment from small portable sprayers to hydraulic sprayers, low-volume ground sprayers, mist blowers, and low-volume aircraft sprayers. Some active ingredients readily dissolve in a liquid excipient. When mixed with an excipient, they form a solution that does not settle out or separate, e.g., a homogenous solution. In embodiments, formulations of these types include an active ingredient, a carrier and/or an excipient, and one or more other ingredients. Solutions can be used in any type of spray er, indoors and outdoors.

In some instances, the composition is formulated as an invert emulsion. An invert emulsion is a water-soluble active ingredient dispersed in an oil excipient. Invert emulsions require an emulsifier that allows the active ingredient to be mixed with a large volume of petroleum-based excipient, usually fuel oil. Invert emulsions aid in reducing drift. With other formulations, some spray drift results when water droplets begin to evaporate before reaching target surfaces; as a result, the droplets become very small and lightweight. Because oil evaporates more slowly than water, invert emulsion droplets shrink less, and more active ingredient reaches the target. Oil further helps to reduce runoff and improve rain resistance. It further serves as a sticker-spreader by improving surface coverage and absorption. Because droplets are relatively large and heavy, it is difficult to get thorough coverage on the undersides of foliage. Invert emulsions are most commonly used along rights-of-way where drift to susceptible non-target areas can be a problem.

A flowable or liquid formulation combines many of the characteristics of emulsifiable concentrates and wettable powders. Manufacturers use these formulations when the active ingredient is a solid that does not dissolve in either water or oil. The active ingredient, impregnated on a substance such as clay, is ground to a very fine powder. The powder is then suspended in a small amount of liquid. The resulting liquid product is quite thick. Flowables and liquids share many of the features of emulsifiable concentrates, and they have similar disadvantages. They require moderate agitation to keep them in suspension and leave visible residues, similar to those of wettable powders.

Flowables/liquids are easy to handle and apply. Because they are liquids, they are subject to spilling and splashing. They contain solid particles, so they contribute to abrasive wear of nozzles and pumps. Flowable and liquid suspensions settle out in their containers. Because flowable and liquid formulations tend to settle, packaging in containers of five gallons or less makes remixing easier.

Aerosol formulations contain one or more active ingredients and a solvent. Most aerosols contain a low percentage of active ingredients. There are two types of aerosol formulations—the ready-to-use type commonly available in pressurized sealed containers and those products used in electrical or gasoline-powered aerosol generators that release the formulation as a smoke or fog.

Ready to use (RTU) aerosol formulations are usually small, self-contained units that release the formulation when the nozzle valve is triggered. The formulation is driven through a fine opening by an inert gas under pressure, creating fine droplets. These products are used in greenhouses, in small areas inside buildings, or in localized outdoor areas. Commercial models, which hold five to 5 pounds of active ingredient, are usually refillable.

Smoke or fog aerosol formulations are not under pressure. They are used in machines that break the liquid formulation into a fine mist or fog (aerosol) using a rapidly whirling disk or heated surface.

In some embodiments, the composition comprises a liquid excipient. In embodiments, a liquid excipient includes, for example, aromatic or aliphatic hydrocarbons (e.g., xylene, toluene, alkylnaphthalene, phenylxylylethane, kerosene, gas oil, hexane, cyclohexane, etc.), halogenated hydrocarbons (e.g., chlorobenzene, dichloromethane, dichloroethane, trichloroethane, etc.), alcohols (e.g., methanol, ethanol, isopropyl alcohol, butanol, hexanol, benzyl alcohol, ethylene glycol, etc.), ethers (e.g., diethyl ether, ethylene glycol dimethyl ether, diethylene glycol monomethyl ether, diethylene glycol monoethyl ether, propylene glycol monomethyl ether, tetrahydrofuran, dioxane, etc.), esters (e.g., ethyl acetate, butyl acetate, etc.), ketones (e.g., acetone, methyl ethyl ketone, methyl isobutyl ketone. cyclohexanone, etc.), nitriles (e.g., acetonitrile, isobutyronitrile, etc.), sulfoxides (e.g., dimethyl sulfoxide, etc.), amides (e.g., N,N-dimethylformamide, N,N-dimethylacetamide, cyclic imides (e.g. N-methylpyrrolidone) alkylidene carbonates (e.g., propylene carbonate, etc.), vegetable oil (e.g., soybean oil, cottonseed oil, etc.), vegetable essential oils (e.g., orange oil, hyssop oil, lemon oil, etc.), or water.

In some embodiments, the composition comprises a gaseous excipient. Gaseous excipients include, for example, butane gas, floron gas, liquefied petroleum gas (LPG), dimethyl ether, and carbon dioxide gas.

In some embodiments, the compositions are provided as a dry formulation. Dry formulations can be divided into two types: ready-to-use and concentrates that must be mixed with water to be applied as a spray. Most dust formulations are ready to use and contain a low percentage of active ingredients (less than about 10 percent by weight), plus a very fine, dry inert excipient (e.g., talc, chalk, clay, nut hulls, or volcanic ash). The size of individual dust particles varies. A few dust formulations are concentrates and contain a high percentage of active ingredients. In some embodiments, these are mixed with dry inert excipients before applying. In some embodiments, dusts are used dry and can easily drift to non-target sites.

In some instances, the composition is formulated as a powder. In some instances, the composition is formulated as a wettable powder. Wettable powders are dry, finely ground formulations that look like dusts. They usually must be mixed with water for application as a spray. A few products, however, can be applied either as a dust or as a wettable powder—the choice is left to the applicator. Wettable powders have about 1 to about 95 percent active ingredient by weight; in some cases, more than about 50 percent. The particles do not dissolve in water. They settle out quickly unless constantly agitated to keep them suspended. They can be used for most pest problems and in most types of spray equipment where agitation is possible. Wettable powders have excellent residual activity. Because of their physical properties, most of the formulation remains on the surface of treated porous materials such as concrete, plaster, and untreated wood. In such cases, only the water penetrates the material.

In some instances, the composition is formulated as a soluble powder. Soluble powder formulations look like wettable powders. However, when mixed with water, soluble powders dissolve readily and form a true solution. After they are mixed thoroughly, no additional agitation is necessary. The amount of active ingredient in soluble powders ranges from about 15 to about 95 percent by weight; in some cases, more than about 50 percent. Soluble powders have all the advantages of wettable powders and none of the disadvantages, except the inhalation hazard during mixing.

In some instances, the composition is formulated as a water-dispersible granule. Water-dispersible granules, also known as dry flowables, are like wettable powders, except instead of being dust-like, they are formulated as small, easily measured granules. Water-dispersible granules must be mixed with water to be applied. Once in water, the granules break apart into fine particles similar to wettable powders. The formulation requires constant agitation to keep it suspended in water. The percentage of active ingredient is high, often as much as 90 percent by weight. Water-dispersible granules share many of the same advantages and disadvantages of wettable powders, except they are more easily measured and mixed. Because of low dust, they cause less inhalation hazard to the applicator during handling.

In some embodiments, the composition comprises a solid excipient. Solid excipients include finely-divided powder or granules of clay (e.g. kaolin clay, diatomaceous earth, bentonite, Fubasami clay, acid clay, etc.), synthetic hydrated silicon oxide, talc, ceramics, other inorganic minerals (e.g., sericite, quartz, sulfur, activated carbon, calcium carbonate, hydrated silica, etc.), a substance which can be sublimated and is in the solid form at room temperature (e.g., 2,4,6-triisopropyl-1,3,5-trioxane, naphthalene, p-dichlorobenzene, camphor, adamantan, etc.); wool; silk: cotton; hemp; pulp; synthetic resins (e.g., polyethylene resins such as low-density polyethylene, straight low-density polyethylene and high-density polyethylene; ethylene-vinyl ester copolymers such as ethylene-vinyl acetate copolymers; ethylene-methacrylic acid ester copolymers such as ethylene-methyl methacrylate copolymers and ethylene-ethyl methacrylate copolymers; ethylene-acrylic acid ester copolymers such as ethylene-methyl acrylate copolymers and ethylene-ethyl acrylate copolymers; ethylene-vinylcarboxylic acid copolymers such as ethylene-acrylic acid copolymers; ethylene-tetracyclododecene copolymers; polypropylene resins such as propylene homopolymers and propylene-ethylene copolymers; poly-4-methylpentene-1, polybutene-1, polybutadiene, polystyrene; acrylonitrile-styrene resins: styrene elastomers such as acrylonitrile-butadiene-styrene resins, styrene-conjugated diene block copolymers, and styrene-conjugated diene block copolymer hydrides: fluororesins: acrylic resins such as poly(methyl methacrylate); polyamide resins such as nylon 6 and nylon 66; polyester resins such as polyethylene terephthalate, polyethylene naphthalate, polybutylene terephthalate, and polycyclohexylenedimethylene terephthalate; polycarbonates, polyacetals, polyacrylsulfones, polyarylates, polyacrylates, hydroxybenzoic acid polyesters, polyetherimides, polyester carbonates, polyphenylene ether resins, polyvinyl chloride, polyvinylidene chloride, polyurethane, and porous resins such as foamed polyurethane, foamed polypropylene, or foamed ethylene, etc.), glasses, metals, ceramics, fibers, cloths, knitted fabrics, sheets, papers, yarn, foam, porous substances, and multifilaments.

In some instances, the composition is provided in a microencapsulated formulation (e.g., a nanocapsule). Microencapsulated formulations are mixed with water and sprayed in the same manner as other sprayable formulations. After spraying, the encapsulation shell or coating breaks down and slowly releases the active ingredient.

In some instances, the composition is provided in a liposome. In some instances, the composition is provided in a vesicle.

In some instances, a composition provided herein includes a surfactant. Surfactants, also called wetting agents and spreaders, physically alter the surface tension of a spray droplet. For a formulation to perform its function properly, a spray droplet must be able to wet the foliage and spread out evenly over a leaf. Surfactants enlarge the area of formulation coverage, thereby increasing exposure to the active agent. Surfactants are particularly important when applying a formulation to waxy or hairy leaves. Without proper wetting and spreading, spray droplets often run off or fail to cover leaf surfaces adequately. Too much surfactant, however, can cause excessive runoff and reduce efficacy.

Surfactants can be classified as anionic, cationic, or nonionic. Formulation activity in the presence of a nonionic surfactant can be quite different from activity in the presence of a cationic or anionic surfactant. Selecting the wrong surfactant can reduce the efficacy of a product and injure the target plant. For example, anionic surfactants are most effective when used with contact pesticides (pesticides that control a pest by direct contact rather than being absorbed systemically). Cationic surfactants are not typically used as stand-alone surfactants because they usually are phytotoxic.

Nonionic surfactants, often used with systemic pesticides, help sprays to penetrate plant cuticles. Nonionic surfactants are compatible with most pesticides, and most EPA-registered pesticides that require a surfactant recommend a nonionic type. Adjuvants include, but are not limited to, stickers, extenders, plant penetrants, compatibility agents, buffers or pH modifiers, drift control additives, defoaming agents, and thickeners.

Among nonlimiting examples of surfactants included in the compositions described herein are alkyl sulfate ester salts, alkyl sulfonates, alkyl aryl sulfonates, alkyl aryl ethers and polyoxyethylenated products thereof, polyethylene glycol ethers, polyvalent alcohol esters and sugar alcohol derivatives. In some embodiments, the surfactant is a nonionic surfactant, a surfactant plus nitrogen source, an organo-silicone surfactant, or a high surfactant oil concentrate.

In formulations and in the use forms prepared from these formulations, the recombinant polynucleotide can, in embodiments, be in a mixture with other active compounds, such as pesticidal agents (e.g., insecticides, sterilants, acaricides, nematicides, molluscicides, or fimgicides, attractants, growth-regulating substances, or herbicides). As used herein, the term “pesticidal agent” refers to any substance or mixture of substances intended for preventing, destroying, repelling, or mitigating any pest. A pesticide can be a chemical substance or biological agent used against pests including insects, mollusks, pathogens, weeds, nematodes, and microbes that compete with humans for food, destroy property, spread disease, or are a nuisance. The term “pesticidal agent” further encompasses other bioactive molecules such as antibiotics, antivirals pesticides, antifungals, antihelminthics, nutrients, pollen, sucrose, and/or agents that stun or slow insect movement.

In instances where the recombinant polynucleotide is applied to plants, a mixture with other known compounds, such as herbicides, fertilizers, growth regulators, safeners, semiochemicals, or else with agents for improving plant properties is also possible.

In another aspect, this disclosure is related to a method of producing a modified plant propagule that comprises at least one plant cell comprising a recombinant RNA molecule. The method, in general, includes the steps of: isolating a plant propagule comprising at least one plant cell comprising a recombinant RNA molecule and a Secoviridae RNA-dependent RNA polymerase (RdRP) from a mixed population of plant cells comprising both plant cells comprising the recombinant RNA molecule and plant cells lacking the recombinant RNA molecule, wherein the recombinant RNA molecule comprises, in 5′ to 3′ order, a 5′ RNA replication element that is capable of being recognized by the Secoviridae RdRP; a cargo RNA sequence; and a 3′ RNA replication element that is capable of being recognized by the Secoviridae RdRP. In certain embodiments, the isolated plant propagule comprising at least one plant cell comprising a recombinant RNA molecule will be free or substantially free of plant cells lacking the recombinant RNA. Such isolated plant propagules which are substantially free of plant cells lacking the recombinant RNA can in certain embodiments comprise plant propagules where at least 75%, 80%, 85%, 90%, 95%, 98%, or 99% of the plant cells in the plant propagule contain the recombinant DNA molecule. In embodiments, the mixed population of plant cells comprise a population of protoplasts or a population of cells in callus, an explant, a plant part, or whole plant. In embodiments, the mixed population of plant cells can comprise a population of plant cells where less than 70%, 60%, 50%, 40%, 30%, 20%, 10%, 5%, or 1% of the plant cells in the population contain the recombinant RNA molecule. In some embodiments, the mixed population of plant cells comprise plant cells comprising the Secoviridae RdRP and plant cells lacking the Secoviridae RdRP. In certain embodiments, the plant cells lacking the Secoviridae RdRP will also lack the recombinant RNA. In other embodiments, the mixed population of plant cells comprise plant cells comprising the Secoviridae RdRP. In certain embodiments, the plant cells comprising the Secoviridae RdRP can further comprise the recombinant RNA. In some embodiments, the cargo RNA sequence comprises RNA that encodes a selectable or scorable marker. In embodiments, the mixed population of plant cells is screened or selected for the presence of the plant cell comprising the recombinant RNA molecule prior to isolating the plant propagule. In such screens, the mixed population of cells or a portion thereof are subjected to an assay for a screenable marker for the presence of the recombinant RNA molecule (e.g., an RNA sequence diagnostic for presence of the recombinant RNA molecule or a polypeptide encoded by the recombinant RNA molecule) and separated from plant cells lacking the recombinant RNA molecule. In some embodiments, the isolation comprises selecting for the plant cell comprising the recombinant RNA molecule prior to isolating the plant propagule. Examples of such selections in instances where the recombinant RNA encodes a selectable marker (e.g., a protein which confers resistance to a selection agent such as an herbicide or antibiotic) can comprise exposing the mixed population of plant cells to a selection agent (e.g., an herbicide or antibiotic) and isolating plant cells which survive exposure to the selection agent. Examples of selectable marker/selection agent combinations include glyphosate-resistant EPSPS enzymes and/or glyphosate oxidases/glyphosate, a bialaphos resistance (bar) or phosphinothricin acyl transferase (pat) enzyme/glufosinate, or a neomycin phosphotransferase (npt)/neomycin or kanamycin. In certain embodiments, the selectable or scorable marker is an RNA aptamer (e.g., a Broccoli aptamer) or a regulatory RNA (e.g., an siRNA, siRNA precursor, miRNA, or miRNA precursor, or a phased siRNA or phased siRNA precursor that downregulates expression of an endogenous gene in the plant, resulting in a detectable phenotype, e.g., bleaching caused by downregulation of a pigment-producing gene). In embodiments, the mixed population is located within a plant or a plant part. In some embodiments, the plant or plant part is screened or selected for presence of the recombinant RNA molecule prior to isolating the plant propagule. In other embodiments, the plant or plant part is screened or selected for systemic presence of the recombinant RNA molecule prior to isolating the plant propagule. In some embodiments, the plant cells, plant, or plant part in the mixed population or that are isolated lack DNA that encodes the recombinant RNA molecule. In embodiments, the plant propagule comprising the recombinant RNA molecule is isolated by detecting the RNA molecule in one or more plant cells comprising the recombinant RNA molecule and separating the one or more plant cells comprising the recombinant RNA molecule from the plant cells lacking the recombinant DNA molecule. In some embodiments, the plant propagule is a mosaic comprising both plant cells comprising the recombinant RNA molecule and plant cells lacking the recombinant RNA molecule. In other embodiments, the modified plant propagule is a mosaic comprising both plant cells comprising the Secoviridae RdRP and plant cells lacking the Secoviridae RdRP. In certain embodiments, at least 99%, 98%, 95%, 90%, 85%, 80%, 70%, 60%, 50%, 40%, 30%, 20%, 10%, 5%, or 1% of the plant cells in the mosaic can comprise the recombinant RNA molecule. In certain embodiments that are particularly advantageous for at least regulatory reasons, the plant propagule lacks DNA that encodes the recombinant RNA molecule. In embodiments, the modified plant propagule comprises the cell comprising the recombinant RNA molecule, or a seed, seedling, ovule, embryo, pollen, root, stem, leaf, shoot, tuber, rhizome, stolon, bulb, explant, or callus comprising the cell comprising the recombinant RNA molecule. Plant propagules made by any of the aforementioned methods and/or incorporating any of the aforementioned features are also provided herein.

In some embodiments, any of the aforementioned methods can further comprise multiplying the cell, seed, seedling, ovule, embryo, pollen, root, stem, leaf, shoot, tuber, rhizome, stolon, bulb, explant, or callus to obtain progeny, wherein the progeny comprise the recombinant RNA molecule. In other embodiments, the multiplying of the cells consists of culturing a plurality of explants obtained from the cell, seed, seedling, ovule, embryo, pollen, root, stem, leaf, shoot, tuber, rhizome, stolon, bulb, explant, or callus. In yet other embodiments, the isolated propagule comprises the cell and the aforementioned methods can further comprise regenerating a plant, seedling, ovule, embryo, pollen, root, stem, leaf, shoot, tuber, rhizome, stolon, bulb, explant, or callus comprising the recombinant RNA from the cell. In still other embodiments, the isolated propagule comprises callus and the aforementioned methods can further comprise regenerating a plant, seedling, ovule, embryo, pollen, root, stem, leaf, shoot, tuber, rhizome, stolon, bulb, or explant comprising the recombinant RNA from said callus. In embodiments, a plant is regenerated and the aforementioned methods can further comprise recovering F₁ seed or F₁ progeny or clonal progeny comprising the recombinant RNA from the plant.

In another aspect, this disclosure is related to a method of providing a synthetic Secoviridae satellite RNA to a plant or plant part by grafting one plant part to another plant part. In certain embodiments, the methods can comprise grafting a scion onto a rootstock comprising any of the aforementioned or otherwise disclosed recombinant DNA molecules and/or recombinant RNA molecules (e.g., a recombinant RNA comprising, in 5′ to 3′ order, a 5′ RNA replication element that is capable of being recognized by a Secoviridae RNA-dependent RNA polymerase (RdRP); a cargo RNA sequence; and a 3′ RNA replication element that is capable of being recognized by the Secoviridae RdRP), wherein at least one cell of the rootstock and/or the scion comprises the Secoviridae RdRP. In certain embodiments, the scion can comprise a plant shoot, an apical or other meristem, a leaf attached to a petiole, or other plant part and the rootstock can comprise roots and aerial portions of the plant including the main stem, secondary stems, leaves, and/or reproductive structures of the plant. In embodiments, DNA that encodes the recombinant RNA molecule is absent in the scion and/or the rootstock. In embodiments, the scion lacks the recombinant RNA molecule prior to grafting. In embodiments, the rootstock comprises the Secoviridae RdRP. In some embodiments, the Secoviridae RdRP is provided by a virus of the family Secoviridae endemic to the rootstock (e.g., a virus of the family Secoviridae which is non-pathogenic and/or commensal). In other embodiments, the Secoviridae RdRP is exogenously provided to the rootstock (e.g., via a DNA expression cassette which is integrated into the chromosomal or plastidic DNA of the rootstock or via a recombinant viral vector comprising DNA or RNA encoding the RdRP). In embodiments, the scion comprises the Secoviridae RdRP. In some embodiments, the RdRP is provided by a virus of the family Secoviridae endemic to the scion (e.g., a virus of the family Secoviridae which is non-pathogenic and/or commensal). In other embodiments, the RdRP is exogenously provided to the scion (e.g., via a DNA expression cassette which is integrated into the chromosomal or plastidic DNA of the scion or via a recombinant viral vector comprising DNA or RNA encoding the RdRP). In embodiments, the Secoviridae RdRP comprises a protein having at least 85%, 90%, 95%, 97%, or 99% sequence identity to SEQ ID NO: 471, 530, or 558-564. In embodiments, the rootstock and/or the scion comprises a heterologous viral coat protein which can encapsidate the recombinant RNA molecule. In some embodiments, the rootstock and/or the scion comprises the recombinant RNA molecule encapsidated by a heterologous viral coat protein.

In another aspect, this disclosure is related to a method of producing a grafted plant comprising a recombinant RNA molecule comprising, in 5′ to 3′ order, a 5′ RNA replication element that is capable of being recognized by a Secoviridae RNA-dependent RNA polymerase (RdRP); a cargo RNA sequence; and a 3′ RNA replication element that is capable of being recognized by an the Secoviridae RdRP. In certain embodiments, the recombinant RNA molecule is provided by contacting the scion, the rootstock, or both the scion and the rootstock with a composition comprising the recombinant RNA molecule prior to grafting the scion onto the rootstock to produce the grafted plant. In certain embodiments, at least one cell of the rootstock and/or the scion comprises a Secoviridae RdRP prior to contacting the scion, the rootstock, or both the scion and the rootstock with the composition. In embodiments, the rootstock comprises the Secoviridae RdRP. In some embodiments, the Secoviridae RdRP is provided by a virus of the family Secoviridae endemic to the rootstock (e.g., a virus of the family Secoviridae which is non-pathogenic and/or commensal). In other embodiments, the Secoviridae RdRP is exogenously provided to the rootstock (e.g., via a DNA expression cassette which is integrated into the chromosomal or plastidic DNA of the rootstock or via a recombinant viral vector comprising DNA or RNA encoding the RdRP). In embodiments, the scion comprises the Secoviridae RdRP. In some embodiments, the RdRP is provided by a virus of the family Secoviridae endemic to the scion (e.g., a virus of the family Secoviridae which is non-pathogenic and/or commensal). In other embodiments, the RdRP is exogenously provided to the scion (e.g., via a DNA expression cassette which is integrated into the chromosomal or plastidic DNA of the scion or via a recombinant viral vector comprising DNA or RNA encoding the RdRP). In embodiments, the Secoviridae RdRP comprises a protein having at least 85%, 90%, 95%, 97%, or 99% sequence identity to SEQ ID NO: 471, 530, or 558-564. In embodiments, DNA that encodes the recombinant RNA molecule is absent in the scion, the rootstock, and/or the grafted plant. The composition can be provided to the scion, the rootstock, or both the scion and the rootstock according to any of the formulations disclosed herein. In some embodiments, the formulation is a liquid, a gel, or a powder. In some embodiments, the formulation is configured to be sprayed on to the scion, the rootstock, or both the scion and the rootstock; to be injected into the scion, the rootstock, or both the scion and the rootstock; to be soaked into the scion, the rootstock, or both the scion and the rootstock; or to be coated onto the scion, the rootstock, or both the scion and the rootstock. In certain embodiments, the contacting comprises dipping the scion, the rootstock, or both the scion and the rootstock into the composition prior to grafting.

In another aspect, this disclosure is related to a method for producing a plant that transmits any of the aforementioned or otherwise disclosed recombinant RNA molecules provided herein to progeny plants or seed. In certain embodiments, the methods include the steps of: isolating an F₁ progeny plant or seed comprising at least one cell comprising a Secoviridae RNA-dependent RNA polymerase (RdRP) and the recombinant RNA molecule comprising, in 5′ to 3′ order, a 5′ RNA replication element that is capable of being recognized by the Secoviridae RdRP); a cargo RNA sequence; and a 3′ RNA replication element that is capable of being recognized by the Secoviridae RdRP from a population of F₁ plants or seed obtained from at least one parent plant comprising the recombinant RNA molecule. In embodiments, the parent plant or a part thereof comprising the plant cell is screened or selected for presence of the recombinant RNA molecule prior to isolating the F₁ progeny plant or seed. In some embodiments, the parent plant or one or more parts thereof are screened for systemic presence of the recombinant RNA molecule prior to isolating the F₁ progeny plants. In other embodiments, floral tissue (e.g., whole flowers or buds, sepal, calyx, or petal), male reproductive tissue (e.g., stamen, anther, or pollen), or female reproductive tissue (e.g., whole fruit, ovary, pericarp, ovule, seed coat, endosperm, or embryo) of the parent plant is screened or selected for presence of the recombinant RNA molecule. In embodiments, F₁ seeds are obtained from a parent plant selected for presence of the recombinant RNA molecule in pericarp tissue. In embodiments, the F₁ progeny plant or seed comprising the cell is isolated by screening the population of F₁ plants or seed obtained from a parent plant for the presence of the recombinant RNA molecule and propagating the F₁ progeny plant or seed comprising the recombinant RNA molecule. In such screens, the progeny plants or seed thereof are subjected to an assay for a screenable marker for the presence of the recombinant RNA (e.g., an RNA sequence diagnostic for presence of the recombinant RNA molecule or a polypeptide encoded by the recombinant RNA molecule) and separated from progeny plants and seed lacking the recombinant RNA progeny plants and seed lacking the recombinant RNA. Such screening assays can be non-destructive assays wherein a portion of the progeny seed or plant is removed and assayed without loss of the viability or ability to propagate the seed or plant tissue comprising the recombinant RNA. In some embodiments, an F₁ seed of the parent plant is non-destructively screened for presence of the recombinant RNA molecule. In other embodiments, the F₁ seed of the parent plant is non-destructively screened by assaying maternally derived or endosperm tissue of the seed for the presence of the recombinant RNA molecule. Methods for non-destructive assays of seed or other plant tissue which can be adapted for such screens include but are not limited to those disclosed in US patent applications US20220221377 and US20210259176, both incorporated herein by reference in their entireties. In some embodiments, the cargo RNA sequence comprises RNA that encodes a selectable or scorable marker. In some embodiments, the recombinant RNA molecule encodes a selectable marker and the F₁ progeny plant or seed comprising the recombinant RNA molecule is isolated by selecting the F₁ progeny plant or seed comprising the recombinant RNA molecule for presence of the selectable marker. Examples of such selections in instances where the recombinant RNA encodes a selectable marker (e.g., a protein which confers resistance to a selection agent such as an herbicide or antibiotic) can comprise exposing the progeny seeds or plants to a selection agent (e.g., an herbicide or antibiotic) and isolating progeny seeds or plants which survive exposure to the selection agent. Examples of selectable marker/selection agent combinations include glyphosate-resistant EPSPS enzymes and/or glyphosate oxidases/glyphosate, a bialaphos resistance (bar) or phosphinothricin acyl transferase (pat) enzymes/glufosinate, and neomycin phosphotransferase (npt)/neomycin or kanamycin. In certain embodiments, the selectable or scorable marker is an RNA aptamer or a regulatory RNA. In some embodiments, the F₁ progeny plant or seed lacks DNA that encodes the recombinant RNA molecule. In other embodiments, the parent plant lacks DNA that encodes the recombinant RNA molecule. In embodiments, the selected F₁ progeny plant transmits the recombinant RNA molecule to at least F₂ progeny. In some embodiments, the F₁ progeny plant or seed population is obtained from a parent plant used as a pollen recipient. In other embodiments, the F₁ progeny plant or seed population is obtained from a parent plant used as a pollen donor. In embodiments, the F₁ progeny plant or seed population is obtained by selfing the parent plant. In other embodiments, the F₁ progeny plant or seed population is obtained from the sexual crossing of two parent plants. In some embodiments, the parent plant that comprises the recombinant RNA molecule is the female parent plant. In other embodiments, the parent plant that comprises the recombinant RNA molecule is the male parent plant, and the recombinant RNA molecule is transmitted in pollen of the male parent plant.

In certain embodiments, the methods can further comprise introducing the recombinant RNA molecule or a polynucleotide encoding the recombinant RNA molecule into a plant cell and obtaining the parent plant comprising the recombinant RNA molecule from the plant cell. In embodiments, the recombinant RNA molecule further comprises at least one additional element selected from the group consisting of: (a) at least one RNA encoding a viral movement protein (MP); (b) at least one tRNA-like sequence; and c) an origin-of-assembly sequence (OAS). In embodiments, a parent and/or plant comprises a heterologous viral coat protein which can encapsidate the recombinant RNA molecule. In some embodiments, the parent and/or progeny plant comprises the recombinant RNA molecule encapsidated by a heterologous viral coat protein.

In another aspect, this disclosure is related to a method of barcoding a plant, plant cell, progeny thereof, or part thereof. The methods comprise providing to the plant or plant cell any of the aforementioned or otherwise disclosed recombinant RNA molecules provided herein, wherein the cargo RNA of the recombinant RNA molecule comprises a barcode RNA molecule, and wherein the plant or plant cell comprises a Secoviridae RdRP. In embodiments, the barcode RNA molecule comprises a sequence that uniquely identifies the plant, plant cell, progeny thereof, or part thereof. In some embodiments, the barcode RNA can be a randomly generated sequence. In some embodiments, the barcode RNA molecule comprises a sequence that is not present in the genome and/or transcriptome of a wild-type plant of the same species, a pathogen thereof, or a symbiont thereof. In some embodiments, the barcode RNA molecule comprises a forward primer binding site and a reverse primer binding site for detection of the barcode RNA molecule. In embodiments, the barcode RNA molecule comprises a non-protein coding sequence. In some embodiments, the barcode RNA sequence is up to about 8 kb in length. In some embodiments, the barcode RNA has a length of 10 to 8000 nucleotides, 20 to 1000 nucleotides, or 50 to 500 nucleotides. In certain embodiments, the barcode RNA molecule has a length of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, or 8000 nucleotides. In embodiments, the plant transmits the recombinant RNA molecule comprising the barcode RNA to progeny. In some embodiments, the plant, plant cell, progeny thereof, or part thereof lacks DNA that encodes the recombinant RNA molecule. In some embodiments, the methods can further comprise isolating an F₁ progeny plant or seed comprising at least one cell comprising the Secoviridae RdRP and the recombinant RNA molecule. In some embodiments, the F₁ progeny plant or seed is obtained from the plant used as a pollen recipient. In other embodiments, the F₁ progeny plant or seed is obtained from the plant used as a pollen donor. In embodiments, the F₁ progeny plant or seed is obtained by selfing the parent plant. In embodiments, the methods can further comprise propagating the plant or plant cell to obtain a plant part or a plant propagule comprising the barcode RNA molecule.

In another aspect, this disclosure is related to a method of identifying a barcoded plant, plant part, or plant cell. The methods comprise screening for the presence of a barcode RNA molecule in the plant, plant part, or plant cell, wherein the plant, plant part, or plant cell comprises any of the aforementioned or otherwise disclosed recombinant RNA molecules provided herein, and wherein the cargo RNA of the recombinant RNA molecule comprises the barcode RNA molecule. In certain embodiments, the methods comprise obtaining a nucleic acid sample from the plant, plant part, or plant cell; and detecting the presence of the barcode RNA molecule in the sample. Assays for detection of a barcode RNA include RNA detection assays (e.g., an RT-PCR assay) using nucleic acid probes and/or primers which can detect the barcode RNA and/or sequencing of the barcode RNA. Such screening assays can be non-destructive assays wherein a portion of the seed or plant is removed and assayed without loss of the viability or ability to propagate the seed or plant tissue comprising the barcode RNA. Methods for non-destructive assays of seed or other plant tissue which can be adapted for such screens include but are not limited to those disclosed in US patent applications US20220221377 and US20210259176, both incorporated herein by reference in their entireties. In some embodiments, a seed of the plant is non-destructively screened for presence of the barcode RNA molecule. In other embodiments, a seedling, ovule, embryo, pollen, root, stem, leaf, shoot, tuber, rhizome, stolon, bulb, explant, or callus is screened for the presence of the barcode RNA molecule.

In certain optional embodiments, the methods disclosed herein are not processes for modifying the germ line or genetic identity of human beings. In certain optional embodiments, the methods disclosed herein are not processes for modifying the genetic identity of animals which are likely to cause them suffering without any substantial medical benefit to man or animal, and are also not drawn to animals resulting from such processes. In certain optional embodiments, the methods disclosed herein are not methods for treatment of the human or animal body by surgery or therapy. In certain optional embodiments, the cells disclosed herein are not human embryos. In certain optional embodiments, the cells disclosed herein are not the human body or its parts, at the various stages of its formation and development. In certain optional embodiments provided herein, the plant cells, plant propagules (e.g., a seed, seedling, ovule, embryo, pollen, root, stem, leaf, shoot, tuber, rhizome, stolon, bulb, explant, or callus), and plants provided herein are not produced by an exclusively biological process. In certain optional embodiments provided herein, the methods for producing plant cells, plant propagules (e.g., a seed, seedling, ovule, embryo, pollen, root, stem, leaf, shoot, tuber, rhizome, stolon, bulb, explant, or callus), and plants provided herein are not exclusively biological processes.

### EMBODIMENTS

Various embodiments of the compositions, systems, and methods described herein are set forth in the following set of numbered embodiments.
1. A recombinant RNA molecule comprising from 5′ terminus to 3′ terminus:
(a) a 5′ RNA replication element recognized by a Secoviridae RNA-dependent RNA polymerase (RdRP);
(b) a cargo RNA molecule; and
(c) a 3′ RNA replication element recognized by the RdRP;
   wherein the 5′ RNA replication element, the cargo RNA molecule, and the 3′ RNA replication element are operably linked and wherein the cargo RNA molecule is heterologous to the 5′ RNA replication element and the 3′ RNA replication element, optionally wherein: (i) the 5′ RNA replication element and the 3′ RNA replication element are obtained from the same Secoviridae genome or from Secoviridae genomes having at least 85%, 90%, 95%, 98%, or 99% sequence identity to one another and which are optionally related; (ii) the 5′ RNA replication element, the 3′ RNA replication element, and the RdRP are obtained from the same Secoviridae genome or from Secoviridae genomes having at least 85%, 90%, 95%, 98%, or 99% sequence identity to one another and which are optionally related; or (iii) the 5′ RNA replication element, the 3′ RNA replication element, and/or the RdRP coding region are obtained from different Secoviridae genomes, and wherein the members of each respective set of the 5′ RNA replication elements, 3′ RNA replication elements, and/or RdRP coding regions have at least 85%, 90%, 95%, 98%, or 99% sequence identity to one another.
2. The recombinant RNA molecule of embodiment 1, wherein:
(a) the 5′ RNA replication element comprises at least one RNA secondary structure provided in Table 1 or adopted by an RNA molecule encoded by SEQ ID NO: 467, 468, 512, 546-551, or 631; and/or
(b) the 3′ RNA replication element comprises at least one RNA secondary structure provided in Table 1 or adopted by an RNA molecule encoded by SEQ ID NO: 469, 470, 520, 521, or 552-557.
3. The recombinant RNA molecule of embodiment 1, wherein:
(a) the 5′ RNA replication element comprises an RNA molecule encoded by SEQ ID NO: 467, 468, 512, 546-551, or 631: a variant thereof encoded by a DNA molecule having at least 70%, 75%, 80%, 85%, 90%, 95%, 98%, or 99% sequence identity to SEQ ID NO: 467, 468, 512, 546-551, or 631; or a variant thereof wherein one or more nucleotides in the RNA secondary structure are substituted with distinct nucleotides which maintain the RNA secondary structure; and/or
(b) the 3′ RNA replication element comprises an RNA molecule encoded by SEQ ID NO: 469, 470, 520, 521, or 552-557, or: a variant thereof encoded by a DNA molecule having at least 70%, 75%, 80%, 85%, 90%, 95%, 98%, or 99% sequence identity to SEQ ID NO: 469, 470, 520, 521, or 552-557; or a variant thereof wherein one of more base-paired residues in the RNA secondary structure are substituted with distinct nucleotides which maintain the RNA secondary structure.
4. The recombinant RNA molecule of embodiment 3, wherein the RNA secondary structure is maintained by substituting nucleotides in the secondary structure which are not base paired with nucleotides which will not base pair, and/or by substituting nucleotides in the secondary structure which are base paired with nucleotides which will base pair.
5. The recombinant RNA molecule of any one of embodiments 1 to 4, wherein:
(a) the 5′ RNA replication element comprises at least a segment of the 5′ untranslated region (UTR) of the Secoviridae genome or a variant thereof having one or more nucleotide substitutions, insertions, and/or deletions, wherein the variant is recognized by the RdRP, optionally wherein the 5′ RNA replication element further comprises a genomic sequence of the virus of the family Secoviridae that is natively located 3′ to and adjacent to the 5′ UTR sequence; and/or
(b) the 3′ RNA replication element comprises at least a segment of the 3′ UTR of the Secoviridae genome or a variant thereof having one or more nucleotide substitutions, insertions, and/or deletions, wherein the variant is recognized by the RdRP, optionally wherein the 3′ RNA replication element further comprises a genomic sequence of the virus of the family Secoviridae that is natively located 5′ to and adjacent to the 3′ UTR sequence, and optionally wherein the Secoviridae genome of (a) and (b) are the same.
6. The recombinant RNA molecule of any one of embodiments 1 to 5, wherein the RNA molecule further comprises at least one of: (i) a tRNA-like element, optionally wherein the tRNA-like element provides for intercellular movement of the RNA and optionally wherein the intercellular movement is mediated by a viral movement protein (MP); (ii) an encapsidation recognition element (ERE), optionally wherein the ERE provides for encapsidation of the RNA by a Secoviridae capsid protein or optionally wherein the ERE provides for encapsidation of the RNA by a non-Secoviridae capsid protein; (iii) an RNA effecter; and/or (iv) RNA encoding a viral movement protein (MP), optionally wherein an internal ribosome entry site (IRES) is operably linked to the RNA encoding the MP.
7. The recombinant RNA molecule of embodiment 6, wherein the tRNA-like element comprises a tRNA-like molecule from an Arabidopsis FT mRNA or is a tRNA-like sequence encoded by a DNA sequence selected from the group consisting of SEQ ID NOs: 76-123, and 466 or is a modified tRNA-like sequence that has at least 90% sequence identity to a scaffold tRNA-like sequence encoded by a DNA sequence selected from the group consisting of SEQ ID NOs: 76-123, and 466 and that maintains the secondary structure of the scaffold tRNA-like sequence, and/or wherein the ERE is a tobacco mosaic virus (TMV) OAS.
8. The recombinant RNA molecule of embodiment 1, wherein the cargo RNA molecule is up to about 8 kb in length.
9. The recombinant RNA molecule of embodiment 1, wherein the cargo RNA molecule comprises: (a) at least one coding sequence, optionally wherein the coding sequence encodes a selectable or scoreable marker; (b) at least one non-coding sequence; or (c) both at least one coding sequence and at least one non-coding sequence.
10. The recombinant RNA molecule of embodiment 1, wherein the cargo RNA molecule comprises at least one coding sequence, and wherein the RNA molecule further comprises an internal ribosome entry site (IRES) which is operably linked to at least one coding sequence, optionally wherein the operably linked IRES is located 5′ and immediately adjacent to the coding sequence.
11. The recombinant RNA molecule of embodiment 1, wherein the cargo RNA molecule comprises at least one non-coding sequence, and wherein the at least one non-coding sequence is a hairpin RNA (hpRNA); an RNA that forms multiple stem-loops; an RNA pseudoknot; an RNA molecule that forms at least partially double-stranded RNA; a small interfering RNA (siRNA) or siRNA precursor; a microRNA (miRNA) or miRNA precursor; a phased RNA or phased RNA precursor; a ribozyme; a ligand-responsive ribozyme (aptazyme); an RNA aptamer; or a long noncoding RNA (lncRNA).
12. The recombinant RNA molecule of embodiment 1, further comprising an RNA comprising encoding at least one ribozyme, optionally wherein the at least one ribozyme is located 5′ to the 5′ RNA replication element or 3′ to the 3 RNA replication element.
13. The recombinant RNA molecule of embodiment 1, further comprising an RNA molecule comprising at least one ligand-responsive ribozyme (aptazyme), optionally wherein the at least one ligand-responsive ribozyme is located 5′ to the 5′ RNA replication element or 3′ to the 3′ RNA replication element.
14. The recombinant RNA molecule of embodiment 1, wherein: (i) the RNA further comprises at least a segment of its reverse complementary RNA molecule; and/or (ii) the recombinant RNA molecule is complexed with one or more RNA binding proteins or encapsidated by a viral capsid protein, optionally wherein the viral capsid protein is a Secoviridae capsid protein, optionally wherein the RNA comprises an ERE which provides for encapsidation of the RNA by the Secoviridae capsid protein, and/or optionally wherein the RNA binding proteins comprise an RNA recognition motif.
15. The recombinant RNA molecule of embodiment 1, wherein the RNA molecule comprises: at least one heterologous RNA virus (HRV) amplicon in sense or antisense orientation to the first 5′ RNA replication element comprising:
I. (i) a heterologous RNA virus (HRV) 5′ replication region (HRV 5′RR); (ii) the cargo RNA molecule; and (iii) the heterologous RNA virus (HRV) 3′ RNA replication region (HRV 3′RR); wherein the HRV 5′ RR and HRV 3′ RR HRV are recognized by a heterologous RNA virus RNA-dependent RNA polymerase (hrvRdRP); and wherein the HRV 5′RR, cargo RNA molecule, and HRV 3′RR are operably linked; or
II. a heterologous RNA virus (HRV) subgenomic promoter operably linked to the cargo RNA molecule; wherein the subgenomic promoter is recognized by a heterologous RNA virus RNA-dependent RNA polymerase (hrvRdRP).
16. The recombinant RNA of embodiment 15, wherein the RNA molecule comprises from 5′ terminus to 3′ terminus:
(a) the 5′ RNA replication element;
(b) the HRV amplicon in antisense orientation to the first 5′ RNA replication element; optionally wherein the HRV amplicon further comprises:
(i) an RNA molecule encoding an HRV RNA-dependent RNA polymerase (hrvRdRP) which is operably linked to the HRV 5′RR and HRV 3′RR, wherein the RNA molecule encoding the HRV RNA-dependent RNA polymerase (hrvRdRP) is optionally operably linked to a subgenomic promoter recognized by the hrvRdRP; or
(ii) an RNA molecule encoding an HRV RNA-dependent RNA polymerase (hrvRdRP) which is operably linked to linked to a subgenomic promoter recognized by the hrvRdRP; and
(c) the 3′ RNA replication element.
17. The recombinant RNA of embodiment 15 or 16, wherein the HRV 5′RR, HRV 3′RR, and hrvRdRP comprise an HRV 5′RR, HRV 3′RR, and hrvRdRP from a virus selected from the group consisting of an Alphaflexivirus, Betaflexivirus, Bromovirus, Celavirus, Closterovirus, Comovirus, Potexvirus, Potyvirus, Tobamovirus, Tombusvirus, Tospoviridac, Trivirinac, Tymovirus, Varicosavirus, and Secoviridae.
18. The recombinant RNA of embodiment 17, wherein: (i) the Bromovirus is a Cucumber Mosaic Virus, Spinach latent virus, Olive latent virus, or Brome mosaic virus; (ii) the Closterovirus is a Citrus tristeza virus or Beet yellows virus; (iii) the Comovirus is Cowpea mosaic virus, Apple latent spherical virus, or Soybean latent spherical virus; (iv) the Potexvirus is Potato virus X or Citrus yellow vein clearing virus; (v) the Potyvirus is a Pepper mottle virus, Bean yellow mosaic virus, Barley stripe mosaic virus, Wheat stripe mosaic virus, Rice yellow mottle virus, Maize dwarf mosaic virus, zucchini yellow mosaic virus, watermelon mosaic virus, or sugar cane mosaic virus; (vi) the Tobamovirus is a Tobacco mosaic virus. Tomato mosaic virus, Tomato brown rugose fruit virus, Turnip vein-clearing virus, or Pepper mild mottle virus; (vii) the Tombusvirus is a Turnip crinkle virus or Tomato bushy stunt virus; (iv) the Tospoviridae is a Tomato spotted wilt virus or watermelon bud necrosis virus; or (viii) the Tymovirus is a Turnip yellow mosaic virus. Citrus yellow vein clearing virus. Potato latent virus, Apple stem grooving virus, or Citrus leaf blotch virus.
19. The recombinant RNA of any one of embodiments 1 to 18, wherein the HRV 5′ RR and the HRV 3′ RR are obtained from the same HRV genome.
20. The recombinant RNA of any one of embodiments 1 to 18, wherein the HRV 5′ RR and the HRV 3′ RR are obtained from distinct HRV genomes.
21. The recombinant RNA molecule of any one of embodiments 1 to 20, wherein:
a) the HRV 5′ RR comprises at least one RNA secondary structure adopted by an RNA encoded by SEQ ID NO: 161 to 185, or 186, or comprises the RNA sequence encoded by SEQ ID NO: 161 to 185, or 186, or comprises a contiguous fragment of at least 80%, 85%, 90%, or 95% of the full sequence of the RNA encoded by SEQ ID NO: 161 to 185, or 186; and/or
(b) the HRV 3′ RR comprises at least one RNA secondary structure adopted by an RNA encoded by SEQ ID NO: 187 to 210, or 211, or comprises the RNA sequence of the RNA encoded by SEQ ID NO: 187 to 210, or 211, or comprises a contiguous fragment of at least 80%, 85%, 90%, or 95% of the full sequence of the RNA encoded by SEQ ID NO: 187 to 210, or 211.
22. The recombinant RNA molecule of any one of embodiments 1 to 20, wherein:
(a) the HRV 5′ RR is encoded by a DNA molecule comprising SEQ ID NO: 161 to 185, or 186; or a variant thereof comprising DNA having at least 70%, 75%, 80%, 85%, 90%, 95%, 98%, or 99% sequence identity to SEQ ID NO: 161 to 185, or 186; or a variant thereof wherein one of more residues in the RNA secondary structure are substituted with distinct nucleotides which maintain the RNA secondary structure; and/or
(b) the HRV 3′ RR is encoded by a DNA molecule comprising SEQ ID NO: 187 to 210, or 211; or a variant thereof comprising DNA having at least 70%, 75%, 80%, 85%, 90%, 95%, 98%, or 99% sequence identity to SEQ ID NO: 187 to 210, or 211; or a variant thereof wherein one of more residues in the RNA secondary structure are substituted with distinct nucleotides which maintain the RNA secondary structure
23. The recombinant RNA molecule of embodiment 15, wherein:
(a) the HRV 3′ RR comprises an RNA molecule containing at least a segment of the 3′ untranslated region (UTR) of the HRV genome, or a variant thereof having one or more nucleotide substitutions, insertions, and/or deletions, wherein the HRV 3′ RR or the variant is recognized by the hrvRdRP, optionally wherein the RNA comprising the HRV 3′ RR further comprises a genomic sequence of the HRV that is natively located 5′ to and adjacent to the 3′ UTR sequence; and/or
(b) the HRV 5′ RR comprises an RNA molecule containing at least a segment of the 3′ untranslated region (UTR) of the HRV genome, or a variant thereof having one or more nucleotide substitutions, insertions, and/or deletions, wherein the HRV 5′ RR or the variant is recognized by the hrvRdRP, optionally wherein the RNA comprising the HRV 5′ RR further comprises a genomic sequence of the HRV that is natively located 3′ to and adjacent to the 5′ UTR sequence.
24 The recombinant RNA molecule of any one of embodiments 1 to 23, wherein the cargo RNA comprises an HRV-inhibitory RNA or encodes an HRV-inhibitory protein, wherein the HRV-inhibitory RNA or HRV-inhibitory protein inhibits infection, movement, transmission, and/or replication of the HRV.
25. The recombinant RNA molecule of any one of embodiments 1 to 24, wherein the cargo RNA comprises an RNA having at least 20 contiguous nucleotides having an identical or complementary sequence to a segment of equivalent length of the genomic RNA of the HRV.
26. The recombinant RNA molecule of any one of embodiments 1 to 24, wherein the cargo RNA comprises an RNA having at least 20 contiguous nucleotides having an identical or complementary sequence to a segment of equivalent length of the genomic RNA of the HRV which does not encode the hrvRdRP.
27. The recombinant RNA molecule of any one of embodiments 1 to 26, wherein:
(i) the HRV is a Cucumber Mosaic Virus and the HRV 5′RR comprises an RNA encoded by the Cucumber Mosaic Virus 5′ RR DNA sequence in Table 7, an RNA encoded by a DNA having at least 85%, 90%, 95%, or 99% sequence identity to the Cucumber Mosaic Virus 5′ RR DNA sequence in Table 7, the HRV 3′ RR comprises an RNA encoded by the Cucumber Mosaic Virus 3′ RR DNA sequence in Table 7, or an RNA encoded by a DNA having at least 85%, 90%, 95%, or 99% sequence identity to the Cucumber Mosaic Virus 3′ RR DNA sequence in Table 7;
(ii) the HRV is a Brome mosaic virus and the HRV 5′RR comprises an RNA encoded by the Brome mosaic virus 5′ RR DNA sequence in Table 7, an RNA encoded by a DNA having at least 85%, 90%, 95%, or 99% sequence identity to the Brome mosaic virus 5′ RR DNA sequence in Table 7, the HRV 3′ RR comprises an RNA encoded by the Brome mosaic virus 3′ RR DNA sequence in Table 7, or an RNA encoded by a DNA having at least 85%, 90%, 95%, or 99% sequence identity to the Brome mosaic virus 3′ RR DNA sequence in Table 7;
(iii) the HRV is a Citrus tristeza virus and the HRV 5′RR comprises an RNA encoded by the Citrus tristeza virus 5′ RR DNA sequence in Table 7, an RNA encoded by a DNA having at least 85%, 90%, 95%, or 99% sequence identity to the Citrus tristeza virus 5′ RR DNA sequence in Table 7, the HRV 3′ RR comprises an RNA encoded by the Citrus tristeza virus 3′ RR DNA sequence in Table 7, or an RNA encoded by a DNA having at least 85%, 90%, 95%, or 99% sequence identity to the Citrus tristeza virus 3′ RR DNA sequence in Table 7, and the RdRP comprises a protein having at least 85%, 90%, 95%, or 99% sequence identity to the Citrus tristeza virus RdRP sequence in Table 7;
(iv) the HRV is Beet yellows virus and the HRV 5′RR comprises an RNA encoded by the Beet yellows virus 5′ RR DNA sequence in Table 7, an RNA encoded by a DNA having at least 85%, 90%, 95%, or 99% sequence identity to the Beet yellows virus 5′ RR DNA sequence in Table 7, the HRV 3′ RR comprises an RNA encoded by the Beet yellows virus 3′ RR DNA sequence in Table 7, or an RNA encoded by a DNA having at least 85%, 90%, 95%, or 99% sequence identity to the Beet yellows virus 3′ RR DNA sequence in Table 7, and the RdRP comprises a protein having at least 85%, 90%, 95%, or 99% sequence identity to the Beet yellows virus RdRP sequence in Table 7;
(v) the HRV is Cowpea mosaic virus and the HRV 5′RR comprises an RNA encoded by the Cowpea mosaic virus 5′ RR DNA sequence in Table 7 or an RNA encoded by a DNA having at least 85%, 90%, 95%, or 99% sequence identity to the Cowpea mosaic virus 5′ RR DNA sequence in Table 7;
(vi) the HRV is Potato virus X and the HRV 5′RR comprises an RNA encoded by the Potato virus X 5′ RR DNA sequence in Table 7, an RNA encoded by a DNA having at least 85%, 90%, 95%, or 99% sequence identity to the Potato virus X 5′ RR DNA sequence in Table 7, the HRV 3′ RR comprises an RNA encoded by the Potato virus X 3′ RR DNA sequence in Table 7, or an RNA encoded by a DNA having at least 85%, 90%, 95%, or 99% sequence identity to the Potato virus X 3′ RR DNA sequence in Table 7, and the RdRP comprises a protein having at least 85%, 90%, 95%, or 99% sequence identity to the Potato virus X RdRP sequence in Table 7;
(vii) the HRV is Pepper mottle virus and the HRV 5′RR comprises an RNA encoded by the Pepper mottle virus 5′ RR DNA sequence in Table 7, an RNA encoded by a DNA having at least 85%, 90%, 95%, or 99% sequence identity to the Pepper mottle virus 5′ RR DNA sequence in Table 7, the HRV 3′ RR comprises an RNA encoded by the Pepper mottle virus 3′ RR DNA sequence in Table 7, or an RNA encoded by a DNA having at least 85%, 90%, 95%, or 99% sequence identity to the Pepper mottle virus 3′ RR DNA sequence in Table 7, and the RdRP comprises a protein having at least 85%, 90%, 95%, or 99% sequence identity to the Pepper mottle virus RdRP sequence in Table 7;
(viii) the HRV is Bean yellow mosaic virus and the HRV 5′RR comprises an RNA encoded by the Bean yellow mosaic virus 5′ RR DNA sequence in Table 7, an RNA encoded by a DNA having at least 85%, 90%, 95%, or 99% sequence identity to the Bean yellow mosaic virus 5′ RR DNA sequence in Table 7, the HRV 3′ RR comprises an RNA encoded by the Bean yellow mosaic virus 3′ RR DNA sequence in Table 7, or an RNA encoded by a DNA having at least 85%, 90%, 95%, or 99% sequence identity to the Bean yellow mosaic virus 3′ RR DNA sequence in Table 7, and the RdRP comprises a protein having at least 85%, 90%, 95%, or 99% sequence identity to the Bean yellow mosaic virus RdRP sequence in Table 7;
(ix) the HRV is Barley stripe mosaic virus and the HRV 5 ′RR comprises an RNA encoded by the Barley stripe mosaic virus 5′ RR DNA sequence in Table 7, an RNA encoded by a DNA having at least 85%, 90%, 95%, or 99% sequence identity to the Barley stripe mosaic virus 5′ RR DNA sequence in Table 7, and the HRV 3′ RR comprises an RNA encoded by the Barley stripe mosaic virus 3′ RR DNA sequence in Table 7, or an RNA encoded by a DNA having at least 85%, 90%, 95%, or 99% sequence identity to the Barley stripe mosaic virus 3′ RR DNA sequence in Table 7;
(x) the HRV is Wheat stripe mosaic virus and the HRV 5′RR comprises an RNA encoded by the Wheat stripe mosaic virus 5′ RR DNA sequence in Table 7, an RNA encoded by a DNA having at least 85%, 90%, 95%, or 99% sequence identity to the Wheat stripe mosaic virus 5′ RR DNA sequence in Table 7, the HRV 3′ RR comprises an RNA encoded by the Wheat stripe mosaic virus 3′ RR DNA sequence in Table 7, or an RNA encoded by a DNA having at least 85%, 90%, 95%, or 99% sequence identity to the Wheat stripe mosaic virus 3′ RR DNA sequence in Table 7, and the RdRP comprises a protein having at least 85%, 90%, 95%, or 99% sequence identity to the Wheat stripe mosaic virus RdRP sequence in Table 7;
(xi) the HRV Rice yellow mottle virus and the HRV 5′RR comprises an RNA encoded by the Rice yellow mottle virus 5′ RR DNA sequence in Table 7, an RNA encoded by a DNA having at least 85%, 90%, 95%, or 99% sequence identity to the Rice yellow mottle virus 5′ RR DNA sequence in Table 7, the HRV 3′ RR comprises an RNA encoded by the Rice yellow mottle virus 3′ RR DNA sequence in Table 7, or an RNA encoded by a DNA having at least 85%, 90%, 95%, or 99% sequence identity to the Rice yellow mottle virus 3′ RR DNA sequence in Table 7, and the RdRP comprises a protein having at least 85%, 90%, 95%, or 99% sequence identity to the Rice yellow mottle virus RdRP sequence in Table 7;
(xii) the HRV is Maize dwarf mosaic virus and the HRV 5′RR comprises an RNA encoded by the Maize dwarf mosaic virus 5′ RR DNA sequence in Table 7, an RNA encoded by a DNA having at least 85%, 90%, 95%, or 99% sequence identity to the Maize dwarf mosaic virus 5′ RR DNA sequence in Table 7, the HRV 3′ RR comprises an RNA encoded by the Maize dwarf mosaic virus 3′ RR DNA sequence in Table 7, or an RNA encoded by a DNA having at least 85%, 90%, 95%, or 99% sequence identity to the Maize dwarf mosaic virus 3′ RR DNA sequence in Table 7, and the RdRP comprises a protein having at least 85%, 90%, 95%, or 99% sequence identity to the Maize dwarf mosaic virus RdRP sequence in Table 7;
(xiii) the HRV is zucchini yellow mosaic virus and the HRV 5′RR comprises an RNA encoded by the Zucchini yellow mosaic virus 5′ RR DNA sequence in Table 7, an RNA encoded by a DNA having at least 85%, 90%, 95%, or 99% sequence identity to the Zucchini yellow mosaic virus 5′ RR DNA sequence in Table 7, the HRV 3′ RR comprises an RNA encoded by the Zucchini yellow mosaic virus 3′ RR DNA sequence in Table 7, or an RNA encoded by a DNA having at least 85%, 90%, 95%, or 99% sequence identity to the Zucchini yellow mosaic virus 3′ RR DNA sequence in Table 7, and the RdRP comprises a protein having at least 85%, 90%, 95%, or 99% sequence identity to the Zucchini yellow mosaic virus RdRP sequence in Table 7;
(xiv) the HRV is watermelon mosaic virus and the HRV 5′RR comprises an RNA encoded by the Watermelon mosaic virus 5′ RR DNA sequence in Table 7, an RNA encoded by a DNA having at least 85%, 90%, 95%, or 99% sequence identity to the Watermelon mosaic virus 5′ RR DNA sequence in Table 7, the HRV 3′ RR comprises an RNA encoded by the Watermelon mosaic virus 3′ RR DNA sequence in Table 7, or an RNA encoded by a DNA having at least 85%, 90%, 95%, or 99% sequence identity to the Watermelon mosaic virus 3′ RR DNA sequence in Table 7, and the RdRP comprises a protein having at least 85%, 90%, 95%, or 99% sequence identity to the Watermelon mosaic virus RdRP sequence in Table 7;
(xv) the HRV is sugarcane mosaic virus and the HRV 5′RR comprises an RNA encoded by the Sugarcane mosaic virus 5′ RR DNA sequence in Table 7, an RNA encoded by a DNA having at least 85%, 90%, 95%, or 99% sequence identity to the Sugarcane mosaic virus 5′ RR DNA sequence in Table 7, the HRV 3′ RR comprises an RNA encoded by the Sugarcane mosaic virus 3′ RR DNA sequence in Table 7, or an RNA encoded by a DNA having at least 85%, 90%, 95%, or 99% sequence identity to the Sugarcane mosaic virus 3′ RR DNA sequence in Table 7, and the RdRP comprises a protein having at least 85%, 90%, 95%, or 99% sequence identity to the Sugarcane mosaic virus RdRP sequence in Table 7;
(xvi) the HRV is Tobacco mosaic virus and the HRV 5′RR comprises an RNA encoded by the Tobacco mosaic virus 5′ RR DNA sequence in Table 7, an RNA encoded by a DNA having at least 85%, 90%, 95%, or 99% sequence identity to the Tobacco mosaic virus 5′ RR DNA sequence in Table 7, the HRV 3′ RR comprises an RNA encoded by the Tobacco mosaic virus 3′ RR DNA sequence in Table 7, or an RNA encoded by a DNA having at least 85%, 90%, 95%, or 99% sequence identity to the Tobacco mosaic virus 3′ RR DNA sequence in Table 7, and the RdRP comprises a protein having at least 85%, 90%, 95%, or 99% sequence identity to the Tobacco mosaic virus RdRP sequence in Table 7;
(xvii) the HRV is Tomato mosaic virus and the HRV 5′RR comprises an RNA encoded by the Tomato mosaic virus 5′ RR DNA sequence in Table 7, an RNA encoded by a DNA having at least 85%, 90%, 95%, or 99% sequence identity to the Tomato mosaic virus 5′ RR DNA sequence in Table 7, the HRV 3′ RR comprises an RNA encoded by the Tomato mosaic virus 3′ RR DNA sequence in Table 7, or an RNA encoded by a DNA having at least 85%, 90%, 95%, or 99% sequence identity to the Tomato mosaic virus 3′ RR DNA sequence in Table 7, and the RdRP comprises a protein having at least 85%, 90%, 95%, or 99% sequence identity to the Tomato mosaic virus RdRP sequence in Table 7;
(xviii) the HRV is Tomato brown rugose fruit virus and the HRV 5′RR comprises an RNA encoded by the Tomato brown rugose fruit virus 5′ RR DNA sequence in Table 7, an RNA encoded by a DNA having at least 85%, 90%, 95%, or 99% sequence identity to the Tomato brown rugose fruit virus 5′ RR DNA sequence in Table 7, the HRV 3′ RR comprises an RNA encoded by the Tomato brown rugose fruit virus 3′ RR DNA sequence in Table 7, or an RNA encoded by a DNA having at least 85%, 90%, 95%, or 99% sequence identity to the Tomato brown rugose fruit virus 3′ RR DNA sequence in Table 7, and the RdRP comprises a protein having at least 85%, 90%, 95%, or 99% sequence identity to the Tomato brown rugose fruit virus RdRP sequence in Table 7;
(xix) the HRV is Turnip vein-clearing virus and the HRV 5′RR comprises an RNA encoded by the Turnip vein-clearing virus 5′ RR DNA sequence in Table 7, an RNA encoded by a DNA having at least 85%, 90%, 95%, or 99% sequence identity to the Turnip vein-clearing virus 5′ RR DNA sequence in Table 7, the HRV 3′ RR comprises an RNA encoded by the Turnip vein-clearing virus 3′ RR DNA sequence in Table 7, or an RNA encoded by a DNA having at least 85%, 90%, 95%, or 99% sequence identity to the Turnip vein-clearing virus 3′ RR DNA sequence in Table 7, and the RdRP comprises a protein having at least 85%, 90%, 95%, or 99% sequence identity to the Turnip vein-clearing virus RdRP sequence in Table 7;
(xx) the HRV is Pepper mild mottle virus and the HRV 5′RR comprises an RNA encoded by the Pepper mild mottle virus 5′ RR DNA sequence in Table 7, an RNA encoded by a DNA having at least 85%, 90%, 95%, or 99% sequence identity to the Pepper mild mottle virus 5′ RR DNA sequence in Table 7, the HRV 3′ RR comprises an RNA encoded by the Pepper mild mottle virus 3′ RR DNA sequence in Table 7, or an RNA encoded by a DNA having at least 85%, 90%, 95%, or 99% sequence identity to the Pepper mild mottle virus 3′ RR DNA sequence in Table 7, and the RdRP comprises a protein having at least 85%, 90%, 95%, or 99% sequence identity to the Pepper mild mottle virus RdRP sequence in Table 7;
(xxi) the HRV is Turnip crinkle virus and the HRV 5′RR comprises an RNA encoded by the Turnip crinkle virus 5′ RR DNA sequence in Table 7, an RNA encoded by a DNA having at least 85%, 90%, 95%, or 99% sequence identity to the Turnip crinkle virus 5′ RR DNA sequence in Table 7, the HRV 3′ RR comprises an RNA encoded by the Turnip crinkle virus 3′ RR DNA sequence in Table 7, or an RNA encoded by a DNA having at least 85%, 90%, 95%, or 99% sequence identity to the Turnip crinkle virus 3′ RR DNA sequence in Table 7, and the RdRP comprises a protein having at least 85%, 90%, 95%, or 99% sequence identity to the Turnip crinkle virus RdRP sequence in Table 7; (xxii) the HRV is Tomato bushy stunt virus and the HRV 5′RR comprises an RNA encoded by the Tomato bushy stunt virus 5′ RR DNA sequence in Table 7, an RNA encoded by a DNA having at least 85%, 90%, 95%, or 99% sequence identity to the Tomato bushy stunt virus 5′ RR DNA sequence in Table 7, the HRV 3′ RR comprises an RNA encoded by the Tomato bushy stunt virus 3′ RR DNA sequence in Table 7, or an RNA encoded by a DNA having at least 85%, 90%, 95%, or 99% sequence identity to the Tomato bushy stunt virus 3′ RR DNA sequence in Table 7, and the RdRP comprises a protein having at least 85%, 90%, 95%, or 99% sequence identity to the Tomato bushy stunt virus RdRP sequence in Table 7;
(xxiii) the HRV is Tomato spotted wilt virus and the HRV 5′RR comprises an RNA encoded by the Tomato spotted wilt virus 5′ RR DNA sequence in Table 7, an RNA encoded by a DNA having at least 85%, 90%, 95%, or 99% sequence identity to the Tomato spotted wilt virus 5′ RR DNA sequence in Table 7, the HRV 3′ RR comprises an RNA encoded by the Tomato spotted wilt virus 3′ RR DNA sequence in Table 7, or an RNA encoded by a DNA having at least 85%, 90%, 95%, or 99% sequence identity to the Tomato spotted wilt virus 3′ RR DNA sequence in Table 7, and the RdRP comprises a protein having at least 85%, 90%, 95%, or 99% sequence identity to the Tomato spotted wilt virus RdRP sequence in Table 7;
(xxiv) the HRV is watermelon bud necrosis virus and the HRV 5′RR comprises an RNA encoded by the Watermelon bud necrosis virus 5′ RR DNA sequence in Table 7, an RNA encoded by a DNA having at least 85%, 90%, 95%, or 99% sequence identity to the Watermelon bud necrosis virus 5′ RR DNA sequence in Table 7, the HRV 3′ RR comprises an RNA encoded by the Watermelon bud necrosis virus 3′ RR DNA sequence in Table 7, or an RNA encoded by a DNA having at least 85%, 90%, 95%, or 99% sequence identity to the Watermelon bud necrosis virus 3′ RR DNA sequence in Table 7, and the RdRP comprises a protein having at least 85%, 90%, 95%, or 99% sequence identity to the Watermelon bud necrosis virus RdRP sequence in Table 7;
(xxv) the HRV is Turnip yellow mosaic virus and the HRV 5′RR comprises an RNA encoded by the Turnip yellow mosaic virus 5′ RR DNA sequence in Table 7, an RNA encoded by a DNA having at least 85%, 90%, 95%, or 99% sequence identity to the Turnip yellow mosaic virus 5′ RR DNA sequence in Table 7, the HRV 3′ RR comprises an RNA encoded by the Turnip yellow mosaic virus 3′ RR DNA sequence in Table 7, or an RNA encoded by a DNA having at least 85%, 90%, 95%, or 99% sequence identity to the Turnip yellow mosaic virus 3′ RR DNA sequence in Table 7, and the RdRP comprises a protein having at least 85%, 90%, 95%, or 99% sequence identity to the Turnip yellow mosaic virus RdRP sequence in Table 7;
(xxvi) the HRV is Spinach latent virus and the HRV 5′RR comprises an RNA encoded by the Spinach latent virus 5′ RR DNA sequence in Table 7, an RNA encoded by a DNA having at least 85%, 90%, 95%, or 99% sequence identity to the Spinach latent virus 5′ RR DNA sequence in Table 7, the HRV 3′ RR comprises an RNA encoded by the Spinach latent virus 3′ RR DNA sequence in Table 7, or an RNA encoded by a DNA having at least 85%, 90%, 95%, or 99% sequence identity to the Spinach latent virus 3′ RR DNA sequence in Table 7, and the RdRP comprises a protein having at least 85%, 90%, 95%, or 99% sequence identity to the Spinach latent virus RdRP sequence of SEQ ID NO: 523;
(xxvii) the HRV is Spinach latent virus and the HRV 5′RR comprises an RNA encoded by the Spinach latent virus 5′ RR DNA sequence in Table 7, an RNA encoded by a DNA having at least 85%. 90%, 95%, or 99% sequence identity to the Spinach latent virus 5′ RR DNA sequence in Table 7, the HRV 3′ RR comprises an RNA encoded by the Spinach latent virus 3′ RR DNA sequence in Table 7, or an RNA encoded by a DNA having at least 85%, 90%, 95%, or 99% sequence identity to the Spinach latent virus 3′ RR DNA sequence in Table 7, and the RdRP comprises a protein having at least 85%, 90%, 95%, or 99% sequence identity to the Spinach latent virus RdRP sequence of SEQ ID NO: 524;
(xxviii) the HRV is Olive latent virus 2 and the HRV 5′RR comprises an RNA encoded by the Olive latent virus 2 5′ RR DNA sequence in Table 7, an RNA encoded by a DNA having at least 85%, 90%, 95%, or 99% sequence identity to the Olive latent virus 2 5′ RR DNA sequence in Table 7, the HRV 3′ RR comprises an RNA encoded by the Olive latent virus 2 3′ RR DNA sequence in Table 7, or an RNA encoded by a DNA having at least 85%, 90%, 95%, or 99% sequence identity to the Olive latent virus 2 3′ RR DNA sequence in Table 7, and the RdRP comprises a protein having at least 85%, 90%, 95%, or 99% sequence identity to the Olive latent virus 2 RdRP sequence in Table 7;
(xxix) the HRV is Citrus yellow vein clearing virus and the HRV 5′RR comprises an RNA encoded by the Citrus yellow vein clearing virus 5′ RR DNA sequence in Table 7, an RNA encoded by a DNA having at least 85%, 90%, 95%, or 99% sequence identity to the Citrus yellow vein clearing virus virus 5′ RR DNA sequence in Table 7, the HRV 3′ RR comprises an RNA encoded by the Citrus yellow vein clearing virus 3′ RR DNA sequence in Table 7, or an RNA encoded by a DNA having at least 85%, 90%, 95%, or 99% sequence identity to the Citrus yellow vein clearing virus 3′ RR DNA sequence in Table 7, and the RdRP comprises a protein having at least 85%, 90%, 95%, or 99% sequence identity to the Citrus yellow vein clearing virus RdRP sequence in Table 7;
(xxx) the HRV is Potato latent virus and the HRV 5′RR comprises an RNA encoded by the Potato latent virus 5′ RR DNA sequence in Table 7, an RNA encoded by a DNA having at least 85%, 90%, 95%, or 99% sequence identity to the Potato latent virus 5′ RR DNA sequence in Table 7, the HRV 3′ RR comprises an RNA encoded by the Potato latent virus 3′ RR DNA sequence in Table 7, or an RNA encoded by a DNA having at least 85%, 90%, 95%, or 99% sequence identity to the Potato latent virus 3′ RR DNA sequence in Table 7, and the RdRP comprises a protein having at least 85%, 90%, 95%, or 99% sequence identity to the Potato latent virus RdRP sequence in Table 7;
(xxxi) the HRV is Apple stem grooving virus and the HRV 5′RR comprises an RNA encoded by the Apple stem grooving virus 5′ RR DNA sequence in Table 7, an RNA encoded by a DNA having at least 85%, 90%, 95%, or 99% sequence identity to the Apple stem grooving virus 5′ RR DNA sequence in Table 7, the HRV 3′ RR comprises an RNA encoded by the Apple stem grooving virus 3′ RR DNA sequence in Table 7, or an RNA encoded by a DNA having at least 85%, 90%, 95%, or 99% sequence identity to the Apple stem grooving virus 3′ RR DNA sequence in Table 7, and the RdRP comprises a protein having at least 85%, 90%, 95%, or 99% sequence identity to the Apple stem grooving virus RdRP sequence in Table 7;
(xxxii) the HRV is Citrus leaf blotch virus and the HRV 5′RR comprises an RNA encoded by the Citrus leaf blotch virus 5′ RR DNA sequence in Table 7, an RNA encoded by a DNA having at least 85%, 90%, 95%, or 99% sequence identity to the Citrus leaf blotch virus 5′ RR DNA sequence in Table 7, the HRV 3′ RR comprises an RNA encoded by the Citrus leaf blotch virus 3′ RR DNA sequence in Table 7, or an RNA encoded by a DNA having at least 85%, 90%, 95%, or 99% sequence identity to the Citrus leaf blotch virus 3′ RR DNA sequence in Table 7, and the RdRP comprises a protein having at least 85%, 90%, 95%, or 99% sequence identity to the Citrus leaf blotch virus RdRP sequence in Table 7;
(xxxiii) the HRV is Apple latent spherical virus and the HRV 5′RR comprises an RNA encoded by the Apple latent spherical virus 5′ RR DNA sequence in Table 7, an RNA encoded by a DNA having at least 85%, 90%, 95%, or 99% sequence identity to the Apple latent spherical virus 5′ RR DNA sequence in Table 7, the HRV 3′ RR comprises an RNA encoded by the Apple latent spherical virus 3′ RR DNA sequence in Table 7, or an RNA encoded by a DNA having at least 85%, 90%, 95%, or 99% sequence identity to the Apple latent spherical virus 3′ RR DNA sequence in Table 7, and the RdRP comprises a protein having at least 85%, 90%, 95%, or 99% sequence identity to the Apple latent spherical virus RdRP sequence in Table 7;
(xxxiv) the HRV is Soybean latent spherical virus and the HRV 5′RR comprises an RNA encoded by the Soybean latent spherical virus 5′ RR DNA sequence in Table 7, an RNA encoded by a DNA having at least 85%, 90%, 95%, or 99% sequence identity to the Soybean latent spherical virus 5′ RR DNA sequence in Table 7, the HRV 3′ RR comprises an RNA encoded by the Soybean latent spherical virus 3′ RR DNA sequence in Table 7, or an RNA encoded by a DNA having at least 85%, 90%, 95%, or 99% sequence identity to the Soybean latent spherical virus 3′ RR DNA sequence in Table 7, and the RdRP comprises a protein having at least 85%, 90%, 95%, or 99% sequence identity to the Soybean latent spherical virus RdRP sequence in Table 7;
(xxxv) the HRV is Celery latent virus and the HRV 5′RR comprises an RNA encoded by the Celery latent virus 5′ RR DNA sequence in Table 7, an RNA encoded by a DNA having at least 85%, 90%, 95%, or 99% sequence identity to the Celery latent virus 5′ RR DNA sequence in Table 7, the HRV 3′ RR comprises an RNA encoded by the Celery latent virus 3′ RR DNA sequence in Table 7, or an RNA encoded by a DNA having at least 85%, 90%, 95%, or 99% sequence identity to the Celery latent virus 3′ RR DNA sequence in Table 7, and the RdRP comprises a protein having at least 85%, 90%, 95%, or 99% sequence identity to the Celery latent virus RdRP sequence in Table 7;
(xxxvi) the HRV is Black grass varicosavirus-like virus and the HRV 5′RR comprises an RNA encoded by the Black grass varicosavirus-like virus 5′ RR DNA sequence in Table 7, an RNA encoded by a DNA having at least 85%, 90%, 95%, or 99% sequence identity to the Black grass varicosavirus-like virus 5′ RR DNA sequence in Table 7, the HRV 3′ RR comprises an RNA encoded by the Black grass varicosavirus-like virus 3′ RR DNA sequence in Table 7, or an RNA encoded by a DNA having at least 85%, 90%, 95%, or 99% sequence identity to the Black grass varicosavirus-like virus 3′ RR DNA sequence in Table 7, and the RdRP comprises a protein having at least 85%, 90%, 95%, or 99% sequence identity to the Black grass varicosavirus-like virus sequence in Table 7; or
(xxxvii) the HRV is Maize suscal virus and the HRV 5′RR comprises an RNA encoded by the Maize suscal virus 5′ RR DNA sequence in Table 7, an RNA encoded by a DNA having at least 85%, 90%, 95%, or 99% sequence identity to the Maize suscal virus 5′ RR DNA sequence in Table 7, the HRV 3′ RR comprises an RNA encoded by the Maize suscal virus 3′ RR DNA sequence in Table 7, or an RNA encoded by a DNA having at least 85%, 90%, 95%, or 99% sequence identity to the Maize suscal virus 3′ RR DNA sequence in Table 7, and the RdRP comprises a protein having at least 85%, 90%, 95%, or 99% sequence identity to the Maize suscal virus sequence in Table 7.
28. The recombinant RNA molecule of any one of embodiments 1 to 27, further comprising an RNA comprising encoding at least one cleavable sequence, optionally wherein the at least one cleavable sequence is located: (i) 5′ to the 5′ RNA replication element or 3′ to the 3′ RNA replication element; and/or (ii) between the 3′ end of the 5′ RNA replication element and the HRV amplicon and/or between the HRV amplicon and the 5′ end of the 3′ RNA replication element, wherein the cleavable sequence is optionally a self-cleaving ribozyme, a self-cleaving inducible ribozyme, or an siRNA or miRNA recognition site.
29. An agricultural formulation comprising the recombinant RNA molecule of any one of embodiments 1 to 28.
30. The agricultural formulation of embodiment 29, wherein the recombinant RNA molecule is complexed with one or more RNA binding proteins or encapsidated by a viral capsid protein, optionally wherein the viral capsid protein is a Secoviridae capsid protein and optionally wherein the RNA comprises an ERE which provides for encapsidation of the RNA by the Secoviridae capsid protein.
31. The agricultural formulation of embodiment 30, wherein the RNA binding proteins comprise an RNA recognition motif.
32. The agricultural formulation of embodiment 30 or 31, wherein the viral capsid protein is heterologous to the virus of the family Secoviridae.
33. The agricultural formulation of anyone of embodiments 29 to 32, wherein the formulation comprises the recombinant RNA molecule and a carrier, an excipient, and/or an adjuvant.
34. A cell comprising the recombinant RNA molecule of any one of embodiments 1 to 28, wherein the cell is a bacterial cell, a fungal cell, a plant cell, an insect cell, or an invertebrate animal cell.
35. The cell of embodiment 34, wherein the cell is a plant cell and DNA which encodes the recombinant RNA molecule is absent from the cell.
36. The cell of embodiment 34, wherein the cell comprises a recombinant DNA molecule which encodes the recombinant RNA molecule.
37. An expression system comprising:
(a) an RNA molecule comprising the recombinant RNA molecule of any one of embodiments 1 to 28; and
(b) a cell containing the recombinant RNA molecule and an RdRP protein that recognizes the 5′ and 3′ RNA replication elements of the recombinant RNA molecule.
38. The expression system of embodiment 37, wherein the recombinant RNA molecule comprises an operably linked encapsidation recognition element (ERE) recognized by a viral capsid protein, and wherein the cell contains the viral capsid protein.
39. The expression system of embodiment 37, wherein the encapsidation recognition element (ERE) is a Secoviridae ERE, wherein the viral capsid protein in the cell is a Secoviridae capsid protein, and wherein the RNA molecule is encapsidated by the Secoviridae capsid protein.
40. The expression system of embodiment 37, 38, or 39, further comprising the reverse complement of the recombinant RNA molecule.
41. The expression system of any one of embodiments 37 to 40, wherein the cell is a bacterial cell, a plant cell, a fungal cell, an insect cell, or an invertebrate animal cell.
42. The expression system of any one of embodiments 37 to 41, wherein the cell further comprises: (i) a viral capsid protein (CP), (ii) an RNA-binding protein (RBP) that can bind to the RNA molecule, optionally wherein the RBP binds to an RNA effecter; (iii) an RNA cleavage agent that cleaves the RNA molecule; (iv) a second RNA-dependent RNA polymerase (RdRP) protein that recognizes an HRV 5′ or 3′ replication region and/or a subgenomic promoter in the RNA molecule (2^{nd} RdRP); (v) a viral movement protein (MP); (v) a heterologous RNA virus (HRV); or (vi) an hrvRdRP, optionally wherein the hrvRdRP recognizes the HRV 5′ or 3′ replication region and/or the subgenomic promoter.
43. The expression system of embodiment 42, wherein the CP, RBP, RdRP, 2^{nd} RdRP, hrvRdRP, and/or the MP is: (a) expressed by a recombinant DNA molecule in the cell; (b) provided exogenously to the cell; (c) expressed by a recombinant RNA molecule in the cell; or (d) expressed by a virus in the cell.
44. The expression system of embodiment 42, wherein the RdRP. CP. RBP, 2^{nd} RdRP, hrvRdRP, and/or the MP protein is heterologous to the cell.
45. The expression system of embodiment 42 or 44, wherein the RdRP, 2^{nd} RdRP, or hrvRdRP protein or a polynucleotide encoding the RdRP, 2^{nd} RdRP, or hrvRdRP protein is (a) expressed by a recombinant DNA molecule in the cell; (b) provided exogenously to the cell; (c) expressed by a recombinant RNA molecule in the cell; or (d) expressed by a virus in the cell.
46. The expression system of any one of embodiments 37 to 45, wherein the cell is a plant cell.
47. The expression system of embodiment 46, wherein the plant cell contains a virus of the family Secoviridae which expresses the RdRP protein that recognizes the 5′ RNA replication element and the 3′ RNA replication element and/or wherein the plant cell contains an HRV that expresses the 2^{nd} RdRP or hrvRdRP protein.
48. The expression system of embodiment 47, wherein the virus of the family Secoviridae occurs naturally in the plant cell.
49. A method of providing a synthetic Secoviridae satellite RNA to a plant, comprising contacting the plant with the recombinant RNA molecule of any one of embodiments 1 to 28.
50. The method of embodiment 49, wherein contacting comprises spraying, dusting, injecting, or soaking the plant or a part thereof with the recombinant RNA molecule or the formulation.
51. The method of embodiment 49 or 50, further comprising providing an hrvRdRP to the plant which recognizes the HRV 5′ or 3′ replication region and/or the subgenomic promoter, optionally wherein the hrvRdRP is provided by introducing a recombinant DNA or RNA encoding the hrvRdRP into the plant or a part thereof.
52. A method of establishing a synthetic Secoviridae satellite RNA in a plant cell, comprising: providing to a plant cell the recombinant RNA molecule of any one of embodiments 1 to 28; wherein the plant cell comprises an RdRP protein that recognizes the 5′ RNA replication element and 3′ RNA replication element, wherein the RNA molecule is optionally comprises an ERE and is encapsidated by a capsid protein, whereby the RdRP protein catalyzes synthesis of the synthetic Secoviridae satellite RNA from the recombinant RNA molecule.
53. The method of embodiment 52, wherein the plant cell comprises a virus of the family Secoviridae and wherein the RdRP protein is provided to the plant cell by the virus of the family Secoviridae.
54. The method of embodiment 52 or 53, wherein the virus of the family Secoviridae is endemic to the plant cell, optionally wherein the virus of the family Secoviridae which is endemic to the plant cell is non-pathogenic and/or commensal.
55. The method of embodiment 52, 53, or 54, wherein the capsid protein comprises a Secoviridae capsid protein and the ERE is recognized by the Secoviridae capsid protein, optionally wherein the recombinant RNA molecule comprises an operably linked encapsidation recognition element (ERE) recognized by a viral capsid protein.
56. The method of any one of embodiments 52 to 55, further comprising providing an hrvRdRP to the plant which recognizes the HRV 5′ or 3′ replication region and/or the subgenomic promoter in the synthetic Secoviridae satellite RNA, optionally wherein the hrvRdRP is provided by introducing a recombinant DNA or RNA encoding the hrvRdRP into the plant or a part thereof.
57. A method of obtaining a phenotypic change in a plant or plant cell, comprising: providing to a plant or plant cell a recombinant RNA molecule of any one of embodiments 1 to 28, wherein the cargo RNA molecule comprises RNA that effects a phenotypic change in the plant or plant cell in comparison to a plant or plant cell lacking the recombinant RNA, wherein the plant or plant cell comprises an RdRP protein that recognizes the 5′ RNA replication element and 3* RNA replication element and catalyzes synthesis of a synthetic Secoviridae RNA from the recombinant RNA molecule, and wherein the cargo RNA molecule effects the phenotypic change.
58. The method of embodiment 57, wherein the RNA that effects a phenotypic change in the plant or plant cell comprises at least one RNA selected from an siRNA or siRNA precursor, a miRNA or miRNA precursor, and a phased siRNA or phased siRNA precursor.
59. The method of embodiment 57, wherein the RNA that effects a phenotypic change in the plant or plant cell comprises a messenger RNA.
60. The method of embodiment 59, wherein the messenger RNA comprises an RNA molecule absent in the genome of the plant or plant cell.
61. The method of embodiment 57, wherein the RNA that effects a phenotypic change in the plant or plant cell comprises an RNA for modifying the genome of the plant or plant cell.
62. The method of embodiment 57, wherein the RNA that effects a phenotypic change in the plant or plant cell comprises an RNA for modifying the transcriptome and/or epigenome of the plant or plant cell, optionally wherein the RNA for modifying the epigenome targets an endogenous plant gene for RNA-induced transcriptional silencing.
63. The method of any one of embodiments 57 to 62, wherein the phenotypic change comprises an increase in the plant's resistance to a pest or pathogen, optionally wherein the pest or pathogen is selected from the group comprising a bacterium, a virus other than a virus of the family Secoviridae, a fungus, an oomycete, and an invertebrate.
64. The method of embodiment 63, wherein the pathogen is a heterologous RNA virus (HRV), optionally wherein the HRV is a virus selected from the group consisting of an Alphaflexivirus, Betaflexivirus, Bromovirus, Celavirus, Closterovirus, Comovirus, Potexvirus, Potyvirus, Tobamovirus, Tombusvirus, Tospoviridae. Trivirinae, Tymovirus, Varicosaviris, and Secoviridae.
65. The method of embodiment 64, wherein: (i) the Bromovirus is a Cucumber Mosaic Virus, Spinach latent virus, Olive latent virus, or Brome mosaic virus; (ii) the Closterovirus is a Citrus tristeza virus or Beet yellow virus; (iii) the Comovirus is Cowpea mosaic virus, Apple latent spherical virus, or Soybean latent spherical virus; (iv) the Potexvirus is Potato virus X or Citrus yellow vein clearing virus; (v) the Potyvirus is a Pepper mottle virus, Bean yellow mosaic virus, Barley stripe mosaic virus, Wheat stripe mosaic virus, Rice yellow mottle virus, Maize dwarf mosaic virus, zucchini yellow mosaic virus, watermelon mosaic virus, or sugar cane mosaic virus; (vi) the Tobamovirus is a Tobacco mosaic virus, Tomato mosaic virus, Tomato brown rugose fruit virus, Turnip vein-clearing virus, or Pepper mild mottle virus; (vii) the Tombusvirus is a Turnip crinkle virus or Tomato bushy stunt virus; (iv) the Tospoviridae is a Tomato spotted wilt virus or watermelon bud necrosis virus; or (viii) the Tymovirus is a Turnip yellow mosaic virus, Citrus yellow vein clearing virus, Potato latent virus, Apple stem grooving virus, or Citrus leaf blotch virus.
66. The method of any one of embodiments 57 to 62, wherein the phenotypic change comprises an increase in the plant's resistance to stress, optionally wherein the stress comprises at least one abiotic stress comprising nutrient stress, light stress, water stress, heat stress, and/or cold stress, or optionally wherein the stress comprises at least one biotic stress comprising crowding, shading, or allelopathy.
67. The method of any one of embodiments 57 to 66, wherein the recombinant RNA molecule is provided to the plant of plant cell in the form of an RNA, an encapsidated RNA, or a formulation thereof.
68. The method of embodiment 67, wherein the encapsidated RNA comprises a synthetic Secoviridae satellite particle.
69. The method of any one of embodiments 57 to 68, wherein the providing comprises contacting the plant or plant cell with the RNA, encapsidated RNA, or formulation thereof, optionally wherein contacting comprises spraying, dusting, injecting, or soaking the plant or plant cell with the RNA, encapsidated RNA, or formulation thereof.
70. The method of any one of embodiments 57 to 69, wherein the recombinant RNA further comprises its reverse complementary RNA molecule.
71. The method of any one of embodiments 57 to 70, further comprising providing an hrvRdRP to the plant which recognizes the HRV 5′ or 3′ replication region and/or the subgenomic promoter in the synthetic Secoviridae satellite RNA, optionally wherein the hrvRdRP is provided by introducing a recombinant DNA or RNA encoding the hrvRdRP into the plant or a part thereof.
72. A method of manufacturing a synthetic Secoviridae satellite particle, comprising combining the recombinant RNA molecule of any one of embodiments 1 to 28 with a viral capsid protein, wherein the recombinant RNA molecule comprises an encapsidation recognition element (ERE), and wherein the ERE provides for encapsidation of the RNA by the viral capsid protein.
73. The method of embodiment 72, wherein the recombinant RNA molecule is combined with a viral capsid protein in a vessel.
74. The method of embodiment 73, wherein the combining comprises
(a) providing to a plant cell the recombinant RNA molecule, wherein the recombinant RNA molecule comprises an encapsidation recognition element (ERE), and wherein the plant cell comprises an RdRP protein that recognizes the 5′ RNA replication element and 3′ RNA replication element catalyzes synthesis of a synthetic Secoviridae satellite RNA from the recombinant RNA molecule and a viral capsid protein, wherein the ERE provides for encapsidation of the RNA by the viral capsid protein; and optionally
(b) isolating the synthetic Secoviridae satellite particle from the plant cell, a plant comprising the plant cell, or from media in which the plant cell or the plant had been grown.
75. The method of embodiment 72, 73, or 74, further comprising the step of formulating the synthetic Secoviridae satellite particle wherein the formulating comprises combining the synthetic Secoviridae satellite particle with a carrier, an excipient, and/or an adjuvant.
76. A plant propagule comprising the recombinant RNA molecule of any one of embodiments 1 to 28 and a Secoviridae RdRP, optionally wherein the plant propagule further comprises a heterologous RNA virus RdRP which recognizes an HRV 5′ or 3′ replication region and/or the subgenomic promoter in the synthetic Secoviridae satellite RNA.
77. The plant propagule of embodiment 76, wherein the plant propagule is a seed, a seedling, root, stem, leaf, shoot, tuber, rhizome, stolon, bulb, explant, embryo, or callus.
78. The plant propagule of embodiment 76 or 77, wherein the plant propagule is a mosaic comprising both plant cells comprising the recombinant RNA molecule and plant cells lacking the recombinant RNA molecule.
79. The plant propagule of embodiment 76, 77, or 78, wherein the plant propagule lacks DNA encoding the recombinant RNA molecule.
80. The plant propagule of any one of embodiments 76 to 79, wherein the plant propagule further comprises a heterologous RNA virus RdRP which recognizes an HRV 5′ or 3′ replication region and/or the subgenomic promoter in the synthetic Secoviridae satellite RNA, and the heterologous RNA virus RdRP is an Alphaflexivirus, Betaflexivirus, Bromovirus, Celavirus, Closterovirus, Comovirus, Potexvirus, Potyvirus, Tobamovirus, Tombusvirus, Tospoviridae, Trivirinae, Tymovirus, Varicosavirus, or Secoviridae RdRP or an RdRP set forth in Table 7.
81. A plant comprising the recombinant RNA molecule of any one of embodiments 1 to 28 and a Secoviridae RdRP, optionally wherein the plant propagule further comprises a heterologous RNA virus RdRP which recognizes an HRV 5′ or 3′ replication region and/or the subgenomic promoter in the synthetic Secoviridae satellite RNA.
82. The plant of embodiment 81, wherein the plant is a monocot or a dicot plant.
83. The plant of embodiment 81, wherein the plant is of the family Amaranthaceae. Asteraceae, Brassicaceae. Caricaceae. Caryophyllaceae, Cupressaceae, Cucurbitaceae, Fabaceae, Gentianaceae, Pinaceae, Poaceae, Rosaceae. Rutaceae, or Solanaceae.
84. The plant of embodiment 81, 82, or 83, wherein the plant lacks DNA that encodes the recombinant RNA molecule.
85. The plant of any one of embodiments 81 to 84, wherein the plant comprises a virus of the family Secoviridae, and wherein the Secoviridae RdRP is provided to the plant cell by the virus of the family Secoviridae, optionally wherein the Secoviridae RdRP comprises a protein having at least 85%, 90%, 95%, 97%, or 99% sequence identity to SEQ ID NO: 471, 530, or 558-564.
86. The plant of any one of embodiments 81 to 85, wherein the virus of the family Secoviridae is endemic to the plant, optionally wherein the endemic virus of the family Secoviridae is non-pathogenic and/or commensal.
87. The plant of any one of embodiments 81 to 86, wherein the Secoviridae RdRP, the 5′ RNA replication element, and/or the 3′ RNA replication element are derived from a virus of the family Secoviridae comprising one or both of the Secoviridae RdRP, 5′ RNA replication element, and/or 3′ RNA replication elements.
88. The plant of any one of embodiments 81 to 87, wherein the RdRP has at least 85%, 90%, 95%, 97%, 98%, or 99% identity to SEQ ID NO: 471, 530, or 558-564, wherein the 5′ RNA replication element has at least 85%, 90%, 95%, 97%, 98%, or 99% identity to the RNA encoded by SEQ ID NO: 467, 468, 512, 546-551, or 631, and/or wherein the 3′ RNA replication element has at least 85%, 90%, 95%, 97%, 98%, or 99% identity to the RNA encoded by SEQ ID NO: 469, 470, 520, 521, or 552-557.
89. The plant of any one of embodiments 81 to 88, wherein the plant is a grafted plant and wherein the rootstock and/or scion of the grafted plant comprise at least one cell comprising the recombinant RNA and the Secoviridae RdRP.
90. The plant of any one of embodiments 81 to 89, wherein the plant is not produced by an essentially biological process.
91. The plant of any one of embodiments 81 to 90, wherein the plant further comprises a heterologous RNA virus (HRV) RdRP which recognizes an HRV 5′ or 3′ replication region and/or the subgenomic promoter in the synthetic Secoviridae satellite RNA, and the heterologous RNA virus RdRP is an Alphaflexivirus, Betaflexivirus, Bromovirus, Celavirus. Closterovirus. Comovirus, Potexvirus, Potyvirus. Tobamovirus. Tombusvirus, Tospoviridae, Trivirinae. Tymovirus, Varicosavirus, or Secoviridae RdRP or an RdRP set forth in Table 7.
92. A Secoviridae satellite system that is self-replicating when introduced into a plant or plant cell, comprising: (a) a recombinant Secoviridae satellite RNA of any one of embodiments 1 to 28; and (b) an exogenous virus of the family Secoviridae that is capable of replication in the plant or plant cells and that encodes the Secoviridae RdRP that recognizes the 5′ and 3′ replicase recognition sequences in the recombinant Secoviridae satellite RNA, optionally wherein the Secoviridae satellite system further comprises a heterologous RNA virus RdRP which recognizes an HRV 5′ or 3′ replication region and/or the subgenomic promoter in the synthetic Secoviridae satellite RNA.
93. The self-replicating Secoviridae satellite system of embodiment 92, wherein the exogenous virus of the family Secoviridae is endemic or native to a different species, variety, or germplasm of plant.
94. The self-replicating Secoviridae satellite system of embodiment 92, wherein the Secoviridae satellite system further comprises a heterologous RNA virus (HRV) RdRP which recognizes an HRV 5′ or 3′ replication region and/or the subgenomic promoter in the synthetic Secoviridae satellite RNA, and the heterologous RNA virus RdRP is optionally an Alphaflexivirus, Betaflexivirus, Bromovirus, Celavirus, Closterovirus, Comovirus, Potexvirus, Potyvirus, Tobamovirus, Tombusvirus, Tospoviridac, Trivirinac, Tymovirus, Varicosavirus, or Secoviridae RdRP or an RdRP set forth in Table 7.
95. A recombinant DNA molecule comprising a first promoter which is operably linked to DNA encoding the RNA molecule of any one of embodiments 1-28.
96. A cell comprising the recombinant DNA molecule of embodiment 95, wherein the cell is a bacterial cell, a fungal cell, a plant cell, an insect cell, or an invertebrate animal cell.
97. A vector for bacterially mediated plant transformation, comprising the recombinant DNA molecule of embodiment 95.
98. The vector of embodiment 97, wherein the bacterium that mediates the plant transformation is an Agrobacterium sp., a Sinorhizobium sp., a Mesorhizobium sp., a Bradyrhizobium sp., Rhizobium sp., or an Ensifer sp. and the vector is adapted for transfonnation with the bacterium.
99. The vector of embodiment 97 or 98, wherein the bacterium that mediates the plant transformation is an Agrobacterium sp., and wherein the vector further comprises T-DNAs flanking the DNA molecule encoding the recombinant RNA molecule.
100. The vector of embodiment 97, 98, or 99, contained within a bacterial or plant cell.
101. An expression system comprising: (a) the recombinant DNA molecule of embodiment 95; and(b) a cell containing the recombinant DNA molecule and an RdRP protein that recognizes the 5′ and 3′ RNA replication elements encoded by the DNA molecule.
102. The expression system of embodiment 101, wherein the recombinant DNA molecule further comprises at least one additional element comprising: (i) DNA encoding at least one RNA encoding a viral movement protein (MP); (ii) DNA encoding at least one tRNA-like molecule; (iii) DNA encoding an encapsidation recognition element (ERE); (iv) DNA encoding an RNA comprising, from 5′ to 3′ and operably linked, a 5′ RNA replication element recognized by a Secoviridae RNA-dependent RNA polymerase (RdRP), the RNA of (i) and optionally an operably linked RNA of (ii) and/or (iii), and a 3′ RNA replication element: (v) DNA encoding an RNA promoter; (vi) DNA encoding an RNA-dependent RNA polymerase (RdRP) that recognizes an HRV 5′ or 3′ replication region and/or a subgenomic promoter; and/or (vii) a DNA molecule comprising a promoter which is operably linked to the DNA encoding the RNA of at least one of (i), (ii), (iii), (iv). (v), or (vi).
103. The expression system of embodiments 101 or 102, wherein the cell is a bacterial cell, a plant cell, a fungal cell, an insect cell, or an invertebrate animal cell, optionally wherein the bacterial cell is an Agrobacterium sp., a Sinorhizobium sp., a Mesorhizobium sp., Braclyrhizobium sp., Rhizobium sp., or an Ensifer sp. cell.
104. The expression system of any one of embodiment 101, 102, or 103, further comprising: (i) a viral capsid protein that can encapsidate an RNA molecule comprising the encapsidation recognition element (ERE); (ii) an RNA-binding protein (RBP) that can bind to the RNA molecule encoded by the DNA molecule, optionally wherein the RBP binds to an RNA effecter; (iii) an RNA cleavage agent that cleaves the RNA molecule; and/or (iv) an RNA promoter dependent RNA polymerase (RdRP) protein that recognizes an HRV 5′ or 3′ replication region and/or a subgenomic promoter recognizes an RNA promoter in the RNA molecule.
105. The expression system of embodiment 104, wherein the viral capsid protein is: (a) expressed by the recombinant DNA molecule in the cell, (b) co-expressed by a second recombinant DNA molecule in the cell; (c) provided exogenously to the cell; or (d) expressed by a virus in the cell.
106. The expression system of embodiment 104 or 105, wherein: (i) the capsid protein, viral movement protein (MP). RdRP protein, and/or the RdRP protein that recognizes an HRV 5′ or 3′ replication region and/or a subgenomic promoter is heterologous to the cell and/or (ii) wherein the RdRP protein or a polynucleotide encoding the RdRP protein is provided exogenously to the cell.
107. The expression system of any one of embodiments 101 to 106, wherein the cell is a plant cell.
108. The expression system of 107, wherein the plant cell contains a virus of the family Secoviridae which expresses the RdRP protein that recognizes the 5′ RNA replication element and the 3′ RNA replication element.
109. The expression system of embodiment 108, wherein the virus of the family Secoviridae occurs naturally in the plant cell.
110. The expression system of any one of embodiments 101 to 109, wherein the recombinant DNA molecule further comprises at least one RNA encoding a viral MP, a tRNA-like molecule from an Arabidopsis FT mRNA, and an encapsidation recognition element comprising a TMV-OAS.
111. An agricultural formulation comprising the expression system of any one of embodiments 101 to 110.
112. The agricultural formulation of embodiment 111, wherein the formulation comprises the expression system and a carrier, an excipient and/or an adjuvant.
113. An agricultural formulation comprising the recombinant DNA molecule of embodiment 95.
114. The agricultural formulation of embodiment 113, wherein the formulation comprises the recombinant DNA molecule and a carrier, an excipient and/or an adjuvant.
115. A method of producing an exogenous polypeptide in a plant or plant cell, comprising: providing a plant or plant cell comprising the recombinant RNA molecule of any one of embodiments 1 to 28 or the recombinant DNA molecule of embodiment 95, wherein the cargo RNA molecule encoded by the RNA or DNA molecule comprises a translatable messenger RNA encoding the exogenous polypeptide, wherein the plant or plant cell comprises an RdRP protein that recognizes the 5′ RNA replication element and the 3′ RNA replication element of the recombinant RNA and that catalyzes synthesis of a synthetic Secoviridae satellite RNA from the recombinant RNA molecule, and wherein the exogenous polypeptide is translated from the translatable messenger; optionally wherein the plant or plant cell further comprises a heterologous RNA virus (HRV) RNA promoter dependent RNA polymerase (hrvRdRP) protein that recognizes an HRV 5′ or 3′ replication region and/or a subgenomic promoter recognizes an RNA promoter in the RNA molecule.
116. The method of embodiment 115, further comprising a first step of providing a population of plants comprising the plant cells comprising the virus of the family Secoviridae which provides the RdRP protein; and then providing the recombinant RNA or DNA molecule, the cell, the vector, the recombinant RNA or DNA molecule, or formulation thereof to the plants comprising the plant cells and optionally further comprising providing the hrvRdRP to the plants comprising the plant cells.
117. The method of embodiment 115 or 116, wherein the virus of the family Secoviridae is exogenously provided to the plant cell and/or wherein an HRV encoding the hrvRdRP is provided to the plant cell.
118. The method of embodiment 115, 116, or 117, wherein the RdRP and/or hrvRdRP protein or a recombinant polynucleotide encoding the RdRP and/or hrvRdRP protein is exogenously provided to the plant cell.
119. The method of any one of embodiments 115 to 118, further comprising a first step of providing a population of plants comprising the plant cells comprising the RdRP protein or the recombinant polynucleotide encoding the RdRP; and then providing the recombinant RNA molecule to the plants comprising the plant cells.

. The method of any one of embodiments 115 to 119, wherein the recombinant RNA molecule has been produced in a fermentation system.
121. The method of any one of embodiments 115 to 120, further comprising the step of determining if the plant cell comprises a virus of the family Secoviridae which can provide the RdRP.
122. A method of producing a modified plant propagule that comprises at least one plant cell comprising a recombinant RNA molecule of embodiment 1 to 28, comprising isolating a plant propagule comprising at least one plant cell comprising the recombinant RNA molecule and a Secoviridae RNA-dependent RNA polymerase (RdRP) from a mixed population of plant cells comprising both plant cells comprising the recombinant RNA molecule and plant cells lacking the recombinant RNA molecule.
123. The method of embodiment 122, wherein the mixed population of plant cells comprise a population of protoplasts or a population of cells in callus or an explant.
124. The method of embodiment 122 or 123, wherein the mixed population of plant cells comprise plant cells comprising the Secoviridae RdRP and plant cells lacking the Secoviridae RdRP.
125. The method of embodiment 122, 123, or 124, wherein the mixed population of plant cells comprise plant cells comprising the Secoviridae RdRP.
126. The method of any one of embodiments 122 to 125, wherein the mixed population of plant cells is screened or selected for the presence of the plant cell comprising the recombinant RNA molecule prior to isolating the plant propagule.
127. The method of any one of embodiments 122 to 126, wherein the isolation comprises selecting for the plant cell comprising the recombinant RNA molecule prior to isolating the plant propagule.
128. The method of any one of embodiments 122 to 127, wherein the mixed population is located within a plant or a plant part.
129. The method of any one of embodiments 122 to 128, wherein the plant or plant part is screened or selected for presence of the recombinant RNA molecule prior to isolating the plant propagule.
130. The method of any one of embodiments 122 to 128, wherein the plant or plant part is screened or selected for systemic presence of the recombinant RNA molecule prior to isolating the plant propagule.
131. The method of any one of embodiments 122 to 130, wherein the recombinant RNA molecule encodes a selectable marker and the plant propagule comprising the recombinant RNA molecule is isolated by selecting for presence of the selectable marker.
132. The method of embodiment 131, further comprising selecting a plant propagule comprising a recombinant RNA molecule wherein the selectable marker has been removed.
133. The method of any one of embodiments 122 to 132, wherein the plant propagule comprising the recombinant RNA molecule is isolated by detecting the RNA molecule in one or more plant cells comprising the recombinant RNA molecule and separating the one or more plant cells comprising the recombinant RNA molecule from the plant cells lacking the recombinant DNA molecule.
134. The method of any one of embodiments 122 to 133, wherein the plant propagule is a mosaic comprising both plant cells comprising the recombinant RNA molecule and plant cells lacking the recombinant RNA molecule.
135. The method of any one of embodiments 122 to 134, wherein the plant propagule is a mosaic comprising both plant cells comprising the Secoviridae RdRP and plant cells lacking the Secoviridae RdRP.
136. The method of any one of embodiments 122 to 134, wherein the plant propagule lacks DNA that encodes the recombinant RNA molecule.
137. The method of any one of embodiments 122 to 136, wherein the plant or plant part lacks DNA that encodes the recombinant RNA molecule.
138. The method of any one of embodiments 122 to 137, wherein the plant propagule comprises the cell, or a seed, seedling, root, stem, leaf, shoot, tuber, rhizome, stolon, bulb, explant, or callus comprising the cell.
139. The method of embodiment 138, further comprising multiplying the cell, seed, seedling, root, stem, leaf, shoot, tuber, rhizome, stolon, bulb, explant, or callus to obtain progeny, wherein the progeny comprise the recombinant RNA molecule.
140. The method of embodiment 139, wherein the multiplying comprises culturing a plurality of explants obtained from the cell, seed, seedling, root, stem, leaf, shoot, tuber, rhizome, stolon, bulb, explant, or callus.
141. The method of any one of embodiments 122 to 140, wherein the isolated propagule comprises the cell and the method further comprises regenerating a plant, seedling, root, stem, leaf, shoot, tuber, rhizome, stolon, bulb, explant, or callus comprising the recombinant RNA from said cell.
142. The method of any one of embodiments 122 to 140, wherein the isolated propagule comprises callus and the method further comprises regenerating a plant, seedling, root, stem, leaf, shoot, tuber, rhizome, stolon, bulb, or explant comprising the recombinant RNA from said callus.
143. The method of embodiment 141 or 142, wherein a plant is regenerated and wherein the method further comprises recovering F₁ seed or F₁ progeny comprising the recombinant RNA from the plant.
144. A method of providing a synthetic Secoviridae satellite RNA to a plant comprising: grafting a scion onto a rootstock comprising recombinant RNA molecule of any one of embodiments 1 to 28, wherein at least one cell of the rootstock and/or the scion comprises the Secoviridae RdRP.
145. The method of embodiment 144, wherein the scion lacks the recombinant RNA molecule prior to grafting.
146. The method of embodiment 144 or 145, wherein the rootstock comprises the Secoviridae RdRP.
147. The method of any one of embodiments 144 to 146, wherein the RdRP is provided by a virus of the family Secoviridae endemic to the rootstock, optionally wherein the virus of the family Secoviridae endemic to the rootstock is non-pathogenic and/or commensal.
148. The method of any one of embodiments 144 to 146, wherein the RdRP is exogenously provided to the rootstock.
149. The method of any one of embodiments 144 to 148, wherein the scion comprises the Secoviridae RdRP.
150. The method of any one of embodiments 144 to 149, wherein the RdRP is provided by a virus of the family Secoviridae endemic to the scion and/or wherein the RdRP is exogenously provided to the scion, optionally wherein the virus of the family Secoviridae endemic to the scion is non-pathogenic and/or commensal.
151. A method for producing a plant that transmits a recombinant RNA molecule to progeny plants or seed comprising isolating an F₁ progeny plant or seed comprising at least one cell comprising a Secoviridae RNA-dependent RNA polymerase (RdRP) and the recombinant RNA molecule of any one of embodiments 1 to 28 from a population of F₁ plants or seed obtained from a parent plant comprising the recombinant RNA molecule.
152. The method of embodiment 151, wherein the F₁ progeny plant or seed comprising the cell is isolated by screening the population of F₁ plants or seed obtained from a parent plant for the presence of the recombinant RNA molecule and propagating the F₁ progeny plant or seed comprising the recombinant RNA molecule.
153. The method of embodiment 151, wherein the recombinant RNA molecule encodes a selectable marker and the F₁ progeny plant or seed comprising the recombinant RNA molecule is isolated by selecting the F₁ progeny plant or seed comprising the recombinant RNA molecule for presence of the selectable marker.
154. The method of any one of embodiments 151 to 153, wherein the F₁ progeny plant or seed lack DNA that encodes the recombinant RNA molecule.
155. The method of any one of embodiments 151 to 154, wherein the parent plant lacks DNA that encodes the recombinant RNA molecule.
156. The method of embodiment 153, wherein the selected F₁ progeny plant transmits the recombinant RNA molecule to at least F₂ progeny.
157. The method of any one of embodiments 151 to 156, wherein the F₁ progeny plant or seed population is obtained from a parent plant used as a pollen recipient.
158. The method of any one of embodiments 151 to 156, wherein the F₁ progeny plant or seed population is obtained from a parent plant used as a pollen donor.
159. The method of any one of embodiments 151 to 156, wherein the F₁ progeny plant or seed population is obtained by selfing the parent plant.
160. The method of any one of embodiments 151 to 159, wherein the parent plant or a part thereof comprising the plant cell is screened or selected for presence of the recombinant RNA molecule prior to isolating the F₁ progeny plant or seed.
161. The method of any one of embodiments 151 to 160, wherein the parent plant or one or more parts thereof are screened for systemic presence of the recombinant RNA molecule prior to isolating the F₁ progeny plants, optionally wherein the part comprises floral tissue or male or female reproductive tissue.
162. The method of any one of embodiments 151 to 161, wherein the pericarp of the parent plant is screened or selected for presence of the recombinant RNA molecule.
163. The method of any one of embodiments 151 to 162, wherein F₁ seeds are obtained from a parent plant selected for presence of the recombinant RNA molecule in pericarp tissue.
164. The method of any one of embodiments 151 to 163, wherein an F₁ seed of the parent plant is non-destructively screened for presence of the recombinant RNA molecule.
165. The method of embodiment 164, wherein the F₁ seed of the parent plant is non-destructively screened by assaying maternally derived or endosperm tissue of the seed for the presence of the recombinant RNA molecule.
166. The method of any one of embodiments 151 to 165, further comprising introducing the recombinant RNA molecule or a polynucleotide encoding the recombinant RNA molecule into a plant cell and obtaining the parent plant comprising the recombinant RNA molecule from the plant cell.
167. The method of any one of embodiments 151 to 166, wherein the propagule, plant, plant part, scion, and/or rootstock comprises a heterologous viral coat protein which can encapsidate the recombinant RNA molecule and/or comprises the recombinant RNA molecule encapsidated by a heterologous viral coat protein.
168. A method of barcoding a plant, plant cell, progeny thereof, or part thereof comprising providing to the plant or plant cell the recombinant RNA molecule of any one of embodiments 1 to 28, wherein the cargo RNA of the recombinant RNA molecule comprises a barcode RNA molecule, and wherein the plant or plant cell comprises a Secoviridae RNA-dependent RNA polymerase (RdRP).
169. The method of embodiment 168, wherein the barcode RNA molecule comprises a sequence that uniquely identifies the plant, plant cell, progeny thereof, or part thereof.
170. The method of embodiment 168 or 169, wherein the barcode RNA molecule comprises a sequence that is not present in the genome and/or transcriptome of a wild-type plant of the same species, a pathogen thereof, or a symbiont thereof.
171. The method of any one of embodiments 168 to 170, wherein the barcode RNA molecule comprises a random sequence.
172. The method of any one of embodiments 168 to 171, wherein the barcode RNA molecule comprises a forward primer binding site and a reverse primer binding site that is not present in the genome and/or transcriptome of a wild-type plant of the same species, a pathogen thereof, or a symbiont thereof.
173. The method of any one of embodiments 168 to 172, wherein the barcode RNA molecule is up to about 8 kb in length.
174. The method of any one of embodiments 168 to 172, wherein the barcode RNA molecule has a length of 10 to 8000 nucleotides, 20 to 1000 nucleotides, or 50 to 500 nucleotides, optionally wherein the barcode RNA molecule has a length of 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, or 8000 nucleotides.
175. The method of any one of embodiments 168 to 174, wherein the barcode RNA molecule comprises a non-protein coding sequence.
176. The method of any one of embodiments 168 to 174, wherein the plant transmits the recombinant RNA molecule to progeny.
177. The method of any one of embodiments 168 to 176, wherein the plant, plant cell, progeny thereof, or part thereof lacks DNA that encodes the recombinant RNA molecule.
178. The method of any one of embodiments 168 to 176, further comprising isolating an F₁ progeny plant or seed comprising at least one cell comprising the Secoviridae RdRP and the recombinant RNA molecule.
179. The method of embodiment 178, wherein the F₁ progeny plant or seed is obtained from the plant used as a pollen recipient.
180. The method of embodiment 178, wherein the F₁ progeny plant or seed is obtained from the plant used as a pollen donor.
181. The method of embodiment 178, wherein the F₁ progeny plant or seed is obtained by selfing the plant.
182. The method of any one of embodiments 168 to 181, further comprising propagating the plant or plant cell to obtain a plant part or a plant propagule comprising the barcode RNA molecule, optionally wherein said propagule comprises callus, tubers, and/or rootstock.
183. A method of identifying a barcoded plant, plant part, or plant cell, the method comprising screening for the presence of a barcode RNA molecule in the plant, plant part, or plant cell, wherein the plant, plant part, or plant cell comprises the recombinant RNA molecule of any one of embodiments 1 to 28, wherein the cargo RNA of the recombinant RNA molecule comprises the barcode RNA molecule.

### SUMMARY OF TABLES

TABLE 1
Secoviridae 5′ and 3′ RNA Replication Element DNA Coding Sequences

| Description¹ | SEQ ID NO² | Dot-Bracket structure of encoded RNA |
|---|---|---|
| Apple latent spherical virus | SEQ ID NO: | ((.((..(((((..((((((.(((....((((..........(((((...((......))...)))))..... |
| (ALSV) segment 1 (NCBI: | 467 | ))))..))).))))))..)))))..(((((...))))).((.........)).......(((((((....... |
| NC_003787.1) | | ..))))))).((.(((((((((....))))))))).))...((((((....)))))).((((((((((. |
| 5′ RNA replication element | | ................(((((((((...))))).)))).(((((((...........))))))).....(((( |
| | | (((((......))))))))).))))))))))......)).)).(((((((((((....).)))).)))) |
| | | )).......((((((.....)))))). |
| Apple latent spherical virus | SEQ ID NO: | (((...(((((..((((((((..(((...((..((...((((.................)))).)).))...) |
| (ALSV) segment 2 (NCBI: | 468 | ))...))))))))..))))).))).(((((..................(((((((......)))))))..... |
| NC_003788.1) | | .....................(((((((.......))))))).....((.(((((....))))).)).....((( |
| 5′ RNA replication element | | .((((((.......)))))).)))..............(((((((....)))))))...))))).......... |
| | | ................ |
| Soybean latent spherical virus | SEQ ID NO: | ............. |
| segment 1 (NCBI: | 512 | |
| NC_032270) | | |
| 5′ RNA replication element | | |
| Soybean latent spherical virus | SEQ ID NO: | .........(((((...))))). |
| segment 2 (NCBI: | 631 | |
| NC_032271) | | |
| 5′ RNA replication element | | |
| Currant latent virus segment | SEQ ID NO: | ((.(((((((((.(((........))).))).)))))).)).((((............................. |
| 1 (NCBI: NC_029038) | 546 | .........))))....... |
| 5′ RNA replication element | | |
| Arabidopsis latent virus-1 | SEQ ID NO: | .........(((((((..(((....((((....))))....)))...))))))).((((((......(((((. |
| segment 1 (NCBI: | 547 | .((.((((((((((..........((((((...(((.(((....))))))...)))))))))))).)))) |
| NC_078590) | | )).......))))).......))))))((((..(((((((.......)))))))..))))........... |
| 5′ RNA replication element | | |
| Artichoke Italian latent virus | SEQ ID NO: | ((((.....))))......((((....((((.((((....((.(((....)))))...)))))))).(((( |
| segment 1 (NCBI: | 548 | (((.........)))).))).........((..(((.((((((...)))))).)))..))....((((...... |
| NC_043684) | | ..)))).((((((((...))))))))(((((((....))))))).))))... |
| 5′ RNA replication element | | |
| Blueberry latent spherical | SEQ ID NO: | ..(((((((......)))))))....................................... |
| virus segment 1 (NCBI: | 549 | |
| NC_038764) | | |
| 5′ RNA replication element | | |
| Grapevine Bulgarian latent | SEQ ID NO: | ..(((((....((((((((((..(((......))).........))))))))))....)))))..((((... |
| virus segment 1 (NCBI: | 550 | ..........)))). |
| NC_015492) | | |
| 5′ RNA replication element | | |
| Strawberry latent ringspot | SEQ ID NO: | ......((((....(((((.......................((((...((.(((((....)))))))...)))) |
| virus segment 1 (NCBI: | 551 | .(((....((.(((.((((((....))))))..)))..))....)))...(((((((....))))))).... |
| NC_006964) | | ...)))))..))))..(((((.((((...(((....))).(((((((....)))))))............... |
| 5′ RNA replication element | | .........)))).)))))............. |
| Apple latent spherical virus | SEQ ID NO: | .....((((.((.(((..((.((((((................(((((((..(((((((((.......(((.( |
| (ALSV) segment 1 (NCBI: | 469, SEQ ID | ((.((((....)))).))).)))........))))))))).........))))))).........((((..... |
| NC_003787.1) | NO: 520 | .......))))......))))))))..))).)).))))... |
| 3′ RNA replication element | | |
| Apple latent spherical virus | SEQ ID NO: | .((((.((.((((.(((.(((((((.......................(((((((...(((((((.((((... |
| (ALSV) segment 2 (NCBI: | 470 | ))))))..)))))....(((((.((.....(((((...))))))).))))))))))))......((((.. |
| NC_003788.1) | | .((....))...)))).))))))).))).)))).)).))))... |
| 3′ RNA replication element | | |
| Soybean latent spherical virus | SEQ ID NO: | ...(((((.(((.(((.(((((((((((((((((...)))).))))))))....))))).))).))). |
| segment 1 (NCBI: | 521 | )))))((((.(((.((((((..((((....)))))))))))))((((((((((..(((((((((..( |
| NC_032270) | | ((((((....)).))))).((((.((.....))))))......((((((.((((((.(((((((((((( |
| 3′ RNA replication element | | (((..............)))))))))))(((((.((....)))))))..................(((((.((( |
| | | ((..((((.......((((((....))))))((((((((........)))).)))))))))))))))))) |
| | | ..)))))))))).)))))).(((((((....)))).)))...(((((...(((........))).((((. |
| | | ((((((((.(((((((.((((..............(((..(((((((((.........)))))...)))).. |
| | | .))).((..(((((((((......)))))))))..)).((((.......)))).((((((((((((((( |
| | | (........(((((((((((((((((((.......)))))))...)))......)))))))))...((((( |
| | | .((.....)))))))..((.((((((.(.......))))))).)).....((((((........)))))).( |
| | | (((.(((((((((((...(((....)))....))))))....))))).))))(((..(((((((((..( |
| | | (((((((((((((....))...)))).))........))).)))..))))))).(((.((.....)).))) |
| | | ...((((((((....))))))).).))..)))...))))))))))))))))..((((((....)))))) |
| | | (((((((.(((((((((....(((.(..(((((......)))))..).(((((((((.((((........) |
| | | ))).((((.((((...((((......))))...))))...))))..((((.........))))(((((.... |
| | | .)))))........))))))))).....((((((((....))))))))))).....((((((........)) |
| | | )))).....((((((.....)))))).(((.(((((((((((.((((.((((((.((((....))))... |
| | | ...(((((....))))).....)))))).))))...)))))))))))..)))...(((.(((......))) |
| | | )))..((((..(((....)))..))))...(((......))).))))))))).)).....))))))))).. |
| | | .))))))).)))))))).)))).))))).))))))))).))))..((((((..((.((((((...))) |
| | | )))..))..)))))).))))))..)))).....(((((((((..((........))....))))))))).. |
| | | .(((((........)))))..((((((((((..(((((((.....))).))))...)))))..))))).( |
| | | ((((.........))))).... |
| Currant latent virus segment | SEQ ID NO: | ......((((..(((...((.(((..((((((((...((((((((...)))).))))((((((((...... |
| 1 (NCBI: NC_029038) | 552 | .......))))))))...............))))))))....))).)).)))..).)))... |
| 3′ RNA replication element | | |
| Arabidopsis latent virus-1 | SEQ ID NO: | ..................(((((.((((((((((((((((.....)))...))))...)).))))..)))..) |
| segment 1 (NCBI: | 553 | )))).(((......((((((.((......))))))))......))).....(((.(((((..((((((((.. |
| NC_078590) | | .....))))...))))..))))).))).............. |
| 3′ RNA replication element | | |
| Artichoke Italian latent virus | SEQ ID NO: | ((.(((((..((((((((((....((...((((...((((..(((.((((...(((((((((((....... |
| segment 1 (NCBI: | 554 | ...........))))))....))))).)))).(((((((((((......)))))...))))))..(((((( |
| NC_043684) | | (.........))))))).......(((....((((((((((..(((....))).((((..(((((((...... |
| 3′ RNA replication element | | .......)))))))))))..))))))))))...))).)))..))))..))))....))....)))))))) |
| | | )).))))).)). |
| Blueberry latent spherical | SEQ ID NO: | .((((((.....)))))).((((((((.((((.....)))).)))(((((((((((.....)))))..( |
| virus segment 1 (NCBI: | 555 | ((((.(((((....)))))..(((((((((....)))..)))))).((.((((((.((...)).))))) |
| NC_038764) | | ).))..((((....)))).((((((......))))))))))).((((((((((........))))))).) |
| 3′ RNA replication element | | ))....(((.(((((....))))))))((((((((...))))))))..(((((((.(((((((((((( |
| | | ((((((((((.(((...((((((.((((.............)))))))))).)))))))))))))))... |
| | | ....)))))))))).))))))).)))))).((((((((((.(((((((((...(((((((((.(((( |
| | | (((..((((........)).))..))))))).........(((((.....))))).....(((((.......)) |
| | | ))).((((((((((.(((.(((((((((((((((((((((((......(((.((((((((...)))) |
| | | )))).)))..))))).....)))))))))...))))).))))...((((((((..............)))) |
| | | ))))(((.((((((((((....)))))(.((......)).)..((((((............)))))).((( |
| | | (((((..((((....))))...))))))))...(((((........))))).((((((((((((..(((( |
| | | (((((....)))))))))...((((((....)))))).))))))))))))....(((((.(((((((( |
| | | (((((...((((...((((((....(((((((.(.....(((((....)))))..))))))))....)))) |
| | | ))....))))...)))))))....))))))))))).)))))..)))((((.......))))..((((((( |
| | | ((..........(((((((...((((.((((((.(((((....)))))))))))...))))..((((.... |
| | | .....))))(((((.....))))).)))))))))))))))).....(((((((....))))))).))).. |
| | | ))))))))))...((..(((.(((....))))))..))..)))))))))))))))))).))..))))) |
| | | )))...((((((....)))))).(((....)))..(((((....)))))(((((.(((....(((((.... |
| | | )))))..))).)))))...(((((......))))).((((((((...........))))))))......((( |
| | | (((.((((((....)))))).)))))).)))))((((((......)))))).. |
| Grapevine Bulgarian latent | SEQ ID NO: | ...(((((((.(((.((((((.((((((.((((.......))))..((((......))))(((...))).( |
| virus segment 1 (NCBI: | 556 | (((((...((....))..))))))...))))))(((((....))))).((((((....))))))))))) |
| NC_015492) | | ).......(((((((.........)))))))..(((((((.((((((....)))))).....(((..((((( |
| 3′ RNA replication element | | (.(((...((.(((((..((.((.((((((..(((((((((((((((.((.((((((((......(((( |
| | | ((.(.(((..(((((.((.((.((..........((((((........))))))(((((.((((((...... |
| | | .....))))))...))))).(((((........))))))))).)).))))).))))))))).)......)) |
| | | )))))).)).))))).(((((....)))))...((((((((((((......)))))........)))))) |
| | | ).(((((((....((((....((((....))))...))))...))))))))))))))))).)))))))) |
| | | ..)).))))).))...))).))))))..))).......))))))).((.((((((((((.((..(((((( |
| | | (.((((((...))))))...............((((....)))).))))))).....)))))))))))))). |
| | | ...))).))))))).((((((((....)))))))).((((((((((.(((((((((...))))))))) |
| | | .(((((((((.((((................(((((((((((..((((((((.(((((((...(((.(((. |
| | | ...)))..)))........))))))).))))))))...))))))))))).(((((((((((.((((.... |
| | | ..)))).)))))))))))...............((((......)))).))))))))))))).....(((((( |
| | | .(...((((((.((((((...(((((((....)))))))...))))))))))))).))))))........ |
| | | ...........)))))))))) |
| Strawberry latent ringspot | SEQ ID NO: | ...(((.............(((((((.((((...(((((...)))))....))))((((....))))...))) |
| virus segment 1 (NCBI: | 557 | )))).((.((((((((..............((((((......))))))...((((((.(((((((....))) |
| NC_006964) | | ))))......((((..(((.......)))..))))((((((......))))))......))))))..(((.( |
| 3′ RNA replication element | | ((.....(((...(((((......)))))(((((..((.((((....)))))).)))))(((((((..(( |
| | | .((((((((((.((.(((.....((((.............)))).....)))))....(((((((.((((... |
| | | .((((((...(((......)))....))))))...))...)).)))))))..)))).))))))))))))) |
| | | )).((((((..(((......)))...)))))).....((((.((......((((((((.....)))))))). |
| | | .....))))))))))))))).)))))))).))..((((((.....)))))).)))... |

¹The NCBI descriptors refer to the National Center for Biotechnology Information database accession number for entries in the world wide web internet database “ncbi[dot]nlm[dot]nih.gov/nuccore.”
²RNA equivalents of the DNA sequences are also contemplated and can be obtained from the DNA sequences provided.

TABLE 2
Viral capsid protein and origin of assembly sequences

| | Origin of assembly | |
|---|---|---|
| Virus¹ | sequence² | Capsid Protein |
| NC_001367.1 \| Tobacco mosaic virus, complete genome\| | SEQ ID NO: 1 | SEQ ID NO: 26 |
| Tobacco mosaic virus | | |
| NC_001556.1 \| Tobacco mild green mosaic virus, | SEQ ID NO: 2 | SEQ ID NO: 27 |
| complete genome\| Tobacco mild green mosaic virus | | |
| NC_002692.1 \|Tomato mosaic virus, complete genome\| | SEQ ID NO: 3 | SEQ ID NO: 28 |
| Tomato mosaic virus | | |
| NC_003630.1 \|Pepper mild mottle virus, complete | SEQ ID NO: 4 | SEQ ID NO: 29 |
| genome\| Pepper mild mottle virus | | |
| NC_004106.1 \|Paprika mild mottle virus, complete | SEQ ID NO: 5 | SEQ ID NO: 30 |
| genome\|Paprika mild mottle virus | | |
| NC_009642.1 \|Bell pepper mottle tobamovirus, complete | SEQ ID NO: 6 | SEQ ID NO: 31 |
| genome\|Bell pepper mottle virus | | |
| NC_022230.1 \|Tomato mottle mosaic virus isolate MX5, | SEQ ID NO: 7 | SEQ ID NO: 32 |
| complete genome\|Tomato mottle mosaic virus | | |
| NC_001728.1 \|Odontoglossum ringspot virus, complete | SEQ ID NO: 8 | SEQ ID NO: 33 |
| genome\|Odontoglossum ringspot virus | | |
| NC_003852.1 \|Obuda pepper virus, complete | SEQ ID NO: 9 | SEQ ID NO: 34 |
| genome\|Obuda pepper virus | | |
| NC_009041.1 \|Rehmannia mosaic virus, complete | SEQ ID NO: 10 | SEQ ID NO: 35 |
| genome\|Rehmannia mosaic virus | | |
| NC_010944.1 \|Brugmansia mild mottle virus, complete | SEQ ID NO: 11 | SEQ ID NO: 36 |
| genome\|Brugmansia mild mottle virus | | |
| NC_022801.1 \|Yellow tailflower mild mottle virus | SEQ ID NO: 12 | SEQ ID NO: 37 |
| isolate Cervantes, complete genome\|Yellow tailflower | | |
| mild mottle virus | | |
| NC_030229.1 \|Tropical soda apple mosaic virus isolate | SEQ ID NO: 13 | SEQ ID NO: 38 |
| Okeechobee, complete genome\|Tropical soda apple | | |
| mosaic virus | | |
| NC_001801.1 \|Cucumber green mottle mosaic virus, | SEQ ID NO: 14 | SEQ ID NO: 39 |
| complete genome\|Cucumber green mottle mosaic virus | | |
| NC_002633.1 \|Cucumber fruit mottle mosaic virus, | SEQ ID NO: 15 | SEQ ID NO: 40 |
| complete genome\|Cucumber fruit mottle mosaic virus | | |
| NC_003610.1 \|Kyuri green mottle mosaic virus, | SEQ ID NO: 16 | SEQ ID NO: 41 |
| complete genome\|Kyuri green mottle mosaic virus | | |
| NC_003878.1 \|Zucchini green mottle mosaic virus, | SEQ ID NO: 17 | SEQ ID NO: 42 |
| complete genome\|Zucchini green mottle mosaic virus | | |
| NC_008614.1 \|Cucumber mottle virus, complete | SEQ ID NO: 18 | SEQ ID NO: 43 |
| genome\|Cucumber mottle virus | | |
| Watermelon green mottle mosaic virus | SEQ ID NO: 19 | SEQ ID NO: 44 |
| NC_001873.1 \|Turnip vein-clearing virus, complete | SEQ ID NO: 20 | SEQ ID NO: 45 |
| genome\|Turnip vein-clearing virus | | |
| NC_002792.2 \|Ribgrass mosaic virus, complete | SEQ ID NO: 21 | SEQ ID NO: 46 |
| genomc\|Ribgrass mosaic virus | | |
| NC_003355.1 \|Wasabi mottle virus, complete | SEQ ID NO: 22 | SEQ ID NO: 47 |
| genomc/Wasabi mottle virus | | |
| NC_004422.1 \|Youcai mosaic virus, complete | SEQ ID NO: 23 | SEQ ID NO: 48 |
| genome\|Youcai mosaic virus | | |
| NC_008365.1 \|Streptocarpus flower break virus, | SEQ ID NO: 24 | SEQ ID NO: 49 |
| complete genome\|Streptocarpus flower break virus | | |
| NC_016442.1 \|Rattail cactus necrosis associated virus, | SEQ ID NO: 25 | SEQ ID NO: 50 |
| complete genome\|Rattail cactus necrosis-associated virus | | |

¹The descriptors “NC_XXXXXX.1” refer to the National Center for Biotechnology Information database accession number for entries in the world wide web internet database “ncbi[dot]nlm[dot]nih.gov/nuccore.”
²RNA equivalents of the DNA sequences are also contemplated and can be obtained from the DNA sequences provided.

TABLE 3
Movement proteins.

| | Movement protein |
|---|---|
| Virus | (MP) |
| NC_001367.1 \|Tobacco mosaic virus, complete genome\|Tobacco mosaic virus | SEQ ID NO: 51 |
| NC_001556.1 \|Tobacco mild green mosaic virus, complete genome\|Tobacco | SEQ ID NO: 52 |
| mild green mosaic virus | |
| NC_002692.1 \|Tomato mosaic virus, complete genome\|Tomato mosaic virus | SEQ ID NO: 53 |
| NC_003630.1 \|Pepper mild mottle virus, complete genome\|Pepper mild mottle | SEQ ID NO: 54 |
| virus | |
| NC_004106.1 \|Paprika mild mottle virus, complete genome\|Paprika mild mottle | SEQ ID NO: 55 |
| virus | |
| NC_009642.1 \|Bell pepper mottle tobamovirus, complete genome\|Bell pepper | SEQ ID NO: 56 |
| mottle virus | |
| NC_022230.1 \|Tomato mottle mosaic virus isolate MX5, complete | SEQ ID NO: 57 |
| genome\|Tomato mottle mosaic virus | |
| NC_001728.1 \|Odontoglossum ringspot virus, complete | SEQ ID NO: 58 |
| genome\|Odontoglossum ringspot virus | |
| NC_003852.1 \|Obuda pepper virus, complete genome\|Obuda pepper virus | SEQ ID NO: 59 |
| NC_009041.1 \|Rehmannia mosaic virus, complete genome\|Rehmannia mosaic | SEQ ID NO: 60 |
| virus | |
| NC_010944.1 \|Brugmansia mild mottle virus, complete genome\|Brugmansia | SEQ ID NO: 61 |
| mild mottle virus | |
| NC_022801.1 \|Yellow tailflower mild mottle virus isolate Cervantes, complete | SEQ ID NO: 62 |
| genome\|Yellow tailflower mild mottle virus | |
| NC_030229.1 \|Tropical soda apple mosaic virus isolate Okeechobee, complete | SEQ ID NO: 63 |
| genome\|Tropical soda apple mosaic virus | |
| NC_001801.1 \|Cucumber green mottle mosaic virus, complete | SEQ ID NO: 64 |
| genome\|Cucumber green mottle mosaic virus | |
| NC_002633.1 \|Cucumber fruit mottle mosaic virus, complete | SEQ ID NO: 65 |
| genome\|Cucumber fruit mottle mosaic virus | |
| NC_003610.1 \|Kyuri green mottle mosaic virus, complete genome\|Kyuri green | SEQ ID NO: 66 |
| mottle mosaic virus | |
| NC_003878.1 \|Zucchini green mottle mosaic virus, complete genome\|Zucchini | SEQ ID NO: 67 |
| green mottle mosaic virus | |
| NC_008614.1 \|Cucumber mottle virus, complete genome\|Cucumber mottle | SEQ ID NO: 68 |
| virus | |
| Watermelon green mottle mosaic virus | SEQ ID NO: 69 |
| NC_001873.1 \|Turnip vein-clearing virus, complete genome\|Turnip vein- | SEQ ID NO: 70 |
| clearing virus | |
| NC_002792.2 \|Ribgrass mosaic virus, complete genome\|Ribgrass mosaic virus | SEQ ID NO: 71 |
| NC_003355.1 \|Wasabi mottle virus, complete genome/Wasabi mottle virus | SEQ ID NO: 72 |
| NC_004422.1 \| Youcai mosaic virus, complete genome\|Youcai mosaic virus | SEQ ID NO: 73 |
| NC_008365.1 \|Streptocarpus flower break virus, complete | SEQ ID NO: 74 |
| genome\|Streptocarpus flower break virus | |
| NC_016442.1 \|Rattail cactus necrosis associated virus, complete genome\|Rattail | SEQ ID NO: 75 |
| cactus necrosis-associated virus | |

¹The descriptors “NC_XXXXXX.1” refer to the National Center for Biotechnology Information database accession number for entries in the world wide web internet database “ncbi[dot]nlm[dot]nih.gov/nuccore.″

TABLE 4
tRNA-like sequences

| Name¹ of Arabidopsis gene | |
|---|---|
| containing tRNA-like | |
| sequence that can confer | |
| cell-to-cell mobility | Sequence² |
| >AT1G59870.1 | SEQ ID NO: 76 |
| >AT1G71697.1 | SEQ ID NO: 77 |
| >AT1G77885.1 | SEQ ID NO: 78 |
| >AT2G04400.1 | SEQ ID NO: 79 |
| >AT2G20230.1 | SEQ ID NO: 80 |
| >AT2G24790.1 | SEQ ID NO: 81 |
| >AT2G32540.1 | SEQ ID NO: 82 |
| >AT3G15850.1 | SEQ ID NO: 83 |
| >AT3G25770.1 | SEQ ID NO: 84 |
| >AT4G15570.1 | SEQ ID NO: 85 |
| >AT4G26050.1 | SEQ ID NO: 86 |
| >AT4G26050.1 | SEQ ID NO: 87 |
| >AT4G27430.2 | SEQ ID NO: 88 |
| >AT4G27430.2 | SEQ ID NO: 89 |
| >AT5G46330.1 | SEQ ID NO: 90 |
| >AT5G54110.1 | SEQ ID NO: 91 |
| >AT5G65730.1 | SEQ ID NO: 92 |
| >AT3G02020.1 | SEQ ID NO: 93 |
| >AT5G63840.1 | SEQ ID NO: 94 |
| >AT2G36930.1 | SEQ ID NO: 95 |
| >AT5G59950.5 | SEQ ID NO: 96 |
| >AT5G20180.1 | SEQ ID NO: 97 |
| >AT5G20180.2 | SEQ ID NO: 98 |
| >AT1G07940.2 | SEQ ID NO: 99 |
| >AT2G14260.2 | SEQ ID NO: 100 |
| >AT3G05520.2 | SEQ ID NO: 101 |
| >AT4G10840.1 | SEQ ID NO: 102 |
| >AT4G10840.2 | SEQ ID NO: 103 |
| >AT1G73650.3 | SEQ ID NO: 104 |
| >AT2G45990.1 | SEQ ID NO: 105 |
| >AT2G45990.3 | SEQ ID NO: 106 |
| >AT2G45990.4 | SEQ ID NO: 107 |
| >AT3G03160.1 | SEQ ID NO: 108 |
| >AT5G64870.1 | SEQ ID NO: 109 |
| >AT1G13600.1 | SEQ ID NO: 110 |
| >AT1G73177.1 | SEQ ID NO: 111 |
| >AT1G07670.1 | SEQ ID NO: 112 |
| >ATIG55490.1 | SEQ ID NO: 113 |
| >AT1G55490.2 | SEQ ID NO: 114 |
| >AT1G55680.1 | SEQ ID NO: 115 |
| >AT2G14120.1 | SEQ ID NO: 116 |
| >AT2G14120.2 | SEQ ID NO: 117 |
| >AT2G14120.3 | SEQ ID NO: 118 |
| >AT2G32730.1 | SEQ ID NO: 119 |
| >AT3G10760.1 | SEQ ID NO: 120 |
| >AT4G10320.1 | SEQ ID NO: 121 |
| >AT4G16990.4 | SEQ ID NO: 122 |
| >AT5G59380.1 | SEQ ID NO: 123 |

¹The descriptors “ATXXXXXXX.X” refer to the Arabidopsis Information Resource (TAIR) database accession number for entries in the world wide web internet database “arabidopsis.org.”

TABLE 5
IRES sequences

| | NCBI | |
|---|---|---|
| IRES source | Accession ID¹ | Sequence² |
| Cardiovirus A | | SEQ ID NO: 124 |
| Tobacco mosaic virus | | SEQ ID NO: 125 |
| Hibiscus chlorotic | | SEQ ID NO: 126 |
| ringspot virus | | |
| Zea mays HSP101 | | SEQ ID NO: 127 |
| Plautia stali intestine virus | AB006531.1 | SEQ ID NO: 128 |
| Kashmir bee virus | NC_004807 | SEQ ID NO: 129 |
| Solenopsis invicta virus | NC_006559 | SEQ ID NO: 130 |
| Triatoma virus | NC_003783 | SEQ ID NO: 131 |
| Cricket paralysis virus | | SEQ ID NO: 132, |
| | | SEQ ID NO: 133 |
| Hepacivirus C | | SEQ ID NO: 134 |
| Turnip crinkle virus | | SEQ ID NO: 135 |
| Turnip vein-clearing virus | | SEQ ID NO: 136 |
| Rhopalosiphum padi virus | | SEQ ID NO: 137 |
| Homo sapiens C-myc | | SEQ ID NO: 138 |
| Israeli acute paralysis virus | NC_009025 | SEQ ID NO: 139 |
| Drosophila C virus | | SEQ ID NO: 140 |
| Black queen cell virus | | SEQ ID NO: 141 |
| Homalodisca coagulata | | SEQ ID NO: 142 |
| virus 1 | | |
| Aphid lethal paralysis virus | | SEQ ID NO: 143 |
| Himetobi P virus | | SEQ ID NO: 144 |
| Mud crab virus | | SEQ ID NO: 145 |
| Canine picodicistrovirus | | SEQ ID NO: 146 |

¹The descriptors “NC_XXXXXX.1” refer to the National Center for Biotechnology Information database accession number for entries in the world wide web internet database “ncbi[dot]nlm[dot]nih.gov/nuccore.”
²RNA equivalents of the DNA sequences are also contemplated and can be obtained from the DNA sequences provided.

TABLE 6
Intron sequences

| Intron number | Sequence |
|---|---|
| 1 | SEQ ID NO: 147 |
| 2 | SEQ ID NO: 148 |
| 3 | SEQ ID NO: 149 |
| 4 | SEQ ID NO: 150 |
| 5 | SEQ ID NO: 151 |
| 6 | SEQ ID NO: 152 |
| 7 | SEQ ID NO: 153 |
| 8 | SEQ ID NO: 154 |
| 9 | SEQ ID NO: 155 |
| 10 | SEQ ID NO: 156 |
| 11 | SEQ ID NO: 157 |
| 12 | SEQ ID NO: 158 |
| 13 | SEQ ID NO: 159 |
| Potato ST-LS1 | SEQ ID NO: 160 |
| intron2 | |

TABLE 7
Heterologous RNA viruses (HRVs) and target viral pathogens

| | | | | RDRP and | RDRP and |
|---|---|---|---|---|---|
| | | | | Replicase | Replicase |
| | NCBI | 5′ RR | 3′ RR | NT | AA |
| Virus name | Accession ID¹ | Sequence² | Sequence² | Sequence | Sequence |
| Cucumber mosaic | NC_002034.1. | SEQ ID | SEQ ID | — | — |
| virus | NC_002035 | NO: 161 | NO: 187 | | |
| | NC_001440.1 | | | | |
| Brome mosaic virus | NC_002026.1, | SEQ ID | SEQ ID | — | — |
| | NC_002027.1, | NO: 162 | NO: 188 | | |
| | NC_002028.2 | | | | |
| Citrus tristeza virus | NC_001661.1 | SEQ ID | SEQ ID | SEQ ID | SEQ ID |
| | (SEQ ID NO: | NO: 163 | NO: 189 | NO: 531 | NO: 223, |
| | 495) | | | | SEQ ID |
| | | | | | NO: 522 |
| Beet yellows virus | NC_001598 | SEQ ID | SEQ ID | — | SEQ ID |
| | | NO: 164 | NO: 190 | | NO: 224 |
| Cowpea mosaic virus | NC_003549.1, | SEQ ID | — | — | — |
| | NC_003550.1 | NO: 165 | | | |
| Potato virus X | NC_011620 | SEQ ID | SEQ ID | — | SEQ ID |
| | | NO: 166 | NO: 191 | | NO: 225 |
| Potato virus A | NC_004039 | SEQ ID | SEQ ID | — | SEQ ID |
| | | NO: 167 | NO: 192 | | NO: 226 |
| Potato virus Y | NC_001616 | SEQ ID | SEQ ID | — | SEQ ID |
| | | NO: 168 | NO: 193 | | NO: 227 |
| Pepper mottle virus | NC_001517 | SEQ ID | SEQ ID | — | SEQ ID |
| | | NO: 169 | NO: 194 | | NO: 228 |
| Bean yellow mosaic | NC_003492 | SEQ ID | SEQ ID | — | SEQ ID |
| virus | | NO: 170 | NO: 195 | | NO: 229 |
| Barley stripe mosaic | NC_003469, | SEQ ID | SEQ ID | — | — |
| virus | NC_003481, | NO: 171 | NO: 196 | | |
| | NC_003478 | | | | |
| Wheat stripe mosaic | NC_040760, | SEQ ID | SEQ ID | — | SEQ ID |
| virus | NC_040761 | NO: 172 | NO: 197 | | NO: 230 |
| Rice yellow mottle | L20893 | SEQ ID | SEQ ID | — | SEQ ID |
| virus | | NO: 173 | NO: 198 | | NO: 231 |
| Maize dwarf mosaic | NC_003377 | SEQ ID | SEQ ID | — | SEQ ID |
| virus | | NO: 174 | NO: 199 | | NO: 232 |
| Zucchini yellow | NC_003224 | SEQ ID | SEQ ID | — | SEQ ID |
| mosaic virus | | NO: 175 | NO: 200 | | NO: 233 |
| Watermelon mosaic | NC_006262 | SEQ ID | SEQ ID | — | SEQ ID |
| virus | | NO: 176 | NO: 201 | | NO: 234 |
| Sugarcane mosaic | KY548506 | SEQ ID | SEQ ID | — | SEQ ID |
| virus | | NO: 177 | NO: 202 | | NO: 235 |
| Tobacco mosaic virus | NC_001367.1 | SEQ ID | SEQ ID | SEQ ID | SEQ ID |
| | | NO: 178 | NO: 203 | NO: 212 | NO: 236 |
| Tomato mosaic virus | NC_002692.1 | SEQ ID | SEQ ID | SEQ ID | SEQ ID |
| | | NO: 179 | NO: 204 | NO: 213 | NO: 237 |
| Turnip vein-clearing | NC_001873.1 | SEQ ID | SEQ ID | SEQ ID | SEQ ID |
| virus | | NO: 180 | NO: 205 | NO: 214 | NO: 238 |
| Pepper mild mottle | NC_003630.1 | SEQ ID | SEQ ID | SEQ ID | SEQ ID |
| virus | | NO: 181 | NO: 206 | NO: 215 | NO: 239 |
| Turnip crinkle virus | NC_003821.3 | SEQ ID | SEQ ID | SEQ ID | SEQ ID |
| | | NO: 182 | NO: 207 | NO: 216, | NO: 240, |
| | | | | SEQ ID | SEQ ID |
| | | | | NO: 217 | NO: 241 |
| Tomato bushy stunt | NC_001554.1 | SEQ ID | SEQ ID | SEQ ID | SEQ ID |
| virus | | NO: 183 | NO: 208 | NO: 218, | NO: 242, |
| | | | | SEQ ID | SEQ ID |
| | | | | NO: 219 | NO: 243 |
| Tomato spotted wilt | KT160280.1, | SEQ ID | SEQ ID | SEQ ID | SEQ ID |
| virus | KT160281.1, | NO: 184 | NO: 209 | NO: 220 | NO: 244 |
| | KT160282.1 | | | | |
| Watermelon bud | NC_038288.1, | SEQ ID | SEQ ID | SEQ ID | SEQ ID |
| necrosis virus | NC_038289.1, | NO: 185 | NO: 210 | NO: 221 | NO: 245 |
| | NC_038290.1 | | | | |
| Turnip yellow mosaic | NC_004063.1 | SEQ ID | SEQ ID | SEQ ID | SEQ ID |
| virus | | NO: 186 | NO: 211 | NO: 222 | NO: 246 |
| Spinach latent virus | NC_003808 | SEQ ID | SEQ ID | SEQ ID | SEQ ID |
| | (SEQ ID NO: | NO: 505 | NO: 513 | NO: 532 | NO: 523 |
| | 496) | | | | |
| Spinach latent virus | NC_003809 | SEQ ID | SEQ ID | SEQ ID | SEQ ID |
| | (SEQ ID NO: | NO: 506 | NO: 514 | NO: 533 | NO: 524 |
| | 497) | | | | |
| Olive latent virus 2 | NC_003674 | SEQ ID | SEQ ID | SEQ ID | SEQ ID |
| | (SEQ ID NO: | NO: 507 | NO: 515 | NO: 534 | NO: 525 |
| | 498) | | | | |
| Citrus yellow vein | NC_026592.1 | SEQ ID | SEQ ID | SEQ ID | SEQ ID |
| clearing virus | (SEQ ID NO: | NO: 508 | NO: 516 | NO: 535 | NO: 526 |
| | 499) | | | | |
| Potato latent virus | NC_011525 | SEQ ID | SEQ ID | SEQ ID | SEQ ID |
| | (SEQ ID NO: | NO: 509 | NO: 517 | NO: 536 | NO: 527 |
| | 500) | | | | |
| Apple stem grooving | NC_001749.2 | SEQ ID | SEQ ID | SEQ ID | SEQ ID |
| virus | (SEQ ID NO: | NO: 510 | NO: 518 | NO: 537 | NO: 528 |
| | 501) | | | | |
| Citrus leaf blotch virus | NC_003877.1 | SEQ ID | SEQ ID | SEQ ID | SEQ ID |
| | (SEQ ID NO: | NO: 511 | NO: 519 | NO: 538 | NO: 529 |
| | 502) | | | | |
| Apple latent spherical | NC_003787.1 | SEQ ID | SEQ ID | SEQ ID | SEQ ID |
| virus | (SEQ ID NO: | NO: 467 | NO: 520 | NO: 472 | NO: 471 |
| | 503) | | | | |
| Soybean latent | NC_032270 | SEQ ID | SEQ ID | SEQ ID | SEQ ID |
| spherical virus | (SEQ ID NO: | NO: 512 | NO: 521 | NO: 539 | NO: 530 |
| | 504) | | | | |
| Tomato brown rugose | NC_028478.1 | SEQ ID | SEQ ID | SEQ ID | SEQ ID |
| fruit virus (ToBRFV) | SEQ ID NO: | NO: 640 | NO: 644 | NO: 652 | NO: 648 |
| | 636 | | | | |
| Celery latent virus | NC_055507.1 | SEQ ID | SEQ ID | SEQ ID | SEQ ID |
| | SEQ ID NO: | NO: 641 | NO: 645 | NO: 653 | NO: 649 |
| | 637 | | | | |
| Black grass | NC_026801.1 | SEQ ID | SEQ ID | SEQ ID | SEQ ID |
| varicosavirus-like | SEQ ID NO: | NO: 642 | NO: 646 | NO: 654 | NO: 650 |
| virus | 638 | | | | |
| Maize suscal virus | MZ270532.1 | SEQ ID | SEQ ID | SEQ ID | SEQ ID |
| | SEQ ID NO: | NO: 643 | NO: 647 | NO: 655 | NO: 651 |
| | 639 | | | | |
| Horseradish latent | NC_018858.1 | | | SEQ ID | SEQ ID |
| virus | SEQ ID NO: | | | NO: 664 | NO: 660 |
| | 656 | | | | |
| Bean latent virus | NC_055589.1 | | | SEQ ID | SEQ ID |
| | SEQ ID NO: | | | NO: 665 | NO: 661 |
| | 657 | | | | |
| Rice latent virus 1 | NC_075432.1 | | | SEQ ID | SEQ ID |
| | SEQ ID NO: | | | NO: 666 | NO: 662 |
| | 658 | | | | |
| Rice latent virus 2 | NC_040537.1 | | | SEQ ID | SEQ ID |
| | SEQ ID NO: | | | NO: 667 | NO: 663 |
| | 659 | | | | |

¹The descriptors “NC_XXXXXX.1” refer to the National Center for Biotechnology Information database accession number for entries in the world wide web internet database “ncbi[dot]nlm[dot]nih.gov/nuccore.”
²RNA equivalents of the DNA sequences are also contemplated and can be obtained from the DNA sequences provided.

TABLE 8
Antiviral Cargo Molecules

| | | | | | Examples |
|---|---|---|---|---|---|
| | NCBI or TAIR¹ | NT | AA | | of Target |
| Gene | Accession ID | Sequence | Sequence | Target | Crops |
| Ty-1 | XM_010323869.3 | SEQ ID | | Tomato yellow leaf | |
| | | NO: 247 | | curl virus | |
| HRT | AT5G43470 | SEQ ID | | Turnip crinkle virus | |
| | | NO: 248 | | | |
| RCY1 | AT2G26430, | SEQ ID | SEQ ID | Cucumber mosaic | Various |
| | AK317155 | NO: 249, | NO: 581 | virus | (broad |
| | | SEQ ID | | | spectrum) |
| | | NO: 572 | | | |
| BcTuR3 | AB498796 | SEQ ID | | Turnip mosaic virus | |
| | | NO: 250 | | | |
| Pvr4 | KT359375.1 | SEQ ID | | Potato virus Y, | |
| | | NO: 251 | | Pepper mottle virus | |
| RTM1 | NM_001331575.1 | SEQ ID | | Tobacco etch virus | |
| | | NO: 252 | | | |
| Sw5b | KY006959.1 | SEQ ID | SEQ ID | Tomato spotted wilt | Various |
| | | NO: 253 | NO: 582 | virus | (broad |
| | | | | | spectrum) |
| N | U15605.1 | SEQ ID | | Tobacco mosaic | |
| | | NO: 254 | | virus, Tomato | |
| | | | | brown rugose fruit | |
| | | | | virus | |
| Tm-2-2 | AF536201.1 | SEQ ID | SEQ ID | Tomato mosaic | Tomato |
| | | NO: 255, | NO: 583 | virus | |
| | | SEQ ID | | | |
| | | NO: 573 | | | |
| L4 | AB523377 | SEQ ID | | Tobacco mosaic | |
| | | NO: 256 | | virus, Tomato | |
| | | | | mosaic virus, | |
| | | | | Tomato brown | |
| | | | | rugose fruit virus, | |
| | | | | Tobacco mild green | |
| | | | | mosaic virus, Bell | |
| | | | | pepper mottle virus, | |
| | | | | Pepper mild mottle | |
| | | | | virus | |
| Rx | AJ011801.1 | SEQ ID | SEQ ID | Potato virus X | Potato |
| | | NO: 257, | NO: 584 | | |
| | | SEQ ID | | | |
| | | NO: 574 | | | |
| JAX1 | AT1G58160 | SEQ ID | | Broad spectrum | |
| | | NO: 258 | | Potexvirus | |
| Nucleotides | M81413 | SEQ ID | | Pepper mild mottle | |
| 3411 to 4388 | | NO: 259 | | virus | |
| of PMMo V | | | | | |
| genomic | | | | | |
| sequence | | | | | |
| TMV coat | NP_597750.1 | SEQ ID | SEQ ID | Plant pathogenic | Tomato |
| protein (CP) | | NO: 575 | NO: 585 | viruses | |
| SMV coat | AB100443 | SEQ ID | SEQ ID | Plant pathogenic | Soybean |
| protein (CP) | | NO: 576 | NO: 586 | viruses | |
| CMV coat | AF350450 | SEQ ID | SEQ ID | Plant pathogenic | Black |
| protein (CP) | | NO: 577 | NO: 587 | viruses | pepper |
| PVX coat | X12804 | SEQ ID | SEQ ID | Plant pathogenic | Potato |
| protein (CP) | | NO: 578 | NO: 588 | viruses | |
| TSWV | NC_002051.1 | SEQ ID | SEQ ID | Plant pathogenic | Tobacco |
| nucleocapsid | | NO: 579 | NO: 589 | viruses | |
| protein (NP) | | | | | |
| Rsv4 | LC325439.1 | SEQ ID | SEQ ID | Soybean mosaic | Soybean |
| | | NO: 580 | NO: 590 | virus | |

¹The descriptors “ATXGXXXXX” refer to the Arabidopsis Information Resource (TAIR) database accession number for entries in the world wide web internet database “arabidopsis.org.” The other descriptors refer to the National Center for Biotechnology Information database accession number for entries in the world wide web internet database “ncbi[dot]nlm[dot]nih.gov/nuccore.”

TABLE 9
Insecticidal or Insect Inhibitory and Nematocidal or Nematode Inhibitory Cargo Molecules

| | NCBI Accession | NT | AA | Cargo | | Examples of |
|---|---|---|---|---|---|---|
| Gene name | ID | Sequence | Sequence | type | Target | Target Crop |
| Vip3Aa | HM132043.2 | SEQ ID | | Protein | Insect | |
| | | NO: 260 | | | (lepidopteran) | |
| Vip3 | KF499114.1 | SEQ ID | | Protein | Insect | |
| | | NO: 261 | | | (lepidopteran) | |
| Vip1 | KF499112.1 | SEQ ID | | Protein | Insect | |
| | | NO: 262 | | | (coleoptera, | |
| | | | | | Hemiptera) | |
| Vip2 | KF499113.1 | SEQ ID | | Protein | Insect | |
| | | NO: 263 | | | (coleoptera, | |
| | | | | | Hemiptera) | |
| Sip1A | DQ350881.1 | SEQ ID | | Protein | Insect | |
| | | NO: 264 | | | (coleoptera) | |
| Spodoptera | HQ829425.1 | SEQ ID | | dsRNA | Insect (Beet | |
| exigua | | NO: 265 | | | armyworm) | |
| integrin beta | | | | | | |
| 1 subunit | | | | | | |
| Pheromone | U08109.1 | SEQ ID | | dsRNA | Insect (Corn | |
| biosynthesis | | NO: 266 | | | earworm) | |
| activating | | | | | | |
| neuropeptide | | | | | | |
| Cry1A | HM132043.2 | SEQ ID | | Protein | Insect | |
| | | NO: 267 | | | (lepidopteran, | |
| | | | | | diptera) | |
| Cry1Ab | KR612322.1 | SEQ ID | | Protein | Gastropoda | |
| | | NO: 268 | | | | |
| Cry1B | EF102874.1 | SEQ ID | | Protein | Insect | |
| | | NO: 269 | | | (lepidopteran, | |
| | | | | | diptera, | |
| | | | | | coleoptera) | |
| Cry1C | EU679502.1 | SEQ ID | | Protein | Insect | |
| | | NO: 270 | | | (lepidopteran, | |
| | | | | | diptera) | |
| Cry1D | JN205068.1 | SEQ ID | | Protein | Insect | |
| | | NO: 271 | | | (lepidopteran) | |
| Cry1Ea | ON508055.1 | SEQ ID | | Protein | Insect | |
| | | NO: 272 | | | (lepidopteran) | |
| Cry1F | EU679501.1 | SEQ ID | | Protein | Insect | |
| | | NO: 273 | | | (lepidopteran) | |
| Cry1I | AF211190.1 | SEQ ID | | Protein | Insect | |
| | | NO: 274 | | | (lepidopteran, | |
| | | | | | coleoptera) | |
| Cry1K | U28801.1 | SEQ ID | | Protein | Insect | |
| | | NO: 275 | | | (lepidopteran) | |
| Cry2A | KJ710647.1 | SEQ ID | | Protein | Insect | |
| | | NO: 276 | | | (lepidopteran, | |
| | | | | | diptera, | |
| | | | | | Hemiptera) | |
| Cry3A | EU332160.1 | SEQ ID | | Protein | Insect | |
| | | NO: 277 | | | (coleoptera, | |
| | | | | | Hemiptera, | |
| | | | | | Hymenoptera) | |
| Cry4A | EF208904.1 | SEQ ID | | Protein | Insect (diptera) | |
| | | NO: 278 | | | | |
| Cry4B | MN539646.1 | SEQ ID | | Protein | Insect (diptera) | |
| | | NO: 279 | | | | |
| Cry5A | EF219060.1 | SEQ ID | | Protein | Insect, | |
| | | NO: 280 | | | Rhabditida | |
| | | | | | (Hymenoptera) | |
| Cry5B | EU822809.1 | SEQ ID | SEQ ID | Protein | Rhabditida, | Tomato, |
| | | NO: 281 | NO: 598 | | root knot | potato, |
| | | | | | nematode | peppers, |
| | | | | | | eggplants, |
| | | | | | | cotton |
| Cry8B | FJ770571.1 | SEQ ID | | Protein | Insect | |
| | | NO: 282 | | | (coleoptera) | |
| Cry8D | AB303980.1 | SEQ ID | | Protein | Insect | |
| | | NO: 283 | | | (lepidopteran, | |
| | | | | | coleoptera) | |
| Cry8F | KC778983.1 | SEQ ID | | Protein | Insect | |
| | | NO: 284 | | | (coleoptera) | |
| Cry8G | FJ198072.1 | SEQ ID | | Protein | Insect | |
| | | NO: 285 | | | (coleoptera) | |
| Cry9A | KX094977.1 | SEQ ID | | Protein | Insect | |
| | | NO: 286 | | | (lepidopteran) | |
| Cry10 | MN539645.1 | SEQ ID | | Protein | Insect (diptera) | |
| | | NO: 287 | | | | |
| Cry11Aa | MK184471.1 | SEQ ID | | Protein | Insect (diptera, | |
| | | NO: 288 | | | Hemiptera) | |
| Cry11B | HQ845255.1 | SEQ ID | | Protein | Insect (diptera) | |
| | | NO: 289 | | | | |
| Cry19A | Y07603.1 | SEQ ID | | Protein | Insect (diptera) | |
| | | NO: 290 | | | | |
| Cry19B | D88381.1 | SEQ ID | | Protein | Insect (diptera) | |
| | | NO: 291 | | | | |
| Cry21A | KC898940.2 | SEQ ID | | Protein | Rhabditida | |
| | | NO: 292 | | | | |
| Cry22A | EU715020.1 | SEQ ID | | Protein | Insect | |
| | | NO: 293 | | | (lepidopteran, | |
| | | | | | coleoptera, | |
| | | | | | Hymenoptera) | |
| Cry32A | GU063850.1 | SEQ ID | | Protein | Insect | |
| | | NO: 294 | | | (lepidopteran) | |
| Cry35A | AY536895.1 | SEQ ID | | Protein | Insect | |
| | | NO: 295 | | | (coleoptera) | |
| Cry35B | AY536894.1 | SEQ ID | | Protein | Insect | |
| | | NO: 296 | | | (coleoptera) | |
| Cry39A | AB074413.2 | SEQ ID | | Protein | Insect (diptera) | |
| | | NO: 297 | | | | |
| Cry51A | MT882335.1 | SEQ ID | | Protein | Insect | |
| | | NO: 298 | | | (lepidopteran) | |
| Cyt1A | EF656359.1 | SEQ ID | | Protein | Insect (diptera, | |
| | | NO: 299 | | | coleoptera) | |
| Cyt2Ba | MK690503.1 | SEQ ID | | Protein | Insect (diptera) | |
| | | NO: 300 | | | | |
| snf7 | XM_028287710.2 | SEQ ID | | dsRNA | Insect (Western | |
| | | NO: 301 | | | corn rootworm) | |
| snf7 | XM_023156793.1 | SEQ ID | | dsRNA | Insect | |
| | | NO: 302 | | | (Colorado | |
| | | | | | potato beetle) | |
| Actin | XM_023168674.1 | SEQ ID | | dsRNA | Insect | |
| | | NO: 303 | | | (Colorado | |
| | | | | | potato beetle) | |
| vATPaseI | XM_023156515.1 | SEQ ID | | dsRNA | Insect | |
| | | NO: 304 | | | (Colorado | |
| | | | | | potato beetle) | |
| Tyrosine | NM_001305520.1 | SEQ ID | | dsRNA | Insect | |
| hydroxylase | | NO: 305 | | | (Diamondback | |
| | | | | | moth) | |
| C002 | XM_015524104.1 | SEQ ID | | dsRNA | Insect (Russian | |
| | | NO: 306 | | | wheat aphid) | |
| hunchback | NM_001162510.1 | SEQ ID | | dsRNA | Insect (Pea | |
| | | NO: 307 | | | Aphid) | |
| Mi-1.2 | AF039682.1 | SEQ ID | SEQ ID | Protein | Root knot | Tomato, |
| | | NO: 591 | NO: 599 | | nematode | potato, |
| | | | | | | peppers, |
| | | | | | | eggplants, |
| | | | | | | cotton |
| Gpa-2 | AF195939.1 | SEQ ID | SEQ ID | Protein | Cyst nematode | Tomato, |
| | | NO: 592 | NO: 600 | | | potato, |
| | | | | | | peppers, |
| | | | | | | eggplants, |
| | | | | | | cotton |
| Rhg1 | NM_001248836.2 | SEQ ID | SEQ ID | Protein | Cyst nematode | Soybean, |
| | | NO: 593 | NO: 601 | | | potato, corn |
| Rhg4 | NM_001249781.3 | SEQ ID | SEQ ID | Protein | Cyst nematode | Soybean, |
| | | NO: 594 | NO: 602 | | | potato, corn |
| Zea mays | NM_001112543.1 | SEQ ID | SEQ ID | Protein | Root knot/ | Banana, |
| cystatin 2 | | NO: 595 | NO: 603 | | burrowing/ | soybean, |
| | | | | | spiral | tomato, |
| | | | | | nematodes | potato, |
| | | | | | | cotton, |
| | | | | | | eggplants |
| Colocasia | AF525880.1 | SEQ ID | SEQ ID | Protein | Root knot | Tomato, |
| esculenta | | NO: 596 | NO: 604 | | nematode, | potato, |
| cysteine | | | | | plant fungal | peppers, |
| proteinase | | | | | pathogens | eggplants, |
| inhibitor | | | | | | cotton |
| (CeCPI) | | | | | | |
| Cry6A | | SEQ ID | SEQ ID | Protein | Root knot | Tomato, |
| (plant-codon | | NO: 597 | NO: 605 | | nematode | potato, |
| optimized) | | | | | | peppers, |
| | | | | | | eggplants, |
| | | | | | | cotton |

¹ The descriptors refer to the National Center for Biotechnology Information database accession number for entries in the world wide web internet database “ncbi[dot]nlm[dot]nih.gov/nuccore.”

TABLE 10
Antifungal and Antibacterial Cargo RNAi Targets

| Cargo (RNAi | NCBI Accession | | | Examples of target |
|---|---|---|---|---|
| target gene) | ID¹ | Sequence | Target pathogen | crop |
| Bc_Chitin | AY515145 | SEQ ID | Botrytis cinerea | bell pepper, tomato |
| synthase 1 | | NO: 308 | | |
| Bc_Chitin | AF494188 | SEQ ID | Botrytis cinerea | bell pepper, tomato |
| synthase 3a | | NO: 309 | | |
| Bc_Chitin | AF529208.1 | SEQ ID | Botrytis cinerea | bell pepper, tomato |
| synthase 3b | | NO: 310 | | |
| Bc Chitin | AY515144 | SEQ ID | Botrytis cinerea | bell pepper, tomato |
| synthase 6 | | NO: 311 | | |
| Bc_DCL1 | XM_024696562.1 | SEQ ID | Botrytis cinerea | bell pepper, tomato |
| | | NO: 312 | | |
| Bc_DCL2 | XM_037334035.1 | SEQ ID | Botrytis cinerea | bell pepper, tomato |
| | | NO: 313 | | |
| Bc_ERGI | XM_001547426.2 | SEQ ID | Botrytis cinerea | bell pepper, tomato |
| | | NO: 314 | | |
| Bc_ERG11 | MT430876.1 | SEQ ID | Botrytis cinerea | bell pepper, tomato |
| | | NO: 315 | | |
| Bc_ERG13 | XM_001552372.2 | SEQ ID | Botrytis cinerea | bell pepper, tomato |
| | | NO: 316 | | |
| Bc_EF2 | XM_024693463 | SEQ ID | Botrytis cinerea | bell pepper, tomato |
| | | NO: 317 | | |
| Fgr_chs3b | AF484941.1 | SEQ ID | Fusarium | wheat, soybean, corn |
| | | NO: 318 | graminearum | |
| Fgr_chs6 | XM_011319518.1 | SEQ ID | Fusarium | wheat, soybean, corn |
| | | NO: 319 | graminearum | |
| Fgr_gls | XM_011329487.1 | SEQ ID | Fusarium | wheat, soybean, corn |
| | | NO: 320 | graminearum | |
| Fgr_Pkc | XM_011329746.1 | SEQ ID | Fusarium | wheat, soybean, corn |
| | | NO: 321 | graminearum | |
| FO_chs5 | AF484941.1 | SEQ ID | Fusarium oxysporum | tomato, tobacco, |
| | | NO: 322 | oxisporum | cucurbits, banana |
| FO_W2 | AB266616.1 | SEQ ID | Fusarium oxysporum | tomato, tobacco, |
| | | NO: 323 | | cucurbits, banana |
| FO_FRP1 | AY673970.1 | SEQ ID | Fusarium oxysporum | tomato, tobacco, |
| | | NO: 324 | | cucurbits, banana |
| FO_OPR | AFQF01002613 | SEQ ID | Fusarium oxysporum | tomato, tobacco, |
| | | NO: 325 | | cucurbits, banana |
| Mo_chs1 | MGG_01802.6 | SEQ ID | Magnaporthe + C19 | rice, wheat, maize |
| | | NO: 326 | | |
| Mo_chs6 | MGG_13013.6 | SEQ ID | Magnaporthe | rice, wheat, maize |
| | | NO: 327 | | |
| Mo_chs7 | MGG_06064.6 | SEQ ID | Magnaporthe | rice. wheat, maize |
| | | NO: 328 | | |
| Sorbitol | NW_003303753.1 | SEQ ID | Phytophthora | potato |
| dehydrogenase | | NO: 329 | infestans | |
| (SDH) | | | | |
| AGO1 | XM_011321673.1 | SEQ ID | Fusarium | wheat. soybean, corn |
| | | NO: 330 | graminearum | |
| AGO2 | XM_011317703.1 | SEQ ID | Fusarium | wheat, soybean, corn |
| | | NO: 331 | graminearum | |
| FgQIP | XM_011328057.1 | SEQ ID | Fusarium | wheat, soybean, corn |
| | | NO: 332 | graminearum | |
| FG_DCL1 | XM_011330473.1 | SEQ ID | Fusarium | wheat, soybean, corn |
| | | NO: 333 | graminearum | |
| FG_DCL2 | XM_011322896.1 | SEQ ID | Fusarium | wheat, soybean, corn |
| | | NO: 334 | graminearum | |
| VD_DCL1 | MK015620.1 | SEQ ID | Verticillium dahliae | potato, peppermint, |
| | | NO: 335 | | eggplant, tomato |
| VD_DCL2 | MK015621.1 | SEQ ID | Verticillium dahliae | potato, peppermint. |
| | | NO: 336 | | eggplant, tomato |
| Suppressor of | XM_028639311.1 | SEQ ID | Verticillium dahliae | potato, peppermint, |
| actin (SAC1) | | NO: 337 | | eggplant, tomato |
| Dynactin | KAH6702269.1 | SEQ ID | Verticillium dahliae, | potato, peppermint, |
| (DCTN1) | | NO: 338 | Rhizoctonia solani | eggplant, tomato |
| Polygalacturonase | XM_043324583.1 | SEQ ID | Rhizoctonia solani | tomato, wheat, |
| (PG) | | NO: 339 | | soy bean, potato |
| Bc_vacuolar | XM_024698195.1 | SEQ ID | Botrytis cinerea | bell pepper, tomato |
| protein sorting 51 | | NO: 340 | | |
| (VPS51) | | | | |
| Bc_dynactin | XM_001550899.2 | SEQ ID | Botrytis cinerea | bell pepper, tomato |
| (DCTN1) | | NO: 341 | | |
| Bc_suppressor of | XM_024697060.1 | SEQ ID | Botrytis cinerea | bell pepper, tomato |
| actin (SAC1) | | NO: 342 | | |
| Ss_DCL1 | XM_001585129.1 | SEQ ID | S. sclerotiorum | soy bean, pea, canola |
| | | NO: 343 | | |
| Ss_DCL2 | XM_001588771.1 | SEQ ID | S. sclerotiorum | soybean, pea, canola |
| | | NO: 344 | | |
| Ss_vacuolar | XM_001590214.1 | SEQ ID | S. sclerotiorum | soy bean, pea, canola |
| protein sorting 51 | | NO: 345 | | |
| (VPS51) | | | | |
| Ss_dynactin | XM_001594287.1 | SEQ ID | S. sclerotiorum | soy bean, pea, canola |
| (DCTN1) | | NO: 346 | | |
| Ss_suppressor of | CP017829.1 | SEQ ID | S. sclerotiorum | soybean, pea, canola |
| actin (SAC1) | | NO: 347 | | |
| Fmk1 | XM_018386908.1 | SEQ ID | Fusarium oxysporum | tomato, tobacco, |
| (FOXG_08140) | | NO: 348 | | cucurbits, banana |
| Hog1 | XM_018384930.1 | SEQ ID | Fusarium oxysporum | tomato, tobacco, |
| (FOXG_06318) | | NO: 349 | | cucurbits, banana |
| PBS2 | XM_018380643.1 | SEQ ID | Fusarium oxysporum | tomato, tobacco, |
| (FOXG 03107) | | NO: 350 | | cucurbits, banana |
| Cfa6-HrpL | | SEQ ID | Pseudomonas | tomato, bean |
| | | NO: 606 | syringae | |

¹The descriptors refer to the National Center for Biotechnology Information database accession number for entries in the world wide web internet database “ncbi[dot]nlm[dot]nih.gov/nuccore.”

TABLE 11
Antifungal and antibacterial cargo protein

| | NCBI Accession | NT | AA | | Examples of |
|---|---|---|---|---|---|
| Cargo | ID¹ | Sequence | Sequence | Target pathogen | target crop |
| Antifungal | 6U6G_A | SEQ ID | SEQ ID | Botrytis cinerea, | tomato, |
| symbiotic peptide | | NO: 351 | NO: 361 | Fusarium spp. | tobacco, bell |
| NCR044 | | | | | pepper |
| Buckwheat | PODKH7 | SEQ ID | SEQ ID | Fusarium | tomato, |
| Antifungal | | NO: 352 | NO: 362 | oxysporum | tobacco, |
| Protein, Fa- | | | | | cucurbits, |
| AMP1 | | | | | banana |
| Buckwheat | PODKH8 | SEQ ID | SEQ ID | Fusarium | tomato, |
| Antifungal | | NO: 353 | NO: 363 | oxysporum | tobacco, |
| Protein, Fa- | | | | | cucurbits. |
| AMP2 | | | | | banana |
| Pepper | AAT35532.1 | SEQ ID | SEQ ID | Fusarium | tomato, rice, |
| Antimicrobial | | NO: 354 | NO: 364 | oxysporum, | bell pepper, |
| Protein CaAMP1 | | | | Alternaria | soy bean |
| | | | | brassicicola, | |
| | | | | Phytophthora | |
| | | | | spp. | |
| Bean CH5B | P36361.1 | SEQ ID | SEQ ID | Alternaria spp., | soybean, rice. |
| chitinase protein | | NO: 355 | NO: 365 | Rhizoctonia | tomato, bell |
| | | | | solani | pepper |
| Rice RC7 | Q7DNA1.1 | SEQ ID | SEQ ID | Rhizoctonia | rice, bell |
| chitinase | | NO: 356 | NO: 366 | solani | pepper, |
| | | | | | tomato. |
| | | | | | soybean |
| Tomato chitinase | Q40114.1 | SEQ ID | SEQ ID | Verticillium | strawberry |
| pcht28 | | NO: 357 | NO: 367 | dahliae | |
| Barley chitinase | M62904.1 | SEQ ID | — | Fusarium | wheat, |
| II | | NO: 358 | | graminearum | soybean, corn |
| Rice chitinase | P25765.2 | SEQ ID | SEQ ID | Rhizoctonia | rice |
| RCH10 | | NO: 359 | NO: 368 | solani, | |
| | | | | Magnaporthe | |
| | | | | oryzae | |
| Arabidopsis | NP_179105.1 | — | SEQ ID | Fusarium | wheat, corn |
| Thionin 2.4 | | | NO: 369 | graminearum, F. | |
| | | | | sporotrichoides | |
| Brassica juncea | AY714982.1 | SEQ ID | — | Rhizoctonia | potato |
| BjCHI1 chitinase | | NO: 360 | | solani | |
| Mungin protein | — | — | SEQ ID | R. solani, | tomato, bell |
| from mung bean | | | NO: 370 | Coprinus | pepper, potato, |
| | | | | comatus. | tomato. |
| | | | | Mycosphaerella | soy bean |
| | | | | arachidicola, B. | |
| | | | | cinerea, F | |
| | | | | oxysporum | |
| Camptothecin | UUY85546.1 | — | SEQ ID | Magnaporthe | rice |
| from | | | NO: 371 | oryzae | |
| Campthothcca | | | | | |
| NaDI defensin | Q8GTM0.1 | — | SEQ ID | Fusarium | cotton, tomato, |
| from Nicotiana | | | NO: 372 | oxysporum f.sp. | bell pepper |
| alata | | | | vasinfectum, | |
| | | | | Botrytis cinerea | |
| Tk-AMP-X1 | CCP19154.1 | — | SEQ ID | Fusarium | wheat, corn |
| from wheat | | | NO: 373 | graminearum | |
| CBP from spindle | AAP35269.1 | — | SEQ ID | Alternaria | brassica crops |
| tree | | | NO: 374 | brassicicola | |
| | | | | MUCL 20297 | |
| Chitinase from | AAP35270.1 | — | SEQ ID | Alternaria | brassica crops |
| spindle tree | | | NO: 375 | brassicicola | |
| | | | | MUCL 20297 | |
| Antimicrobial | B3EWQ1.1 | — | SEQ ID | Fusarium | banana, |
| peptide 1, | | | NO: 376 | oxysporum | tomato, |
| ToAMP1 from | | | | TSKHA-4 | cucurbits |
| wigg flower | | | | | |
| Antimicrobial | B3EWQ2.1 | — | SEQ ID | Fusarium | banana. |
| peptide 2, | | | NO: 377 | oxysporum | tomato, |
| ToAMP2 | | | | TSKHA-4 | cucurbits |
| Antimicrobial | B3EWQ3.1 | — | SEQ ID | Fusarium | banana, |
| peptide 3, | | | NO: 378 | oxysporum | tomato, |
| ToAMP3 | | | | TSKHA-4 | cucurbits |
| Defensin Tk- | P84963.1 | — | SEQ ID | Alternaria | brassica crops |
| AMP-D1 | | | NO: 379 | consortialis | |
| Defensin Ec- | P86519.1 | — | SEQ ID | Fusarium | banana, |
| AMP-D2 | | | NO: 380 | oxysporum | tomato, |
| | | | | | cucurbits |
| Defensin Ec- | P86518.1 | — | SEQ ID | Fusarium | wheat, corn |
| AMP-D1 | | | NO: 381 | graminearum | |
| Antimicrobial | AAB22102.1 | — | SEQ ID | Bipolaris | wheat |
| peptide Ar-AMP | | | NO: 382 | sorokiniana | |
| AtCDR1 | NM_122858.3 | SEQ ID | SEQ ID | P. syringae, | Citrus, tomato |
| | | NO: 607 | NO: 619 | Liberibacter sp. | |
| PtCDR2 | Consensus | SEQ ID | | P. syringae, | Citrus, tomato |
| | sequence | NO: 608 | | Liberibacter sp. | |
| PtCDR8 | Consensus | SEQ ID | | P. syringae, | Citrus, tomato |
| | sequence | NO: 609 | | Liberibacter sp. | |
| cecropin B | NM_001102561.1 | SEQ ID | SEQ ID | P. syringae, | Citrus, tomato, |
| | | NO: 610 | NO: 620 | Liberibacter sp., | grapevines |
| | | | | plant fungal | |
| | | | | pathogens | |
| BP100 | | SEQ ID | SEQ ID | P. syringae, | Citrus, tomato, |
| | | NO: 611 | NO: 621 | Liberibacter sp. | grapevines |
| CA-3.1 | | SEQ ID | SEQ ID | P. syringae, | Citrus, tomato, |
| | | NO: 612 | NO: 622 | Liberibacter sp. | grapevines |
| Medicago | AY313166.1 | SEQ ID | SEQ ID | P. syringae, | Citrus, tomato, |
| truncatula | | NO: 613 | NO: 623 | Liberibacter sp., | grapevines |
| defensin (Def1) | | | | plant fungal | |
| | | | | pathogens | |
| RsAFP2 | KF498667.1 | SEQ ID | SEQ ID | plant fungal | Citrus, tomato, |
| | | NO: 614 | NO: 624 | pathgens | grapevines |
| Arabidopsis | NM_106233.4 | SEQ ID | SEQ ID | plant fungal | Grapevines, |
| thaliana defensin | | NO: 615 | NO: 625 | and/or bacterial | tomato |
| 1.1 (PDF1.1) | | | | pathogens | |
| JLA-9 | | SEQ ID | SEQ ID | P. syringae, | Grapevines, |
| | | NO: 616 | NO: 626 | Liberibacter sp. | tomato, potato, |
| | | | | | citrus |
| Darobactin | | SEQ ID | SEQ ID | P. syringae, | Grapevines, |
| | | NO: 617 | NO: 627 | Liberibacter sp. | tomato, potato, |
| | | | | | citrus |
| Urechistachykinin | NM_122858.3 | SEQ ID | SEQ ID | P. syringae, | Grapevines, |
| II | | NO: 618 | NO: 628 | Liberibacter sp. | tomato, potato, |
| | | | | | citrus |

¹The descriptors refer to the National Center for Biotechnology Information database accession number for entries in the world wide web internet database “ncbi[dot]nlm[dot]nih.gov/nuccore.”

TABLE 12
Resistance Gene

| | | | NCBI or | | | |
|---|---|---|---|---|---|---|
| | | | TAIR | | | |
| Gene | Source | Effector | Accession | NT | AA | |
| name | species | Target | ID1 | Sequence | Sequence | Target |
| Bph1/9 | Oryza sativa | — | KX681919.1 | SEQ ID | — | Nilaparvata |
| | | | | NO: 383 | | lugens |
| Bph14 | Oryza sativa | — | FJ941067.1 | SEQ ID | — | Nilaparvata |
| | | | | NO: 384 | | lugens |
| TAO1 | Arabidopsis | AvrB | NM_ | SEQ ID | — | Pseudomonas |
| | thaliana | | 001344554 | NO: 385 | | syringae |
| CAR1 | Arabidopsis | AvrE, | AT1G50180 | SEQ ID | — | Pseudomonas |
| | thaliana | HopAA | | NO: 386 | | syringae |
| RPS5 | Arabidopsis | AvrPphB | AT1G12220 | SEQ ID | — | Pseudomonas |
| | thaliana | | | NO: 387 | | syringae |
| RPS2 | Arabidopsis | AvrRpm1, | AT3G03600 | SEQ ID | — | Pseudomonas |
| | thaliana | AvrRpt2 | | NO: 388 | | syringae |
| ZAR1 | Arabidopsis | HopO, | AT3G50950 | SEQ ID | — | Pseudomonas |
| | thaliana | HopX, | | NO: 389 | | syringae, |
| | | HopBA1, | | | — | Xanthomonas |
| | | HopZ1a, | | | | campestris |
| | | HopF2a, | | | | |
| | | AvrAc | | | | |
| RPS6 | Arabidopsis | HopA1 | AT4G31700 | SEQ ID | — | Pseudomonas |
| | thaliana | | | NO: 390 | | syringae |
| SUMM2 | Arabidopsis | HopAI1 | AT1G12280 | SEQ ID | — | Pseudomonas |
| | thaliana | | | NO: 391 | | syringae |
| BAR1 | Arabidopsis | HopB | AT5G18360 | SEQ ID | — | Pseudomonas |
| | thaliana | | | NO: 392 | | syringae |
| CaPtr1 | Capsicum | AvrRpt2, | — | — | — | Pseudomonas |
| | annuum | RipBN | | | | syringae, |
| | | | | | | Ralstonia |
| | | | | | | pseudo- |
| | | | | | | solanacearum |
| Bs2 | Capsicum | AvrBs2 | AF202179.1 | SEQ ID | — | Xanthomonas |
| | chacoense | | | NO: 393 | | campestris pv. |
| | | | | | | vesicatoria |
| Rpg1b | Glycine max | AvrB | NM_ | SEQ ID | — | Pseudomonas |
| | | | 001353616.2 | NO: 394 | | savastanoi |
| Rpg1r | Glycine max | AvrRpm1 | KF958751 | SEQ ID | — | Pseudomonas |
| | | | | NO: 395 | | syringae |
| Pbr1.b | Hordeum | AvrPphB | MH595618 | SEQ ID | — | Pseudomonas |
| | vulgare | | | NO: 396 | | savastanoi |
| Pbr1.c | Hordeum | AvrPphB | MH595619 | SEQ ID | — | Pseudomonas |
| | vulgare | | | NO: 397 | | savastanoi |
| FB_MR5 | Malus x | AvrRpt2_ | CCH50986.1 | — | SEQ ID | Erwinia |
| | robusta | EA | | | NO: 407 | amylovora |
| NbPtr1 | Nicotiana | AvrRpt2, | — | — | — | Pseudomonas |
| | benthamiana | RipBN | | | | syringae, |
| | | | | | | Ralstonia |
| | | | | | | pseudo- |
| | | | | | | solanacearum |
| Roq1 | Nicotiana | HopQ1-1, | MF773579.1 | SEQ ID | — | Pseudomonas |
| | benthamiana | XopQ, RipB | | NO: 398 | | syringae, |
| | | | | | | Xanthomonas, |
| | | | | | | Ralstonia |
| | | | | | | solanacearum |
| NbZAR1 | Nicotiana | XopJ4 | MH532570.1 | SEQ ID | — | Xanthomonas |
| | benthamiana | | | NO: 399 | | perforans |
| Rpa1 | Nicotiana | AvrRpm1_ | — | — | — | Pseudomonas |
| | tabacum | Psy, | | | | syringae |
| | | AvrRpm1_ | | | | |
| | | Psa | | | | |
| NitabPtr1 | Nicotiana | AvrRpt2, | — | — | — | Pseudomonas |
| | tabacum | RipBN | | | | syringae, |
| | | | | | | Ralstonia |
| | | | | | | pseudo- |
| | | | | | | solanacearum |
| NtomPtrl | Nicotiana | AvrRpt2, | — | — | — | Pseudomonas |
| | tomentosi- | RipBN | | | | syringae, |
| | formis | | | | | Ralstonia |
| | | | | | | pseudo- |
| | | | | | | solanacearum |
| Xo1 | Oryza sativa | pthXo1, | MT395901.1 | SEQ ID | — | Xanthomonas |
| | | tal2g, | | NO: 400 | | oryzae pv. |
| | | tal8, tal2, | | | | oryzae, |
| | | tal1c, | | | | Xanthomonas |
| | | tal5, talC, | | | | oryzae pv. |
| | | AvrXa10, | | | | oryzicola |
| | | AvrXa7 | | | | |
| Xa1 | Oryza sativa | pthXo1, | — | — | — | Xanthomonas |
| | | Tal9d, Tal4 | | | | oryzae pv. |
| | | | | | | oryzae |
| Ptr1 | Solanum | AvrRpt2, | — | — | — | Pseudomonas |
| | lycopersicum | RipBN | | | | syringae, |
| | | | | | | Ralstonia |
| | | | | | | pseudo- |
| | | | | | | asolanacerum |
| Bs4 | Solanum | AvrBs4, | NM_ | SEQ ID | — | Xanthomonas |
| | lycopersicum | Hax3, Hax4, | 001329525.1 | NO: 401 | | |
| | | AvrBs3 | | | | |
| Prf | Solanum | AvrPto, | — | — | — | Pseudomonas |
| | lycopersicum | AvrPtoB | | | | syringae |
| StrPtr1 | Solanum | AvrRpt2, | — | — | — | Pseudomonas |
| | tuberosum | RipBN | | | | syringae, |
| | | | | | | Ralstonia |
| | | | | | | pseudos- |
| | | | | | | olanacearum |
| Rxo1 | Zea mays | AvrRxo1- | AY935244.1 | SEQ ID | — | Xanthomonas |
| | | ORF1 | | NO: 402 | | oryzae, |
| | | | | | | Burkholderia |
| | | | | | | andropogonis |
| RRS1-S | Arabidopsis | AvrRps4 | — | — | — | Pseudomonas |
| | thaliana | | | | | syringae, |
| | | | | | | Ralstonia |
| | | | | | | solanacearum, |
| | | | | | | Colletotrichum |
| | | | | | | higginsianum |
| RPS4 | Arabidopsis | AvrRps4, | — | — | — | Pseudomonas |
| | thaliana | PopP2 | | | | syringae, |
| | | | | | | Ralstonia |
| | | | | | | solanacearum, |
| | | | | | | Colletotrichum |
| | | | | | | higginsianum |
| RRS1-R | Arabidopsis | AvrRps4, | — | — | — | Pseudomonas |
| | thaliana | PopP2 | | | | syringae, |
| | | | | | | Ralstonia |
| | | | | | | solanacearum, |
| | | | | | | Colletotrichum |
| | | | | | | higginsianum |
| RRS1B | Arabidopsis | PopP2 | — | — | — | Pseudomonas |
| | thaliana | | | | | syringae, |
| | | | | | | Ralstonia |
| | | | | | | solanacearum, |
| | | | | | | Colletotrichum |
| | | | | | | higginsianum |
| RPS4B | Arabidopsis | PopP2 | — | — | — | Pseudomonas |
| | thaliana | | | | | syringae, |
| | | | | | | Ralstonia |
| | | | | | | solanacearum, |
| | | | | | | Colletotrichum |
| | | | | | | higginsianum |
| LOV1 | Arabidopsis | Victorin | — | — | — | Cochliobolus |
| | thaliana | | | | | victoriae |
| WRR4A | Arabidopsis | CCG28, | — | — | — | Albugo candida |
| | thaliana | CCG30, | | | | (Ac2V) |
| | | CCG33, | | | | |
| | | CCG40, | | | | |
| | | CCG67, | | | | |
| | | CCG71, | | | | |
| | | CCG79, | | | | |
| | | CCG104 | | | | |
| WRR4B | Arabidopsis | CCG45, | — | — | — | Albugo candida |
| | thaliana | CCG57, | | | | (Ac2V) |
| | | CCG61, | | | | |
| | | CCG70 | | | | |
| Fom-2 | Cucumis | AvrFom2 | — | — | — | Fusarium |
| | melo | | | | | oxysporum f. |
| | | | | | | sp. melonis |
| MLA1 | Hordeum | AVRa1 | — | — | — | Blumeria |
| | vulgare | | | | | graminis f. sp. |
| | | | | | | hordei |
| MLA10 | Hordeum | AVRa10 | — | — | — | Blumeria |
| | vulgare | | | | | graminis f. sp. |
| | | | | | | hordei |
| MLA13 | Hordeum | AVRa13 | — | — | — | Blumeria |
| | vulgare | | | | | graminis f. sp. |
| | | | | | | hordei |
| MLA22 | Hordeum | AVRa22 | — | — | — | Blumeria |
| | vulgare | | | | | graminis f. sp. |
| | | | | | | hordei |
| MLA7 | Hordeum | AVRa7 | — | — | — | Blumeria |
| | vulgare | | | | | graminis f. sp. |
| | | | | | | hordei |
| MLA9 | Hordeum | AVRa9 | — | — | — | Blumeria |
| | vulgare | | | | | graminis f. sp. |
| | | | | | | hordei |
| L2 | Linum | AvrL2-A | — | — | — | Melampsora |
| | usitatissimum | | | | | lini |
| L6 | Linum | AvrL567-A, | — | — | — | Melampsora |
| | usitatissimum | AvrL567-B, | | | | lini |
| | | AvrL567-D, | | | | |
| | | AvrL567-E, | | | | |
| | | AvrL567-F, | | | | |
| | | AvrL567-J, | | | | |
| | | AvrL567-L | | | | |
| L7 | Linum | AvrL567-A, | — | — | — | Melampsora |
| | usitatissimum | AvrL567-B, | | | | lini |
| | | AvrL567-D, | | | | |
| | | AvrL567-E, | | | | |
| | | AvrL567-F, | | | | |
| | | AvrL567-J, | | | | |
| | | AvrL567-L | | | | |
| L5 | Linum | AvrL567-A, | — | — | — | Melampsora |
| | usitatissimum | AvrL567-B, | | | | lini |
| | | AvrL567-E, | | | | |
| | | AvrL567-F, | | | | |
| | | AvrL567-J, | | | | |
| | | AvrL567-L | | | | |
| M | Linum | AvrM; ABB | | | | Melampsora |
| | usitatissimum | 96264; | | | | lini |
| | | 16326930 | | | | |
| P | Linum | AvrP; ACD4 | — | — | — | Melampsora |
| | usitatissimum | 9721; | | | | lini |
| | | 19633228 | | | | |
| P2 | Linum | AvrP123; | | | | Melampsora |
| | usitatissimum | ABB96267; | | | | lini |
| | | 16326930 | | | | |
| Pi54 | Oryza sativa | Avr-Pi54 | KM043887.1 | SEQ ID | — | Magnaporthe |
| | | | | NO: 403 | | oryzae |
| Pi9 | Oryza sativa | AvrPi9 | MZ327710.1 | SEQ ID | — | Magnaporthe |
| | | | | NO: 404 | | oryzae |
| Pia-2 | Oryza sativa | Avr-Pia, | — | — | — | Magnaporthe |
| | | Avr1-CO39 | | | | oryzae |
| Pib | Oryza sativa | AvrPiB | AB013449.2 | SEQ ID | — | Magnaporthe |
| | | | | NO: 405 | | oryzae |
| Pi5-1 | Oryza sativa | AvrPii | EU869185.1 | SEQ ID | — | Magnaporthe |
| | | | | NO: 406 | | oryzae |

¹The descriptors “ATXGXXXXX” refer to the Arabidopsis Information Resource (TAIR) database accession number for entries in the world wide web internet database “arabidopsis.org.” The other descriptors refer to the National Center for Biotechnology Information database accession number for entries in the world wide web internet database “ncbi[dot]nlm[dot]nih.gov/nuccore.”

TABLE 13
Bioactive Plant peptides

| Peptide name | Sequence | Function |
|---|---|---|
| miPEP172c | SEQ ID | Increase nodulation in soybean |
| | NO: 408 | (improved nitrogen fixation, |
| | | improved yield) |
| miPEP171d | SEQ ID | Increase adventitious root |
| | NO: 409 | formation in grapevine |
| BomiPEP397a | SEQ ID | Increase foliar surface (36%) |
| | NO: 410 | and root development in cabbage |
| AtmiPEP397a | SEQ ID | Increase root development in |
| | NO: 411 | Arabidopsis |
| BvmiPEP164b | SEQ ID | Growth inhibition of Barbarea |
| | NO: 412 | vulgaris (a weed) |
| AtmiPEP164b | SEQ ID | Root growth inhibition in |
| | NO: 413 | Arabidopsis |

TABLE 14
Ribozymes

| Ribozyme | NCBI Accession ID | Rfam ID | Sequence¹ |
|---|---|---|---|
| Twister | KV767472.1 | RF03160 | SEQ ID NO: 414 |
| | (20283-20218) | | |
| Twister | CM001069.2 | RF03160 | SEQ ID NO: 415 |
| | (36212529-36212469) | | |
| Twister | — | RF03160; | SEQ ID NO: 416 |
| | | URS0000D6C74A_4558 | |
| | | (1-52) | |
| Twister | — | RF03160; | SEQ ID NO: 417 |
| | | URS0000D69167_4558 | |
| | | (1-52) | |
| Twister | — | RF03160; | SEQ ID NO: 418 |
| | | URS0000D66A77_4558 | |
| | | (1-52) | |
| Twister | AP014967.1 | RF03160 | SEQ ID NO: 419 |
| | (27291338-27291417) | | |
| Hammerhead | — | — | SEQ ID NO: 420 |
| Hammerhead | — | — | SEQ ID NO: 421 |
| targeting pepper | | | |
| phytoene | | | |
| desaturase | | | |
| GlmS | FJ935779.1 | RF00234 | SEQ ID NO: 422 |
| | (2022-2192) | | |
| HDV | — | — | SEQ ID NO: 423 |
| VS_ribozyme | 4R4V | — | SEQ ID NO: 424 |
| Hairpin | D00721.1 (306-255) | RF00173 | SEQ ID NO: 425 |
| Hairpin | M21212.1 (157-106) | RF00173 | SEQ ID NO: 426 |
| Hairpin | M14879.1 (224-175) | RF00173 | SEQ ID NO: 427 |
| Hairpin | D00685.1 (306-255) | RF00173 | SEQ ID NO: 428 |
| Hairpin | D00721.1 (306-255) | RF00173 | SEQ ID NO: 429 |
| Hairpin | M17439.1 (226-177) | RF00173 | SEQ ID NO: 430 |
| Hairpin | — | RF04190; | SEQ ID NO: 431 |
| | | URS000080E2EB_32630 | |
| | | (1-68) | |
| Group I self- | AP014966.1 | RF00028 | SEQ ID NO: 432 |
| splicing intron | (20467201-20466676) | | |
| Group I self- | ADWL01007159.1 | RF00028 | SEQ ID NO: 433 |
| splicing intron | (31477-32002) | | |
| Group II self- | ASAF01074251.1 | RF00029 | SEQ ID NO: 434 |
| splicing intron | (11469-11396 | | |
| Group II self- | CM001068.2 | RF00029 | SEQ ID NO: 435 |
| splicing intron | (57579158-57579287) | | |
| Group II self- | CAJW011389724.1 | RF00029 | SEQ ID NO: 436 |
| splicing intron | (98-22) | | |
| Group II self- | CAJW011360013.1 | RF00029 | SEQ ID NO: 437 |
| splicing intron | (116-194) | | |
| Group II self- | KL543661.1 | RF00029 | SEQ ID NO: 438 |
| splicing intron | (216201-216123) | | |

¹RNA equivalents of the DNA sequences are also contemplated and can be obtained from the DNA sequences provided.

TABLE 15
RNA Aptamers

| RNA aptamer | Sequence¹ |
|---|---|
| Pepper | SEQ ID NO: 439 |
| Broccoli | SEQ ID NO: 440 |
| Corn-SAM | SEQ ID NO: 441 |
| Mango | SEQ ID NO: 442 |
| Mango II | SEQ ID NO: 443 |
| Mango III | SEQ ID NO: 444 |
| Mango III (A10U) | SEQ ID NO: 445 |
| Mango IV | SEQ ID NO: 446 |
| Squash | SEQ ID NO: 447 |
| Chili | SEQ ID NO: 448 |
| Spinach | SEQ ID NO: 449 |
| Spinach2 | SEQ ID NO: 450 |
| Orange broccoli | SEQ ID NO: 451 |
| DIR2s | SEQ ID NO: 452 |

¹DNA equivalents of the RNA sequences are also contemplated and can be obtained from the RNA sequences provided.

TABLE 16
Subgenomic promoters

| Subgenomic promoter | Sequence¹ |
|---|---|
| Pea early-browning virus CP | SEQ ID NO: 453 |
| Tobacco rattle virus strain TCM CP* | SEQ ID NO: 454 |
| Tobacco rattle virus strain PSG CP* | SEQ ID NO: 455 |
| Tobacco rattle virus strain PLB CP* | SEQ ID NO: 456 |
| Tobacco rattle virus strain PRV CP* | SEQ ID NO: 457 |
| Pea early-browning virus 12K | SEQ ID NO: 458 |
| Pea early-browning virus 29.6K | SEQ ID NO: 459 |
| Pea early-browning virus 29K | SEQ ID NO: 460 |
| Tobacco mosaic virus CP | SEQ ID NO: 461 |
| Tobacco mosaic virus MP | SEQ ID NO: 462 |
| Cucumber mosaic virus CP | SEQ ID NO: 463 |

¹RNA equivalents of the DNA sequences are also contemplated and can be obtained from the DNA sequences provided.
*See Goulden et al. (1990) Nucleic Acids Res., 18:4507-4512, DOI: 10.1093/NAR/18.15.4507.

TABLE 17
Secoviridae segment 2 genomes and polyprotein and CP sequences

| | 5′ RNA | | Polyprotein | | |
|---|---|---|---|---|---|
| | Replication | | DNA | | CP DNA |
| | Element | Polyprotein | Coding | CP Protein | Coding |
| Secoviridae Genome | Sequence | Sequence | Sequence | Sequence | Sequence |
| Apple latent spherical | SEQ ID | SEQ ID | SEQ ID | SEQ ID | SEQ ID |
| virus (ALSV) segment 2 | NO: 468 | NO: 632 | NO: 634 | NOs: 473, | NOs: 474, |
| (NCBI: NC_003788.1) | | | | 475, and | 476, and |
| SEQ ID NO: 629 | | | | 477 | 478 |
| Soybean latent spherical | SEQ ID | SEQ ID | SEQ ID | | |
| virus segment 2 | NO: 631 | NO: 633 | NO: 635 | | |
| (NCBI: NC_032271.1) | | | | | |
| SEQ ID NO: 630 | | | | | |

TABLE 18
Secoviridae segment 1 genomes and RDRP sequences

| | 5′ RNA | | |
|---|---|---|---|
| | Replication | RdRP | |
| | Element | Protein | RdRP DNA |
| Secoviridae Genome | Sequence | Sequence | Coding Sequence |
| Apple latent spherical | SEQ ID | SEQ ID | SEQ ID |
| virus (ALSV) segment 1 | NO: 467 | NO: 471, | NO: 472, |
| (NCBI: NC_003787.1) | | SEQ ID | SEQ ID |
| SEQ ID NO: 503 | | NO: 558 | NO: 565 |
| Soybean latent | SEQ ID | SEQ ID | SEQ ID |
| spherical virus segment 1 | NO: 512 | NO: 530 | NO: 539 |
| (NCBI: NC_032270) | | | |
| SEQ ID NO: 504 | | | |
| Currant latent virus | SEQ ID | SEQ ID | SEQ ID |
| segment 1 | NO: 546 | NO: 559 | NO: 566 |
| (NCBI: NC_029038) | | | |
| SEQ ID NO: 540 | | | |
| Arabidopsis latent | SEQ ID | SEQ ID | SEQ ID |
| virus-1 segment 1 | NO: 547 | NO: 560 | NO: 567 |
| (NCBI: NC_078590) | | | |
| SEQ ID NO: 541 | | | |
| Artichoke Italian | SEQ ID | SEQ ID | SEQ ID |
| latent virus segment 1 | NO: 548 | NO: 561 | NO: 568 |
| (NCBI: NC_043684) | | | |
| SEQ ID NO: 542 | | | |
| Blueberry latent | SEQ ID | SEQ ID | SEQ ID |
| spherical virus segment 1 | NO: 549 | NO: 562 | NO: 569 |
| (NCBI: NC_038764) | | | |
| SEQ ID NO: 543 | | | |
| Grapevine Bulgarian | SEQ ID | SEQ ID | SEQ ID |
| latent virus segment 1 | NO: 550 | NO: 563 | NO: 570 |
| (NCBI: NC_015492) | | | |
| SEQ ID NO: 544 | | | |
| Strawberry latent | SEQ ID | SEQ ID | SEQ ID |
| ringspot virus segment 1 | NO: 551 | NO: 564 | NO: 571 |
| (NCBI: NC 006964) | | | |
| SEQ ID NO: 545 | | | |

TABLE 19
Secoviridae Genome 5′ and 3′ RNA Replication Element Pairs and RdRP

| | 5′ RNA | 3′ RNA | | |
|---|---|---|---|---|
| | Replication | Replication | | |
| Secoviridae | Element | Element | RdRP Protein | Plant Host |
| Genome | SEQ ID NO | SEQ ID NO | SEQ ID NO | Examples |
| Apple latent | SEQ ID | SEQ ID | SEQ ID | Malus |
| spherical virus | NO: 467 | NO: 469, | NO: 471, | domestica |
| (ALSV) segment 1 | | SEQ ID | SEQ ID | |
| (NCBI: NC_003787.1) | | NO: 520 | NO: 558 | |
| SEQ ID NO: 503 | | | | |
| Soybean latent | SEQ ID | SEQ ID | SEQ ID | Glycine max |
| spherical virus | NO: 512 | NO: 521 | NO: 530 | |
| segment 1 | | | | |
| (NCBI: NC_032270) | | | | |
| SEQ ID NO: 504 | | | | |
| Currant latent virus | SEQ ID | SEQ ID | SEQ ID | Ribes rubrum |
| segment 1 | NO: 546 | NO: 552 | NO: 559 | |
| (NCBI: NC_029038) | | | | |
| SEQ ID NO: 540 | | | | |
| Arabidopsis latent | SEQ ID | SEQ ID | SEQ ID | Arabidopsis |
| virus-1 segment 1 | NO: 547 | NO: 553 | NO: 560 | thaliana |
| (NCBI: NC_078590) | | | | |
| SEQ ID NO: 541 | | | | |
| Artichoke Italian | SEQ ID | SEQ ID | SEQ ID | Cynara |
| latent virus | NO: 548 | NO: 554 | NO: 561 | cardunculus |
| segment 1 | | | | |
| (NCBI: NC_043684) | | | | |
| SEQ ID NO: 542 | | | | |
| Blueberry latent | SEQ ID | SEQ ID | SEQ ID | Vaccinia sp. |
| spherical virus | NO: 549 | NO: 555 | NO: 562 | |
| segment 1 | | | | |
| (NCBI: NC_038764) | | | | |
| SEQ ID NO: 543 | | | | |
| Grapevine | SEQ ID | SEQ ID | SEQ ID | Vitis vinifera |
| Bulgarian latent | NO: 550 | NO: 556 | NO: 563 | |
| virus segment 1 | | | | |
| (NCBI: NC_015492) | | | | |
| SEQ ID NO: 544 | | | | |
| Strawberry latent | SEQ ID | SEQ ID | SEQ ID | Fragaria sp. |
| ringspot virus | NO: 551 | NO: 557 | NO: 564 | |
| segment 1 | | | | |
| (NCBI: NC_006964) | | | | |
| SEQ ID NO: 545 | | | | |

### EXAMPLES

### Example 1. An Antiviral Secoviridae Satellite with an Imbedded HRV Amplicon and RNAi Cargo

A Secoviridae satellite (COMSAT) that carries an antiviral inhibitory RNA (RNAi) cargo is generally provided in 5′ to 3′ orientation as follows: (i) 5′ replication element from a virus of the family Secoviridae; (ii) a heterologous RNA virus (HRV) 5′ replication region (HRV 5′RR); (iii) an antiviral RNA molecule which induces an RNAi response; (iv) the heterologous RNA virus (HRV) 3′ RNA replication region (HRV 3′RR); and (v) and the 3′ RNA replication element from a virus of the family Secoviridae.

COMSATs comprising Secoviridae 5′ and 3′ RNA replication elements flanking a TMV amplicon containing a cargo RNA containing a Pepper mild mottle virus (PMMoV) RNAi inducing sequence (e.g., a PMMoV sequence which can form a dsRNA) are provided as SEQ ID NO: 479 and 480. The elements of the satellites are set forth in Table 20.

TABLE 20

| | Nucleotide | Nucleotide |
|---|---|---|
| | position in SEQ | position in SEQ |
| Genetic element | ID NO: 479 | ID NO: 480 |
| Secoviridae 5′ RNA | 1-384 | 1-311 |
| replication element | | |
| TMV 5′ replication | 385-452 | 312-379 |
| region | | |
| Antiviral RNAi cargo | 453-1430 | 380-1357 |
| (PMMoV RNAi inducing | | |
| sequence) | | |
| TMV 3′ replication | 1431-1634 | 1358-1561 |
| region | | |
| ZmHSP101 IRES (optional) | 1635-1781 | 1562-1708 |
| TMV MP (optional) | 1782-2588 | 1709-2515 |
| Ileu tRNA | 2589-2670 | 2516-2597 |
| Secoviridae 3′ RNA | 2671-2858 | 2598-2784 |
| replication element | | |

### Example 2. An Antiviral Secoviridae Satellite with an Imbedded HRV Amplicon and Antiviral Protein Cargo

A Secoviridae satellite (COMSAT) with an imbedded HRV amplicon that carries an antiviral protein cargo is generally provided in 5′ to 3′ orientation as follows: (i) 5′ replication element from a virus of the family Secoviridae; (ii) a heterologous RNA virus (HRV) 5′ replication region (HRV 5′RR); (iii) an antiviral protein cargo; (iv) the heterologous RNA virus (HRV) 3′ RNA replication region (HRV 3′RR); and (v) the 3′ RNA replication element from a virus of the family Secoviridae.

COMSATs comprising Secoviridae 5′ and 3′ RNA replication elements flanking a tobacco mosaic virus (TMV) amplicon containing an antiviral cargo protein containing the coding sequence of the N gene of tobacco are provided as SEQ ID NO: 481 and 482. This satellite includes TMV 5′ and 3′ replication region sequences (HRV 5′ and 3′ RR sequences) to promote secondary amplification by TMV, which is a pathogenic tobamovirus. The elements of this satellite are set forth in Table 21. Similar antiviral Secoviridae satellites are designed for secondary amplification by tomato mosaic virus or tomato brown rugose fruit virus (which are also pathogenic tobamoviruses), and include an N protein (SEQ ID NO:254), L4 protein (SEQ ID NO:256), or a combination of N and L4 protein as the antiviral cargo sequence(s), and replacing the HRV 5′ and 3′ RR sequences of TMV with the HRV 5′ and 3′ RR sequences of either tomato mosaic virus or tomato brown rugose fruit virus as provided in Table 7, to promote secondary amplification by tomato mosaic virus or tomato brown rugose fruit virus, respectively. Solanaceous plants containing the virus of the order Secoviridae (or that otherwise are provided with the appropriate Secoviridae RdRP, e.g., through transient or transgenic expression) and that are provided with these antiviral Secoviridae satellites are expected to exhibit resistance to tobacco mosaic virus, tomato mosaic virus, or tomato brown rugose fruit virus, respectively.

TABLE 21

| | Nucleotide | Nucleotide |
|---|---|---|
| | position in SEQ | position in SEQ |
| Genetic element | ID NO: 481 | ID NO: 482 |
| Secoviridae 5′ RNA | 1-384 | 1-311 |
| replication element | | |
| TMV 5′ replication | 385-452 | 312-379 |
| region | | |
| PSVI IRES | 453-648 | 380-575 |
| Antiviral protein cargo | 649-4083 | 576-4010 |
| (coding sequence of | | |
| N gene) | | |
| TMV 3′ replication | 4084-4287 | 4011-4214 |
| region | | |
| ZmHSP101 IRES (optional) | 4288-4434 | 4215-4361 |
| TMV MP (optional) | 4435-5241 | 4362-5168 |
| Ileu tRNA | 5242-5323 | 5169-5250 |
| Secoviridae 3′ RNA | 5324-5511 | 5251-5437 |
| replication element | | |

### Example 3. An Antiviral Secoviridae Satellite with RNAi Cargo

A Secoviridae satellite (COMSAT) that carries an antiviral RNAi cargo is generally provided in 5′ to 3′ orientation as follows: (i) 5′ replication element from a virus of the family Secoviridae; (ii) an antiviral RNA molecule which induces an RNAi response; and (iii) the 3′ RNA replication element from a virus of the family Secoviridae.

COMSATs comprising Secoviridae 5′ and 3′ RNA replication elements flanking a cargo RNA containing a Pepper mild mottle virus (PMMoV) RNAi inducing sequence (e.g., a PMMoV sequence which can form a dsRNA) are provided as SEQ ID NO: 483 and 484. The elements of the satellites are set forth in Table 22.

TABLE 22

| | Nucleotide | Nucleotide |
|---|---|---|
| | position in SEQ | position in SEQ |
| Genetic element | ID NO: 483 | ID NO: 484 |
| Secoviridae 5′ RNA | 1-384 | 1-311 |
| replication element | | |
| Antiviral RNAi cargo | 385-1362 | 312-1289 |
| (PMMoV RNAi inducing | | |
| sequence) | | |
| ZmHSP101 IRES | 1363-1509 | 1290-1436 |
| TMV MP | 1510-2316 | 1437-2243 |
| Ileu tRNA | 2317-2398 | 2244-2325 |
| Secoviridae 3′ RNA | 2399-2586 | 2326-2612 |
| replication element | | |

### Example 4. An Antiviral Secoviridae Satellite with Antiviral Protein Cargo

A Secoviridae satellite (COMSAT) that carries an antiviral protein cargo is generally provided in 5′ to 3′ orientation as follows: (i) 5′ replication element from a virus of the family Secoviridae; (ii) an antiviral protein cargo; and (iii) the 3′ RNA replication element from a virus of the family Secoviridae.

COMSATs comprising Secoviridae 5′ and 3′ RNA replication elements flanking an antiviral protein cargo containing the coding sequence of the N gene of tobacco are provided as SEQ ID NO: 485 and 486. The elements of the satellites are set forth in Table 23.

TABLE 23

| | Nucleotide | Nucleotide |
|---|---|---|
| | position in SEQ | position in SEQ |
| Genetic element | ID NO: 485 | ID NO: 486 |
| Secoviridae 5′ RNA | 1-384 | 1-311 |
| replication element | | |
| PSVI IRES | 385-580 | 312-507 |
| Antiviral protein cargo | 581-4015 | 508-3942 |
| (coding sequence of | | |
| N gene) | | |
| ZmHSP101 IRES | 4016-4162 | 3943-4089 |
| TMV MP | 4163-4969 | 4090-4896 |
| Ileu tRNA | 4970-5051 | 4897-4978 |
| Secoviridae 3′ RNA | 5052-5239 | 4979-5165 |
| replication element | | |

### Example 5. Assaying Plants Comprising Antiviral Secoviridae Satellite with Antiviral Cargo for Antiviral Activity

Antiviral COMSATs (e.g., of Examples 1 to 4) or control RNAs (e.g., RNAs lacking the antiviral cargo) are delivered into target host plants, such as pepper or tomato plants via micro-bombardment (“biolistic” delivery) using a “gene gun” or by bacterially mediated (e.g., by Agrobacterium) transient expression. Host plants lacking the appropriate endogenous Secoviridae virus can otherwise be provided with the appropriate Secoviridae RdRP, e.g., through transient or transgenic expression. Plants, e.g., pepper and tomato plants, are grown in growth chambers using a long-day photoperiod (16 h light/8 h dark) with light intensity 100-150 micromole, until seedlings are about 2 weeks old. Antiviral COMSATs are prepared either as in vitro transcribed (IVT) products (capped or uncapped), or as Agrobacterium binary vectors. For micro-bombardment, the IVT product is coated onto the surface of gold nanoparticles which are precipitated on inner surface of bullet tubes; these are accelerated at the abaxial surface of the seedlings' leaves with helium pressure from 100 to 180 psi. For agroinfiltration, COMSAT-carrying clones in binary vector are transformed into Agrobacterium GV 2260, and the transformed Agrobacterium is selected by growing at 28° C. under appropriate antibiotic selection. The positive transformants are verified by PCR and then grown at 28° C. overnight in liquid LB medium with appropriate antibiotics plus rifamycin to maintain the Agrobacterium and prevent contamination. Agrobacterium cells are collected by centrifugation and resuspended in MMA buffer at an OD600 of 0.2, incubated at 28° C. on a shaker for 2 hours, and then infiltrated into the abaxial sides of the leaves. Control plants are treated in the same way but with non-COMSAT control IVT transcripts, or empty buffers. At different timepoints after COMSAT delivery, tissues from systemic leaves (leaves distal to the treated leaves) are collected and subjected to RNA extraction, followed by cDNA syntheses. COMSAT titers are monitored in systemic tissue by qRT-PCR with COMSAT-specific primers.

The efficacy of an antiviral COMSAT can be tested by challenging the COMSAT-treated plants with the viral pathogen of interest (e.g., cucumber mosaic virus, CMV, or tobacco mosaic virus, TMV), for example, by mechanical infection. Infectious preparations of such acute viral pathogens are prepared in planta (e.g., in Nicotiana benthamiana) or in the form of GFP-fused infectious clones in a binary vector or T7-based vector. Suitable viral inoculums can be prepared as infectious sap extracted from an infected plant, as an Agrobacterium-based inoculum, or as IVT products. The inoculum is introduced into leaves of the COMSAT-treated plants by rub-inoculation (for infectious sap or IVT product), by agroinfiltration (for GFP-fused infectious clone in binary vector), or by micro-bombardment (for IVT product or plasmid of infectious clone in binary vector). For rub-inoculation, the infectious sap or IVT products are respectively diluted 3 times or to 50 ng/microliter in phosphate buffer 0.05M, pH 7.4 and dropped onto leaf adaxial surfaces which is pre-dusted with an abrasive such as carborundum or bentonite. The inoculum is gently spread on the abrasive-dusted leaf surface by gloved fingers or cotton buds; after 30 seconds the inoculated leaf is washed with water. Micro-bombardment of infectious clones is conducted following previously described methods (see. e.g., delivery techniques described in PCT/US22/78963; see also Bio-Rad Tech Note 2531 “Inoculation of Viral RNA and cDNA to Potato and Tobacco Plants Using the Helios™ Gene Gun”). Infiltration with an Agrobacterium-based viral pathogen inoculum is performed using methods similar to that used for COMSAT infiltration, but the Agrobacterium suspension is diluted at OD600 of 0.1.

Symptoms and titre of the acute viral pathogens are monitored in the plants, typically over time to confirm progress or decline of viral infection. Effectiveness of antiviral COMSATs is evaluated by comparing relative titres of systemic infected viruses in antiviral COMSAT-treated plants and the control plants. Tissues from inoculated leaves and systemic leaves (distal to the inoculated leaves) are collected, total RNA is extracted, optionally followed by cDNA synthesis. The viral titre can be quantitatively measured by qRT-PCR with virus-specific primers. Viral titre or presence can be measured by immunoassay methods; for example, Tobacco mosaic virus (TMV) is routinely detected in plants using a commercially available strip assay (Agdia ImmunoStrip® for TMV Agdia, Inc., Elkhart, IN, USA). Alternatively, viral titre is qualitatively evaluated by viral disease symptoms, or by proxy measurement (e.g., measuring GFP expressed by GFP-fused viral constructs). Generally, qRT-PCR measurements are normalized to endogenous reference gene controls (e.g., actin, tubulin, ubiquitin-3, GADPH, or translation elongation factor EF1a, used individually or preferably in multiples. e.g., at least 3 reference genes or at least three deltaCt values). The viral titre (as fold changed, relative to an endogenous reference gene) is compared to that of controls.

### Example 6. An Insect Inhibitory Secoviridae Satellite with Insecticidal Protein or RNAi Cargo

A Secoviridae satellite (COMSAT) that carries an insecticidal RNAi cargo is generally provided in 5′ to 3′ orientation as follows: (i) 5′ replication element from a virus of the family Secoviridae; (ii) an insecticidal RNA molecule which induces an RNAi response; and (iii) the 3′ RNA replication element from a virus of the family Secoviridae.

COMSATs comprising Secoviridae 5′ and 3′ RNA replication elements flanking a cargo RNA containing an RNAi inducing sequence that targets Colorado potato beetle are provided as SEQ ID NO: 487 and 488. The elements of the satellites are set forth in Table 24.

TABLE 24

| | Nucleotide | Nucleotide |
|---|---|---|
| | position in SEQ | position in SEQ |
| Genetic element | ID NO: 487 | ID NO: 488 |
| Secoviridae 5′ RNA | 1-384 | 1-311 |
| replication element | | |
| Insecticidal RNAi | 385-872 | 312-799 |
| cargo (targeting | | |
| Colorado potato | | |
| beetle) | | |
| ZmHSP101 IRES | 873-1019 | 800-946 |
| TMV MP | 1020-1826 | 947-1753 |
| Ileu tRNA | 1827-1908 | 1754-1835 |
| Secoviridae 3′ RNA | 1909-2096 | 1836-2022 |
| replication element | | |

A Secoviridae satellite (COMSAT) that carries an insecticidal protein cargo is generally provided in 5′ to 3′ orientation as follows: (i) 5′ replication element from a virus of the family Secoviridae; (ii) an insecticidal protein cargo; and (iii) the 3′ RNA replication element from a virus of the family Secoviridae.

COMSATs comprising Secoviridae 5′ and 3′ RNA replication elements flanking an insecticidal cargo protein containing the coding sequence of the Vip3Aa gene are provided as SEQ ID NO: 489 and 490. The elements of the satellites are set forth in Table 25.

TABLE 25

| | Nucleotide | Nucleotide |
|---|---|---|
| | position in SEQ | position in SEQ |
| Genetic element | ID NO: 489 | ID NO: 490 |
| Secoviridae 5′ RNA | 1-384 | 1-311 |
| replication element | | |
| PSVI IRES | 385-580 | 312-507 |
| Insecticidal protein | 581-2950 | 508-2877 |
| cargo (coding sequence | | |
| of Vip3 Aa gene) | | |
| ZmHSP101 IRES | 2951-3097 | 2878-3024 |
| TMV MP | 3098-3904 | 3025-3831 |
| Ileu tRNA | 3905-3986 | 3832-3913 |
| Secoviridae 3′ RNA | 3987-4174 | 3914-4100 |
| replication element | | |

COMSATS of Table 24 and Table 25 are established in host plants essentially as set forth in Example 5. Host plants lacking the appropriate endogenous Secoviridae virus can otherwise be provided with the appropriate Secoviridae RdRP, e.g., through transient or transgenic expression. The efficacy of an insecticidal or insect inhibitory COMSAT can be tested by challenging the COMSAT and control treated plants with the insect of interest (e.g., Colorado Potato Beetle for plants treated with the Table 24 COMSAT or Fall Armyworm, Corn Earworm, or Black Cutworm for plants treated with the Table 25 COMSAT).

### Example 7. An Antifungal Secoviridae Satellite with Antifungal Protein or RNAi Cargo

A Secoviridae satellite (COMSAT) that carries an antifungal RNAi cargo is generally provided in 5′ to 3′ orientation as follows: (i) 5′ replication element from a virus of the family Secoviridae; (ii) an antifungal RNA molecule which induces an RNAi response; and (iii) the 3′ RNA replication element from a virus of the family Secoviridae.

COMSATs comprising Secoviridae 5′ and 3′ RNA replication elements flanking a cargo RNA containing an RNAi inducing sequence that targets a DCL gene of Botrytis cinerea are provided as SEQ ID NO: 491 and 492. The elements of the satellites are set forth in Table 26.

TABLE 26

| | Nucleotide | Nucleotide |
|---|---|---|
| | position in SEQ | position in SEQ |
| Genetic element | ID NO: 491 | ID NO: 492 |
| Secoviridae 5′ RNA | 1-384 | 1-311 |
| replication element | | |
| Antifungal RNAi cargo | 385-733 | 312-660 |
| (targeting DCL gene of | | |
| Botrytis cinerea) | | |
| ZmHSP101 IRES | 734-880 | 661-807 |
| TMV MP | 881-1687 | 808-1614 |
| Ileu tRNA | 1688-1769 | 1615-1696 |
| Secoviridae 3′ RNA | 1770-1957 | 1697-1883 |
| replication element | | |

A Secovirida satellite (COMSAT) that carries an antifungal protein cargo is generally provided in 5′ to 3′ orientation as follows: (i) 5′ replication element from a virus of the family Secoviridae; (ii) an antifungal protein cargo; and (iii) the 3′ RNA replication element from a virus of the family Secoviridae.

COMSATs comprising Secoviridae 5′ and 3′ RNA replication elements flanking an antifungal cargo protein containing the coding sequence of the CaAMP1 gene are provided as SEQ ID NO: 493 and 494. The elements of the satellites are set forth in Table 27.

TABLE 27

| | Nucleotide | Nucleotide |
|---|---|---|
| | position in SEQ | position in SEQ |
| Genetic element | ID NO: 493 | ID NO: 494 |
| Secoviridae 5′ RNA | 1-384 | 1-311 |
| replication element | | |
| PSVI IRES | 385-580 | 312-507 |
| Antifungal cargo (coding | 581-1141 | 508-1068 |
| sequence of CaAMP1 gene) | | |
| ZmHSP101 IRES | 1142-1288 | 1069-1215 |
| TMV MP | 1289-2095 | 1216-2022 |
| Ileu tRNA | 2096-2177 | 2023-2104 |
| Secoviridae 3′ RNA | 2178-2365 | 2105-2291 |
| replication element | | |

A COMSAT construct is built by Golden Gate assembly of synthesized cargos and other COMSAT components synthesized from a commercial vendor. The plasmid is used as a template for a PCR reaction to amplify the expression cassette with a T7 promoter. This PCR product is used as the template for an in vitro transcription reaction using a MEGAscript™ T7 Transcription Kit from Thermo Fisher Scientific. The in vitro synthesized RNA or protein cargo is then transformed into leaves of the target crop using gold nanoparticles from Bio-Rad according to the guidelines specified in the Helios gene gun manual or via Agrobacterium mediated transformation. Host plants lacking the appropriate endogenous Secoviridae virus can otherwise be provided with the appropriate Secoviridae RdRP, e.g., through transient or transgenic expression. Replication and persistence of delivered RNA or expression of protein in local leaves can be determined by RT-PCR from local tissues at 2-weeks post transformation. Systemic movement of RNA/protein is determined by qRT-PCR/ELISA in the sample collected from distal untreated leaf samples after 1-, 2-, and 3-months post transformation.

To validate the efficacy of an antifungal COMSAT, in vitro and in vivo fungal bioassays are performed. An antifumgal in vitro assay is performed according to Duanis-Assaf et. al. (Plant Biotechnol J. 2022 January:20(1):226-237). An in vivo detached leaf assay is performed according to Li et. al. (Mol Plant Microbe Interact. 2019 December; 32(12):1649-1664). Disease phenotypes are evaluated in COMSAT and control plants by measuring a lesion area developed by the fungus in inoculated leaves at 3- and 6-days post inoculation.

### Example 8. Replication of a Secoviridae COMSAT in Tomato

A DNA molecule encoding an RNA molecule containing a 5′ replicase recognition sequence from a virus of the family Secoviridae; Dicer-like 1 and Dicer-like 2 fragments; ZmHSP101 IRES; TMV movement protein: Flowering Locus T from Arabidopsis thaliana; and a 3′ replicase recognition sequence from a virus of the family Secoviridae is cloned into a pUC19 plasmid backbone and transformed into E. coli DH5a (New England Biolabs; C29871). The plasmid is extracted, and PCR is used to add a T7 promotor sequence. In-vitro transcription is used to synthesize the RNA molecule, which is then purified using a Monarch® RNA Cleanup Kit (50 μg) (New England Biolabs; T2040L). RNA quantity is measured using a Nanodrop spectrophotometer (Thermo Fisher Scientific; 840274200) and the intactness is quality controlled through gel electrophoresis. The synthesized and quality-controlled RNA molecule is precipitated onto gold nanoparticles and fired into 4-week-old Solanum lycopersicum ‘Early Girl’ leaves using a gene gun. Host plants lacking the appropriate endogenous Secoviridae virus can otherwise be provided with the appropriate Secoviridae RdRP, e.g., through transient or transgenic expression. The following leaf tissue samples are collected at 2-week and 4-week timepoints: fired leaf #1; fired leaf #2; nearest adjacent leaflet; opposite leaf, terminal leaflet; and apical leaf growth. RNA is extracted with the MagMax Plant RNA Isolation kit (Thermo Fisher Scientific; A33899) and quality controlled through Nanodrop spectrophotometer (Thermo Fisher Scientific; 840274200) and RNA ScreenTape Analysis (Agilent; 5067-5576). Quality controlled RNA is used for cDNA synthesis and qRT-PCR is used to amplify the Dicer-like 1 and Dicer-like 2 fragments and reference gene fragment RPL2. Delta-delta Ct analysis is applied to determine LogFold change of the Secoviridae COMSAT in the tissues compared to non-Secoviridae COMSAT containing Solanum lycopersicum ‘Early Girl’ plant tissues.

### Example 9. Secoviridae COMSAT with cDNA-Based Cargo Expression Amplification

A DNA molecule encoding an RNA molecule containing a 5′ replicase recognition sequence from a virus of the family Secoviridae; Cricket paralysis virus IRES; Avian leukosis virus reverse transcriptase; tRNA primer binding site (tRNA-trp); CaMV 35s promoter; a protein cargo (Superfolder GFP); ZmHSP101 IRES; Tobacco mosaic virus movement protein; and a 3′ replicase recognition site from a virus of the family Secoviridae is cloned into a pUC19 plasmid backbone and is transformed into E. coli DH5a (New England Biolabs; C29871). The plasmid is extracted, and PCR is used to add a T7 promotor sequence. In-vitro transcription is used to synthesize the RNA molecule, which is then purified using a Monarch® RNA Cleanup Kit (50 μg) (New England Biolabs; T2040L). RNA quantity is measured using a Nanodrop spectrophotometer (Thermo Fisher Scientific; 840274200) and the intactness is quality controlled through gel electrophoresis. The synthesized and quality-controlled RNA molecule is precipitated onto gold nanoparticles and fired into the 4-week-old Solanum lycopersicum ‘Early Girl’ leaves using a gene gun. Host plants lacking the appropriate endogenous Secoviridae virus can otherwise be provided with the appropriate Secoviridae RdRP, e.g., through transient or transgenic expression. The following leaf tissue samples are collected at 2-week and 4-week timepoints: fired leaf #1: fired leaf #2; nearest adjacent leaflet: opposite leaf, terminal leaflet; and apical leaf growth. Protein (GFP) expression is quantified. Total protein is extracted from collected tissue samples using the Pierce Plant Total Protein Extraction Kit (Thermo Fisher Scientific; A44056) according to manufacturer specifications, and quality controlled by Nanodrop spectrophotometry paired with SDS-PAGE to verify extraction yield and integrity. GFP expression is quantified relative to other protein expressing COMSAT designs as well as non-transfected tissue using the Abcam GFP ELISA Kit (ab171581) according to manufacturer specifications.

### Example 10. Detection of Satellite RNA Negative-Sense Strand

Where only the positive-sense strand of a satellite RNA is provided to a plant or plant cell (e.g., by delivery of the RNA itself or by delivery of DNA encoding the satellite RNA's positive-sense strand), replication of the satellite RNA in a plant or plant cell can be verified by detection of the satellite RNA's negative-sense strand, for example, by using a negative-sense-strand-specific Taqman® assay.

In one non-limiting example, a bell pepper endornavirus (BPEV) satellite of 2357 nucleotides was constructed with the following elements in 5′ to 3′ orientation: (1) BPEV 5′ RNA replication element; (2) a first IRES sequence (PSVI IRES, with the addition of spacer nucleotides on the 5′ end); (3) RNA encoding a reporter protein (GFP): (4) a second IRES sequence (ZmHSP IRES); (5) a viral movement protein; (6) a zebrafish sequence (as a non-plant heterologous sequence for detection purposes): (7) an isoleucine tRNA sequence; (8) BPEV 3′ RNA replication element. Similar satellites can be designed and constructed using alternative genetic elements such as those provided elsewhere in this specification, such as pairs of 5′ and 3′ RNA replication elements from a virus of the family Secoviridae, other pairs of HRV 5′ and 3′ replication region sequences, and/or other cargo sequences.

The positive-sense strand of this BPEV satellite RNA was produced through in vitro transcription (IVT), and biolistically delivered with a gene gun into bell pepper plant leaves. Samples were collected 4 weeks post gene gun firing from a local leaf. Samples underwent RNA extraction, library prep, and Illumina®, sequencing using a stranded kit (NEBNext® Ultra™ II Directional RNA Library Prep Kit for Illumina®, catalogue numbers E7760S or E7760L, New England Biolabs, Ipswich, MA) with added DMSO treatment and plant rRNA depletion. Reads were mapped against the BPEV satellite RNA's sequence and negative-sense reads were identified using the Integrative Genomics Viewer (IGV) browser (Robinson et al., Bioinformatics, 39(1) (2023), btac830). Negative-sense reads mapping to the BPEV satellite were present, confirming that replication of the BPEV satellite had occurred. BPEV negative-sense reads were also identified in BPEV infected plants that served as a positive control for the analysis. The above-described negative strand detection method can be adapted for use in the detection of negative strand RNA produced by Secoviridae satellite RNAs disclosed herein.

### Example 11. Detection of Satellite RNA Negative-Sense Strand in a Tomato Plant

Where only the positive-sense strand of a satellite RNA is provided to a plant or plant cell (e.g., by delivery of the RNA itself or by delivery of DNA encoding the satellite RNA's positive-sense strand), replication of the satellite RNA in a plant or plant cell can be verified by detection of the satellite RNA's negative-sense strand, for example, by using a negative-sense-strand-specific Taqman® assay.

Negative sense GFP produced by a satellite RNA containing a GFP protein encoding cargo sequences is detected using Tagman® R) qPCR. The reference sequence that can be used is an endogenous tomato gene. “Tom06” (NCBI Gene ID 101249087, ubiquitin-conjugating enzyme E2-17 kDa, from Solanum lycopersicum). Taqman® qPCR primers and probes were ordered from Integrated DNA Technology, Coralville, IA. The negative sense GFP probe is labeled with FAM520 dye with ZEN/Iowa Black FQ quencher. The Tom06 probe is labeled with SUN5544 with ZEN/Iowa Black FQ quencher. Primers and probes are used at a 2:1 ratio. cDNA is synthesized via reverse transcription (30 minutes at 60 degrees C., then 10 minutes at 85 degrees C.) using Maxima H Minus First Strand cDNA synthesis Kit (K1652, Thermo Fisher Scientific, Waltham MA) using the RT primers in Table 28. This was followed by treatment (30 minutes at 37 degrees C., then 1 minute at 80 degrees C.) with thermolabile exonuclease I (E1050 New England Biolabs, Ipswich MA) to remove excess RT primers. The synthesized cDNA was cleaned with RNAClean XP beads (A63987 Beckman Coulter, Brea CA). Tagman® qPCR is carried out using Taqman® Advanced Fast MasterMix (444965, Thermo Fisher Scientific, Waltham MA) and the primer and probes in Table 29.

TABLE 28
Description of Reverse Transcriptase Primers

| RT Primer Name | RT Primer Sequence |
|---|---|
| negative GFP reverse | ccactggattcagcgaagttATGCG |
| transcription primer | TAAAGGCGAAGAG |
| | (SEQ ID NO: 668) |
| Tom06 (solanaceous | CTGAGTCCAGCTACGAGCAG |
| housekeeping reverse | (SEQ ID NO: 669) |
| transcription primer) | |

TABLE 29
Description of qPCR primers and probes

| qPCR Primer/Probe Name | qPCR Primer/Probe Sequence |
|---|---|
| negative GFP forward primer | CCACTGGATTCAGCGAAGT (SEQ ID NO: 670) |
| negative GFP reverse primer | AACTTCAGCGTCAGTTTACCA (SEQ ID NO: 671) |
| negative GFP probe | /6- |
| | FAM/CGAGGGTGA/ZEN/AGGTGACGCAACTA/3IABkFQ/ |
| | (SEQ ID NO: 672) |
| Tom06 forward primer | CCTGCTGTCCATCTGCTCTC (SEQ ID NO: 673) |
| Tom06 reverse primer | CTGAGTCCAGCTACGAGCAG (SEQ ID NO: 674) |
| Tom06 probe | /5SUN/ACAAGACTG/ZEN/ACAGGGCCAAA/3IABkFQ/ |
| | (SEQ ID NO: 675) |

### Other Embodiments

Although the foregoing disclosure has been described in some detail by way of illustration and example for purposes of clarity of understanding, the descriptions and examples should not be construed as limiting the scope of the disclosure.

Other embodiments are within the claims.

What is claimed is:

1. A recombinant RNA molecule comprising from 5′ terminus to 3′ terminus:
(a) a 5′ RNA replication element recognized by a Secoviridae RNA-dependent RNA polymerase (RdRP);
(b) a cargo RNA molecule; and
(c) a 3′ RNA replication element recognized by the RdRP;
wherein the 5′ RNA replication element, the cargo RNA molecule, and the 3′ RNA replication element are operably linked; wherein the cargo RNA molecule is heterologous to the 5′ RNA replication element and the 3′ RNA replication element; and, optionally, wherein: (i) the 5′ RNA replication element and the 3′ RNA replication element are obtained from the same Secoviridae genome or from Secoviridae genomes having at least 85%, 90%, 95%, 98%, or 99% sequence identity to one another; (ii) the 5′ RNA replication element, the 3′ RNA replication element, and the RdRP are obtained from the same Secoviridae genome or from Secoviridae genomes having at least 85%, 90%, 95%, 98%, or 99% sequence identity to one another; or (iii) the 5′ RNA replication element, the 3′ RNA replication element, and/or the RdRP coding region are obtained from different Secoviridae genomes, and wherein the members of each respective set of the 5′ RNA replication elements, 3′ RNA replication elements, and/or the RdRP coding regions have at least 85%, 90%, 95%, 98%, or 99% sequence identity to one another.

2. The recombinant RNA molecule of claim 1, wherein:
(a) the 5′ RNA replication element comprises at least one RNA secondary structure provided in Table 1 or adopted by an RNA molecule encoded by SEQ ID NO: 467, 468, 512, 546-551, or 631; and/or
(b) the 3′ RNA replication element comprises at least one RNA secondary structure provided in Table 1 or adopted by an RNA molecule encoded by SEQ ID NO: 469, 470, 520, 521, or 552-557.

3. The recombinant RNA molecule of claim 1, wherein:
(a) the 5′ RNA replication element comprises an RNA molecule encoded by SEQ ID NO: 467, 468, 512, 546-551, or 631; a variant thereof encoded by a DNA molecule having at least 70%, 75%, 80%, 85%, 90%, 95%, 98%, or 99% sequence identity to SEQ ID NO: 467, 468, 512, 546-551, or 631; or a variant thereof wherein one or more nucleotides in the RNA secondary structure are substituted with distinct nucleotides which maintain the RNA secondary structure; and/or
(b) the 3′ RNA replication element comprises an RNA molecule encoded by SEQ ID NO: 469, 470, 520, 521, or 552-557, or; a variant thereof encoded by a DNA molecule having at least 70%, 75%, 80%, 85%, 90%, 95%, 98%, or 99% sequence identity to SEQ ID NO: 469, 470, 520, 521, or 552-557; or a variant thereof wherein one of more base-paired residues in the RNA secondary structure are substituted with distinct nucleotides which maintain the RNA secondary structure.

4. The recombinant RNA molecule of claim 3, wherein the RNA secondary structure is maintained by substituting nucleotides in the secondary structure which are not base paired with nucleotides which will not base pair, and/or by substituting nucleotides in the secondary structure which are base paired with nucleotides which will base pair.

5. The recombinant RNA molecule of claim 1, wherein:
(a) the 5′ RNA replication element comprises at least a segment of the 5′ untranslated region (UTR) of the Secoviridae genome or a variant thereof having one or more nucleotide substitutions, insertions, and/or deletions, wherein the variant is recognized by the RdRP, optionally wherein the 5′ RNA replication element further comprises a genomic sequence of the virus of the family Secoviridae that is natively located 3′ to and adjacent to the 5′ UTR sequence; and/or
(b) the 3′ RNA replication element comprises at least a segment of the 3′ UTR of the Secoviridae genome or a variant thereof having one or more nucleotide substitutions, insertions, and/or deletions, wherein the variant is recognized by the RdRP, optionally wherein the 3′ RNA replication element further comprises a genomic sequence of the virus of the family Secoviridae that is natively located 5′ to and adjacent to the 3′ UTR sequence, and optionally wherein the Secoviridae genome of (a) and (b) are the same.

6. The recombinant RNA molecule of claim 1, wherein the RNA molecule further comprises at least one of: (i) a tRNA-like element, optionally wherein the tRNA-like element provides for intercellular movement of the RNA and optionally wherein the intercellular movement is mediated by a viral movement protein (MP); (ii) an encapsidation recognition element (ERE), optionally wherein the ERE provides for encapsidation of the RNA by a Secoviridae capsid protein or optionally wherein the ERE provides for encapsidation of the RNA by a non-Secoviridae capsid protein; (iii) an RNA effecter; and/or (iv) RNA encoding a viral movement protein (MP), optionally wherein an internal ribosome entry site (IRES) is operably linked to the RNA encoding the MP.

7. The recombinant RNA molecule of claim 6, wherein the tRNA-like element comprises a tRNA-like molecule from an Arabidopsis FT mRNA or is a tRNA-like sequence encoded by a DNA sequence selected from the group consisting of SEQ ID NOs: 76-123, and 466 or is a modified tRNA-like sequence that has at least 90% sequence identity to a scaffold tRNA-like sequence encoded by a DNA sequence selected from the group consisting of SEQ ID NOs: 76-123, and 466 and that maintains the secondary structure of the scaffold tRNA-like sequence, and/or wherein the ERE is a tobacco mosaic virus (TMV) OAS.

8. The recombinant RNA molecule of claim 1, wherein the cargo RNA molecule is up to about 8 kb in length.

9. The recombinant RNA molecule of claim 1, wherein the cargo RNA molecule comprises: (a) at least one coding sequence, optionally wherein the coding sequence encodes a selectable or scoreable marker: (b) at least one non-coding sequence; or (c) both at least one coding sequence and at least one non-coding sequence.

10. The recombinant RNA molecule of claim 1, wherein the cargo RNA molecule comprises at least one coding sequence, and wherein the RNA molecule further comprises an internal ribosome entry site (IRES) which is operably linked to at least one coding sequence, optionally wherein the operably linked IRES is located 5′ and immediately adjacent to the coding sequence.

11. The recombinant RNA molecule of claim 1, wherein the cargo RNA molecule comprises at least one non-coding sequence, and wherein the at least one non-coding sequence is a hairpin RNA (hpRNA); an RNA that forms multiple stem-loops; an RNA pseudoknot; an RNA molecule that forms at least partially double-stranded RNA; a small interfering RNA (siRNA) or siRNA precursor: a microRNA (miRNA) or miRNA precursor: a phased RNA or phased RNA precursor: a ribozyme; a ligand-responsive ribozyme (aptazyme); an RNA aptamer; or a long noncoding RNA (lncRNA).

12. The recombinant RNA molecule of claim 1, further comprising an RNA comprising encoding at least one ribozyme, optionally wherein the at least one ribozyme is located 5′ to the 5′ RNA replication element or 3′ to the 3′ RNA replication element.

13. The recombinant RNA molecule of claim 1, further comprising an RNA molecule comprising at least one ligand-responsive ribozyme (aptazyme), optionally wherein the at least one ligand-responsive ribozyme is located 5′ to the 5′ RNA replication element or 3′ to the 3′ RNA replication element.

14. The recombinant RNA molecule of claim 1, wherein: (i) the RNA further comprises at least a segment of its reverse complementary RNA molecule; and/or (ii) the recombinant RNA molecule is complexed with one or more RNA binding proteins or encapsidated by a viral capsid protein, optionally wherein the viral capsid protein is a Secoviridae capsid protein, optionally wherein the RNA comprises an ERE which provides for encapsidation of the RNA by the Secoviridae capsid protein, and/or optionally wherein the RNA binding proteins comprise an RNA recognition motif.

15. The recombinant RNA molecule of claim 1, wherein the RNA molecule comprises:
at least one heterologous RNA virus (HRV) amplicon in sense or antisense orientation to the first 5′ RNA replication element comprising:
I. (i) a heterologous RNA virus (HRV) 5′ replication region (HRV 5′RR); (ii) the cargo RNA molecule; and (iii) the heterologous RNA virus (HRV) 3′ RNA replication region (HRV 3′RR); wherein the HRV 5′ RR and HRV 3′ RR HRV are recognized by a heterologous RNA virus RNA-dependent RNA polymerase (hrvRdRP); and wherein the HRV 5′RR, cargo RNA molecule, and HRV 3′RR are operably linked; or
II. a heterologous RNA virus (HRV) subgenomic promoter operably linked to the cargo RNA molecule; wherein the subgenomic promoter is recognized by a heterologous RNA virus RNA-dependent RNA polymerase (hrvRdRP).

16. The recombinant RNA of claim 15, wherein the RNA molecule comprises from 5′ terminus to 3′ terminus:
(a) the 5′ RNA replication element;
(b) the HRV amplicon in antisense orientation to the first 5′ RNA replication element; optionally wherein the HRV amplicon further comprises:
(i) an RNA molecule encoding an HRV RNA-dependent RNA polymerase (hrvRdRP) which is operably linked to the HRV 5′RR and HRV 3′RR, wherein the RNA molecule encoding the HRV RNA-dependent RNA polymerase (hrvRdRP) is optionally operably linked to a subgenomic promoter recognized by the hrvRdRP; or
(ii) an RNA molecule encoding an HRV RNA-dependent RNA polymerase (hrvRdRP) which is operably linked to linked to a subgenomic promoter recognized by the hrvRdRP; and
(c) the 3′ RNA replication element.

17. The recombinant RNA of claim 15, wherein the HRV 5′RR, HRV 3′RR, and hrvRdRP comprise an HRV 5′RR, HRV 3′RR, and hrvRdRP from a virus selected from the group consisting of an Alphaflexivirus, Betaflexivirus, Bromovirus, Celavirus, Closterovirus, Comovirus, Potexvirus, Potyvirus, Tobamovirus, Tombusvirus, Tospoviridae, Trivirinae, Tymovirus, Varicosavirus, and Secoviridae.

18. The recombinant RNA of claim 17, wherein: (i) the Bromovirus is a Cucumber Mosaic Virus, Spinach latent virus, Olive latent virus, or Brome mosaic virus; (ii) the Closterovirus is a Citrus tristeza virus or Beet yellow virus; (iii) the Comovirus is Cowpea mosaic virus, Apple latent spherical virus, or Soybean latent spherical virus; (iv) the Potexvirus is Potato virus X or Citrus yellow vein clearing virus; (v) the Potyvirus is a Pepper mottle virus, Bean yellow mosaic virus, Barley stripe mosaic virus, Wheat stripe mosaic virus, Rice yellow mottle virus, Maize dwarf mosaic virus, zucchini yellow mosaic virus, watermelon mosaic virus, or sugar cane mosaic virus; (vi) the Tobamovirus is a Tobacco mosaic virus, Tomato mosaic virus, Tomato brown rugose fruit virus, Turnip vein-clearing virus, or Pepper mild mottle virus; (vii) the Tombusvirus is a Turnip crinkle virus or Tomato bushy stunt virus; (iv) the Tospoviridae is a Tomato spotted wilt virus or watermelon bud necrosis virus; or (viii) the Tymovirus is a Turnip yellow mosaic virus, Citrus yellow vein clearing virus, Potato latent virus, Apple stem grooving virus, or Citrus leaf blotch virus.

19. The recombinant RNA of claim 15, wherein the HRV 5′ RR and the HRV 3′ RR are obtained from the same HRV genome.

20. The recombinant RNA of claim 15, wherein the HRV 5′ RR and the HRV 3′ RR are obtained from distinct HRV genomes.

21. The recombinant RNA molecule of claim 15, wherein:
(a) the HRV 5′ RR comprises at least one RNA secondary structure adopted by an RNA encoded by SEQ ID NO: 161 to 185, or 186, or comprises the RNA sequence encoded by SEQ ID NO: 161 to 185, or 186, or comprises a contiguous fragment of at least 80%, 85%, 90%, or 95% of the full sequence of the RNA encoded by SEQ ID NO: 161 to 185, or 186; and/or
(b) the HRV 3′ RR comprises at least one RNA secondary structure adopted by an RNA encoded by SEQ ID NO: 187 to 210, or 211, or comprises the RNA sequence encoded by SEQ ID NO: 187 to 210, or 211, or comprises a contiguous fragment of at least 80%, 85%, 90%, or 95% of the full sequence of the RNA encoded by SEQ ID NO: 187 to 210, or 211.

22. The recombinant RNA molecule of claim 15, wherein:
(a) the HRV 5′ RR is encoded by a DNA molecule comprising SEQ ID NO: 161 to 185, or 186; or a variant thereof comprising DNA having at least 70%, 75%, 80%, 85%, 90%, 95%, 98%, or 99% sequence identity to SEQ ID NO: 161 to 185, or 186; or a variant thereof wherein one of more residues in the RNA secondary structure are substituted with distinct nucleotides which maintain the RNA secondary structure; and/or
(b) the HRV 3′ RR is encoded by a DNA molecule comprising SEQ ID NO: 187 to 210, or 211; or a variant thereof comprising DNA having at least 70%, 75%, 80%, 85%, 90%, 95%, 98%, or 99% sequence identity to SEQ ID NO: 187 to 210, or 211; or a variant thereof wherein one of more residues in the RNA secondary structure are substituted with distinct nucleotides which maintain the RNA secondary structure.

23. The recombinant RNA molecule of claim 15, wherein:
(a) the HRV 3′ RR comprises an RNA molecule containing at least a segment of the 3′ untranslated region (UTR) of the HRV genome, or a variant thereof having one or more nucleotide substitutions, insertions, and/or deletions, wherein the HRV 3′ RR or the variant is recognized by the hrvRdRP, optionally wherein the RNA comprising the HRV 3′ RR further comprises a genomic sequence of the HRV that is natively located 5′ to and adjacent to the 3′ UTR sequence; and/or
(d) the HRV 5′ RR comprises an RNA molecule containing at least a segment of the 3′ untranslated region (UTR) of the HRV genome, or a variant thereof having one or more nucleotide substitutions, insertions, and/or deletions, wherein the HRV 5′ RR or the variant is recognized by the hrvRdRP, optionally wherein the RNA comprising the HRV 5′ RR further comprises a genomic sequence of the HRV that is natively located 3′ to and adjacent to the 5′ UTR sequence.

24. The recombinant RNA molecule of claim 15, wherein the cargo RNA comprises an HRV-inhibitory RNA or encodes an HRV-inhibitory protein, wherein the HRV-inhibitory RNA or HRV-inhibitory protein inhibits infection, movement, transmission, and/or replication of the HRV.

25. The recombinant RNA molecule of claim 15, wherein the cargo RNA comprises an RNA having at least 20 contiguous nucleotides having an identical or complementary sequence to a segment of equivalent length of the genomic RNA of the HRV.

26. The recombinant RNA molecule of claim 15, wherein the cargo RNA comprises an RNA having at least 20 contiguous nucleotides having an identical or complementary sequence to a segment of equivalent length of the genomic RNA of the HRV which does not encode the hrvRdRP.

27. The recombinant RNA molecule of claim 15, wherein:
(i) the HRV is a Cucumber Mosaic Virus and the HRV 5′RR comprises an RNA encoded by the Cucumber Mosaic Virus 5′ RR DNA sequence in Table 7, an RNA encoded by a DNA having at least 85%, 90%, 95%, or 99% sequence identity to the Cucumber Mosaic Virus 5′ RR DNA sequence in Table 7, the HRV 3′ RR comprises an RNA encoded by the Cucumber Mosaic Virus 3′ RR DNA sequence in Table 7, or an RNA encoded by a DNA having at least 85%, 90%, 95%, or 99% sequence identity to the Cucumber Mosaic Virus 3′ RR DNA sequence in Table 7;
(ii) the HRV is a Brome mosaic virus and the HRV 5′RR comprises an RNA encoded by the Brome mosaic virus 5′ RR DNA sequence in Table 7, an RNA encoded by a DNA having at least 85%, 90%, 95%, or 99% sequence identity to the Brome mosaic virus 5′ RR DNA sequence in Table 7, the HRV 3′ RR comprises an RNA encoded by the Brome mosaic virus 3′ RR DNA sequence in Table 7, or an RNA encoded by a DNA having at least 85%, 90%, 95%, or 99% sequence identity to the Brome mosaic virus 3′ RR DNA sequence in Table 7;
(iii) the HRV is a Citrus tristeza virus and the HRV 5′RR comprises an RNA encoded by the Citrus tristeza virus 5′ RR DNA sequence in Table 7, an RNA encoded by a DNA having at least 85%, 90%, 95%, or 99% sequence identity to the Citrus tristeza virus 5′ RR DNA sequence in Table 7, the HRV 3′ RR comprises an RNA encoded by the Citrus tristeza virus 3′ RR DNA sequence in Table 7, or an RNA encoded by a DNA having at least 85%, 90%, 95%, or 99% sequence identity to the Citrus tristeza virus 3′ RR DNA sequence in Table 7, and the RdRP comprises a protein having at least 85%, 90%, 95%, or 99% sequence identity to the Citrus tristeza virus RdRP sequence in Table 7;
(iv) the HRV is Beet yellows virus and the HRV 5′RR comprises an RNA encoded by the Beet yellows virus 5′ RR DNA sequence in Table 7, an RNA encoded by a DNA having at least 85%, 90%, 95%, or 99% sequence identity to the Beet yellows virus 5′ RR DNA sequence in Table 7, the HRV 3′ RR comprises an RNA encoded by the Beet yellows virus 3′ RR DNA sequence in Table 7, or an RNA encoded by a DNA having at least 85%, 90%, 95%, or 99% sequence identity to the Beet yellows virus 3′ RR DNA sequence in Table 7, and the RdRP comprises a protein having at least 85%, 90%, 95%, or 99% sequence identity to the Beet yellows virus RdRP sequence in Table 7;
(v) the HRV is Cowpea mosaic virus and the HRV 5′RR comprises an RNA encoded by the Cowpea mosaic virus 5′ RR DNA sequence in Table 7 or an RNA encoded by a DNA having at least 85%, 90%, 95%, or 99% sequence identity to the Cowpea mosaic virus 5′ RR DNA sequence in Table 7;
(vi) the HRV is Potato virus X and the HRV 5′RR comprises an RNA encoded by the Potato virus X 5′ RR DNA sequence in Table 7, an RNA encoded by a DNA having at least 85%, 90%, 95%, or 99% sequence identity to the Potato virus X 5′ RR DNA sequence in Table 7, the HRV 3′ RR comprises an RNA encoded by the Potato virus X 3′ RR DNA sequence in Table 7, or an RNA encoded by a DNA having at least 85%, 90%, 95%, or 99% sequence identity to the Potato virus X 3′ RR DNA sequence in Table 7, and the RdRP comprises a protein having at least 85%, 90%, 95%, or 99% sequence identity to the Potato virus X RdRP sequence in Table 7;
(vii) the HRV is Pepper mottle virus and the HRV 5′RR comprises an RNA encoded by the Pepper mottle virus 5′ RR DNA sequence in Table 7, an RNA encoded by a DNA having at least 85%, 90%, 95%, or 99% sequence identity to the Pepper mottle virus 5′ RR DNA sequence in Table 7, the HRV 3′ RR comprises an RNA encoded by the Pepper mottle virus 3′ RR DNA sequence in Table 7, or an RNA encoded by a DNA having at least 85%, 90%, 95%, or 99% sequence identity to the Pepper mottle virus 3′ RR DNA sequence in Table 7, and the RdRP comprises a protein having at least 85%, 90%, 95%, or 99% sequence identity to the Pepper mottle virus RdRP sequence in Table 7;
(viii) the HRV is Bean yellow mosaic virus and the HRV 5′RR comprises an RNA encoded by the Bean yellow mosaic virus 5′ RR DNA sequence in Table 7, an RNA encoded by a DNA having at least 85%, 90%, 95%, or 99% sequence identity to the Bean yellow mosaic virus 5′ RR DNA sequence in Table 7, the HRV 3′ RR comprises an RNA encoded by the Bean yellow mosaic virus 3′ RR DNA sequence in Table 7, or an RNA encoded by a DNA having at least 85%, 90%, 95%, or 99% sequence identity to the Bean yellow mosaic virus 3′ RR DNA sequence in Table 7, and the RdRP comprises a protein having at least 85%, 90%, 95%, or 99% sequence identity to the Bean yellow mosaic virus RdRP sequence in Table 7;
(ix) the HRV is Barley stripe mosaic virus and the HRV 5′RR comprises an RNA encoded by the Barley stripe mosaic virus 5′ RR DNA sequence in Table 7, an RNA encoded by a DNA having at least 85%, 90%, 95%, or 99% sequence identity to the Barley stripe mosaic virus 5′ RR DNA sequence in Table 7, and the HRV 3′ RR comprises an RNA encoded by the Barley stripe mosaic virus 3′ RR DNA sequence in Table 7, or an RNA encoded by a DNA having at least 85%, 90%, 95%, or 99% sequence identity to the Barley stripe mosaic virus 3′ RR DNA sequence in Table 7;
(x) the HRV is Wheat stripe mosaic virus and the HRV 5′RR comprises an RNA encoded by the Wheat stripe mosaic virus 5′ RR DNA sequence in Table 7, an RNA encoded by a DNA having at least 85%, 90%, 95%, or 99% sequence identity to the Wheat stripe mosaic virus 5′ RR DNA sequence in Table 7, the HRV 3′ RR comprises an RNA encoded by the Wheat stripe mosaic virus 3′ RR DNA sequence in Table 7, or an RNA encoded by a DNA having at least 85%, 90%, 95%, or 99% sequence identity to the Wheat stripe mosaic virus 3′ RR DNA sequence in Table 7, and the RdRP comprises a protein having at least 85%, 90%, 95%, or 99% sequence identity to the Wheat stripe mosaic virus RdRP sequence in Table 7;
(xi) the HRV Rice yellow mottle virus and the HRV 5′RR comprises an RNA encoded by the Rice yellow mottle virus 5* RR DNA sequence in Table 7, an RNA encoded by a DNA having at least 85%, 90%, 95%, or 99% sequence identity to the Rice yellow mottle virus 5′ RR DNA sequence in Table 7, the HRV 3′ RR comprises an RNA encoded by the Rice yellow mottle virus 3′ RR DNA sequence in Table 7, or an RNA encoded by a DNA having at least 85%, 90%, 95%, or 99% sequence identity to the Rice yellow mottle virus 3′ RR DNA sequence in Table 7, and the RdRP comprises a protein having at least 85%, 90%, 95%, or 99% sequence identity to the Rice yellow mottle virus RdRP sequence in Table 7;
(xii) the HRV is Maize dwarf mosaic virus and the HRV 5′RR comprises an RNA encoded by the Maize dwarf mosaic virus 5′ RR DNA sequence in Table 7, an RNA encoded by a DNA having at least 85%, 90%, 95%, or 99% sequence identity to the Maize dwarf mosaic virus 5′ RR DNA sequence in Table 7, the HRV 3′ RR comprises an RNA encoded by the Maize dwarf mosaic virus 3′ RR DNA sequence in Table 7, or an RNA encoded by a DNA having at least 85%, 90%, 95%, or 99% sequence identity to the Maize dwarf mosaic virus 3′ RR DNA sequence in Table 7, and the RdRP comprises a protein having at least 85%, 90%, 95%, or 99% sequence identity to the Maize dwarf mosaic virus RdRP sequence in Table 7;
(xiii) the HRV is zucchini yellow mosaic virus and the HRV 5′RR comprises an RNA encoded by the Zucchini yellow mosaic virus 5′ RR DNA sequence in Table 7, an RNA encoded by a DNA having at least 85%, 90%, 95%, or 99% sequence identity to the Zucchini yellow mosaic virus 5′ RR DNA sequence in Table 7, the HRV 3′ RR comprises an RNA encoded by the Zucchini yellow mosaic virus 3′ RR DNA sequence in Table 7, or an RNA encoded by a DNA having at least 85%, 90%, 95%, or 99% sequence identity to the Zucchini yellow mosaic virus 3′ RR DNA sequence in Table 7, and the RdRP comprises a protein having at least 85%, 90%, 95%, or 99% sequence identity to the Zucchini yellow mosaic virus RdRP sequence in Table 7;
(xiv) the HRV is watermelon mosaic virus and the HRV 5′RR comprises an RNA encoded by the Watermelon mosaic virus 5′ RR DNA sequence in Table 7, an RNA encoded by a DNA having at least 85%, 90%, 95%, or 99% sequence identity to the Watermelon mosaic virus 5′ RR DNA sequence in Table 7, the HRV 3′ RR comprises an RNA encoded by the Watermelon mosaic virus 3′ RR DNA sequence in Table 7, or an RNA encoded by a DNA having at least 85%, 90%, 95%, or 99% sequence identity to the Watermelon mosaic virus 3′ RR DNA sequence in Table 7, and the RdRP comprises a protein having at least 85%, 90%, 95%, or 99% sequence identity to the Watermelon mosaic virus RdRP sequence in Table 7;
(xv) the HRV is sugarcane mosaic virus and the HRV 5′RR comprises an RNA encoded by the Sugarcane mosaic virus 5′ RR DNA sequence in Table 7, an RNA encoded by a DNA having at least 85%, 90%, 95%, or 99% sequence identity to the Sugarcane mosaic virus 5′ RR DNA sequence in Table 7, the HRV 3′ RR comprises an RNA encoded by the Sugarcane mosaic virus 3′ RR DNA sequence in Table 7, or an RNA encoded by a DNA having at least 85%, 90%, 95%, or 99% sequence identity to the Sugarcane mosaic virus 3′ RR DNA sequence in Table 7, and the RdRP comprises a protein having at least 85%, 90%, 95%, or 99% sequence identity to the Sugarcane mosaic virus RdRP sequence in Table 7;
(xvi) the HRV is Tobacco mosaic virus and the HRV 5′RR comprises an RNA encoded by the Tobacco mosaic virus 5′ RR DNA sequence in Table 7, an RNA encoded by a DNA having at least 85%, 90%, 95%, or 99% sequence identity to the Tobacco mosaic virus 5′ RR DNA sequence in Table 7, the HRV 3′ RR comprises an RNA encoded by the Tobacco mosaic virus 3′ RR DNA sequence in Table 7, or an RNA encoded by a DNA having at least 85%, 90%, 95%, or 99% sequence identity to the Tobacco mosaic virus 3′ RR DNA sequence in Table 7, and the RdRP comprises a protein having at least 85%, 90%, 95%, or 99% sequence identity to the Tobacco mosaic virus RdRP sequence in Table 7;
(xvii) the HRV is Tomato mosaic virus and the HRV 5′RR comprises an RNA encoded by the Tomato mosaic virus 5′ RR DNA sequence in Table 7, an RNA encoded by a DNA having at least 85%, 90%, 95%, or 99% sequence identity to the Tomato mosaic virus 5′ RR DNA sequence in Table 7, the HRV 3′ RR comprises an RNA encoded by the Tomato mosaic virus 3′ RR DNA sequence in Table 7, or an RNA encoded by a DNA having at least 85%, 90%, 95%, or 99% sequence identity to the Tomato mosaic virus 3′ RR DNA sequence in Table 7, and the RdRP comprises a protein having at least 85%, 90%, 95%, or 99% sequence identity to the Tomato mosaic virus RdRP sequence in Table 7;
(xviii) the HRV is Tomato brown rugose fruit virus and the HRV 5′RR comprises an RNA encoded by the Tomato brown rugose fruit virus 5′ RR DNA sequence in Table 7, an RNA encoded by a DNA having at least 85%, 90%, 95%, or 99% sequence identity to the Tomato brown rugose fruit virus 5′ RR DNA sequence in Table 7, the HRV 3′ RR comprises an RNA encoded by the Tomato brown rugose fruit virus 3′ RR DNA sequence in Table 7, or an RNA encoded by a DNA having at least 85%, 90%, 95%, or 99% sequence identity to the Tomato brown rugose fruit virus 3′ RR DNA sequence in Table 7, and the RdRP comprises a protein having at least 85%, 90%, 95%, or 99% sequence identity to the Tomato brown rugose fruit virus RdRP sequence in Table 7;
(xix) the HRV is Turnip vein-clearing virus and the HRV 5′RR comprises an RNA encoded by the Turnip vein-clearing virus 5′ RR DNA sequence in Table 7, an RNA encoded by a DNA having at least 85%, 90%, 95%, or 99% sequence identity to the Turnip vein-clearing virus 5′ RR DNA sequence in Table 7, the HRV 3′ RR comprises an RNA encoded by the Turnip vein-clearing virus 3′ RR DNA sequence in Table 7, or an RNA encoded by a DNA having at least 85%, 90%, 95%, or 99% sequence identity to the Turnip vein-clearing virus 3′ RR DNA sequence in Table 7, and the RdRP comprises a protein having at least 85%, 90%, 95%, or 99% sequence identity to the Turnip vein-clearing virus RdRP sequence in Table 7;
(xx) the HRV is Pepper mild mottle virus and the HRV 5′RR comprises an RNA encoded by the Pepper mild mottle virus 5′ RR DNA sequence in Table 7, an RNA encoded by a DNA having at least 85%, 90%, 95%, or 99% sequence identity to the Pepper mild mottle virus 5′ RR DNA sequence in Table 7, the HRV 3′ RR comprises an RNA encoded by the Pepper mild mottle virus 3′ RR DNA sequence in Table 7, or an RNA encoded by a DNA having at least 85%, 90%, 95%, or 99% sequence identity to the Pepper mild mottle virus 3′ RR DNA sequence in Table 7, and the RdRP comprises a protein having at least 85%, 90%, 95%, or 99% sequence identity to the Pepper mild mottle virus RdRP sequence in Table 7;
(xxi) the HRV is Turnip crinkle virus and the HRV 5′RR comprises an RNA encoded by the Turnip crinkle virus 5′ RR DNA sequence in Table 7, an RNA encoded by a DNA having at least 85%, 90%, 95%, or 99% sequence identity to the Turnip crinkle virus 5′ RR DNA sequence in Table 7, the HRV 3′ RR comprises an RNA encoded by the Turnip crinkle virus 3′ RR DNA sequence in Table 7, or an RNA encoded by a DNA having at least 85%, 90%, 95%, or 99% sequence identity to the Turnip crinkle virus 3′ RR DNA sequence in Table 7, and the RdRP comprises a protein having at least 85%, 90%, 95%, or 99% sequence identity to the Turnip crinkle virus RdRP sequence in Table 7;
(xxii) the HRV is Tomato bushy stunt virus and the HRV 5′RR comprises an RNA encoded by the Tomato bushy stunt virus 5′ RR DNA sequence in Table 7, an RNA encoded by a DNA having at least 85%, 90%, 95%, or 99% sequence identity to the Tomato bushy stunt virus 5′ RR DNA sequence in Table 7, the HRV 3′ RR comprises an RNA encoded by the Tomato bushy stunt virus 3′ RR DNA sequence in Table 7, or an RNA encoded by a DNA having at least 85%, 90%, 95%, or 99% sequence identity to the Tomato bushy stunt virus 3′ RR DNA sequence in Table 7, and the RdRP comprises a protein having at least 85%, 90%, 95%, or 99% sequence identity to the Tomato bushy stunt virus RdRP sequence in Table 7;
(xxiii) the HRV is Tomato spotted wilt virus and the HRV 5′RR comprises an RNA encoded by the Tomato spotted wilt virus 5′ RR DNA sequence in Table 7, an RNA encoded by a DNA having at least 85%, 90%, 95%, or 99% sequence identity to the Tomato spotted wilt virus 5′ RR DNA sequence in Table 7, the HRV 3′ RR comprises an RNA encoded by the Tomato spotted wilt virus 3′ RR DNA sequence in Table 7, or an RNA encoded by a DNA having at least 85%, 90%, 95%, or 99% sequence identity to the Tomato spotted wilt virus 3′ RR DNA sequence in Table 7, and the RdRP comprises a protein having at least 85%, 90%, 95%, or 99% sequence identity to the Tomato spotted wilt virus RdRP sequence in Table 7;
(xxiv) the HRV is watermelon bud necrosis virus and the HRV 5′RR comprises an RNA encoded by the Watermelon bud necrosis virus 5′ RR DNA sequence in Table 7, an RNA encoded by a DNA having at least 85%, 90%, 95%, or 99% sequence identity to the Watermelon bud necrosis virus 5′ RR DNA sequence in Table 7, the HRV 3′ RR comprises an RNA encoded by the Watermelon bud necrosis virus 3′ RR DNA sequence in Table 7, or an RNA encoded by a DNA having at least 85%, 90%, 95%, or 99% sequence identity to the Watermelon bud necrosis virus 3′ RR DNA sequence in Table 7, and the RdRP comprises a protein having at least 85%, 90%, 95%, or 99% sequence identity to the Watermelon bud necrosis virus RdRP sequence in Table 7;
(xxv) the HRV is Turnip yellow mosaic virus and the HRV 5′RR comprises an RNA encoded by the Turnip yellow mosaic virus 5′ RR DNA sequence in Table 7, an RNA encoded by a DNA having at least 85%, 90%, 95%, or 99% sequence identity to the Turnip yellow mosaic virus 5′ RR DNA sequence in Table 7, the HRV 3′ RR comprises an RNA encoded by the Turnip yellow mosaic virus 3′ RR DNA sequence in Table 7, or an RNA encoded by a DNA having at least 85%, 90%, 95%, or 99% sequence identity to the Turnip yellow mosaic virus 3′ RR DNA sequence in Table 7, and the RdRP comprises a protein having at least 85%, 90%, 95%, or 99% sequence identity to the Turnip yellow mosaic virus RdRP sequence in Table 7;
(xxvi) the HRV is Spinach latent virus and the HRV 5 ′RR comprises an RNA encoded by the Spinach latent virus 5′ RR DNA sequence in Table 7, an RNA encoded by a DNA having at least 85%, 90%, 95%, or 99% sequence identity to the Spinach latent virus 5′ RR DNA sequence in Table 7, the HRV 3′ RR comprises an RNA encoded by the Spinach latent virus 3′ RR DNA sequence in Table 7, or an RNA encoded by a DNA having at least 85%, 90%, 95%, or 99% sequence identity to the Spinach latent virus 3′ RR DNA sequence in Table 7, and the RdRP comprises a protein having at least 85%, 90%, 95%, or 99% sequence identity to the Spinach latent virus RdRP sequence of SEQ ID NO: 523;
(xxvii) the HRV is Spinach latent virus and the HRV 5′RR comprises an RNA encoded by the Spinach latent virus 5′ RR DNA sequence in Table 7, an RNA encoded by a DNA having at least 85%, 90%, 95%, or 99% sequence identity to the Spinach latent virus 5′ RR DNA sequence in Table 7, the HRV 3′ RR comprises an RNA encoded by the Spinach latent virus 3′ RR DNA sequence in Table 7, or an RNA encoded by a DNA having at least 85%, 90%, 95%, or 99% sequence identity to the Spinach latent virus 3′ RR DNA sequence in Table 7, and the RdRP comprises a protein having at least 85%, 90%, 95%, or 99% sequence identity to the Spinach latent virus RdRP sequence of SEQ ID NO: 524:
(xxviii) the HRV is Olive latent virus 2 and the HRV 5′RR comprises an RNA encoded by the Olive latent virus 2 5′ RR DNA sequence in Table 7, an RNA encoded by a DNA having at least 85%, 90%, 95%, or 99% sequence identity to the Olive latent virus 2 5′ RR DNA sequence in Table 7, the HRV 3′ RR comprises an RNA encoded by the Olive latent virus 2 3′ RR DNA sequence in Table 7, or an RNA encoded by a DNA having at least 85%, 90%, 95%, or 99% sequence identity to the Olive latent virus 2 3′ RR DNA sequence in Table 7, and the RdRP comprises a protein having at least 85%, 90%, 95%, or 99% sequence identity to the Olive latent virus 2 RdRP sequence in Table 7;
(xxix) the HRV is Citrus yellow vein clearing virus and the HRV 5′RR comprises an RNA encoded by the Citrus yellow vein clearing virus 5′ RR DNA sequence in Table 7, an RNA encoded by a DNA having at least 85%, 90%, 95%, or 99% sequence identity to the Citrus yellow vein clearing virus virus 5′ RR DNA sequence in Table 7, the HRV 3′ RR comprises an RNA encoded by the Citrus yellow vein clearing virus 3′ RR DNA sequence in Table 7, or an RNA encoded by a DNA having at least 85%, 90%, 95%, or 99% sequence identity to the Citrus yellow vein clearing virus 3′ RR DNA sequence in Table 7, and the RdRP comprises a protein having at least 85%, 90%, 95%, or 99% sequence identity to the Citrus yellow vein clearing virus RdRP sequence in Table 7;
(xxx) the HRV is Potato latent virus and the HRV 5′RR comprises an RNA encoded by the Potato latent virus 5′ RR DNA sequence in Table 7, an RNA encoded by a DNA having at least 85%, 90%, 95%, or 99% sequence identity to the Potato latent virus 5′ RR DNA sequence in Table 7, the HRV 3′ RR comprises an RNA encoded by the Potato latent virus 3′ RR DNA sequence in Table 7, or an RNA encoded by a DNA having at least 85%, 90%, 95%, or 99% sequence identity to the Potato latent virus 3′ RR DNA sequence in Table 7, and the RdRP comprises a protein having at least 85%, 90%, 95%, or 99% sequence identity to the Potato latent virus RdRP sequence in Table 7;
(xxxi) the HRV is Apple stem grooving virus and the HRV 5′RR comprises an RNA encoded by the Apple stem grooving virus 5′ RR DNA sequence in Table 7, an RNA encoded by a DNA having at least 85%, 90%, 95%, or 99% sequence identity to the Apple stem grooving virus 5′ RR DNA sequence in Table 7, the HRV 3′ RR comprises an RNA encoded by the Apple stem grooving virus 3′ RR DNA sequence in Table 7, or an RNA encoded by a DNA having at least 85%, 90%, 95%, or 99% sequence identity to the Apple stem grooving virus 3′ RR DNA sequence in Table 7, and the RdRP comprises a protein having at least 85%, 90%, 95%, or 99% sequence identity to the Apple stem grooving virus RdRP sequence in Table 7;
(xxxii) the HRV is Citrus leaf blotch virus and the HRV 5′RR comprises an RNA encoded by the Citrus leaf blotch virus 5′ RR DNA sequence in Table 7, an RNA encoded by a DNA having at least 85%, 90%, 95%, or 99% sequence identity to the Citrus leaf blotch virus 5′ RR DNA sequence in Table 7, the HRV 3′ RR comprises an RNA encoded by the Citrus leaf blotch virus 3′ RR DNA sequence in Table 7, or an RNA encoded by a DNA having at least 85%, 90%, 95%, or 99% sequence identity to the Citrus leaf blotch virus 3′ RR DNA sequence in Table 7, and the RdRP comprises a protein having at least 85%, 90%, 95%, or 99% sequence identity to the Citrus leaf blotch virus RdRP sequence in Table 7;
(xxxiii) the HRV is Apple latent spherical virus and the HRV 5′RR comprises an RNA encoded by the Apple latent spherical virus 5′ RR DNA sequence in Table 7, an RNA encoded by a DNA having at least 85%, 90%, 95%, or 99% sequence identity to the Apple latent spherical virus 5′ RR DNA sequence in Table 7, the HRV 3′ RR comprises an RNA encoded by the Apple latent spherical virus 3′ RR DNA sequence in Table 7, or an RNA encoded by a DNA having at least 85%, 90%, 95%, or 99% sequence identity to the Apple latent spherical virus 3′ RR DNA sequence in Table 7, and the RdRP comprises a protein having at least 85%, 90%, 95%, or 99% sequence identity to the Apple latent spherical virus RdRP sequence in Table 7;
(xxxiv) the HRV is Soybean latent spherical virus and the HRV 5′RR comprises an RNA encoded by the Soybean latent spherical virus 5′ RR DNA sequence in Table 7, an RNA encoded by a DNA having at least 85%, 90%, 95%, or 99% sequence identity to the Soybean latent spherical virus 5′ RR DNA sequence in Table 7, the HRV 3′ RR comprises an RNA encoded by the Soybean latent spherical virus 3′ RR DNA sequence in Table 7, or an RNA encoded by a DNA having at least 85%, 90%, 95%, or 99% sequence identity to the Soybean latent spherical virus 3′ RR DNA sequence in Table 7, and the RdRP comprises a protein having at least 85%, 90%, 95%, or 99% sequence identity to the Soybean latent spherical virus RdRP sequence in Table 7;
(xxxv) the HRV is Celery latent virus and the HRV 5′RR comprises an RNA encoded by the Celery latent virus 5′ RR DNA sequence in Table 7, an RNA encoded by a DNA having at least 85%, 90%, 95%, or 99% sequence identity to the Celery latent virus 5′ RR DNA sequence in Table 7, the HRV 3′ RR comprises an RNA encoded by the Celery latent virus 3′ RR DNA sequence in Table 7, or an RNA encoded by a DNA having at least 85%, 90%, 95%, or 99% sequence identity to the Celery latent virus 3′ RR DNA sequence in Table 7, and the RdRP comprises a protein having at least 85%, 90%, 95%, or 99% sequence identity to the Celery latent virus RdRP sequence in Table 7;
(xxxvi) the HRV is Black grass varicosavirus-like virus and the HRV 5′RR comprises an RNA encoded by the Black grass varicosavirus-like virus 5′ RR DNA sequence in Table 7, an RNA encoded by a DNA having at least 85%, 90%, 95%, or 99% sequence identity to the Black grass varicosavirus-like virus 5′ RR DNA sequence in Table 7, the HRV 3′ RR comprises an RNA encoded by the Black grass varicosavirus-like virus 3′ RR DNA sequence in Table 7, or an RNA encoded by a DNA having at least 85%, 90%, 95%, or 99% sequence identity to the Black grass varicosavirus-like virus 3′ RR DNA sequence in Table 7, and the RdRP comprises a protein having at least 85%, 90%, 95%, or 99% sequence identity to the Black grass varicosavirus-like virus sequence in Table 7; or
(xxxvii) the HRV is Maize suscal virus and the HRV 5′RR comprises an RNA encoded by the Maize suscal virus 5′ RR DNA sequence in Table 7, an RNA encoded by a DNA having at least 85%, 90%, 95%, or 99% sequence identity to the Maize suscal virus 5′ RR DNA sequence in Table 7, the HRV 3′ RR comprises an RNA encoded by the Maize suscal virus 3′ RR DNA sequence in Table 7, or an RNA encoded by a DNA having at least 85%, 90%, 95%, or 99% sequence identity to the Maize suscal virus 3′ RR DNA sequence in Table 7, and the RdRP comprises a protein having at least 85%, 90%, 95%, or 99% sequence identity to the Maize suscal virus sequence in Table 7.

28. The recombinant RNA molecule of claim 15, further comprising an RNA comprising encoding at least one cleavable sequence, optionally wherein the at least one cleavable sequence is located: (i) 5′ to the 5′ RNA replication element or 3′ to the 3′ RNA replication element; and/or (ii) between the 3′ end of the 5′ RNA replication element and the HRV amplicon and/or between the HRV amplicon and the 5′ end of the 3′ RNA replication element, wherein the cleavable sequence is optionally a self-cleaving ribozyme, a self-cleaving inducible ribozyme, or an siRNA or miRNA recognition site.

29. An agricultural formulation comprising the recombinant RNA molecule of any one of claims 1 to 28.

30. The agricultural formulation of claim 29, wherein the recombinant RNA molecule is complexed with one or more RNA binding proteins or encapsidated by a viral capsid protein, optionally wherein the viral capsid protein is a Secoviridae capsid protein and optionally wherein the RNA comprises an ERE which provides for encapsidation of the RNA by the Secoviridae capsid protein.

31. The agricultural formulation of claim 30, wherein the RNA binding proteins comprise an RNA recognition motif

32. The agricultural formulation of claim 30, wherein the viral capsid protein is heterologous to the Secoviridae.

33. The agricultural formulation of claim 29, wherein the formulation comprises the recombinant RNA molecule and a carrier, an excipient, and/or an adjuvant.

34. A cell comprising the recombinant RNA molecule of any one of claims 1 to 28, wherein the cell is a bacterial cell, a fungal cell, a plant cell, an insect cell, or an invertebrate animal cell.

35. The cell of claim 34, wherein the cell is a plant cell and DNA which encodes the recombinant RNA molecule is absent from the cell.

36. The cell of claim 34, wherein the cell comprises a recombinant DNA molecule which encodes the recombinant RNA molecule.

37. An expression system comprising:
(a) an RNA molecule comprising the recombinant RNA molecule of any one of claims 1 to 28; and
(b) a cell containing the recombinant RNA molecule and an RdRP protein that recognizes the 5′ and 3′ RNA replication elements of the recombinant RNA molecule.

38. The expression system of claim 37, wherein the recombinant RNA molecule comprises an operably linked encapsidation recognition element (ERE) recognized by a viral capsid protein, and wherein the cell contains the viral capsid protein.

39. The expression system of claim 37, wherein the encapsidation recognition element (ERE) is a Secoviridae ERE, wherein the viral capsid protein in the cell is a Secoviridae capsid protein, and wherein the RNA molecule is encapsidated by the Secoviridae capsid protein.

40. The expression system of claim 37, further comprising the reverse complement of the recombinant RNA molecule.

41. The expression system of claim 37, wherein the cell is a bacterial cell, a plant cell, a fungal cell, an insect cell, or an invertebrate animal cell.

42. The expression system of claim 37, wherein the cell further comprises: (i) a viral capsid protein (CP), (ii) an RNA-binding protein (RBP) that can bind to the RNA molecule, optionally wherein the RBP binds to an RNA effecter; (iii) an RNA cleavage agent that cleaves the RNA molecule; (iv) a second RNA-dependent RNA polymerase (RdRP) protein that recognizes an HRV 5′ or 3′ replication region and/or a subgenomic promoter in the RNA molecule (2^{nd} RdRP); (v) a viral movement protein (MP); (v) a heterologous RNA virus (HRV); or (vi) an hrvRdRP, optionally wherein the hrvRdRP recognizes the HRV 5′ or 3′ replication region and/or the subgenomic promoter.

43. The expression system of claim 42, wherein the CP, RBP, RdRP, 2^{nd} RdRP, hrvRdRP, and/or the MP is: (a) expressed by a recombinant DNA molecule in the cell; (b) provided exogenously to the cell; (c) expressed by a recombinant RNA molecule in the cell; or (d) expressed by a virus in the cell.

44. The expression system of claim 42, wherein the RdRP, CP, RBP, 2^{nd} RdRP, hrvRdRP, and/or the MP protein is heterologous to the cell.

45. The expression system of claim 42, wherein the RdRP, 2^{nd} RdRP, or hrvRdRP protein or a polynucleotide encoding the RdRP, 2^{nd} RdRP, or hrvRdRP protein is (a) expressed by a recombinant DNA molecule in the cell; (b) provided exogenously to the cell; (c) expressed by a recombinant RNA molecule in the cell; or (d) expressed by a virus in the cell.

46. The expression system of claim 37, wherein the cell is a plant cell.

47. The expression system of claim 46, wherein the plant cell contains a virus of the family Secoviridae which expresses the RdRP protein that recognizes the 5′ RNA replication element and the 3′ RNA replication element and/or wherein the plant cell contains an HRV that expresses the 2^{nd} RdRP or hrvRdRP protein.

48. The expression system of claim 47, wherein the virus of the family Secoviridae occurs naturally in the plant cell.

49. A method of providing a synthetic Secoviridae satellite RNA to a plant, comprising contacting the plant with the recombinant RNA molecule of any one of claims 1 to 28.

50. The method of claim 49, wherein contacting comprises spraying, dusting, injecting, or soaking the plant or a part thereof with the recombinant RNA molecule or the formulation.

51. The method of claim 49, further comprising providing an hrvRdRP to the plant which recognizes the HRV 5′ or 3′ replication region and/or the subgenomic promoter, optionally wherein the hrvRdRP is provided by introducing a recombinant DNA or RNA encoding the hrvRdRP into the plant or a part thereof.

52. A method of establishing a synthetic Secoviridae satellite RNA in a plant cell, comprising: providing to a plant cell the recombinant RNA molecule of any one of claims 1 to 28;
wherein the plant cell comprises an RdRP protein that recognizes the 5′ RNA replication element and 3′ RNA replication element, wherein the RNA molecule is optionally comprises an ERE and is encapsidated by a capsid protein,
whereby the RdRP protein catalyzes synthesis of the synthetic Secoviridae satellite RNA from the recombinant RNA molecule.

53. The method of claim 52, wherein the plant cell comprises a virus of the family Secoviridae and wherein the RdRP protein is provided to the plant cell by the virus of the family Secoviridae.

54. The method of claim 52, wherein the virus of the family Secoviridae is endemic to the plant cell, optionally wherein the virus of the family Secoviridae which is endemic to the plant cell is non-pathogenic and/or commensal.

55. The method of claim 52, wherein the capsid protein comprises a Secoviridae capsid protein and the ERE is recognized by the Secoviridae capsid protein, optionally wherein the recombinant RNA molecule comprises an operably linked encapsidation recognition element (ERE) recognized by a viral capsid protein.

56. The method of claim 52, further comprising providing an hrvRdRP to the plant which recognizes the HRV 5′ or 3′ replication region and/or the subgenomic promoter in the synthetic Secoviridae satellite RNA, optionally wherein the hrvRdRP is provided by introducing a recombinant DNA or RNA encoding the hrvRdRP into the plant or a part thereof.

57. A method of obtaining a phenotypic change in a plant or plant cell, comprising: providing to a plant or plant cell a recombinant RNA molecule of any one of claims 1 to 28, wherein the cargo RNA molecule comprises RNA that effects a phenotypic change in the plant or plant cell in comparison to a plant or plant cell lacking the recombinant RNA, wherein the plant or plant cell comprises an RdRP protein that recognizes the 5′ RNA replication element and 3′ RNA replication element and catalyzes synthesis of a synthetic Secoviridae RNA from the recombinant RNA molecule, and wherein the cargo RNA molecule effects the phenotypic change.

58. The method of claim 57, wherein the RNA that effects a phenotypic change in the plant or plant cell comprises at least one RNA selected from an siRNA or siRNA precursor, a miRNA or miRNA precursor, and a phased siRNA or phased siRNA precursor.

59. The method of claim 57, wherein the RNA that effects a phenotypic change in the plant or plant cell comprises a messenger RNA.

60. The method of claim 59, wherein the messenger RNA comprises an RNA molecule absent in the genome of the plant or plant cell.

61. The method of claim 57, wherein the RNA that effects a phenotypic change in the plant or plant cell comprises an RNA for modifying the genome of the plant or plant cell.

62. The method of claim 57, wherein the RNA that effects a phenotypic change in the plant or plant cell comprises an RNA for modifying the transcriptome and/or epigenome of the plant or plant cell, optionally wherein the RNA for modifying the epigenome targets an endogenous plant gene for RNA-induced transcriptional silencing.

63. The method of claim 57, wherein the phenotypic change comprises an increase in the plant's resistance to a pest or pathogen, optionally wherein the pest or pathogen is selected from the group comprising a bacterium, a virus other than a virus of the family Secoviridae, a fungus, an oomycete, and an invertebrate.

64. The method of claim 63, wherein the pathogen is a heterologous RNA virus (HRV), optionally wherein the HRV is a virus selected from the group consisting of an Alphaflexivirus, Betaflexivirus, Bromovirus, Celavirus, Closterovirus, Comovirus, Potexvirus, Potyvirus, Tobamovirus, Tombusvirus, Tospoviridae, Trivirinae, Tymovirus, Varicosavirus, and Secoviridae.

65. The method of claim 64, wherein: (i) the Bromovirus is a Cucumber Mosaic Virus, Spinach latent virus, Olive latent virus, or Brome mosaic virus; (ii) the Closterovirus is a Citrus tristeza virus or Beet yellow virus; (iii) the Comovirus is Cowpea mosaic virus, Apple latent spherical virus, or Soybean latent spherical virus; (iv) the Potexvirus is Potato virus X or Citrus yellow vein clearing virus; (v) the Potyvirus is a Pepper mottle virus, Bean yellow mosaic virus, Barley stripe mosaic virus, Wheat stripe mosaic virus, Rice yellow mottle virus, Maize dwarf mosaic virus, zucchini yellow mosaic virus, watermelon mosaic virus, or sugar cane mosaic virus; (vi) the Tobamovirus is a Tobacco mosaic virus, Tomato mosaic virus, Tomato brown rugose fruit virus, Turnip vein-clearing virus, or Pepper mild mottle virus; (vii) the Tombusvirus is a Turnip crinkle virus or Tomato bushy stunt virus; (iv) the Tospoviridae is a Tomato spotted wilt virus or watermelon bud necrosis virus; or (viii) the Tymovirus is a Turnip yellow mosaic virus, Citrus yellow vein clearing virus, Potato latent virus, Apple stem grooving virus, or Citrus leaf blotch virus.

66. The method of claim 57, wherein the phenotypic change comprises an increase in the plant's resistance to stress, optionally wherein the stress comprises at least one abiotic stress comprising nutrient stress, light stress, water stress, heat stress, and/or cold stress, or optionally wherein the stress comprises at least one biotic stress comprising crowding, shading, or allelopathy.

67. The method of claim 57, wherein the recombinant RNA molecule is provided to the plant of plant cell in the form of an RNA, an encapsidated RNA, or a formulation thereof.

68. The method of claim 67, wherein the encapsidated RNA comprises a synthetic Secoviridae satellite particle.

69. The method of claim 57, wherein the providing comprises contacting the plant or plant cell with the RNA, encapsidated RNA, or formulation thereof, optionally wherein contacting comprises spraying, dusting, injecting, or soaking the plant or plant cell with the RNA, encapsidated RNA, or formulation thereof.

70. The method of claim 57, wherein the recombinant RNA further comprises its reverse complementary RNA molecule.

71. The method of claim 57, further comprising providing an hrvRdRP to the plant which recognizes the HRV 5′ or 3′ replication region and/or the subgenomic promoter in the synthetic Secoviridae satellite RNA, optionally wherein the hrvRdRP is provided by introducing a recombinant DNA or RNA encoding the hrvRdRP into the plant or a part thereof.

72. A method of manufacturing a synthetic Secoviridae satellite particle, comprising combining the recombinant RNA molecule of any one of claims 1 to 28 with a viral capsid protein, wherein the recombinant RNA molecule comprises an encapsidation recognition element (ERE), and wherein the ERE provides for encapsidation of the RNA by the viral capsid protein.

73. The method of claim 72, wherein the recombinant RNA molecule is combined with a viral capsid protein in a vessel.

74. The method of claim 73, wherein the combining comprises
(a) providing to a plant cell the recombinant RNA molecule, wherein the recombinant RNA molecule comprises an encapsidation recognition element (ERE), and wherein the plant cell comprises an RdRP protein that recognizes the 5′ RNA replication element and 3′ RNA replication element catalyzes synthesis of a synthetic Secoviridae satellite RNA from the recombinant RNA molecule and a viral capsid protein, wherein the ERE provides for encapsidation of the RNA by the viral capsid protein; and optionally
(b) isolating the synthetic Secoviridae satellite particle from the plant cell, a plant comprising the plant cell, or from media in which the plant cell or the plant had been grown.

75. The method of claim 72, further comprising the step of formulating the synthetic Secoviridae satellite particle wherein the formulating comprises combining the synthetic Secoviridae satellite particle with a carrier, an excipient, and/or an adjuvant.

76. A plant propagule comprising the recombinant RNA molecule of any one of claims 1 to 28 and a Secoviridae RdRP, optionally wherein the plant propagule further comprises a heterologous RNA virus RdRP which recognizes an HRV 5′ or 3′ replication region and/or the subgenomic promoter in the synthetic Secoviridae satellite RNA.

77. The plant propagule of claim 76, wherein the plant propagule is a seed, a seedling, root, stem, leaf, shoot, tuber, rhizome, stolon, bulb, explant, embryo, or callus.

78. The plant propagule of claim 76, wherein the plant propagule is a mosaic comprising both plant cells comprising the recombinant RNA molecule and plant cells lacking the recombinant RNA molecule.

79. The plant propagule of claim 76, wherein the plant propagule lacks DNA encoding the recombinant RNA molecule.

80. The plant propagule of claim 76, wherein the plant propagule further comprises a heterologous RNA virus RdRP which recognizes an HRV 5′ or 3′ replication region and/or the subgenomic promoter in the synthetic Secoviridae satellite RNA, and the heterologous RNA virus RdRP is an Alphaflexivirus, Betaflexivirus, Bromovirus, Celavirus, Closterovirus, Comovirus, Potexvirus, Potyvirus, Tobamovirus, Tombusvirus, Tospoviridae, Trivirinae, Tymovirus, Varicosavirus, or Secoviridae RdRP or an RdRP set forth in Table 7.

81. A plant comprising the recombinant RNA molecule of any one of claims 1 to 28 and a Secoviridae RdRP, optionally wherein the plant propagule further comprises a heterologous RNA virus RdRP which recognizes an HRV 5′ or 3′ replication region and/or the subgenomic promoter in the synthetic Secoviridae satellite RNA.

82. The plant of claim 81, wherein the plant is a monocot or a dicot plant.

83. The plant of claim 81, wherein the plant is of the family Amaranthaceae, Asteraceae, Brassicaceae, Caricaceae, Caryophyllaceae, Cucurbitaceae, Cupressaceae, Fabaceae, Gentianaceae, Pinaceae, Poaceae, Rosaceae, Rutaceae, or Solanaceae.

84. The plant of claim 81, wherein the plant lacks DNA that encodes the recombinant RNA molecule.

85. The plant of claim 81, wherein the plant comprises a virus of the family Secoviridae, and wherein the Secoviridae RdRP is provided to the plant cell by the virus of the family Secoviridae, optionally wherein the Secoviridae RdRP comprises a protein having at least 85%, 90%, 95%, 97%, or 99% sequence identity to SEQ ID NO: 471, 530, or 558-564.

86. The plant of claim 81, wherein the virus of the family Secoviridae is endemic to the plant, optionally wherein the endemic virus of the family Secoviridae is non-pathogenic and/or commensal.

87. The plant of claim 81, wherein the Secoviridae RdRP, the 5′ RNA replication element, and/or the 3′ RNA replication element are derived from a virus of the family Secoviridae comprising one or both of the Secoviridae RdRP, 5′ RNA replication element, and/or 3′ RNA replication elements.

88. The plant of claim 81, wherein the RdRP has at least 85%, 90%, 95%, 97%, 98%, or 99% identity to SEQ ID NO: 471, 530, or 558-564, wherein the 5′ RNA replication element has at least 85%, 90%, 95%, 97%, 98%, or 99% identity to the RNA encoded by SEQ ID NO: 467, 468, 512, 546-551, or 631, and/or wherein the 3′ RNA replication element has at least 85%, 90%, 95%, 97%, 98%, or 99% identity to the RNA encoded by SEQ ID NO: 469, 470, 520, 521, or 552-557.

89. The plant of claim 81, wherein the plant is a grafted plant and wherein the rootstock and/or scion of the grafted plant comprise at least one cell comprising the recombinant RNA and the Secoviridae RdRP.

90. The plant of claim 81, wherein the plant is not produced by an essentially biological process.

91. The plant of claim 81, wherein the plant further comprises a heterologous RNA virus (HRV) RdRP which recognizes an HRV 5′ or 3′ replication region and/or the subgenomic promoter in the synthetic Secoviridae satellite RNA, and the heterologous RNA virus RdRP is an Alphaflexivirus, Betaflexivirus, Bromovirus, Celavirus, Closterovirus, Comovirus, Potexvirus, Potyvirus, Tobamovirus, Tombusvirus, Tospoviridae, Trivirinae, Tymovirus, Varicosavirus, or Secoviridae RdRP or an RdRP set forth in Table 7.

92. A Secoviridae satellite system that is self-replicating when introduced into a plant or plant cell, comprising: (a) a recombinant Secoviridae satellite RNA of any one of claims 1 to 28; and (b) an exogenous virus of the family Secoviridae that is capable of replication in the plant or plant cells and that encodes the Secoviridae RdRP that recognizes the 5′ and 3′ replicase recognition sequences in the recombinant Secoviridae satellite RNA, optionally wherein the Secoviridae satellite system further comprises a heterologous RNA virus RdRP which recognizes an HRV 5′ or 3′ replication region and/or the subgenomic promoter in the synthetic Secoviridae satellite RNA.

93. The self-replicating Secoviridae satellite system of claim 92, wherein the exogenous virus of the family Secoviridae is endemic or native to a different species, variety, or germplasm of plant.

94. The self-replicating Secoviridae satellite system of claim 92, wherein the Secoviridae satellite system further comprises a heterologous RNA virus (HRV) RdRP which recognizes an HRV 5′ or 3′ replication region and/or the subgenomic promoter in the synthetic Secoviridae satellite RNA, and the heterologous RNA virus RdRP is optionally an Alphaflexivirus, Betaflexivirus, Bromovirus, Celavirus, Closterovirus, Comovirus, Potexvirus, Potyvirus, Tobamovirus, Tombusvirus, Tospoviridae, Trivirinae, Tymovirus, Varicosavirus, or Secoviridae RdRP or an RdRP set forth in Table 7.
